(19) United States
(12) Patent Application Publication
(10) Pub. No.: US 2026/0137692 A1
(43) Pub. Date: May 21, 2026

(54) METHODS AND COMPOSITIONS FOR TREATING AND MANAGING ALZHEIMER'S DISEASE

(71) Applicant: PHARMATROPHIX, INC., Menlo Park, CA (US)

(72) Inventor: Frank M. Longo, Menlo Park, CA (US)

(21) Appl. No.: 19/393,335

(22) Filed: Nov. 18, 2025

Related U.S. Application Data
(63) Continuation of application No. PCT/US25/52888, filed on Oct. 28, 2025.
(60) Provisional application No. 63/712,958, filed on Oct. 28, 2024.

(51) Int. Cl. A61K 31/5375; A61P 25/28; G01N 33/68

(52) U.S. Cl. CPC A61K 31/5375; A61P 25/28; G01N 33/6896; G01N 2800/2821; G01N 2800/52

(57) ABSTRACT

The present disclosure provides methods of diagnosing, characterizing, or treating diseases (e.g., Alzheimer's Disease) associated with phosphorylated tau proteins, including p-tau217 and p-tau243, in subjects in need thereof.

### CROSS-REFERENCE TO RELATED APPLICATIONS

This application is a continuation of International Application No. PCT/US2025/052888, filed Oct. 28, 2025, which claims the benefit of and priority to U.S. Provisional Application No. 63/712,958, filed on Oct. 28, 2024, the contents of each of which are hereby incorporated by reference herein in their entirety for all purposes.

### STATEMENT REGARDING FEDERALLY SPONSORED RESEARCH OR DEVELOPMENT

This invention was made with Government support under NIA AD Pilot Trial 1R01AG051596, awarded by the National Institute of Health (NIH). The Government has certain rights in this invention.

### BACKGROUND

According to the Alzheimer's Association, some 5.4 million individuals in the United States (US) have Alzheimer's Disease (AD) and more than 95% of these are aged 65 years or older. One in 8 Americans age 65 and older, and almost half of those age 85 and older have AD. In addition to the incalculable human costs for individuals and their families, the estimated amount that was paid out to healthcare providers, nursing homes and hospice for Alzheimer's care in 2015 was $226 billion. With aging demographics, the number of affected individuals and the cost of care are in the early stages of an exponential rise with costs expected to reach $1.1 trillion by 2050.

Among the top 10 causes of death in the US, AD is the only cause that has no treatment options proven to prevent, delay, cure, or even slow its progression. Approved treatments are limited to amelioration of cognitive symptoms without having significant effects on underlying neurodegeneration and the disease process. The first approved class of symptomatic treatments consists of the acetylcholinesterase inhibitors (donepezil, rivastigmine, and galantamine) with the most recent FDA approval in this class occurring in 2001. A second class of symptomatic treatment was defined by the drug memantine, an N-methyl-D-aspartate receptor antagonist, approved by the FDA in 2003. These drugs have, at best, modest effects on cognition, overall function, and caregiver ratings, and no clearly detectable effects on AD progression. The goal of achieving a therapy that slows the underlying progression of neuronal degeneration has largely focused on efforts to reduce the accumulation of amyloid beta (Aβ) either by inhibition of enzymes necessary for Aβ production or by immunization against the Aβ peptide. Enzyme inhibition has been limited by toxic effects and recent phase 3 results from immunization approaches have so far been disappointing. The first, and so far only, amyloid antibody obtaining FDA approval (2021) is aducanumab. The extent to which aducanumab has a clinical effect in slowing disease progression is a controversial topic. It is clear that aducanumab confers considerable risk for brain edema and brain hemorrhage as side effects. New therapeutic strategies are needed to address this debilitating disease.

Primary features of AD include the accumulation of amyloid plagues and tau protein tangles along with failure of synaptic function, the degeneration of neuronal synapses, gliosis/neuro-inflammation. The p75 neurotrophin receptor has theoretical and complex links to many of these processes with potentially positive or negative effects and it is not known if its modulation could affect any aspect of AD or its fluid or imaging biomarkers.

### SUMMARY

The present disclosure provides an insight that modulation of the p75^{NTR} neurotrophin receptor (“p75^{NTR}”) can impact tau pathology in humans. P75^{NTR} modulation therapy is not associated with a decrease in beta amyloid, and beta amyloid has often been described as a “trigger” that initiates tau pathology in the development of Alzheimer's Disease, so, prior to the present disclosure, it was not known whether p75^{NTR} modulation therapy could impact tau pathology and/or whether tau pathology might indicate responsiveness to, or effectiveness of, p75^{NTR} modulation therapy.

Tau is a phosphoprotein primarily responsible for providing stabilization to microtubules in axons and dendrites. Various pathological states are associated with tau hyperphosphorylation, which is associated with tau dysfunction, synaptic impairment, and degeneration of neurons. Certain forms of tau protein, including certain phosphorylated tau species (including, for example, p-tau181, p-tau205, p-tau217, p-tau231, p-tau243, etc.), can be detectable in human blood, and may be associated with one or more neurological diseases, disorders or conditions, including Alzheimer's Disease (AD) and Amyotrophic Lateral Sclerosis (ALS).

The present disclosure establishes for the first time that p75^{NTR} modulation can impact tau pathology in humans, and furthermore that tau pathology biomarkers, specifically including those detectable in blood, serum, or cerebrospinal fluid (CSF) can be useful biomarkers for responsiveness to p75^{NTR} modulation. The present disclosure specifically teaches that certain pathology-associated phosphorylated tau forms may be useful as biomarkers of responsiveness to p75^{NTR} modulation, and demonstrates usefulness of p-tau217 in this respect. Other phosphorylated tau forms, particularly those detectable in serum (e.g., p-tau181, p-tau231, p-tau243, etc.) may be useful in accordance with the present disclosure, particularly p-tau243.

Thus, in some embodiments, the present disclosure provides technologies in which detection of a tau pathology biomarker, and particularly a blood, serum, or CSF tau pathology biomarker, is used to indicate likely responsiveness to p75^{NTR} modulation and/or to monitor effectiveness of p75^{NTR} modulation therapy. In specific embodiments, the present disclosure demonstrates that p-tau217, detected in blood, serum, or CSF can be a biomarker for responsiveness to p75^{NTR} modulation therapy for treatment of AD.

In some embodiments, a tau pathology biomarker useful in accordance with the present disclosure is one that has been demonstrated to correlate with p75^{NTR} modulation. Alternatively or additionally, in some embodiments, a tau pathology biomarker useful in accordance with the present disclosure is one that has been demonstrated to correlate with one or more features of brain pathology, e.g., as may have been determined by imaging and/or by assessment of one or more CSF components known to be associated with such brain pathology. In embodiments of particular interest, a tau pathology biomarker useful in accordance with the present disclosure is one that has been demonstrated both to correlate with p75^{NTR} modulation and to correlate with one or more features of brain pathology, e.g., as may have been determined by imaging and/or by assessment of one or more CSF components known to be associated with such brain pathology, In some embodiments, a tau pathology biomarker useful in accordance with the present disclosure is p-tau217. In some embodiments, a tau pathology biomarker useful in accordance with the present disclosure is p-tau243. In some embodiments, a tau pathology biomarker useful in accordance with the present disclosure is p-tau181. In some embodiments, a tau pathology biomarker useful in accordance with the present disclosure is p-tau231.

Thus, disclosed herein, in various embodiments, is a method of treating AD by administering a p75^{NTR} modulation therapy to a subject in whom a tau pathology biomarker (e.g., a phosphorylated tau species associated with tau pathology) has been detected. In some embodiments, such tau pathology biomarker has been detected in a blood sample, e.g., a serum sample, from the subject. In some embodiments, such tau pathology biomarker has been detected in a CSF sample from the subject. In some embodiments, the tau pathology biomarker is a level of a phosphorylated tau species that has been determined to be indicative of the presence of AD pathology in the brain, and/or that has otherwise been determined to indicate likely responsiveness to p75^{NTR} therapy (e.g., that the subject would benefit from treatment with the p75^{NTR} therapy); in some embodiments, such phosphorylated tau species is p-tau217, p-tau243, p-tau181, p-tau231, and in particular embodiments it is p-tau217. In some embodiments, the biomarker is a level of p-tau217. In some embodiments, a level of p-tau217 (e.g., plasma level) that is at least 0.180 pg/mL (e.g., at least 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.30, 0.40 pg/mL or more) (e.g., as measured by Lumipulse) or an analogous level in blood or CSF is indicative of the presence of AD in the subject and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator. In some embodiments, administration of the p75^{NTR} modulator to a subject (e.g., a subject with AD) results in a level of p-tau217 (e.g., plasma level) that is less than 0.320 pg/mL (e.g., as measured by Lumipulse). In some embodiments, administration of the p75^{NTR} modulator to a subject (e.g., a subject with AD) results in a level of p-tau217 (e.g., plasma level) that is less than 0.180 pg/mL (e.g., as measured by Lumipulse). In some embodiments, a level of p-tau217 of at least 4.2% p-tau217 relative to total tau or an analogous level in blood or CSF is indicative of the presence of Alzheimer's Disease pathology in the brain and/or of likely responsiveness to a p75^{NTR} modulator. In some embodiments, a decrease in this level can indicate responsiveness to a p75^{NTR} modulator. In some embodiments, a level of p-tau217 of at least 4.2% p-tau217 relative to total tau, e.g., as measured by mass spectrometry, or an analogous level in blood or CSF is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator. In some embodiments, a level of p-tau217 of at least 7.3 pg/mL, at least 0.27 pg/mL, at least 0.273 pg/mL, at least 0.399 pg/mL, at least 0.180 pg/mL or an analogous level in blood or CSF is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator. In some embodiments, a level of p-tau217 of at least 7.3 pg/mL, e.g., as measured by an electrochemiluminescence immunoassay (e.g., an S-PLEX assay), or an analogous level in blood or CSF is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator. In some embodiments, a level of p-tau217 of at least 0.27 pg/mL, e.g., as measured by an electrochemiluminescence immunoassay (e.g., a Meso Scale Discovery assay), or an analogous level in blood or CSF is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator. In some embodiments, a level of p-tau217 of at least 0.273 pg/mL, e.g., as measured by an electrochemiluminescence immunoassay (e.g., a Meso Scale Discovery assay) or an analogous level in blood or CSF is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator. In some embodiments, a level of p-tau217 of at least 0.399 pg/mL, e.g., as measured by an electrochemiluminescence immunoassay (e.g., a Meso Scale Discovery assay), or an analogous level in blood or CSF is indicative of the presence of Alzheimer's Disease pathology in the brain and/or of likely responsiveness to a p75^{NTR} modulator. In some embodiments, a decrease in this level can indicate responsiveness to a p75^{NTR} modulator. In some embodiments, a level of p-tau217 that or an analogous level in blood or CSF is indicative of the presence of Alzheimer's Disease pathology in the brain and/or of likely responsiveness to a p75^{NTR} modulator (e.g., indicative the subject would benefit from treatment with the p75^{NTR} modulator) is at least 0.180 pg/mL, e.g., as measured by Lumipulse. In some embodiments, a decrease in this level can indicate responsiveness to a p75^{NTR} modulator. In certain embodiments, a plasma ratio of p-tau217 to plasma β-amyloid (1-42 (“Aβ42”) of at least 0.00738 (e.g., as measured by Lumipulse) G pTau 217/0-Amyloid 1-42 Plasma Ratio assay or an analogous level in blood or CSF is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator. In certain embodiments, a plasma ratio of p-tau217 to plasma β-amyloid (1-42 (“Aβ42”) of at least 0.00371 (e.g., as measured by Lumipulse) G pTau 217/0-Amyloid 1-42 Plasma Ratio assay or an analogous level in blood or CSF is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator.

In some embodiments, a plasma level of p-tau243 of at least 0.2 pg/mL, e.g., as measured by Quanterix Simoa® immunoassay, or an analogous level in blood or CSF is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator.

In some embodiments, a CSF ratio of p-tau181 to CSF Aβ42″ of at least 0.037 to at least 0.09 (e.g., at least 0.037, 0.038, 0.039, 0.040, 0.041, 0.042, 0.043, 0.044, 0.045, 0.046, 0.047, 0.048, 0.049, 0.050, 0.051, 0.052, 0.053, 0.054, 0.055, 0.056, 0.057, 0.058, 0.059, 0.060, 0.061, 0.062, 0.063, 0.064, 0.065, 0.066, 0.067, 0.068, 0.069, 0.070, 0.071, 0.072, 0.073, 0.074, 0.075, 0.076, 0.077, 0.078, 0.079, 0.080, 0.081, 0.082, 0.083, 0.084, 0.085, 0.086, 0.087, 0.088, 0.089, or 0.090), e.g., as measured by Elecsys® Phospho-Tau (181P) CSF assay, or an analogous level in blood or plasma is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator. In certain embodiments, a CSF ratio of p-tau181 to CSF Aβ42 of at least 0.037, e.g., as measured by Elecsys® Phospho-Tau (181P) CSF assay, or an analogous level in blood or plasma is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator. In certain embodiments, a CSF ratio of p-tau181 to CSF Aβ42 of at least 0.09, e.g., as measured by Elecsys® Phospho-Tau (181P) CSF assay, or an analogous level in blood or plasma is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator. In some embodiments, a plasma level of p-tau181 of at least 0.934 pg/mL, e.g., as measured by Elecsys® Phospho-Tau (181P) Plasma assay, or an analogous level in blood or CSF is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator.

In some embodiments, a plasma level of p-tau231 of at least 0.2 pg/mL, e.g., as measured by Quanterix Simoa® immunoassay, or an analogous level in blood or CSF is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator.

Alternatively or additionally, in some embodiments, provided herein is a method of treating AD with p75^{NTR} that involves monitoring a tau pathology biomarker over time, e.g., by determining a tau pathology biomarker (e.g., level of a phosphorylated tau species such as p-tau181, p-tau217, p-tau231, p-tau243, and particularly p-tau217 or p-tau243, and most particularly p-tau217) in a subject who has received or is receiving p75^{NTR} therapy. In some embodiments, such biomarker has previously been determined in the subject, so that a change in such biomarker over time can be assessed. In some embodiments, such biomarker is detected multiple times over the course of therapy. In some embodiments, such biomarker is detected at regular intervals, e.g., every 3 months, 6 months, 12 months, 24 months; alternatively or additionally, in some embodiments such biomarker is detected intermittently, e.g., at the discretion of a physician, for example in light of detected changes.

In some embodiments, therapy administered to the subject may be adjusted if a change in such biomarker is achieved. For example, where such biomarker is a level of a phosphorylated tau species (e.g., p-tau217, p-tau243, p-tau181, p-tau231, or, in particular, p-tau217 or p-tau243), if such level has decreased or remained stable over time, administered therapy (e.g., administered p75^{NTR} therapy and/or other therapy such subject is receiving) may be maintained or, in some embodiments may be reduced or paused if desired. Alternatively, if such level has increased over time, then administered therapy (e.g., administered p75^{NTR} therapy and/or other therapy such subject is receiving) may be altered, for example by increasing dosing (e.g., individual dose amount and/or frequency of dosing, whether at a maintained dose amount or a different dose amount) and/or by adding an additional therapy.

In some embodiments, the present disclosure provides methods of treating AD by administering p75^{NTR} therapy to a subject determined to have a tau pathology biomarker (e.g., a level of a phosphorylated tau species such as p-tau217 or p-tau243), and then monitoring such biomarker and optionally adjusting therapy (e.g., such p75^{NTR} therapy) administered to such subject in light of a detected change in such biomarker (e.g., an increase or decrease in level of such phosphorylated tau species, such as p-tau217 or p-tau243).

In preferred embodiments, a tau pathology biomarker as described herein (e.g., a level of a phosphorylated tau species, and specifically a level of p-tau217, p-tau243, p-tau181, or p-tau231, most particularly p-tau217 or p-tau243) is determined in a blood (e.g., plasma) or CSF sample from a subject.

Disclosed herein, in various embodiments, is a method of treating Alzheimer's Disease in a subject identified as having a level of tau protein phosphorylated at p-tau217 or a level of p-tau243 that is indicative of the presence of Alzheimer's Disease pathology in the brain, and/or of likely responsiveness to a p75^{NTR} modulator (e.g., indicative the subject would benefit from treatment with the p75^{NTR} modulator), the method comprising administering an effective amount of a p75^{NTR} modulator to the subject.

Also disclosed herein, in various embodiments, is a method of treating Alzheimer's Disease in a subject in need thereof, the method comprising: (a) obtaining or having obtained a biological sample from the subject; (b) measuring or having measured a level of p-tau217 or a level of p-tau243; and (c) if the subject has a level of p-tau217 or p-tau243 that is indicative of the presence of Alzheimer's Disease pathology in the brain, and/or of likely responsiveness to a p75^{NTR} modulator (e.g., indicative the subject would benefit from treatment with the p75^{NTR} modulator), then administering an effective amount of a p75^{NTR} modulator.

In various embodiments, disclosed is a method of treating Alzheimer's Disease in a subject receiving therapy with a p75^{NTR} modulator, the method comprising (a) obtaining or having obtained a biological sample from the subject; (b) measuring or having measured a level of tau protein phosphorylated at a threonine at amino acid 217 (p-tau217) or p-tau243; (c) comparing the measured level of p-tau217 or p-tau243 with a prior level determined for the subject; and (d) if the determined level and the prior level (i) are reasonably comparable, continuing the therapy; or (ii) materially differ, reflecting progress of the subject's Alzheimer's Disease, modifying the therapy, for example by increasing frequency and/or amount of administered doses of the p75^{NTR} modulator or administering an alternative or additional therapy. In some embodiments, the subject whose therapy is monitored is one whose p-tau217 or p-tau243 level was measured in an initial measurement (e.g., and determined to be at a level that is indicative of the presence of Alzheimer's Disease pathology in the brain and/or of likely responsiveness to a p75^{NTR} modulator (e.g., indicative the subject would benefit from treatment with the p75^{NTR} modulator)) prior to administration of the p75^{NTR} modulator; in some such embodiments, such initial measurement is the prior level to which the determined level is compared.

In various embodiments, disclosed herein is a method of treating Alzheimer's Disease in a subject in need thereof, the method comprising: (a) obtaining or having obtained a biological sample (e.g., plasma) from the subject; (b) measuring or having measured a level of p-tau217 or p-tau243 in the sample prior to treatment of the subject with a p75^{NTR} modulator; (c) administering or having administered an effective amount of the p75^{NTR} modulator to the subject; (d) measuring or having measured a level of p-tau217 or p-tau243 in the sample following treatment of the subject with the p75^{NTR} modulator; (e) determining whether the level of p-tau217 or p-tau243 in the sample is indicative of responsiveness of the subject to the p75^{NTR} modulator; and (f) adjusting the frequency and/or amount of the p75^{NTR} modulator to be administered to the subject based on the responsiveness of the subject determined in step (d). In some embodiments, the adjusting of step (e) comprises reducing the frequency and/or dose of the p75^{NTR} modulator. In some embodiments, reducing the frequency and/or amount of the p75^{NTR} modulator is in response to the subject exhibiting or having exhibited adverse side effects to the p75^{NTR} modulator. In some embodiments, the adjusting of step (e) comprises increasing the frequency and/or dose of the p75^{NTR} modulator. In some embodiments, increasing the frequency and/or amount of the p75^{NTR} modulator is in response to the subject exhibiting low responsiveness to treatment with the p75^{NTR} modulator. In some embodiments, low responsiveness to treatment with the p75^{NTR} modulator is indicated by pathologically high levels of p-tau217 or p-tau243 in the subject. In some embodiments, the subject further has one or more of a level of tau protein phosphorylated at p-tau243, a level of tau protein phosphorylated at p-tau181, or a level of tau protein phosphorylated at p-tau231, that is each indicative of the presence of Alzheimer's Disease pathology in the brain.

The present disclosure further provides an insight that one or more proteomic marker sets may be useful to monitor modulation of p75^{NTR} For example, without wishing to be bound by any particular theory, the present disclosure proposes that ability to assess pathway-level changes distributed across multiple proteins may provide particular advantages, for example relative to assessment of one protein at a time.

The present disclosure demonstrates that p75^{NTR} modulation can affect the proteome of various cellular and/or tissue compartments, including the blood brain barrier (BBB), plasma, synapse, axons, dendrites, among others, thus establishing that proteomic assessments can provide information relevant to p75^{NTR} modulation. The present disclosure demonstrates that neuron-enriched modules (M2: Postsynaptic, M5: Glycolysis, M6: Axonogenesis), a microglia-enriched module (M3: Collagen), and/or a module representative of blood-brain barrier dysfunction (M1: BBB/Plasma) may be particularly useful. Alternatively or additionally, in some embodiments, the ubiquitination (M4) proteomic module and/or the glycolysis (M5) proteomic module may usefully be assessed to monitor p75^{NTR} modulation, including effectiveness of p75^{NTR} modulation therapy as described herein. In some embodiments, provided methods involving administration of a p75^{NTR} modulator can delay or prevent progression of Alzheimer's Disease in the subject. In some embodiments, such delaying or preventing progression comprises (e.g., can be assessed by detecting) reduced levels of one or both of a ubiquitination proteomic module and a glycolysis proteomic module, for example compared to a subject not receiving the p75^{NTR} modulator. In some embodiments, the delaying or preventing progression comprises reduced levels of one or more of a glycolysis proteomic module, a postsynaptic proteomic module, and an axonogenesis proteomic module compared to a subject not receiving the p75^{NTR} modulator. In some embodiments, the delaying or preventing progression comprises increased levels of one or both of a BBB proteomic module and a collagen proteomic module compared to a subject not receiving the p75^{NTR} modulator. In some embodiments, the levels of one or both of the ubiquitination proteomic module, the glycolysis proteomic module, the postsynaptic proteomic module, the axonogenesis proteomic module, the BBB proteomic module, and the collagen proteomic module are measured from the cerebrospinal fluid of the subject. In some embodiments, the ubiquitination proteomic module comprises measurements of one or more protein markers selected from the group consisting of: UCHL1, YWHAZ, YWHAG, PGK1, YWHAE, GDI1, PPIA, PGAM1, UBE2N, GLO1, NME1, YWHAB, PTPA, HSP90AA1, ENO1, GLOD4, MIF, FABP3, HAGH, LDHA, PEA15, GSR, TBCA, CFL1, SNCB, SH3BGRL3, TPT1, SCRN1, UBE2L3, PPP3CA, LASP1, PSMB7, CAND1, TXNRD1, SNCA, MAT2A, GPI, YWHAH, ST13, PRDX1, PPP1R7, FKBP1A, PSMB6, DDT, CKB, NEFM, OTUB1, MAPK1, PSMA3, TAGLN3, GAPDH, ATOX1, FAM49B, GLRX, NEDD8, PSMA4, PSMB4, PRDX3, NAXD, PSMA1, ITGB2, PSMA6, CZIB, GSTO1, FSCN1, ENOPH1, MAT2B, AKR1B1, RAD23A, DPYSL2, HSPA1A, TWF2, SNCG, PPP3R1, PSMB1, PSMA7, GABARAP, MAP1B, PLEC, CALB2, UFM1, CHI3L1, TBCB, AQP4, APOE4, CORO1C, NEFL, COTL1, FABP7, YKT6, ESD, THOP1, ATP6V1E1, PSMA5, ME1, MARCKSL1, BPNT1, CACYBP, ADD1, FDPS, CHIT1, MAP2, GSTP1, BIN1, TXNL1, FABP5, UBQLN2, IMPA1, ANGPTL4, CAP2, HEBP1, and AAMDC. In some embodiments, the glycolysis proteomic module comprises measurements of one or more protein markers selected from the group consisting of: MAPT, NRGN, GAP43, PKM, GOT1, ALDOA, MDH1, UBB, CALM1, BASP1, HSPA8, SOD1, DDAH1, PITHD1, LDHB, NPEPPS, PEBP1, DYNLL2, RAD23B, CPLX2, GDA, STMN1, GSS, PARK7, GMFB, SYN1, HPRT1, DTD1, SYT1, UBE2V1, AP2B1, SKP1, PKIA, PPP5C, TXN, NSF, SOD2, TALDO1, DLD, SMOC1, TMSB4X, PDXP, NUTF2, ALDOC, DDB1, CEND1, TOLLIP, SMOC2, PRKAR1A, CRYM, NIF3L1, GABARAPL2, PFN2, GGCT, CAMK2A, TPI1, GOT2, NAXE, PREP, NUDC, RTN1, DNM1, CYCS, SPON1, HSPE1, SPP1, ENO2, HDHD2, LAMP5, DLST, DNAJB2, SCN2B, RIDA, PSAT1, EPB41L1, BLMH, RPH3A, MARCKS, CYR61, HK1, CAMK2D, ACYP2, VSNL1, CKMT1A, TXNDC17, MDH2, S100A1, STX1B, CAMK2B, ATP6AP2, LHPP, STX8, EIF4B, TPD52, SNAP91, VAPA, PRKAR1B, TNFRSF12A, SERPINE2, SELENOW, VASN, MT2A, AP1B1, DBI, HSPA2, C1orf232, NAV1, SLC1A2, QDPR, B2M, PGM1 and CAMK2G.

In some embodiments, the postsynaptic proteomic module comprises measurements of one or more protein markers selected from the group consisting of: C1QL3, C4orf48, DPP10, ENDOD1, and SUSD5. In some embodiments, the axonogenesis proteomic module comprises measurements of one or more protein markers selected from the group consisting of: GALNT18, PVR, SEMA4D, CNTNAP2, and RTN4RL2. In some embodiments, the BBB proteomic module comprises measurements of one or more protein markers selected from the group consisting of: OGN, SFRP4, PDGFRL, METRNL, and MYOC. In some embodiments, the collagen proteomic module comprises measurements of one or more protein markers selected from the group consisting of: MATN2, PTK7, FMOD, SEMA3D, and B3GNT9.

In some embodiments, delaying or preventing progression comprises one or more of ameliorating, delaying, or preventing symptoms of Alzheimer's Disease compared to a subject not receiving the p75^{NTR} modulator. In some embodiments, symptoms are assessed by neurological testing selected from the group consisting of: the Alzheimer's Disease Assessment Scale-Cognitive (ADAS-Cog), the Mini Mental Status Exam (MMSE), the Amunet spatial navigation testing, the Clinical Global Impression (CGI) scale, the Geriatric Depression Scale (GDS), the neurological testing battery (NTB), Clinical Dementia Rating-Sum of Boxes (CDR-SB), Alzheimer's Disease Assessment Scale-Cognitive Subscale 13 (ADAS-Cog13), Alzheimer's Disease Cooperative Study-instrumental Activities of Daily Living for Mild Cognitive Impairment (ADCS-iADL), Neuropsychiatric Inventory (NPI), Alzheimer's Disease Composite Score (ADCOMS), and combinations thereof. In some embodiments, symptoms are assessed by one or both of Alzheimer's Disease Assessment Scale-Cognitive Subscale 13 (ADAS-Cog13) and Mini Mental Status Exam (MMSE). In some embodiments, the subject exhibits one or both of improved cognition or improved function compared to one or more of a patient receiving no therapy (e.g., no p75^{NTR} modulation therapy), a patient receiving a placebo therapy, or a patient receiving a therapy other than a p75^{NTR} modulator. In some embodiments, improved cognition comprises improved visuospatial ability.

Also disclosed herein, in various embodiments, is a method of reducing a level of a tau pathology biomarker (e.g., p-tau217 or p-tau243) in a subject, the method comprising administering to the subject an effective amount of a of a p75^{NTR} modulator. In some embodiments, the method further comprises reducing a level of one or more of p-tau181, or p-tau231 in the subject.

In some embodiments, the subject to whom a p75^{NTR} modulator is administered in accordance with the present disclosure does not have amyloid pathology.

In some embodiments, the p75^{NTR} modulator is a compound represented by Formula (Ia):

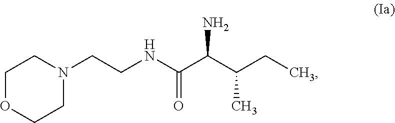 [SMILES: CC[C@H](C)[C@H](N)C(=O)NCCN1CCOCC1]

or a pharmaceutically acceptable salt thereof.

In some embodiments, the level of tau pathology biomarker (e.g., p-tau217, p-tau243, p-tau181, or p-tau231; particularly p-tau217 or p-tau243) is measured relative to total tau protein; in some embodiments, the level of tau pathology biomarker (e.g., p-tau217, p-tau243, p-tau181, or p-tau231; particularly p-tau217 or p-tau243) is determined as an absolute value. In some embodiments, the tau pathology biomarker (e.g., p-tau217, p-tau243, p-tau181, or p-tau231; particularly p-tau217 or p-tau243) is measured by a method comprising Lumipulse, Elecsys, Simoa, ELISA, immunoblot, immunoprecipitation, electrochemiluminescence (e.g., a Meso Scale Discovery assay or an S-PLEX assay), magnetic pull-down, or liquid chromatography-mass spectrometry. In some embodiments, a measurement of at least 4.2% p-tau217 relative to total tau, e.g., as measured by mass spectrometry, is indicative of the presence of Alzheimer's Disease pathology in the brain, and/or of likely responsiveness to p75^{NTR} modulation therapy (e.g., indicative the subject would benefit from treatment with the p75^{NTR} modulator). In some embodiments, a decrease in this level can indicate responsiveness to a p75^{NTR} modulator. In some embodiments, a measurement of at least 0.180 pg/mL (e.g., as measured by Lumipulse) is indicative of the presence of Alzheimer's Disease pathology in the brain, and/or of likely responsiveness to p75^{NTR} modulation therapy (e.g., indicative the subject would benefit from treatment with the p75^{NTR} modulator). In some embodiments, a decrease in this level can indicate responsiveness to a p75^{NTR} modulator. In some embodiments, a measurement of at least 0.27 pg/mL, as measured by an electrochemiluminescence immunoassay (e.g., a Meso Scale Discovery assay), is indicative of the presence of Alzheimer's Disease pathology in the brain, and/or of likely responsiveness to p75^{NTR} modulation therapy (e.g., indicative the subject would benefit from treatment with the p75^{NTR} modulator). In some embodiments, a decrease in this level can indicate responsiveness to a p75^{NTR} modulator. In some embodiments, a measurement of at least 0.273 pg/mL, as measured by an electrochemiluminescence immunoassay (e.g., a Meso Scale Discovery assay), is indicative of the presence of Alzheimer's Disease pathology in the brain, and/or of likely responsiveness to p75^{NTR} modulation therapy (e.g., indicative the subject would benefit from treatment with the p75^{NTR} modulator). In some embodiments, a decrease in this level can indicate responsiveness to a p75^{NTR} modulator. In some embodiments, a measurement of at least 0.399 pg/mL, as measured by an electrochemiluminescence immunoassay (e.g., a Meso Scale Discovery assay), is indicative of the presence of Alzheimer's Disease pathology in the brain, and/or of likely responsiveness to p75^{NTR} modulation therapy (e.g., indicative the subject would benefit from treatment with the p75^{NTR} modulator). In some embodiments, a decrease in this level can indicate responsiveness to a p75^{NTR} modulator. In some embodiments, a measurement of at least 7.3 pg/mL, as measured by an electrochemiluminescence immunoassay (e.g., a Meso Scale Discovery assay), is indicative of the presence of Alzheimer's Disease pathology in the brain, and/or of likely responsiveness to p75^{NTR} modulation therapy (e.g., indicative the subject would benefit from treatment with the p75^{NTR} modulator). In some embodiments, a decrease in this level can indicate responsiveness to a p75^{NTR} modulator.

In some embodiments, the p75^{NTR} modulator is administered in an amount of about 200 mg to about 400 mg. In particular, as exemplified herein, in some embodiments, a p75^{NTR} modulator of Formula (Ia) is administered in an amount of about 200 mg to about 400 mg (referencing the free base or free acid form of the compound, as is common in the art, so that a pharmaceutically acceptable salt of such compound may be administered in an amount that is not exactly 200 mg or 400 mg, but that is understood to correlate with 200 mg or 400 mg of the free base or free acid form); in light of this provided exemplification and general understanding in the art, a skilled person, reading the present disclosure, will appreciate that, in some embodiments, a p75^{NTR} modulator is administered in an amount that corresponds to about 200 mg to about 400 mg of a compound of Formula (Ia) (i.e., of the free base form of the compound of Formula (Ia)). Moreover, a skilled person, reading the present disclosure in light of the art will appreciate that, in many embodiments, a p75^{NTR} modulator administered in accordance with the present disclosure is administered according to a dosing regimen that includes a plurality of doses; in many embodiments, such dosing regimen is one that has been determined, when administered to a relevant population (e.g., to a population of subjects suffering from or susceptible to AD and having a tau pathology biomarker as described herein), to achieve a particular determined desirable endpoint (e.g., a certain degree of improvement in a relevant biomarker or characteristic of AD in a meaningful percentage of such population); thus, in many embodiments, a p75^{NTR} modulator is administered in at least one dose having an indicated amount (e.g., an amount corresponding to about 200 mg or about 400 mg of the free base form of the compound of Formula (Ia). In some embodiments, the p75^{NTR} modulator is administered in an amount of (e.g., in one or more doses of) about 400 mg (e.g., in an amount that corresponds to about 400 mg of the free base form of the compound of Formula (Ia).

In some embodiments, a p75^{NTR} modulator is administered twice daily.

In some embodiments, a p75^{NTR} modulator is administered orally, buccally, rectally, parenterally, ophthalmically, or via inhalation. In some embodiments, the p75^{NTR} modulator is administered orally.

Also provided herein, in various embodiments, is a method for determining Alzheimer's Disease (AD) activity in a subject, the method comprising: (a) obtaining or having obtained a biological sample from the subject; (b) measuring or having measured a level of p-tau217 or p-tau243; and (c) generating a prediction of AD progression, wherein a high level of p-tau217 or p-tau243 is indicative of a progression of AD, wherein the level of p-tau217 or p-tau243 is not correlated with amyloid pathology.

In some embodiments, the method further comprises measuring or having measured a level of one or more of p-tau181, or p-tau231, wherein a high level of one or more of p-tau181, or p-tau231 is indicative of a progression of AD.

In some embodiments, progression is based on increased levels of one or both of a ubiquitination proteomic module, a glycolysis proteomic module, a postsynaptic proteomic module, and an axonogenesis proteomic module. In some embodiments, the progression is based on reduced levels of one or both of a BBB proteomic module and a collagen proteomic module compared to a subject not receiving the p75^{NTR} modulator. In some embodiments, levels of one or more of the ubiquitination proteomic module, the glycolysis proteomic module, the postsynaptic proteomic module, the axonogenesis proteomic module, the BBB proteomic module, and the collagen proteomic module are measured from the cerebrospinal fluid of the subject. In some embodiments, the ubiquitination proteomic module comprises measurements of one or more protein markers selected from the group consisting of: UCHL1, YWHAZ, YWHAG, PGK1, YWHAE, GDI1, PPIA, PGAM1, UBE2N, GLO1, NME1, YWHAB, PTPA, HSP90AA1, ENO1, GLOD4, MIF, FABP3, HAGH, LDHA, PEA15, GSR, TBCA, CFL1, SNCB, SH3BGRL3, TPT1, SCRN1, UBE2L3, PPP3CA, LASP1, PSMB7, CAND1, TXNRD1, SNCA, MAT2A, GPI, YWHAH, ST13, PRDX1, PPP1R7, FKBP1A, PSMB6, DDT, CKB, NEFM, OTUB1, MAPK1, PSMA3, TAGLN3, GAPDH, ATOX1, FAM49B, GLRX, NEDD8, PSMA4, PSMB4, PRDX3, NAXD, PSMA1, ITGB2, PSMA6, CZIB, GSTO1, FSCN1, ENOPH1, MAT2B, AKR1B1, RAD23A, DPYSL2, HSPA1A, TWF2, SNCG, PPP3R1, PSMB1, PSMA7, GABARAP, MAP1B, PLEC, CALB2, UFM1, CHI3L1, TBCB, AQP4, APOE4, CORO1C, NEFL, COTL1, FABP7, YKT6, ESD, THOP1, ATP6V1E1, PSMA5, ME1, MARCKSL1, BPNT1, CACYBP, ADD1, FDPS, CHIT1, MAP2, GSTP1, BIN1, TXNL1, FABP5, UBQLN2, IMPA1, ANGPTL4, CAP2, HEBP1, and AAMDC. In some embodiments, the glycolysis proteomic module comprises measurements of one or more protein markers selected from the group consisting of: MAPT, NRGN, GAP43, PKM, GOT1, ALDOA, MDH1, UBB, CALM1, BASP1, HSPA8, SOD1, DDAH1, PITHD1, LDHB, NPEPPS, PEBP1, DYNLL2, RAD23B, CPLX2, GDA, STMN1, GSS, PARK7, GMFB, SYN1, HPRT1, DTD1, SYT1, UBE2V1, AP2B1, SKP1, PKIA, PPP5C, TXN, NSF, SOD2, TALDO1, DLD, SMOC1, TMSB4X, PDXP, NUTF2, ALDOC, DDB1, CEND1, TOLLIP, SMOC2, PRKAR1A, CRYM, NIF3L1, GABARAPL2, PFN2, GGCT, CAMK2A, TPI1, GOT2, NAXE, PREP, NUDC, RTN1, DNM1, CYCS, SPON1, HSPE1, SPP1, ENO2, HDHD2, LAMP5, DLST, DNAJB2, SCN2B, RIDA, PSAT1, EPB41L1, BLMH, RPH3A, MARCKS, CYR61, HK1, CAMK2D, ACYP2, VSNL1, CKMT1A, TXNDC17, MDH2, S100A1, STX1B, CAMK2B, ATP6AP2, LHPP, STX8, EIF4B, TPD52, SNAP91, VAPA, PRKAR1B, TNFRSF12A, SERPINE2, SELENOW, VASN, MT2A, AP1B1, DBI, HSPA2, C1orf232, NAV1, SLC1A2, QDPR, B2M, PGM1 and CAMK2G.

In some embodiments, the postsynaptic proteomic module comprises measurements of one or more protein markers selected from the group consisting of: C1QL3, C4orf48, DPP10, ENDOD1, and SUSD5. In some embodiments, the axonogenesis proteomic module comprises measurements of one or more protein markers selected from the group consisting of: GALNT18, PVR, SEMA4D, CNTNAP2, and RTN4RL2. In some embodiments, the BBB proteomic module comprises measurements of one or more protein markers selected from the group consisting of: OGN, SFRP4, PDGFRL, METRNL, and MYOC. In some embodiments, the collagen proteomic module comprises measurements of one or more protein markers selected from the group consisting of. MATN2, PTK7, FMOD, SEMA3D, and B3GNT9.

In some embodiments, the biological sample is a plasma sample, a serum sample, a whole blood sample, or a cerebrospinal fluid sample. In some embodiments, the biological sample is a plasma sample. In some embodiments, the biological sample is a cerebrospinal fluid sample.

### BRIEF DESCRIPTION OF THE DRAWINGS

These and other features, aspects, and advantages of the present disclosure will become better understood with regard to the following description and accompanying drawings. The patent or application file contains as least one drawing executed in color. Copies of this patent or patent application publication with color drawing(s) will be provided by the Office upon request and payment of the necessary fee.

FIG. 1 is a diagram showing the administration schedule of a Phase IIa trial for LM11A-31-BHS-PK (a salt of Formula (Ia)) in patients with mild to moderate Alzheimer's Disease.

FIG. 2 illustrates a randomization table.

FIG. 3 illustrates a randomization process.

FIG. 4 illustrates plasma aminoethyl morpholine metabolite (AEM)-pharmacokinetic (PK) data from 200 mg safety population from visit 2, 3, 4 and 5. Values below the limit of quantification (BLQ) were treated as BLQ/2.

FIG. 5 depicts plasma AEM-PK data from 400 mg safety population from visit 2, 3, 4 and 5. Values below the limit of quantification (BLQ) were treated as BLQ/2.

FIG. 6 depicts plasma LM11A-31-BHS-PK data from 200 mg safety population from visit 2, 3, 4 and 5. Values below the limit of quantification (BLQ) were treated as BLQ/2.

FIG. 7 depicts plasma LM11A-31-BHS-PK data from 400 mg safety population from visit 2, 3, 4 and 5. Values below the limit of quantification (BLQ) were treated as BLQ/2.

FIG. 8 depicts CSF LM11A-31-BHS-PK data from 200 mg and 400 mg safety population from visit 5.

FIG. 9 depicts plasma AEM levels (Y-axis) compared to CSF LM11A-31-BHS levels (X-axis).

FIG. 10 depicts plasma LM11A-31-BHS levels (Y-axis) compared to CSF LM11 A-31-BHS levels (X-axis).

FIG. 11 depicts a flow diagram showing disposition of patients in the Phase IIa trial.

FIG. 12 depicts a bar chart showing reasons for withdrawal within the subject groups within the Phase IIa trial.

FIG. 13 depicts a bar chart showing the distribution of sex within the three treatment groups of the Phase IIa trial.

FIG. 14 depicts a bar chart showing the distribution of age within the three treatment groups of the Phase IIa trial.

FIG. 15 depicts a bar chart showing the distribution of the HIS between the dosage groups of the safety population.

FIG. 16 depicts a bar chart showing ApoE distribution within the different groups of the safety population.

FIG. 17 depicts ApoE distribution within the different groups of the safety population.

FIG. 18 depicts ApoE distribution within the different groups of the intention to treat population.

FIG. 19 depicts ApoE distribution within the different groups of the per protocol population.

FIGS. 20A-20G depict the percentage of blood pressure interpretations for the diastolic blood pressure (DBP), systolic blood pressure (SBP) of the different dosage groups and overall for different timepoints, the early discontinuation visit (EDV) and the last observation carried forward (LOCF) for the different dosage groups in the Phase IIa trial. The groups include screening (FIG. 20A), Baseline (FIG. 20B), week 4 (FIG. 20C), week 12 (FIG. 20D), week 26 (FIG. 20E), EDV (FIG. 20F), and LOCF (FIG. 20G).

FIGS. 21A-21F depict the percentage of ECG interpretations for the different dosage groups and overall for different timepoints, the early discontinuation visit (EDV) and the last observation carried forward (LOCF) in the Phase IIa trial. The groups include screening (FIG. 21A), week 4 (FIG. 21B), week 12 (FIG. 21C), week 26 (FIG. 21D), EDV (FIG. 21E), and LOCF (FIG. 21F).

FIG. 22 depicts a line graph of the eosinophils values of the safety population receiving any dosage of LM11A-31-BHS in the Phase IIa trial.

FIG. 23 depicts a line graph of the eosinophils values of the safety population receiving placebo in the Phase IIa trial.

FIG. 24 depicts a clustered bar graph of eosinophil Means ([/μl]) for visits per placebo and verum groups in the Phase IIa trial.

FIG. 25 depicts mechanism engagement and biomarkers.

FIG. 26 depicts preclinical studies in mice point to degenerative mechanisms and human biomarkers directly relevant to those respective degenerative mechanisms.

FIG. 27 depicts estimating human brain exposure to drug.

FIG. 28 depicts Phase IIa RCT safety and exploratory endpoint trial.

FIG. 29A depicts an exemplary Safety/PK defined population (LM11A-31 200 mg bid 78, LM11A-31 400 mg bid 83, Placebo control 81 patients). FIGS. 29B-29E illustrate observations in regard to age, MMSE, ratio of Aβ42 to Aβ40 (Aβ42/40), and ratio of phosphorylated tau to Aβ42 (p-tau/Aβ42), respectively.

FIG. 30 depicts safety results. 17 subjects left the trial because of adverse events (AEs).

FIGS. 31A-31D depict the progression as indicated by biomarkers and clinical testing of the placebo group in the population of mild-moderate AD over the 26-week study period. Four outcome domains include: CSF biomarkers (FIG. 31A), sMRI (FIG. 31B), cognition (FIG. 31C), and FDG-PET (FIG. 31D).

FIGS. 32A-32C depict exploratory outcomes for domain 1-CSF AD core biomarkers related with Ab 42 (FIG. 32A) or Aβ40 (FIG. 32B), and related to modulation the p75 receptor (FIG. 32C) by the drug and the effect of this modulation on APP processing to Ab 42 and resulting CSF levels of Ab 42.

FIGS. 33A-33B depict exploratory outcomes for CFS AD core biomarkers related with Ab. FIG. 33A illustrates placebo vs. combined drug; and FIG. 33B illustrates placebo vs. different doses (200 mg and 400 mg).

FIGS. 34A-34B depict exploratory outcomes for domain 1-CSF AD core biomarkers related with CSF tau levels: tau (FIG. 34A) and p-tau (FIG. 34B).

FIGS. 35A-35B depict exploratory outcomes for CFS AD core biomarkers related with tau and p-tau. FIG. 35A illustrates placebo vs. combined drug; and FIG. 35B illustrates placebo vs. different doses (200 mg and 400 mg).

FIGS. 36A-36B depict exploratory outcomes for domain 1-CSF pre-synaptic biomarkers related with levels of SYT1 (FIG. 36A) and SNAP-25 (FIG. 36B).

FIGS. 37A-37B depict exploratory outcomes for CFS pre-synaptic biomarkers related with SYT1 and SNAP-25. FIG. 37A illustrates placebo vs. combined drug; and FIG. 37B illustrates placebo vs. different doses (200 mg and 400 mg).

FIG. 38 depicts exploratory outcomes for domain 1-CSF post-synaptic biomarkers related with neurogranin.

FIGS. 39A-39B depict exploratory outcomes for CFS AD post-synaptic biomarkers related with NG36. FIG. 39A illustrates placebo vs. combined drug; and FIG. 39B illustrates placebo vs. different doses (200 mg and 400 mg).

FIGS. 40A-40B depict exploratory outcomes for domain 1-CSF glial/neuroinflammatory biomarkers related with sTREM2 (FIG. 40A) and YKL-40 (FIG. 40B).

FIGS. 41A-41B depict exploratory outcomes for CSF glial biomarkers related with sTREM2 and YKL-40. FIG. 41A illustrates placebo vs. combined drug; and FIG. 41B illustrates placebo vs. different doses (200 mg and 400 mg).

FIG. 42 depicts exploratory outcomes for domain 2 using Hippocampal sMRI (structural MR).

FIG. 43 depicts preprocessing strategy for analysis of structural MRI using voxel-based morphometry. Time 1 indicates pretreatment MRI imaging and time 2 indicates post treatment MRI imaging.

FIG. 44 depicts T1-weighted structural MRI using whole brain voxel-wise analysis. It is demonstration of flexible factorial generalized linear model (GLM) analysis matrix for brain voxel-wise structural analysis.

FIG. 45 depicts whole brain results of T1-weighted structural MRI analysis.

FIG. 46A-46C depict T1-weighted structural MRI using Group×Time interaction patterns. FIG. 46A shows the MRI image. FIG. 46B illustrates placebo vs. combined drug; and FIG. 46C illustrates placebo vs. different doses (200 mg and 400 mg).

FIG. 47 depicts T1-weighted structural MRI using the MCS (Monte Carlo Simulation) analysis and dose effects.

FIGS. 48A-48C depict cortical thickness changes in anterior cingulate region area 33 prime. FIG. 48B illustrates low dose vs. placebo; and FIG. 48C illustrates high dose vs. placebo.

FIGS. 49A-49B depict exploratory outcomes for domain 3 related with cognition: ADAS-13 (FIG. 49A) and ADAS-11 (FIG. 49B).

FIGS. 50A-50B depict exploratory outcomes for cognition AD core markers related with ADAS. FIG. 50A illustrates placebo vs. combined drug; and FIG. 50B illustrates placebo vs. different doses (200 mg and 400 mg).

FIG. 51A depicts exploratory outcomes for domain 4 related with FDG-PET as a marker of synaptic function. FIG. 51B illustrates MCIMaskSROI APC for the placebo and treatment groups.

FIG. 52 depicts exploratory outcomes for domain 4 FDG-PET with post-hoc MCS.

FIG. 53 depicts Co-registration to MRI and SUVR using FDG PET.

FIG. 54 depicts FDG PET using whole brain voxel-wise analysis.

FIG. 55 depicts whole brain results of FDG PET.

FIGS. 56A-56C depicts FDG PET using Group×Time interaction patterns. FIG. 56A shows the image. FIG. 56B illustrates placebo vs. combined drug; and FIG. 56C illustrates placebo vs. different doses (200 mg and 400 mg).

FIG. 57 depicts FDG PET using MCS analysis.

FIG. 58 depicts summary of the biomarkers showing a statistically significant effect of drug on multiple mechanisms and the ability of the drug to engage with and affect targeted disease mechanisms.

FIGS. 59A-59D depict summary of the placebo progression measured by the measures in the 4 outcome domains and the effects and trends of the drug on slowing disease progression. Four outcome domains include: CSF biomarkers (FIG. 59A), sMRI (FIG. 59B), cognition (FIG. 59C), and FDG-PET (FIG. 59D).

FIG. 60 depicts a schematic of p75 neurotrophin receptor (p75^{NTR}) signaling effects on neurons, glia, and proteinopathies.

FIG. 61 shoes depictions of voxels within the brain showing reduced longitudinal degeneration (left panels) as measured by sMRI and glucose hypometabolism as measured by FDG PET (right panels) after treatment with LM11A-31.

FIGS. 62A and 62B depict schematics of exemplary methods for protein detection. FIG. 62A depicts a schematic of a liquid chromatography-mass spectrometry (LC-MS) method, which can be used, e.g., for the detection of p-tau217. FIG. 62A depicts a schematic of a tandem mass tag-based mass spectrometry (TMT-MS) method, which can be used, e.g., for the detection of CSF proteomic modules.

FIG. 63 depicts a schematic of an exemplary process for the establishment of cerebrospinal fluid (CSF) proteomic modules.

FIG. 64 depicts a graph of annual percent change in p-tau271 levels in patients treated with placebo or LM11A-31.

FIGS. 65A and 65B depict cognitive measures in patients after 26 weeks of treatment with placebo or LM11A-31. FIG. 65A depicts ADAS-Cog-13 scores. FIG. 65A depicts MMSE scores.

FIG. 66 depicts a protein co-expression network of proteomic modules based on similar expression patterns across samples (left panel), and exemplary graphs detailing eigenprotein expression of glycolysis (top right panel) and ubiquitination (bottom right panel) proteomic modules.

FIG. 67 depicts graphs of baseline correlation of p-tau217 levels with indicated Alzheimer's Disease, synaptic, and cognitive markers.

FIGS. 68A and 68B depict graphs of baseline correlation of p-tau217 levels with indicated CSF proteomic modules. FIG. 68A depicts correlation of p-tau217 levels with the ubiquitination proteomic module. FIG. 68B depicts correlation of p-tau217 levels with the glycolysis proteomic module.

FIGS. 69A-69C depicts levels of tandem mass tag-labeled proteins in patient CSF samples following treatment with placebo or LM11A-31. FIG. 69A depicts levels of YKL-40. FIG. 69B depicts levels of total tau (t-tau). FIG. 69C depicts levels of neurogranin (NRGN).

FIGS. 70A-70D depict graphs showing eigenprotein values of the M5 glycolysis proteomic module in patient CSF samples. FIG. 70A depicts eigenprotein values for all proteins in Alzheimer's Disease CSF samples versus control in a reference cohort. FIG. 70B depicts eigenprotein values for all proteins in patient CSF samples following treatment with placebo or LM11A-31. FIG. 70C depicts eigenprotein values for statistically significant proteins (p<0.05) in Alzheimer's Disease CSF samples versus control in a reference cohort. FIG. 70D depicts eigenprotein values for statistically significant proteins (p<0.05) in patient CSF samples following treatment with placebo or LM11A-31.

FIGS. 71A-71D depict graphs showing eigenprotein values of the ubiquitination proteomic module in patient CSF samples. FIG. 71A depicts eigenprotein values for all proteins in Alzheimer's Disease CSF samples versus control in a reference cohort. FIG. 71B depicts eigenprotein values for all proteins in patient CSF samples following treatment with placebo or LM11A-31. FIG. 71C depicts eigenprotein values for statistically significant proteins (p<0.05) in Alzheimer's Disease CSF samples versus control in a reference cohort. FIG. 71D depicts eigenprotein values for statistically significant proteins (p<0.05) in patient CSF samples following treatment with placebo or LM11A-31.

FIG. 72 depicts graphs showing the baseline correlation of p-tau217 with longitudinal changes in neuroimaging and cognition markers.

FIG. 73 depicts a correlation matrix showing Spearman's correlation analyses of relationships between biomarker and cognitive variables at Phase IIa study baseline across participants from the intention to treat population (n=242). For each analysis, participants were included if they had baseline data for both variables that passed all quality control and outlier analyses. Correlations were corrected for multiple comparisons using the False Discovery Rate (FDR), and correlations that did not survive correction for multiple comparisons with Q<0.05 were omitted (white squares). Black lines within the correlation matrix highlight within-domain correlations. BBB: blood-brain barrier; ECM: extra-cellular matrix.

FIG. 74A is a volcano plot showing the differential change of individual CSF proteins over 26 weeks in the LM11A-31 group (n=93) compared to the placebo group (n=54). Sample sizes differed across proteins due to detectability of individual proteins in different subjects. When applying an uncorrected significance threshold of P<0.05 (grey dashed line), 241 proteins differed significantly between LM11A-31 and placebo. Labels indicate the gene name of the proteins above the 90th percentile from the analysis, ordered by P value. FIG. 74B is a plot showing synaptic enrichment analysis of proteins that decreased in abundance with LM11A-31 treatment (dots in the top left quadrant) using SynGO. Enrichment analysis for each ontology term was conducted using a one-tailed greater than Fisher's exact test, with all brain expressed genes as the background gene set. Enrichment statistics were plotted using the −log 10 of the Q-value (i.e., the FDR-corrected P values), with a threshold of Q<0.01. Log transformed values were rounded to the nearest whole number as indicated in the legend. Only first-level ontology terms or significantly enriched terms were labelled. Overrepresented terms within the ontology were shown in corresponding grayscale tone and listed in Table 4. Terms which had less than three mapped genes were not tested for enrichment (N.A., light grey). Terms that were tested for enrichment but not significant (N.S.) are shown in dark grey. Corresponding enrichment analyses of proteins which increased in abundance with treatment were conducted and no terms were significantly enriched at the threshold used above. Plots were modified from the plotting tool at syngoportal.org/.

FIG. 75 is a series of plots showing longitudinal progression on CSF proteomic co-expression modules. CSF proteomic module creation, anticipated direction of worsening, cell-type enrichment, and top GO terms were defined in an independent cohort by Bangs et al. bioRxiv 2025.03.14.643332 (2025). Modules were organized according to the cluster dendrogram from which they were derived, with more closely related modules being separated by fewer branch points. Wilcoxon rank sum tests compared longitudinal changes in the 10 CSF proteomic modules in the placebo (n=54) and LM11A-31 (n=93) groups. Grey dashed lines indicate comparisons which are insignificant. Black dotted lines indicate trend-level effects (P<0.1), and black solid lines indicate significant effects (P<0.05). The top five most significantly altered proteins, based on P-values from FIG. 74B, are shown for modules with a significant or trend-level effect of LM11A-31 treatment. Notches on the box plots approximate a 95% confidence interval of the median. Statistics were performed two-sided with a significance threshold of P<0.05. Analyses were uncorrected given the exploratory nature of the trial. Note that y-axes differ across plots. BBB: blood-brain barrier; ECM: extra-cellular matrix.

FIG. 76 is a plot showing proteomic modules from the Emory CSF TMT network are highly preserved in the LM11A-31 trial TMT dataset. Unbiased network analysis (WGCNA) of the Emory ADRC CSF TMT dataset (Bangs et al.) identified 10 protein co-expression modules based on shared biological processes and cellular expression patterns. Module preservation analysis using the LM11A-31 trial dataset as the test set revealed high Z-summary scores, indicating that these modules retain both their internal structure and connectivity patterns within this cohort. This suggests that these modules represent biologically robust features of the AD CSF proteome. Modules with Z-summary scores greater than or equal to 1.96 (q=0.05) are considered preserved, while those with Z-summary scores greater than or equal to 10 (q=1e-23) are considered highly preserved.

FIG. 77 is a plot showing LM11A-31 treatment attenuates longitudinal increases in plasma phosphorylated tau. Wilcoxon rank sum tests examined differences in the annual percent change of plasma biomarkers in the placebo or LM11A-31 group. Notches on the box plots approximate a 95% confidence interval of the median. Statistics were performed two-sided with a significance threshold of P<0.05. Analyses are uncorrected given the exploratory nature of the trial.

FIGS. 78A-78D are a series of plots showing LM11A-31 treatment preserves visuospatial abilities, as measured by composite cognitive domain scores. Linear mixed models examined longitudinal changes under placebo and drug in four cognitive domains: memory (FIG. 78A), visuospatial (FIG. 78B), executive function (FIG. 78C), and language (FIG. 78D). Fixed effects were group (placebo, drug), visit (baseline, 12-week and 26-week) and their interactions. Subject random intercepts were included in the model, and the total number of days between the first and final visit were included as a covariate. Plots show the least squares mean of each group derived from the mixed models. Error bars represent the standard errors. * indicates a significant (P<0.05) group by visit interaction effect.

FIG. 79 depicts the ¹H NMR spectrum of the compound of Formula (I).

FIG. 80 depicts the XRPD of the compound of Formula (I) Form 1.

FIG. 81 depicts the XRPD comparison of the compound of Formula (I) Form 1 and the compound of Formula (I) reference standard.

FIG. 82 depicts the DSC and TGA of the compound of Formula (I) Form 1.

FIG. 83 depicts a GEN Form A microscopic image.

FIG. 84 depicts a GEN Form A microscopic image.

FIG. 85 depicts a GEN Form A microscopic image.

FIG. 86 depicts the XRPD of ADI (adipic salt) Form A prepared in EtOAc.

FIG. 87 depicts the ¹H NMR spectrum of ADI Form A prepared in EtOAc.

FIG. 88 depicts the DSC and TGA overlay of ADI Form A prepared in EtOAc.

FIG. 89 depicts the DVS analysis of ADI Form A prepared in EtOAc, weight vs relative humidity.

FIG. 90 depicts the DV analysis of ADI Form A prepared in EtOAc, weight vs time.

FIG. 91 depicts the XRPD of ADI Form A prepared in THF.

FIG. 92 depicts the ¹H NMR spectrum of ADI Form A prepared in THF.

FIG. 93 depicts the DSC and TGA overlay of ADI Form A prepared in THF.

FIG. 94 depicts the DVS analysis of ADI Form A prepared in THF, weight vs relative humidity.

FIG. 95 depicts the DVS analysis of ADI Form A prepared in THF, weight vs time.

FIG. 96 depicts microscopy images of ADI Form A samples.

FIG. 97 depicts stability sample of GEN (gentisic salt) Form A after 1 week.

FIG. 98 depicts the solution aspect for gentisate solubility experiments, from low pH (left) to high (right).

FIG. 99 depicts the solubility curve of GEN Form A in IPA.

FIG. 100 depicts the solubility curve of GEN Form A in EtOH.

FIG. 101 depicts the parity plot for GEN Form A solubility model in EtOAc-EtOH.

FIG. 102 depicts the solubility model for GEN Form A in EtOAc-EtOH.

FIG. 103 depicts the HPLC chromatogram of a sample of the compound of Formula (I) used to prepare various salts.

FIG. 104 depicts the XRPD of GEN Form A.

FIG. 105 depicts the ¹H NMR spectrum of GEN Form A.

FIG. 106 depicts the DSC and TGA overlay of GEN Form A.

FIG. 107 depicts the DVS analysis of GEN Form A, weight vs relative humidity.

FIG. 108 depicts the DVS analysis of GEN Form A, weight vs time.

FIG. 109 depicts the HPLC chromatogram of GEN Form A.

FIG. 110 depicts the solubility curve of ADI Form A in 2-MeTHF

FIG. 111 depicts the solubility curve of ADI Form A in EtOAc.

FIG. 112 depicts the XRPD of GEN Form B.

FIG. 113 depicts the temperature and reflectance profile of ADI Form A.

### DETAILED DESCRIPTION

The present disclosure generally relates to methods of diagnosing, characterizing, or treating diseases (e.g., Alzheimer's Disease) associated with tau protein phosphorylated at a threonine at amino acid 217 (p-tau217) or with tau protein phosphorylated at a leucine at amino acid 243 (p-tau243) in subjects in need thereof. In various embodiments, the present disclosure provides a method of treating Alzheimer's Disease in a subject identified as having a high level of tau protein phosphorylated at p-tau217 or p-tau243, the method comprising administering an effective amount of a p75^{NTR} modulator to the subject. In various embodiments, the present disclosure provides a method of treating Alzheimer's Disease in a subject in need thereof, the method comprising (a) obtaining or having obtained a biological sample from the subject; (b) measuring or having measured a level of p-tau217 or p-tau243; and (c) if the patient has a high level of p-tau217 or p-tau243, then administering an effective amount of a p75^{NTR} modulator. In various embodiments, the present disclosure provides a method of reducing levels of p-tau217 or p-tau243 in a subject, the method comprising administering to the subject an effective amount of a of a p75^{NTR} modulator. In various embodiments, the present disclosure provides a method of treating a disease associated with high levels of p-tau217 or p-tau243 in a subject in need thereof, the method comprising administering to the subject an effective amount of a p75^{NTR} modulator. In various embodiments, the present disclosure provides, a method for determining Alzheimer's Disease (AD) activity in a subject, the method comprising: (a) obtaining or having obtained a biological sample from the subject; (b) measuring or having measured a level of p-tau217 or p-tau243; (c) generating a prediction of AD progression, wherein a high level of p-tau217 or p-tau243 is indicative of a progression of AD, wherein the level of p-tau217 or p-tau243 is not correlated with amyloidosis.

### Definitions

the terms “a,” “an,” and “the” refer to “one or more” when used in this application, including the claims. Thus, for example, reference to “a carrier” includes mixtures of one or more carriers, two or more carriers, and the like.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the present specification and attached claims are approximations that can vary depending upon the desired properties sought to be obtained by the present application. Generally the term “about,” as used herein when referring to a measurable value such as an amount of weight, time, dose, etc. is meant to encompass in one example variations of ±20% or ±10%, in another example ±5%, in another example ±1%, and in yet another example ±0.1% from the specified amount, as such variations are appropriate to perform the disclosed method.

The term “alkyl,” alone or in combination, refers to an optionally substituted straight-chain or branched-chain alkyl radical having from 1 to 20 carbon atoms. The term also includes optionally substituted straight-chain or branched-chain alkyl radicals having from 1 to 6 carbon atoms as well as those having from 1 to 4 carbon atoms. Examples of alkyl radicals include methyl, ethyl, n-propyl, isopropyl, n-butyl, isobutyl, sec-butyl, tert-butyl, tert-amyl, pentyl, hexyl, heptyl, octyl and the like. “Branched” refers to an alkyl group in which a lower alkyl group, such as methyl, ethyl or propyl, is attached to a linear alkyl chain. “Lower alkyl” refers to an alkyl group having 1 to 8 carbon atoms (i.e., a C₁₋₈ alkyl), e.g., 1, 2, 3, 4, 5, 6, 7, or 8 carbon atoms. “Higher alkyl” refers to an alkyl group having about 10 to 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls. Alkyl groups can be optionally substituted.

The term “alkenyl,” alone or in combination, refers to an optionally substituted straight-chain or branched-chain hydrocarbon radical having one or more carbon-carbon double-bonds and having from 2 to 18 carbon atoms. Alkenyl includes optionally substituted straight-chain or branched-chain hydrocarbon radicals having one or more carbon-carbon double bonds and having from 2 to 6 carbon atoms such as from 2 to 4 carbon atoms. Examples of alkenyl radicals include ethenyl, propenyl, butenyl, 1,4-butadienyl and the like. Suitable alkenyl groups include allyl. The terms “allylic group” or “allyl” refer to the group CH₂HC═CH₂ and derivatives thereof formed by substitution. Thus, the terms alkenyl and/or substituted alkenyl include allyl groups, such as but not limited to, allyl, methylallyl, di-methylallyl, and the like. The term “allylic position” or “allylic site” refers to the saturated carbon atom of an allylic group. Thus, a group, such as a hydroxyl group or other substituent group, attached at an allylic site can be referred to as “allylic.” “1-alkenyl” refers to alkenyl groups where the double bond is between the first and second carbon atom.

The term “alkynyl,” alone or in combination, refers to an optionally substituted straight-chain or branched-chain hydrocarbon radical having one or more carbon-carbon triple-bonds and having from 2 to 12 carbon atoms. Alkynyl includes optionally substituted straight-chain or branched-chain hydrocarbon radicals having one or more carbon-carbon triple bonds and having from 2 to 6 carbon atoms such as from 2 to 4 carbon atoms. Examples of alkynyl radicals include ethynyl, propynyl, butynyl and the like. “1-alkynyl” refers to alkynyl groups where the triple bond is between the first and second carbon atom.

“Cycloalkyl” refers to a non-aromatic mono- or multicyclic ring system of 3 to 10 carbon atoms, e.g., 3, 4, 5, 6, 7, 8, 9, or 10 carbon atoms, such as from 3 to 6 carbon atoms. The cycloalkyl group can be optionally partially unsaturated. The cycloalkyl group also can be optionally substituted as defined herein. Representative monocyclic cycloalkyl rings include, but are not limited to, cyclopropyl, cyclobutyl, cyclopentyl, cyclohexyl, cycloheptyl, and the like. Further, the cycloalkyl group can be optionally substituted with a linking group, such as an alkylene group as defined hereinabove, for example, methylene, ethylene, propylene, and the like. In such cases, the cycloalkyl group can be referred to as, for example, cyclopropylmethyl, cyclobutylmethyl, and the like. Additionally, multicyclic cycloalkyl rings include adamantyl, octahydronaphthyl, decalin, camphor, camphane, and noradamantyl.

The term “heterocycloalkyl” refers to a cyclic group of 3 to 6 atoms, or 3 to 10 atoms, containing at least one heteroatom. In one aspect, these groups contain 1 to 3 heteroatoms. Suitable heteroatoms include, for example, oxygen, sulfur, and nitrogen. Heterocyclic groups may be attached through any substitutable atom, such as a nitrogen or through a carbon atom in the ring. Suitable heterocyclic groups include pyrrolidinyl, morpholino, morpholinoethyl, and pyridyl. Such groups may be substituted.

The term “aryl” refers to aromatic groups which have 5-14 ring atoms and at least one ring having a conjugated pi electron system and includes carbocyclic aryl, heteroaryl and biaryl groups, all of which may be optionally substituted. The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. In particular embodiments, the term “aryl” includes cyclic aromatic comprising about 5 to about 10 carbon atoms, e.g., 5, 6, 7, 8, 9, or 10 carbon atoms, and including 5- and 6-membered hydrocarbon and heterocyclic aromatic rings. Examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like, all optionally substituted. Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto. Unless otherwise specified, substituents on an aryl group may be independently selected at each occurrence from alkyl, aryl, aralkyl, hydroxyl, alkoxyl, haloalkyl, aryloxyl, aralkyloxyl, carboxyl, acyl, halo, nitro, alkoxycarbonyl, aryloxycarbonyl, aralkoxycarbonyl, acyloxyl, acylamino, carbamoyl, alkylcarbamoyl, dialkylcarbamoyl, arylthio, alkylthio, and —NR′R″, wherein R′ and R″ can each be independently hydrogen, alkyl, substituted alkyl, aryl, substituted aryl, and aralkyl.

A structure represented generally by a formula such as:

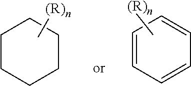

as used herein refers to a 6-carbon ring structure comprising a substituent R group, wherein the R group can be present or absent, and when present, one or more R groups can each be substituted on one or more available carbon atoms of the ring structure. The presence or absence of the R group and number of R groups is determined by the value of the integer n. Each R group, if more than one, is substituted on an available carbon of the ring structure rather than on another R group. For example, the structure:

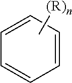

wherein n is an integer from 0 to 2 comprises compound groups including, but not limited to:

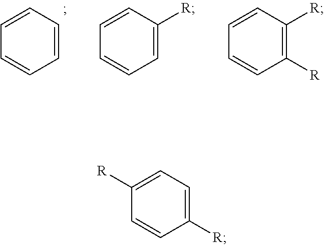

and the like.

The structure:

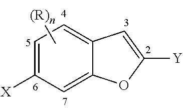

wherein n is one (1) comprises compound groups including:

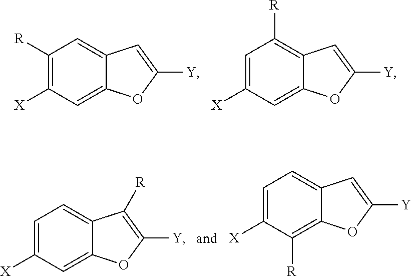

wherein the one (1) R substituent can be attached at any carbon on the benzofuran parent structure not occupied by another designated substituent, as in this case carbon 6 is substituted by X and carbon 2 is substituted by Y.

A dashed line representing a bond in a cyclic ring structure indicates that the bond can be either present or absent in the ring.

“Carbocyclic aryl” groups are groups wherein the ring atoms on the aromatic ring are carbon atoms. Carbocyclic aryl groups include monocyclic carbocyclic aryl groups and polycyclic or fused compounds such as optionally substituted naphthyl groups.

“Heteroaryl” groups are groups having from 1 to 4 heteroatoms as ring atoms in the aromatic ring and the remainder of the ring atoms being carbon atoms. Suitable heteroatoms include oxygen, sulfur, nitrogen, and selenium. Suitable heteroaryl groups include furanyl, thienyl, pyridyl, pyrrolyl, N-lower alkyl pyrrolyl, pyridyl-N-oxide, pyrimidyl, pyrazinyl, imidazolyl, and the like, all optionally substituted.

The phrase “carbocyclic ring” refers to a saturated or unsaturated monocyclic or bicyclic ring in which all atoms of all rings are carbon. Thus, the term includes cycloalkyl and carbocyclic aryl rings.

The phrase “heterocyclic ring” refers to a saturated or unsaturated monocyclic or bicyclic ring having from 1 to 4 heteroatoms as ring atoms in the aromatic ring and the remainder of the ring atoms being carbon atoms. Thus, the term includes heterocycloalkyl and heteroaryl rings.

In certain embodiments, “optionally substituted” or “substituted” includes groups substituted by one or more substituents independently selected from halogen, —NO₂, —CN, —OR¹⁰⁰, —SR¹⁰⁰, —N(R¹⁰⁰)₂, —S(═O)R¹⁰⁰, —S(═O)₂R¹⁰⁰, —S(═O)₂N(R¹⁰⁰)₂, —NR¹⁰⁰S(═O)₂R¹⁰⁰, —C(O)R¹⁰⁰, —C(O)OR¹⁰⁰, —OC(O)R¹⁰⁰, —OC(O)OR¹⁰⁰, —OC(O)N(R¹⁰⁰)₂, —NR¹⁰⁰C(O)R¹⁰⁰, —C(O)N(R¹⁰⁰)₂, ═O, ═S, ═N(R¹⁰⁰), —P(O)(OR¹⁰⁰)₂, —OP(O)(OR¹⁰⁰)₂; C1-10 alkyl, C₂₋₁₀ alkenyl, and C₂₋₁₀ alkynyl, each of which is independently optionally substituted at each occurrence with one or more substituents selected from halogen, —NO₂, —CN, —OR¹⁰⁰, —SR¹⁰⁰, —N(R¹⁰⁰)₂, —S(═O)R¹⁰⁰, —S(═O)₂R¹⁰⁰, —S(═O)₂N(R¹⁰⁰)₂, —NR¹⁰⁰S(═O)₂R¹⁰⁰, —C(O)R¹⁰⁰, —C(O)OR¹⁰⁰, —OC(O)R¹⁰⁰, —OC(O)OR¹⁰⁰, —OC(O)N(R¹⁰⁰)₂, —NR¹⁰⁰C(O)R¹⁰⁰, —C(O)N(R¹⁰⁰)₂, ═O, ═S, ═N(R¹⁰⁰), —P(O)(OR¹⁰⁰)₂, —OP(O)(OR¹⁰⁰)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle is independently optionally substituted with one or more substituents selected from halogen, —NO₂, —CN, —OR¹⁰⁰, —SR¹⁰⁰, —N(R¹⁰⁰)₂, —S(═O)R¹⁰⁰, —S(═O)₂R¹⁰⁰, —S(═O)₂N(R¹⁰⁰)₂, —NR¹⁰⁰S(═O)₂R¹⁰⁰, —C(O)R¹⁰⁰, —C(O)OR¹⁰⁰, —OC(O)R¹⁰⁰, —OC(O)OR¹⁰⁰, —OC(O)N(R¹⁰⁰)₂, —NR¹⁰C(O)R¹⁰⁰, —C(O)N(R¹⁰⁰)₂, ═O, ═S, ═N(R¹⁰⁰), —P(O)(OR¹⁰⁰)₂, —OP(O)(OR¹⁰⁰)₂, C₁₋₆ alkyl, C₁₋₆ haloalkyl, C₂₋₆ alkenyl, and C₂₋₆ alkynyl, wherein R¹⁰⁰ at each occurrence is independently selected from hydrogen; and C₁₋₂₀ alkyl, C₂₋₂₀ alkenyl, C₂₋₂₀ alkynyl, C₃₋₁₂ carbocycle and 3- to 12-membered heterocycle, each of which may be optionally substituted by halogen, —CN, —NO₂, —OH and —OCH₃. In certain embodiments, the term “optionally substituted” or “substituted” includes groups substituted by one to four substituents, independently selected from lower alkyl, lower aryl, lower aralkyl, lower alicyclic, heterocycloalkyl, hydroxyl, lower alkoxy, lower aryloxy, perhaloalkoxy, aralkoxy, heteroaryl, heteroaryloxy, heteroarylalkyl, heteroaralkoxy, azido, amino, guanidino, amidino, halo, lower alkylthio, oxo, acylalkyl, carboxy esters, carboxyl,-carboxamido, nitro, acyloxy, aminoalkyl, alkylaminoaryl, alkylaryl, alkylaminoalkyl, alkoxyaryl, arylamino, aralkylamino, phosphono, sulfonyl, -carboxamidoalkylaryl, -carboxamidoaryl, hydroxyalkyl, haloalkyl, alkylaminoalkylcarboxy-, aminocarboxamidoalkyl-, cyano, lower alkoxyl, lower perhaloalkyl, and arylalkyloxyalkyl.

“Tautomers” are structurally distinct isomers that interconvert by tautomerization. “Tautomerization” is a form of isomerization and includes prototropic or proton shift tautomerization, which is considered a subset of acid base chemistry. “Prototropic tautomerization” or “proton shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto enol tautomerization. A specific example of keto enol tautomerization is the interconversion of pentane 2,4 dione and 4 hydroxypent 3 en 2 one tautomers. Another example of tautomerization is phenol keto tautomerization. A specific example of phenol keto tautomerization is the interconversion of pyridin 4 ol and pyridin 4(1H) one tautomers.

In some embodiments, a compound described by, or utilized in accordance with, presently disclosed subject matter contain a linking group. As used herein, the term “linking group” comprises a chemical moiety which is bonded to two or more other chemical moieties to form a stable structure. In certain embodiments, the linking group, e.g., methylene, ethylene, links a moiety, e.g., an aryl or heteroaryl group, to the remainder of the structure.

“Alkylene” refers to a straight or branched bivalent aliphatic hydrocarbon group having from 1 to 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group can be optionally substituted with one or more substituents. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to 20, e.g., 0, 1, 2, 3, 4, 5, 6, 7, 8, 9, 10, 11, 12, 13, 14, 15, 16, 17, 18, 19, or 20, and R is hydrogen or lower alkyl. In certain embodiments, an alkylene group has from 1 to 6 carbon atoms such as from 1 to 3 carbon atoms.

The term “alkenylene” denotes a straight or branched bivalent aliphatic hydrocarbon group having from 2 to 20 carbon atoms with at least one carbon-carbon double bond. The alkenylene group can be optionally substituted with one or more substituents. Representative alkenylene groups include, but are not limited to, ethenylene, propenylene, 1- or 2-butenylene, 1- or 2-pentylene, and the like.

As used herein, the term “acyl” refers to an group represented by R—C(═O), wherein R is, for example, an alkyl or an aryl group as defined herein. Specific examples of acyl groups include acetyl and benzoyl.

“Alkoxyl” refers to an alkyl-O— group wherein alkyl is as previously described. The term “alkoxyl” as used herein can refer to C₁₋₂₀ inclusive, linear, branched, or cyclic, saturated or unsaturated oxo-hydrocarbon chains, including, for example, methoxyl, ethoxyl, propoxyl, isopropoxyl, butoxyl, t-butoxyl, and pentoxyl.

“Aryloxyl” refers to an aryl-O— group wherein the aryl group is as previously described, including a substituted aryl. The term “aryloxyl” as used herein can refer to optionally substituted phenyloxyl.

“Aralkyl” refers to an aryl-alkyl- group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl.

“Dialkylamino” refers to an —NRR′ group wherein each of R and R′ is independently selected from optionally substituted alkyl groups as previously described. Exemplary alkylamino groups include ethylmethylamino, dimethylamino, and diethylamino.

“Alkoxycarbonyl” refers to an alkoxyl-C(O)— group. Exemplary alkoxycarbonyl groups include methoxycarbonyl, ethoxycarbonyl, butyloxycarbonyl, and t-butyloxycarbonyl.

“Aryloxycarbonyl” refers to an aryloxyl-CO— group. Exemplary aryloxycarbonyl groups include phenoxycarbonyl and naphthoxycarbonyl.

“Aralkoxycarbonyl” refers to an aralkyl-O—CO— group. An exemplary aralkoxycarbonyl group is benzyloxycarbonyl.

“Carbamoyl” refers to an H₂N—CO— group.

“Alkylcarbamoyl” refers to a R′RN—CO— group wherein one of R and R′ is hydrogen and the other of R and R′ is optionally substituted alkyl as previously described.

“Dialkylcarbamoyl” refers to a R′RN—CO— group wherein each of R and R′ is independently optionally substituted alkyl as previously described.

“Acyloxyl” refers to an acyl-O— group wherein acyl is as previously described.

“Acylamino” refers to an acyl-NH— group wherein acyl is as previously described.

The term “amino” refers to the —NH₂ group and amino may be optionally substituted.

The term “carbonyl” refers to the —C(O)— group.

The term “carboxyl” refers to the —COOH group.

The term “cyano” refers to the —CN group.

The terms “halo,” “halide,” or “halogen” as used herein refer to fluoro, chloro, bromo, and iodo groups.

The term “hydroxyl” refers to the —OH group.

The term “hydroxyalkyl” refers to an alkyl group substituted with one or more —OH substituents.

The term “haloalkyl” refers to an alkyl group with one or more independently selected halogen substituents.

The term “mercapto” refers to the —SH group.

The term “oxo” refers to ═O.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term “treatment” as used herein covers any treatment of a disease and/or condition in an animal or mammal, particularly a human, and includes: (i) preventing a disease, disorder and/or condition and/or symptoms from occurring in a person which can be predisposed to the disease, disorder and/or condition, or at risk for being exposed to an agent that can cause the disease, disorder, and/or condition and/or symptoms; but, has not yet been diagnosed as having it; (ii) inhibiting the disease, disorder and/or condition, and/or symptoms i.e., arresting its development; (iii) slowing progress of the disease, disorder and/or condition, and/or symptoms; and (iv) relieving the disease, disorder and/or condition, and/or symptoms i.e., causing regression of the disease, disorder and/or condition; (v) augmenting a mechanism, such as modulating p75^{NTR} signaling, that can lead to reduced symptoms and improved function.

The terms “modulate” and “modulation” as used herein are used in the common manner of the field as to regulate or adjust to a certain degree.

The term “derivative” as used herein refers to a compound chemically modified so as to differentiate it from a parent compound. Such chemical modifications can include, for example, replacement of hydrogen by an alkyl, acyl, or amino group. A derivative compound can be modified by, for example, glycosylation, PEGylation, or any similar process that retains at least one biological or immunological function of the compound from which it was derived.

The term “hydrophilicity” is used in the common manner of the field as having an affinity for water; readily absorbing and/or dissolving in water.

The term “lipophilicity” is used in the common manner of the field as having an affinity for, tending to combine with, or capable of dissolving in lipids.

The term “amphipathicity,” as used herein, describes a structure having discrete hydrophobic and hydrophilic regions. Thus, one portion of the structure interacts favorably with aqueous and other polar media, while another portion of the structure interacts favorably with non-polar media.

The term “solubility” as used herein, describes the maximum amount of solute that will dissolve in a given amount of solvent at a specified temperature.

The term “bioavailability” as used herein refers to the systemic availability, blood/plasma levels, of a given amount of compound administered to a subject. The term further encompasses the rate and extent of absorption of compound that reaches the site of action.

The term “prodrug” as used herein refers to any compound that when administered to a biological system generates the drug substance (a biologically active compound) in steps involving, for example, spontaneous chemical reaction(s), enzyme catalyzed chemical reaction(s), or both.

Where the compounds as described herein have at least one asymmetric center, they may accordingly exist as enantiomers. Where the compounds possess two or more asymmetric centers, they may additionally exist as diastereoisomers. It is to be understood that all such stereoisomers and mixtures thereof in any proportion are encompassed within the scope of the present invention. Where the compounds possess geometrical isomers, all such isomers and mixtures thereof in any proportion are encompassed within the scope of the present invention. Where so indicated in the claims herein, if a single enantiomer of the potentially optically active heterocyclic compounds disclosed is desired, for either health or efficacy reasons, preferably it is present in an enantiomeric excess of at least about 80%, or at least about 90%, or at least about 95%, or at least about 98%, or at least about 99%, or at least about 99.5%.

Polymorphism can be characterized as the ability of a compound to crystallize into different crystal forms, while maintaining the same chemical formula. A crystalline polymorph of a given drug substance is chemically identical to any other crystalline polymorph of that drug substance in containing the same atoms bonded to one another in the same way, but differs in its crystal forms, which can affect one or more physical properties, such as stability, solubility, melting point, bulk density, flow properties, bioavailability, etc.

The term “composition” denotes one or more substance in a physical form, such as solid, liquid, gas, or a mixture thereof. One example of composition is a pharmaceutical composition, i.e., a composition related to, prepared for, or used in medical treatment.

The term “carboxylic acid” refers to an organic acid characterized by one or more carboxyl groups, such as acetic acid and oxalic acid. “Sulfonic acid” refers to an organic acid with the general formula of R—(S(O)₂—OH)ₙ, wherein R is an organic moiety and n is an integer above zero, such as 1, 2, and 3. The term “polyhydroxy acid” refers to a carboxylic acid containing two or more hydroxyl groups. Examples of polyhydroxy acid include, but are not limited to, lactobionic acid, gluconic acid, and galactose.

“Neurotrophin mimetic compound” denotes an organic compound that resembles the biological function or activity of neurotrophin.

As used herein, “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

“Salts” include derivatives of an active agent, wherein the active agent is modified by making acid or base addition salts thereof. Preferably, the salts are pharmaceutically acceptable salts. Such salts include, but are not limited to, pharmaceutically acceptable acid addition salts, pharmaceutically acceptable base addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine dicyclohexylamine and the like. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Standard methods for the preparation of pharmaceutically acceptable salts and their formulations are well known in the art, and are disclosed in various references, including for example, Remington: The Science and Practice of Pharmacy, A. Gennaro, ed., 20th edition, Lippincott, Williams & Wilkins, Philadelphia, PA.

As used herein, “solvate” means a complex formed by solvation (the combination of solvent molecules with molecules or ions of the active agent of the present invention), or an aggregate that consists of a solute ion or molecule (the active agent of the present invention) with one or more solvent molecules. In the present invention, the preferred solvate is hydrate. Examples of hydrate include, but are not limited to, hemihydrate, monohydrate, dihydrate, trihydrate, hexahydrate, etc. It should be understood by one of ordinary skill in the art that the pharmaceutically acceptable salt of the present compound may also exist in a solvate form. The solvate is typically formed via hydration which is either part of the preparation of the present compound or through natural absorption of moisture by the anhydrous compound of the present invention. Solvates including hydrates may be consisting in stoichiometric ratios, for example, with two, three, four salt molecules per solvate or per hydrate molecule. Another possibility, for example, that two salt molecules are stoichiometric related to three, five, seven solvent or hydrate molecules. Solvents used for crystallization, such as alcohols, especially methanol and ethanol; aldehydes; ketones, especially acetone; esters, e.g. ethyl acetate; may be embedded in the crystal grating. Preferred are pharmaceutically acceptable solvents.

The term “substantially similar” as used herein means an analytical spectrum, such as XRD pattern, Raman spectroscopy, and etc., which resembles the reference spectrum to a great degree in both the peak locations and their intensity.

The terms “excipient,” “carrier,” and “vehicle” are used interchangeably throughout this application and denote a substance with which a compound of the present disclosure is administered.

As described herein, the dataset indicated as “a combined drug” are derived from corresponding analyses that combine both datasets indicated as “200 mg” and “400 mg” for the purpose of statistical tests.

As described herein, unless specified otherwise, the term “baseline” or “corresponding reference,” when used in describing the effect(s) of a therapeutic agent may refer to a corresponding measurement determined prior to the treatment, a placebo control where a biologically inert substance is used, or a corresponding cohort or subject determined to have identical or meaningfully similar conditions.

The term “neurodegeneration biomarkers or indicators” as used herein can refer to “biomarkers of degeneration, neuroinflammation or gliosis.”

The term “tau (t)” as used herein can broadly include various modified or aggregates forms, for example, including pathological tau and phosphorylated tau.

As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

As used herein, the term “corresponding to” refers to a relationship between two or more entities. For example, the term “corresponding to” may be used to designate the position/identity of a structural element in a compound or composition relative to another compound or composition (e.g., to an appropriate reference compound or composition). Alternatively or additionally, the term “corresponding to” may be used to refer to an amount of a chemical compound, or form thereof, that shares a relevant feature (e.g., an ability to achieve a particular biological or pharmacological effect) as achieved by a reference amount of another compound or form. Those of skill in the art will appreciate that, in some instances, the term “corresponding to” may be used to describe an event or entity that shares a relevant similarity with another event or entity (e.g., an appropriate reference event or entity).

Those skilled in the art will appreciate that the term “dosage form” (or “unit dosage form”) may be used to refer to a physically discrete unit of an active agent for administration to a subject. Typically, each such unit contains a predetermined quantity of active agent (or precursor thereof, particularly which may be converted to active agent upon administration). In some embodiments, such quantity is a unit dosage amount (or a whole fraction thereof) appropriate for administration in accordance with a dosing regimen that has been determined to correlate with a desired or beneficial outcome when administered to a relevant population. Those of ordinary skill in the art appreciate that the total amount of a therapeutic composition or agent administered to a particular subject is determined by one or more attending physicians and may involve administration of multiple dosage forms.

Those skilled in the art will appreciate that the term “dosing regimen”, as used herein, may refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Two events or entities are “associated” with one another, as that term is used herein, if the presence, level, degree, type and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of, susceptibility to, severity of, stage of, etc. the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

p75^{NTR} Modulators

As noted herein, the present disclosure provides an insight that modulation of the p75 neurotrophin receptor (“p75^{NTR}”) can impact tau pathology in humans, and establishes that tau pathology biomarkers, specifically including certain phosphorylated forms of tau protein, can be useful biomarkers for p75^{NTR} modulation therapy (i.e., for expected responsiveness to and/or for monitoring impact of administration of a p75^{NTR} modulator(s)).

Those skilled in the art, reading the present disclosure, will appreciate the extent to which they are applicable to those agents that may be determined to interact with or impact level (e.g., expression), form, and/or activity of p75^{NTR}

In one aspect, the compounds as described in the present disclosure may be used as a p75^{NTR} modulator used in the methods as disclosed herein.

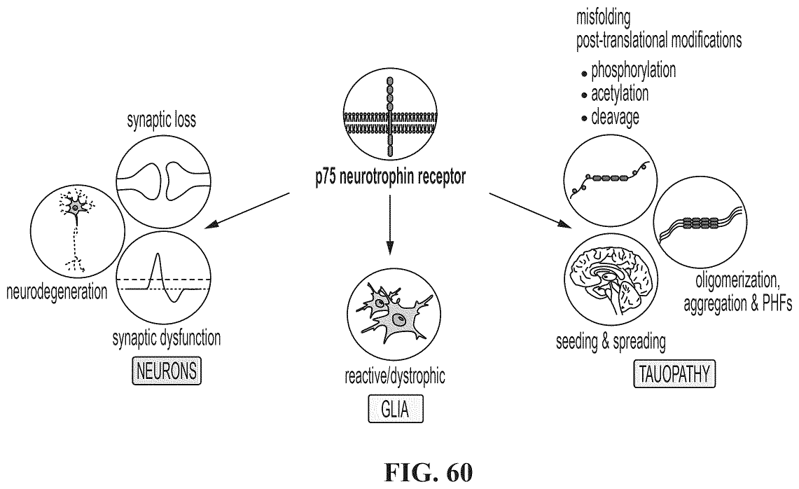

The p75^{NTR} is a member of the tumor necrosis factor family. p75^{NTR} is active in neurons and glia, and is associated with proteinopathies (FIG. 60). Although p75^{NTR} has traditionally been known as a ‘death receptor’, it can also determine synaptic and cellular fate. p75^{NTR} regulates a broad intracellular signaling network that has considerable overlap with degenerative signaling networks active in AD.

In one aspect, the p75^{NTR} modulator used in the methods as disclosed herein is a compound represented by Formula III:

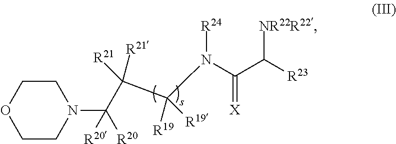

or a salt thereof, wherein:
X is CH₂, NH, O or S;
s is 0, 1, 2, 3 or 4;
each of R¹⁹, R^{19′}, R²⁰, R^{20′}, R²¹, R^{21′}, R²², R^{22′} and R²⁴ is independently selected at each occurrence from hydrogen and optionally substituted alkyl; or
R²⁰ and R^{20′} taken together form ═O, ═S, or ═CH₂; or
R²⁰ and R²¹ taken together with the atoms to which they are attached form an optionally substituted cycloalkyl; or
R²⁰ and R²¹ taken together with the atoms to which they are attached form an optionally substituted aryl; or
R¹⁹ and R²⁰ taken together with the atoms to which they are attached form an optionally substituted cycloalkyl; or
R¹⁹ and R²⁰ taken together with the atoms to which they are attached form an optionally substituted aryl; and
R²³ is hydrogen, optionally substituted alkyl, optionally substituted cycloalkyl or optionally substituted aryl.

In certain embodiments, for a compound or salt of Formula III, s is 0, 1 or 2. In an exemplary embodiment, s is 0.

In certain embodiments, for a compound or salt of Formula III, X is NH, O or S. In an exemplary embodiment, X is O.

For a compound or salt of Formula III, when R¹⁹, R^{19′}, R²⁰, R^{20′}, R²¹, R^{21′}, R²², R^{22′} or R²⁴ is optionally substituted, the optional substituents may be independently selected at each occurrence from halogen, —NO₂, —CN, —OR¹⁰⁰, —SR¹⁰⁰, —N(R¹⁰⁰)₂, —S(═O)R¹⁰⁰, —S(═O)₂R¹⁰⁰, —S(═O)₂N(R¹⁰⁰)₂, —NR¹⁰⁰S(═O)₂R¹⁰⁰, —C(O)R¹⁰⁰, —C(O)OR¹⁰⁰, —OC(O)R¹⁰⁰, —OC(O)OR¹⁰⁰, —OC(O)N(R¹⁰⁰)₂, —NR¹⁰⁰C(O)R¹⁰⁰, —C(O)N(R¹⁰⁰)₂, ═O, ═S, ═N(R¹⁰⁰), —P(O)(OR¹⁰⁰)₂, —OP(O)(OR¹⁰⁰)₂; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR¹⁰⁰, —SR¹⁰⁰, —N(R¹⁰⁰)₂, —S(═O)R¹⁰⁰, —S(═O)₂R¹⁰⁰, —S(═O)₂N(R¹⁰⁰)₂, —NR¹⁰⁰S(═O)₂R¹⁰⁰, —C(O)R¹⁰⁰, —C(O)OR¹⁰⁰, —OC(O)R¹⁰⁰, —OC(O)OR¹⁰⁰, —OC(O)N(R¹⁰⁰)₂, —NR¹⁰⁰C(O)R¹⁰⁰, —C(O)N(R¹⁰⁰)₂, ═O, ═S, ═N(R¹⁰⁰), —P(O)(OR¹⁰⁰)₂, —OP(O)(OR¹⁰⁰)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle is independently optionally substituted with one or more substituents selected from halogen, —NO₂, —CN, —OR¹⁰⁰, —SR¹⁰⁰, —N(R¹⁰⁰)₂, —S(═O)R¹⁰⁰, —S(═O)₂R¹⁰⁰, —S(═O)₂N(R¹⁰⁰)₂, —NR¹⁰⁰S(═O)₂R¹⁰⁰, —C(O)R¹⁰⁰, —C(O)OR¹⁰⁰, —OC(O)R¹⁰⁰, —OC(O)OR¹⁰⁰, —OC(O)N(R¹⁰⁰)₂, —NR¹⁰C(O)R¹⁰⁰, —C(O)N(R¹⁰⁰)₂, ═O, ═S, ═N(R¹⁰⁰), —P(O)(OR¹⁰⁰)₂, —OP(O)(OR¹⁰⁰)₂, C₁₋₆ alkyl, C₁₋₆ haloalkyl, C₂₋₆ alkenyl, and C₂₋₆ alkynyl, wherein R¹⁰⁰ at each occurrence is independently selected from hydrogen; and C₁₋₂₀ alkyl, C₂₋₂₀ alkenyl, C₂₋₂₀ alkynyl, C₃₋₁₂ carbocycle and 3- to 12-membered heterocycle, each of which may be optionally substituted by halogen, —CN, —NO₂, —OH and —OCH₃.

For a compound or salt of Formula III, when R¹⁹, R^{19′}, R²⁰, R^{20′}, R²¹, R^{21′}, R²², R^{22′} or R²⁴ is optionally substituted, the optional substituents may be independently selected at each occurrence from halogen, —NO₂, —CN, —OR¹⁰⁰, —SR¹⁰⁰, —N(R¹⁰⁰)₂, —S(═O)R¹⁰⁰, —S(═O)₂R¹⁰⁰, —S(═O)₂N(R¹⁰⁰)₂, —NR¹⁰⁰S(═O)₂R¹⁰⁰, —C(O)R¹⁰⁰, —C(O)OR¹⁰⁰, —OC(O)R¹⁰⁰, —OC(O)OR¹⁰⁰, —OC(O)N(R¹⁰⁰)₂, —NR¹⁰⁰C(O)R¹⁰⁰, —C(O)N(R¹⁰⁰)₂, ═O, ═S, ═N(R¹⁰⁰), —P(O)(OR¹⁰⁰)₂, —OP(O)(OR¹⁰⁰)₂; C₁-10 alkyl optionally substituted at each occurrence with one or more substituents selected from halogen, —NO₂, —CN, —OR¹⁰⁰, —SR¹⁰⁰, and —N(R¹⁰⁰)₂; and C₃₋₁₂ carbocycle and 3- to 12-membered heterocycle, wherein each C₃₋₁₂ carbocycle and 3- to 12-membered heterocycle is independently optionally substituted with one or more substituents selected from halogen, —NO₂, —CN, —OR¹⁰⁰, —SR¹⁰⁰, —N(R¹⁰⁰)₂, C₁₋₆ alkyl, and C1-6 haloalkyl, wherein R¹⁰⁰ at each occurrence is independently selected from hydrogen; and C₁₋₂₀ alkyl, C₂₋₂₀ alkenyl, C₂₋₂₀ alkynyl, C₃₋₁₂ carbocycle and 3- to 12-membered heterocycle, each of which may be optionally substituted by halogen, —CN, —NO₂, —OH and —OCH₃.

In some embodiments, for a compound or salt of Formula III, s is selected from 1, 2, 3 or 4 and R¹⁹ and R^{19′} are independently selected at each occurrence from hydrogen and optionally substituted C₁-C₆ alkyl. In certain embodiments, s is selected from 1 or 2 and R¹⁹ and R^{19′} are independently selected at each occurrence from hydrogen and optionally substituted C₁-C₃ alkyl.

In certain embodiments, for a compound or salt of Formula III, R²⁰ and R^{20′} are independently selected from hydrogen and optionally substituted C₁-C₆ alkyl. In certain embodiments, R²⁰ and R^{20′} are independently selected from hydrogen and optionally substituted C₁-C₃ alkyl. In certain embodiments, R²⁰ and R^{20′} are each hydrogen.

In certain embodiments, for a compound or salt of Formula III, R²¹ and R^{21′} are independently selected from hydrogen and optionally substituted C₁-C₆ alkyl. In certain embodiments, R²¹ and R^{21′} are independently selected from hydrogen and optionally substituted C₁-C₃ alkyl. In certain embodiments, R²¹ and R^{21′} are each hydrogen.

In certain embodiments, for a compound or salt of Formula III, R²² and R^{22′} are independently selected from hydrogen and optionally substituted C₁-C₆ alkyl. In certain embodiments, R²² and R^{22′} are independently selected from hydrogen and optionally substituted C₁-C₃ alkyl. In certain embodiments, R²² and R^{22′} are each hydrogen.

In certain embodiments, for a compound or salt of Formula III, R²³ is selected from hydrogen, optionally substituted C₁-C₆ alkyl, optionally substituted cycloalkyl and optionally substituted aryl. In certain embodiments, R²³ is selected from optionally substituted C₁-C₆ alkyl, such as optionally substituted C₂-C₅ alkyl, such as optionally substituted C₃-C₅ alkyl, such as optionally substituted C₄ alkyl. In certain embodiments, R²³ is C₃-C₅ alkyl, such as C₄ alkyl. R²³ may be represented by the following structure:

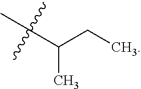 [SMILES: *C(C)CC]

In certain embodiments, for a compound or salt of Formula III, R²⁴ is hydrogen or optionally substituted C₁-C₆ alkyl alkyl. In certain embodiments, R²⁴ is selected from hydrogen and optionally substituted C₁-C₃ alkyl. In certain embodiments, R²⁴ is hydrogen.

In certain embodiments, for a compound or salt of Formula III, s is 0, X is O or S, R²⁰ and R^{20′} are independently selected from hydrogen and optionally substituted C₁-C₃ alkyl, R²¹ and R^{21′} are independently selected from hydrogen and optionally substituted C₁-C₃ alkyl, R²² and R^{22′} are independently selected from hydrogen and optionally substituted C₁-C₃ alkyl, R²³ is optionally substituted C₂-C₅ alkyl, and R²⁴ is selected from hydrogen and optionally substituted C₁-C₃ alkyl.

In certain embodiments, for a compound or salt of Formula III, s is 0, X is O or S, R²⁰ and R^{20′} are each hydrogen, R²¹ and R^{21′} are each hydrogen, R²² and R^{22′} are independently selected from hydrogen and optionally substituted C₁-C₃ alkyl, R²³ is optionally substituted C₂-C₅ alkyl, and R²⁴ is selected from hydrogen and optionally substituted C₁-C₃ alkyl.

In certain embodiments, for a compound or salt of Formula III, s is 0, X is O, R²⁰ and R^{20′} are independently selected from hydrogen and optionally substituted C₁-C₃ alkyl, R²¹ and R^{21′} are independently selected from hydrogen and optionally substituted C₁-C₃ alkyl, R²² and R^{22′} are each hydrogen, R²³ is optionally substituted C₄ alkyl, and R²⁴ is hydrogen.

In certain embodiments, the p75^{NTR} modulator used in the methods as described herein is a compound represented by Formula I:

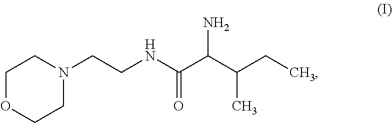 [SMILES: CCC(C)C(N)C(=O)NCCN1CCOCC1]

or a pharmaceutically acceptable salt thereof.

The compound of Formula I may be represented by Formula Ia:

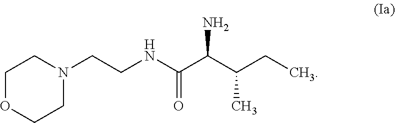 [SMILES: CC[C@H](C)[C@H](N)C(=O)NCCN1CCOCC1]

or a pharmaceutically acceptable salt thereof.

“LM11A-31” as used herein refers to a compound of Formula (Ia) or a pharmaceutically acceptable salt thereof.

Included in the present disclosure are salts, particularly pharmaceutically acceptable salts, of the compounds of Formula (I) and (Ia). The compounds as described in the present disclosure (e.g., the p75^{NTR} modulators used in the methods described herein) can react with any of a number and inorganic and organic acids to form salts. In preferred embodiments, the compound of Formula (I) or (Ia) is an acid addition salt.

Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

In some embodiments, a salt form is selected based on amenability to manufacture at a scale appropriate to the condition to be treated. In certain embodiments, a salt form is characterized by a particular flowability, storage stability and/or other feature.

In certain embodiments, the salt of the compound of Formula (I) or (Ia) is a sulfate salt, a bisulfate salt or a combination thereof. That is, in certain embodiments, where the compound of Formula (I) or (Ia) is utilized, it is utilized in its sulfate or bisulfate salt form.

In certain embodiments the salt of the compound of Formula (I) is represented by Formula (II):

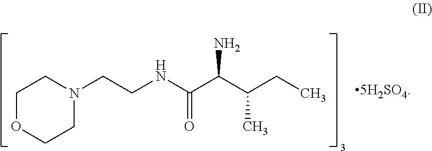

### Salts

In some embodiments, a compound for use in accordance with the methods disclosed herein is provided and/or utilized in a pharmaceutically acceptable salt form; in some such embodiments, a compound that is provided and/or utilized is a pharmaceutically acceptable salt of a compound of Formula (I):

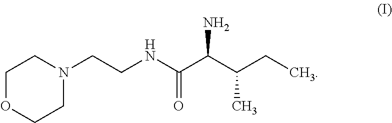 [SMILES: CC[C@H](C)[C@H](N)C(=O)NCCN1CCOCC1]

In some embodiments, the salt is a hydrogensulfate (H₂SO₄) salt.

In some embodiments, the hydrogensulfate salt is a bishydrogensulfate salt.

In some embodiments, the hydrogensulfate salt is:

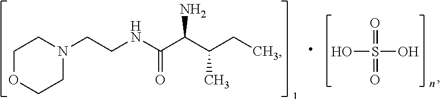

wherein n is about 1 to about 2. In some embodiments, n is 1.4.

In some embodiments, the salt is an adipic salt or gentisic salt.

In some embodiments, the salt is an adipic salt.

In some embodiments, the adipic salt is:

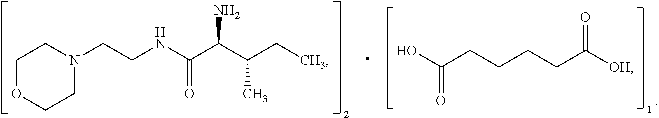

In some embodiments, the adipic salt is:

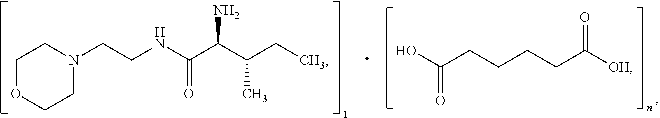

wherein n is about 0.5 to about 2. In some embodiments, n is 0.5.

In some embodiments, the salt is a gentisic salt.

In some embodiments, the gentisic salt is:

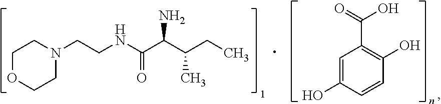

wherein n is about 0.5 to about 2. In some embodiments, n is 1.

In some embodiments, a compound that is utilized in accordance with the present disclosure is provided and/or utilized in a solid form, for example in a crystal form (e.g., hydrate, solvate and/or anhydrous crystal form).

### ADI Form A

In one aspect, an adipic salt of a compound of Formula (I):

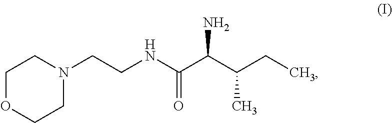 [SMILES: CC[C@H](C)[C@H](N)C(=O)NCCN1CCOCC1]

is provided and/or utilized in a crystalline form. In some embodiments, a particular crystalline form may be detected, for example in a composition, by detection of a set of characteristic peaks.

In some embodiments, such a crystalline form exhibits an X-ray powder diffraction pattern comprising peaks at the following diffraction angles (2θ): 8.4±0.2, 19.8±0.2, and 21.0±0.2.

In some embodiments, such a crystalline form exhibits an X-ray powder diffraction pattern comprising peaks at the following diffraction angles (2θ): 8.4±0.2, 15.4±0.2, 19.8±0.2, 21.0±0.2, and 22.6±0.2.

In some embodiments, the crystalline form exhibits an X-ray powder diffraction pattern comprising peaks at the following diffraction angles (2θ): 8.4±0.2, 13.2±0.2, 15.4±0.2, 18.3±0.2, 18.9±0.2, 19.8±0.2, 21.0±0.2, 21.4±0.2, 22.6±0.2, and 24.4±0.2.

In some embodiments, the crystalline form exhibits an X-ray powder diffraction pattern comprising peaks selected from Table 1.

TABLE 1
XRPD Peak List of ADI Form A

| | Position | Intensity | |
|---|---|---|---|
| Peak | (°2-theta) | (Counts) | Rel. Int. (%) |
| 1 | 5.2 | 4133.0 | 17.9 |
| 2 | 8.4 | 23033.0 | 100.0 |
| 3 | 9.6 | 1906.0 | 8.3 |
| 4 | 10.5 | 2333.0 | 10.1 |
| 5 | 13.2 | 9590.0 | 41.6 |
| 6 | 15.4 | 14772.0 | 64.1 |
| 7 | 16.3 | 3142.0 | 13.6 |
| 8 | 16.9 | 4659.0 | 20.2 |
| 9 | 17.7 | 2502.0 | 10.9 |
| 10 | 17.9 | 4350.0 | 18.9 |
| 11 | 18.3 | 7107.0 | 30.9 |
| 12 | 18.9 | 7899.0 | 34.3 |
| 13 | 19.5 | 3750.0 | 16.3 |
| 14 | 19.8 | 17982.0 | 78.1 |
| 15 | 20.0 | 8076.0 | 35.1 |
| 16 | 20.5 | 4796.0 | 20.8 |
| 17 | 20.8 | 4293.0 | 18.6 |
| 18 | 21.0 | 14823.0 | 64.4 |
| 19 | 21.4 | 6063.0 | 26.3 |
| 20 | 21.7 | 1879.0 | 8.2 |
| 21 | 22.0 | 1471.0 | 6.4 |
| 22 | 22.6 | 9945.0 | 43.2 |
| 23 | 22.9 | 1349.0 | 5.9 |
| 24 | 23.8 | 2566.0 | 11.1 |
| 25 | 24.4 | 5028.0 | 21.8 |
| 26 | 24.8 | 3060.0 | 13.3 |
| 27 | 25.5 | 2883.0 | 12.5 |
| 28 | 26.4 | 1553.0 | 6.7 |
| 29 | 26.6 | 1437.0 | 6.2 |
| 30 | 27.6 | 1584.0 | 6.9 |
| 31 | 29.0 | 1622.0 | 7.0 |
| 32 | 30.2 | 1256.0 | 5.5 |
| 33 | 30.6 | 2282.0 | 9.9 |
| 34 | 32.3 | 1158.0 | 5.0 |
| 35 | 32.6 | 1053.0 | 4.6 |
| 36 | 37.2 | 2147.0 | 9.3 |
| 37 | 37.6 | 1675.0 | 7.3 |
| 38 | 38.0 | 1185.0 | 5.1 |
| 39 | 38.2 | 1503.0 | 6.5 |
| 40 | 38.5 | 1158.0 | 5.0 |

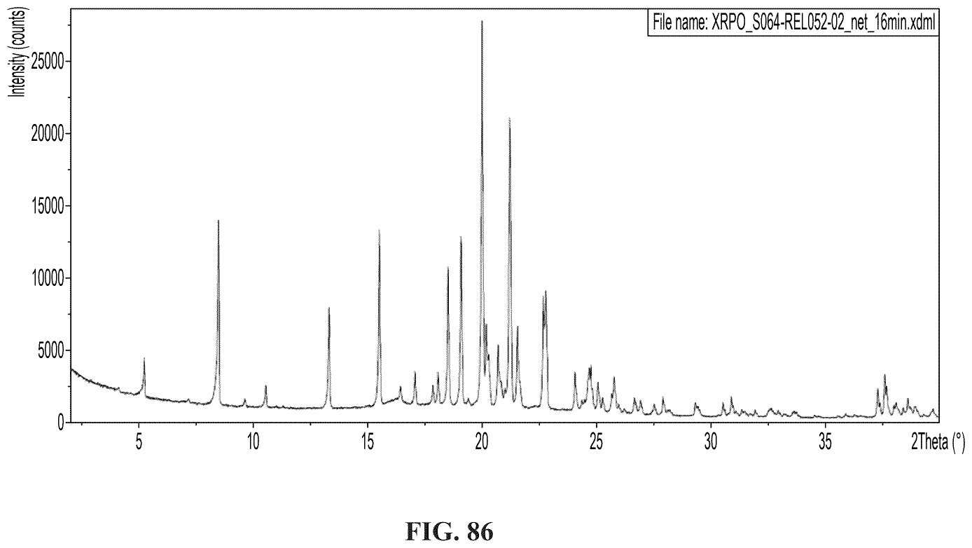

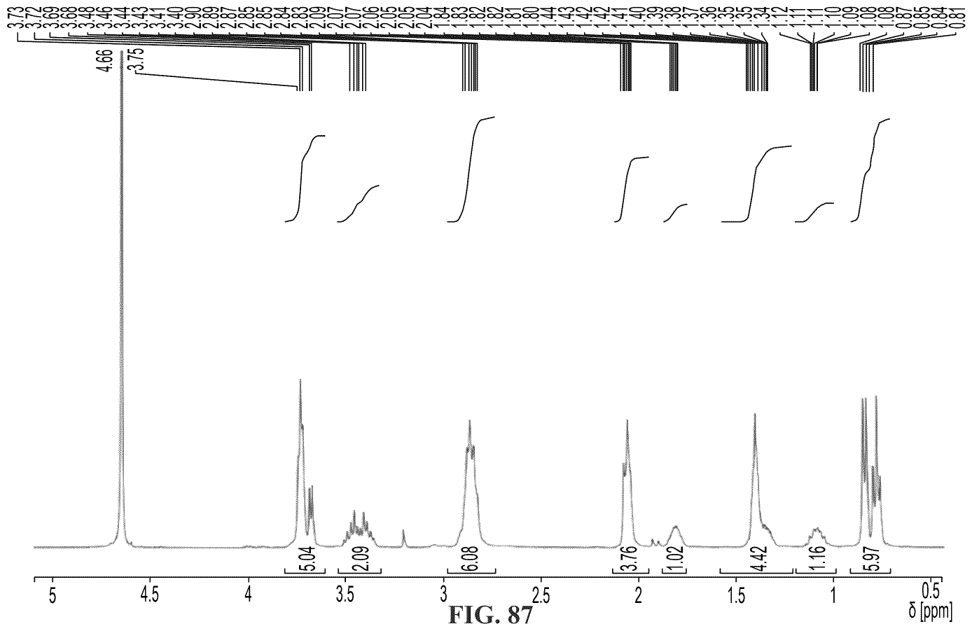

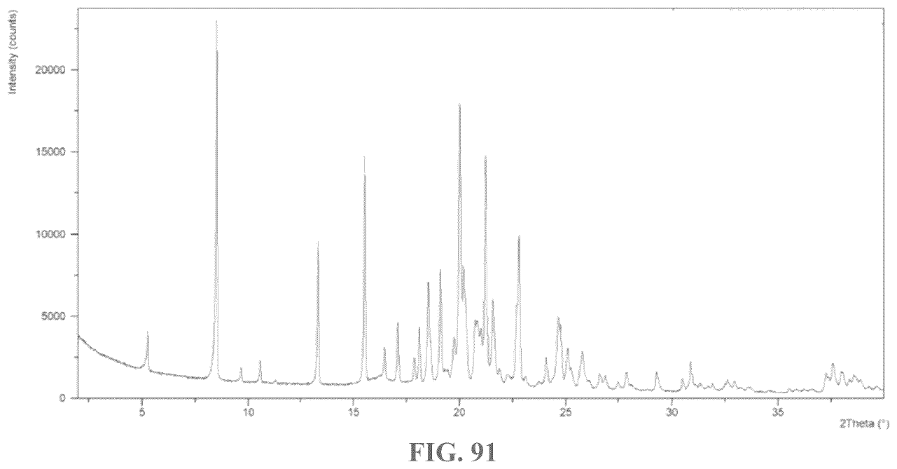

In some embodiments, the crystalline form exhibits an X-ray powder diffraction pattern substantially the same as depicted in FIG. 86 or FIG. 91.

In some embodiments, the crystalline form has a melting point onset as determined by differential scanning calorimetry at about 104° C.

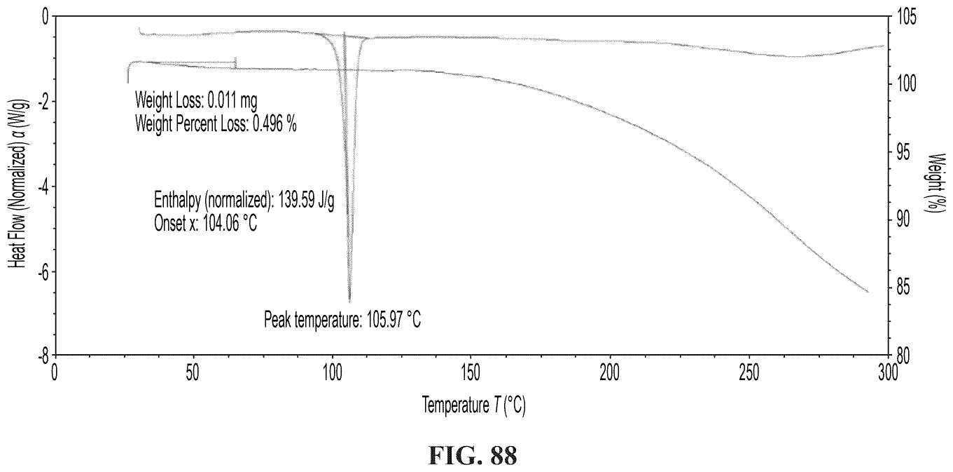

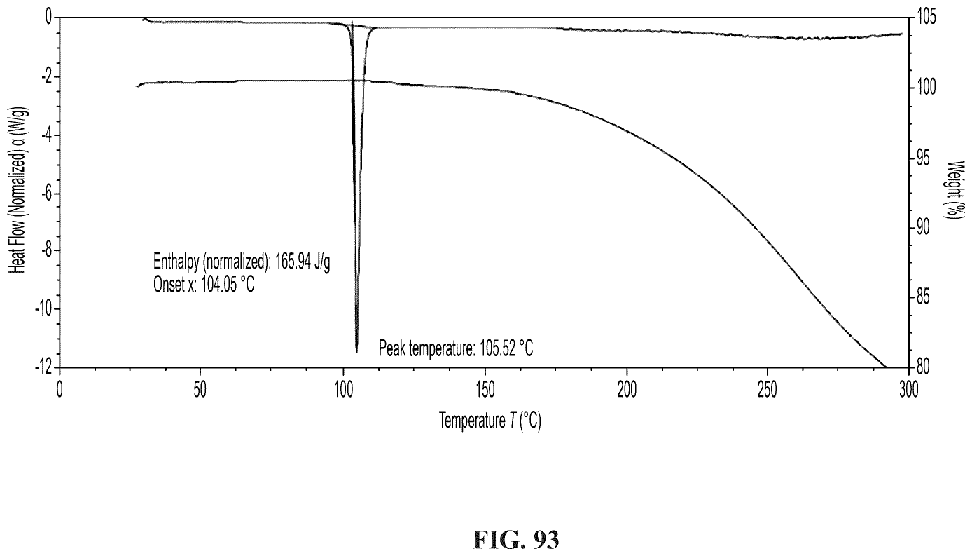

In some embodiments, the crystalline form has a differential scanning calorimetry curve substantially the same as shown in FIG. 88 or FIG. 93.

### GEN Form A

In one aspect, a gentisic salt of a compound of Formula (I):

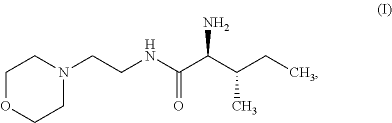 [SMILES: CC[C@H](C)[C@H](N)C(=O)NCCN1CCOCC1]

is provided, and/or utilized in a crystalline form. In some embodiments, a particular crystalline form may be detected, for example in a composition, by detection of a set of characteristic peaks.

In some embodiments, the crystalline form exhibits an X-ray powder diffraction pattern comprising peaks at the following diffraction angles (2θ): 7.9±0.2, 17.3±0.2, and 23.1±0.2.

In some embodiments, the crystalline form exhibits an X-ray powder diffraction pattern comprising peaks at the following diffraction angles (2θ): 7.9±0.2, 17.3±0.2, 22.1±0.2, 23.1±0.2, and 24.5±0.2.

In some embodiments, the crystalline form exhibits an X-ray powder diffraction pattern comprising peaks at the following diffraction angles (2θ): 7.9±0.2, 11.0±0.2, 15.8±0.2, 17.3±0.2, 17.6±0.2, 20.6±0.2, 22.1±0.2, 23.1±0.2, 24.5±0.2, and 26.1±0.2.

In some embodiments, the crystalline form exhibits an X-ray powder diffraction pattern comprising peaks selected from Table 2.

TABLE 2
XRPD Peak List of GEN Form A

| | Position | Intensity | |
|---|---|---|---|
| Peak | (°2-theta) | (Counts) | Rel. Int. (%) |
| 1 | 7.9 | 57314.0 | 100.0 |
| 2 | 11.0 | 10734.0 | 18.7 |
| 3 | 15.8 | 15284.0 | 26.7 |
| 4 | 17.3 | 44989.0 | 78.5 |
| 5 | 17.6 | 27128.0 | 47.3 |
| 6 | 20.6 | 19929.0 | 34.8 |
| 7 | 21.0 | 5427.0 | 9.5 |
| 8 | 22.1 | 32651.0 | 57.0 |
| 9 | 22.4 | 5356.0 | 9.3 |
| 10 | 23.1 | 44381.0 | 77.4 |
| 11 | 23.9 | 2932.0 | 5.1 |
| 12 | 24.5 | 33595.0 | 58.6 |
| 13 | 25.1 | 1852.0 | 3.2 |
| 14 | 26.1 | 12022.0 | 21.0 |
| 15 | 27.0 | 8379.0 | 14.6 |
| 16 | 28.2 | 6554.0 | 11.4 |
| 17 | 28.8 | 4259.0 | 7.4 |
| 18 | 29.8 | 4469.0 | 7.8 |
| 19 | 31.5 | 3069.0 | 5.4 |
| 20 | 31.8 | 1677.0 | 2.9 |
| 21 | 32.1 | 1885.0 | 3.3 |
| 22 | 32.8 | 2057.0 | 3.6 |
| 23 | 34.1 | 2021.0 | 3.5 |
| 24 | 35.0 | 3160.0 | 5.5 |
| 25 | 35.8 | 2468.0 | 4.3 |
| 26 | 36.8 | 2513.0 | 4.4 |
| 27 | 37.8 | 3993.0 | 7.0 |
| 28 | 38.4 | 1925.0 | 3.4 |

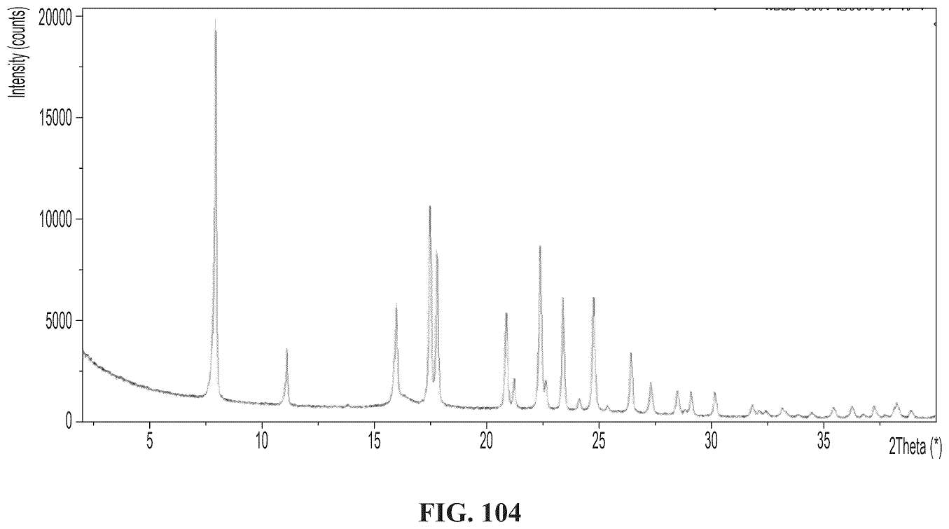

In some embodiments, the crystalline form exhibits an X-ray powder diffraction pattern substantially the same as depicted in FIG. 104.

In some embodiments, the crystalline form has a melting point onset as determined by differential scanning calorimetry at about 167° C.

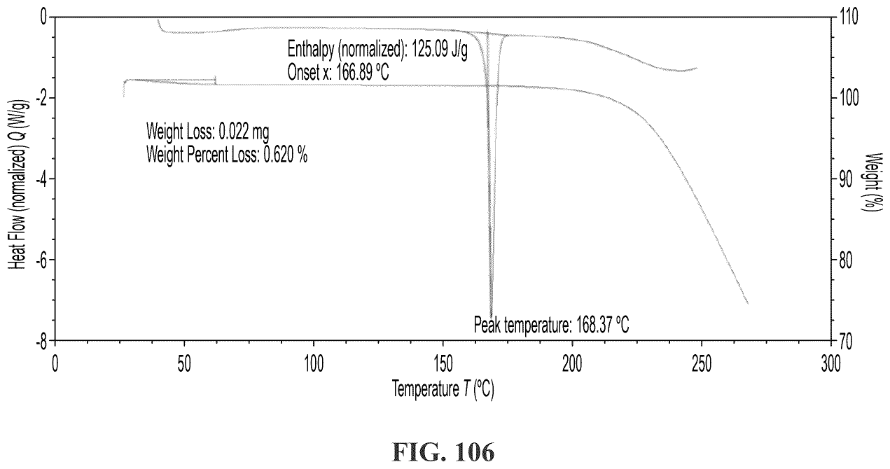

In some embodiments, the crystalline form has a differential scanning calorimetry curve substantially the same as shown in FIG. 106.

### GEN Form B

In some embodiments, a gentisic salt of a compound of Formula (I):

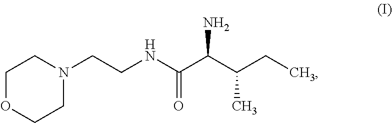 [SMILES: CC[C@H](C)[C@H](N)C(=O)NCCN1CCOCC1]

is provided and/or utilized in a crystalline form. In some embodiments, a particular crystalline form may be detected, for example in a composition, by detection of a set of characteristic peaks.

In some embodiments, such a crystalline form exhibits an X-ray powder diffraction pattern comprising peaks at the following diffraction angles (2θ): 18.6±0.2, 18.9±0.2, and 19.5±0.2. In some embodiments, such a crystalline form exhibits an X-ray powder diffraction pattern comprising peaks at the following diffraction angles (2θ): 17.0±0.2, 18.6±0.2, 18.9±0.2, 19.5±0.2, and 20.6±0.2.

In some embodiments, the crystalline form exhibits an X-ray powder diffraction pattern comprising peaks at the following diffraction angles (2θ): 7.7±0.2, 16.3±0.2, 16.7±0.2, 17.0±0.2, 18.6±0.2, 18.9±0.2, 19.5±0.2, 20.6±0.2, 21.2±0.2, and 22.8±0.2.

In some embodiments, the crystalline form exhibits an X-ray powder diffraction pattern comprising some peaks selected from Table 3.

TABLE 3
XRPD Peak List of GEN Form B

| | Position | Intensity | |
|---|---|---|---|
| Peak | (°2-theta) | (Counts) | Rel. Int. (%) |
| 1 | 4.6 | 2370.0 | 32.2 |
| 2 | 5.9 | 3017.0 | 41.0 |
| 3 | 6.9 | 1531.0 | 20.8 |
| 4 | 7.7 | 5467.0 | 74.3 |
| 5 | 8.4 | 1557.0 | 21.1 |
| 6 | 8.9 | 1670.0 | 22.7 |
| 7 | 9.5 | 1752.0 | 23.8 |
| 8 | 10.3 | 1781.0 | 24.2 |
| 9 | 10.8 | 1631.0 | 22.2 |
| 10 | 12.3 | 1875.0 | 25.5 |
| 11 | 15.2 | 2672.0 | 36.3 |
| 12 | 15.6 | 3119.0 | 42.4 |
| 13 | 15.9 | 3638.0 | 49.4 |
| 14 | 16.3 | 3949.0 | 53.6 |
| 15 | 16.7 | 4796.0 | 65.1 |
| 16 | 17.0 | 6091.0 | 82.7 |
| 17 | 18.6 | 6429.0 | 87.3 |
| 18 | 18.9 | 6891.0 | 93.6 |
| 19 | 19.5 | 7362.0 | 100.0 |
| 20 | 20.6 | 6234.0 | 84.7 |
| 21 | 21.2 | 4710.0 | 64.0 |
| 22 | 22.8 | 3906.0 | 53.1 |
| 23 | 23.1 | 2954.0 | 40.1 |
| 24 | 23.4 | 2797.0 | 38.0 |
| 25 | 23.6 | 2794.0 | 38.0 |

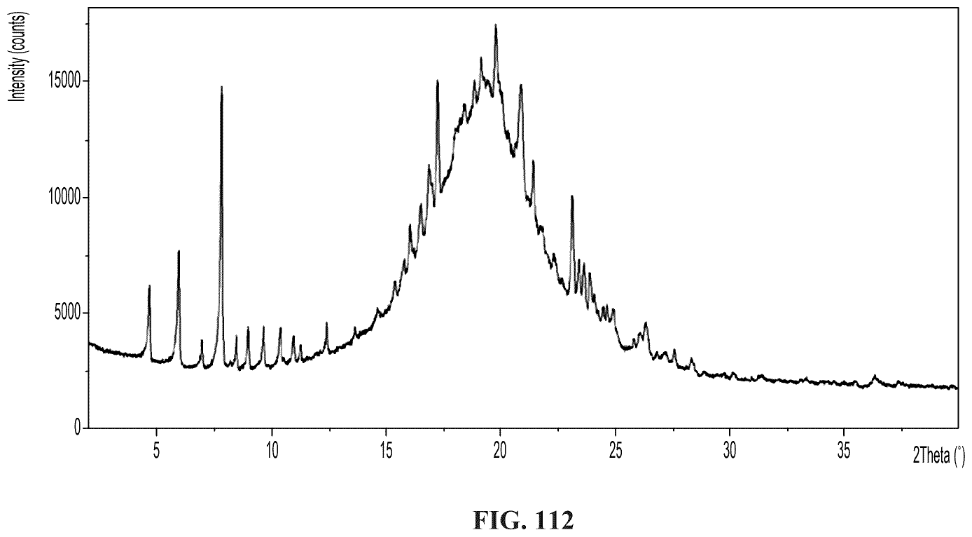

In some embodiments, the crystalline form exhibits an X-ray powder diffraction pattern substantially the same as depicted in FIG. 112.

### Synthesis

Procedures for making compounds described herein are provided below. Starting materials used in the following schemes can be purchased or prepared by methods described in the chemical literature, or by adaptations thereof, using methods known by those skilled in the art. The order in which the steps are performed can vary depending on the groups introduced and the reagents used, but would be apparent to those skilled in the art.

In some embodiments, a procedure for compounds described herein may include one or more steps of recrystallizing which compounds, for example, may include: combining the crystalline compound with a solvent thereby forming a suspension; heating the suspension thereby forming a clear mixture; and cooling the clear mixture thereby forming a precipitate. In some embodiments, the solvent is 2-methyltetrahydrofuran or isopropyl acetate. In some embodiments, the solvent is 2-methyltetrahydrofuran. In some embodiments, the ratio of crystalline compound to solvent is about 50 mg of the crystalline compound to 0.5 mL of the solvent. In some embodiments, the suspension is heated from 25° C. to 80° C. In some embodiments, the suspension is cooled from 80° C. to 25° C.

### Pharmaceutical Compositions

Those skilled in the art are aware of a variety of technologies useful in formulating pharmaceutical compositions, for example using one or more physiologically acceptable carriers including excipients and auxiliaries which facilitate processing of the active compounds into preparations which can be used pharmaceutically. Those skilled in the art will appreciate that route of administration influences nature of formulation, and components included. Exemplary discussion of available excipients typically utilized for pharmaceutical compositions may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure

Pharmaceutical compositions including a compound described herein may be manufactured using technologies, such as, by way of example only, mixing, dissolving, granulating, dragee-making, levigating, emulsifying, encapsulating, entrapping or compression processes.

In many embodiments, pharmaceutical compositions will include and/or will deliver at least one compound of Formulas (III), (I), (Ia), or (II) described herein, as an active ingredient in free-base form, or in a pharmaceutically acceptable salt form.

Those skilled in the art will appreciate that compounds such as those of Formulas (III), (I), (Ia), or (II), or pharmaceutically acceptable salts thereof, described herein can typically adopt one or more crystalline forms (also known as polymorphs); in many embodiments, such crystalline forms are utilized in unit dosage forms as described herein, and/or are used in manufacturing such unit dosage forms, and/or for storage of active, e.g., prior to formulation into a unit dosage form. Moreover, those skilled in the art will further appreciate that, in some embodiments, compounds such as those of Formulas (III), (I), (Ia), or (II), or pharmaceutically acceptable salts thereof, may have active metabolites that share the same type of activity as observed for the original “parent” compound. In some embodiments, a compound utilized in accordance with the present disclosure (e.g., included in and/or delivered by a pharmaceutical composition in accordance with the present disclosure) may be a metabolite of a compound of Formulas (III), (I), (Ia), or (II), or pharmaceutically acceptable salts thereof.

In some situations, compounds may exist as tautomers. All tautomers are included within the scope of the compounds presented herein.

Additionally, those skilled in the art will appreciate that compounds described herein can exist in unsolvated as well as solvated forms with pharmaceutically acceptable solvents such as water, ethanol, and the like. In some embodiments, one or more p75^{NTR} modulator compounds as described herein is utilized in a solvated form.

In certain embodiments, compositions utilized in accordance with the present disclosure may also include one or more preservatives to inhibit microbial activity. Suitable preservatives include quaternary ammonium compounds such as benzalkonium chloride, cetyltrimethylammonium bromide and cetylpyridinium chloride.

Pharmaceutical preparations for oral use can be obtained in some embodiments by mixing one or more solid excipients with one or more of the compounds described herein, e.g., compounds of Formulas (III), (I), (Ia), or (II), optionally grinding the resulting mixture, and processing the mixture of granules, after adding suitable auxiliaries, if desired, to obtain tablets, pills, or capsules. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include, for example, push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

In some embodiments, solid dosage forms for use in accordance with the present disclosure may be in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. Additionally, pharmaceutical formulations of the compounds described herein may be administered as a single capsule or in multiple capsule dosage form. In some embodiments, the pharmaceutical composition is administered in two, or three, or four, capsules or tablets.

In some embodiments, solid dosage forms, e.g., tablets, effervescent tablets, and capsules, are prepared by mixing particles of a compound of Formulas (III), (I), (Ia), or (II) described herein (optionally in a pharmaceutically acceptable salt form), with one or more pharmaceutical excipients to form a bulk blend composition. When referring to these bulk blend compositions as homogeneous, it is meant that the particles of the compound of Formulas (III), (I), (Ia), or (II) described herein, are dispersed evenly throughout the composition so that the composition may be subdivided into equally effective unit dosage forms, such as tablets, pills, and capsules. Individual unit dosages may also include film coatings, which disintegrate upon oral ingestion or upon contact with diluent.

In some embodiments a pharmaceutical composition for use in accordance with the present disclosure may contain less than 10 wt % of excipients. In some embodiments a pharmaceutical composition may contain less than 5 wt % of excipients, such as less than 4 wt % of excipients, such as less than 3 wt % of excipients, such as less than 2 wt % of excipients. In certain embodiments, a pharmaceutical composition contains less than 2 wt % of excipients. In some embodiments, a pharmaceutical composition consists essentially of a compound of Formulas (III), (I), (Ia), or (II), which, as already described herein, may be in a pharmaceutically acceptable salt form.

The disclosure provides for a pharmaceutical composition, wherein the composition comprises from about 10 milligram (mg) (free base weight) to about 1,000 mg (free base weight) of a salt of Formulas (III), (I), (Ia), or (II). The disclosure further provides for a pharmaceutical composition, wherein the composition comprises from about 10 mg (free base weight) to about 800 mg (free base weight) of a salt of Formulas (III), (I), (Ia), or (II). The disclosure further provides for a pharmaceutical composition, wherein the composition comprises from about 10 mg (free base weight) to about 600 mg (free base weight) of a salt of Formulas (III), (I), (Ia), or (II). The disclosure further provides for a pharmaceutical composition, wherein the composition comprises from about 10 mg (free base weight) to about 500 mg (free base weight) of a salt of Formulas (III), (I), (Ia), or (II).

As referred to herein, the “free base weight” refers to a calculated mass of the free base, based upon the mass of the salt in the composition. For example, to obtain a pharmaceutical composition with 200 mg (free base weight) of the salt of Formula (II), 334 mg of the salt of Formula (II) is added to the composition. The following equation may be used to calculate the weight of the free base from the weight of the salt of Formula (II):

$$( weight ( g ) of salt of Formula ( II ) ) \div ( MW ( g / mol ) of salt of Formula ( II ) ) \times 3 \times ( MW of free base ) = weight of the free base$$

MW of salt of Formula (II): 1220.43 g/mol
MW of free base (compound of Formula I): 243.35
   Methods of Treating, Diagnosing, or Characterizing Diseases Associated with Phosphorylated Tau Proteins

In various embodiments, the present disclosure provides methods for diagnosing, characterizing, prognosing, preventing, treating, ameliorating, managing, delaying onset, or slowing progression of diseases associated with tau protein phosphorylated, e.g., at threonine 217 (p-tau217), at a leucine at amino acid 243 (p-tau243), at a threonine at amino acid 181 (p-tau181), and/or at a threonine at amino acid 231 (p-tau231).

As described herein, the present disclosure establishes that modulation of p75^{NTR} can impact one or more tau pathology biomarkers, specifically including phosphorylated tau proteins characterized by p-tau217, p-tau243, p-tau181, and/or p-tau231. In various embodiments, the present disclosure provides methods for diagnosing, characterizing, prognosing, preventing, treating, ameliorating, managing, delaying onset, or slowing progression of diseases identified as having a level (e.g., a plasma level) of tau protein phosphorylated at, e.g., p-tau217, p-tau243, p-tau181, and/or p-tau231, that is indicative of the presence of Alzheimer's Disease pathology in the brain and/or of likely responsiveness to a p75^{NTR} modulator (e.g., indicative the subject would benefit from treatment with the p75^{NTR} modulator). In various embodiments, the present disclosure provides methods for diagnosing, characterizing, prognosing, preventing, treating, ameliorating, managing, delaying onset, or slowing progression of diseases identified as having a high level (e.g., a plasma level) of tau protein phosphorylated at, e.g., p-tau217, p-tau243, p-tau181, and/or p-tau231. In various embodiments, the present disclosure provides a method of reducing a level of p-tau217 or p-tau243 in a subject, the method comprising administering to the subject an effective amount of a of a p75^{NTR} modulator. In some embodiments, the method additionally or alternatively comprises reducing a level of p-tau181, and/or p-tau231 in the subject, the method comprising administering to the subject an effective amount of a of a p75^{NTR} modulator.

Levels (e.g., plasma levels) of p-tau217 have been associated with amyloid pathology in the brain of patients with Alzheimer's Disease, but has generally been considered a passenger effect of amyloid pathology. In some embodiments, a subject of a method of the present disclosure does not have amyloid pathology. The present disclosure is based, in part, on the surprising discovery that p-tau217, among other tau phosphorylation sites (e.g., p-tau243, p-tau181, and/or p-tau231), can be targeted in the treatment of disease (e.g., Alzheimer's Disease) independent of amyloid pathology. The present disclosure is also based, in part, on the surprising discovery that plasma p-tau217 levels can be reduced by treatment with a p75^{NTR} modulator (e.g., a compound of Formula (Ia)) that is not targeted to lowering levels of brain amyloid accumulation.

In some embodiments, a subject has a level of plasma p-tau217 that is indicative of the presence of Alzheimer's Disease pathology in the brain, and/or of likely responsiveness to a p75^{NTR} modulator (e.g., indicative the subject would benefit from treatment with the p75^{NTR} modulator), prior to treatment. In some embodiments, a method of treatment comprises steps of (a) obtaining or having obtained (e.g., via a third-party) a sample from a patient; (b) measuring or having measured levels of plasma p-tau217 in the patient; and (c) if the subject has a high level of p-tau217, then administering an effective amount of a p75^{NTR} modulator (e.g., a compound of Formula (Ia)) to the subject. In some embodiments, levels of plasma p-tau217 are measured over the course of treatment to assess disease progression and/or to assess effectiveness of administered therapy (e.g., with a p75^{NTR} modulator).

In some embodiments, a subject has a level of plasma p-tau243 that is indicative of the presence of Alzheimer's Disease pathology in the brain, and/or of likely responsiveness to a p75^{NTR} modulator (e.g., indicative the subject would benefit from treatment with the p75^{NTR} modulator), prior to treatment. In some embodiments, a method of treatment comprises steps of (a) obtaining or having obtained (e.g., via a third-party) a sample from a patient; (b) measuring or having measured levels of plasma p-tau243 in the patient; and (c) if the subject has a high level of p-tau243, then administering an effective amount of a p75^{NTR} modulator (e.g., a compound of Formula (Ia)) to the subject. In some embodiments, levels of plasma p-tau243 are measured over the course of treatment to assess disease progression and/or to assess effectiveness of administered therapy (e.g., with a p75^{NTR} modulator).

In some embodiments, a subject has a level of plasma p-tau181 that is indicative of the presence of Alzheimer's Disease pathology in the brain, and/or of likely responsiveness to a p75^{NTR} modulator (e.g., indicative the subject would benefit from treatment with the p75^{NTR} modulator), prior to treatment. In some embodiments, a method of treatment comprises steps of (a) obtaining or having obtained (e.g., via a third-party) a sample from a patient; (b) measuring or having measured levels of plasma p-tau181 in the patient; and (c) if the subject has a high level of p-tau181, then administering an effective amount of a p75^{NTR} modulator (e.g., a compound of Formula (Ia)) to the subject. In some embodiments, levels of plasma p-tau181 are measured over the course of treatment to assess disease progression and/or to assess effectiveness of administered therapy (e.g., with a p75^{NTR} modulator).

In some embodiments, a subject has a level of plasma p-tau231 that is indicative of the presence of Alzheimer's Disease pathology in the brain, and/or of likely responsiveness to a p75^{NTR} modulator (e.g., indicative the subject would benefit from treatment with the p75^{NTR} modulator), prior to treatment. In some embodiments, a method of treatment comprises steps of (a) obtaining or having obtained (e.g., via a third-party) a sample from a patient; (b) measuring or having measured levels of plasma p-tau231 in the patient; and (c) if the subject has a high level of p-tau231, then administering an effective amount of a p75^{NTR} modulator (e.g., a compound of Formula (Ia)) to the subject. In some embodiments, levels of plasma p-tau231 are measured over the course of treatment to assess disease progression and/or to assess effectiveness of administered therapy (e.g., with a p75^{NTR} modulator).

In various embodiments, the present disclosure provides methods of determining Alzheimer's Disease activity in a subject. “Determining Alzheimer's Disease activity” can include, e.g., diagnosing, prognosing, monitoring, or characterizing Alzheimer's Disease in the subject. In some embodiments, a provided method of determining Alzheimer's Disease activity in a subject comprises: obtaining or having obtained a biological sample from the subject; measuring or having measured a level of p-tau217; generating a prediction of AD progression, wherein a high level of p-tau217 is indicative of a progression of AD. In some embodiments, the level of p-tau217 is not correlated with amyloid pathology.

In some embodiments, a provided method of determining Alzheimer's Disease activity in a subject comprises: obtaining or having obtained a biological sample from the subject; measuring or having measured a level of p-tau243; generating a prediction of AD progression, wherein a high level of p-tau243 is indicative of a progression of AD. In some embodiments, the level of p-tau243 is not correlated with amyloid pathology.

In some embodiments, a provided method of determining Alzheimer's Disease activity in a subject comprises: obtaining or having obtained a biological sample from the subject; measuring or having measured a level of p-tau181; generating a prediction of AD progression, wherein a high level of p-tau181 is indicative of a progression of AD. In some embodiments, the level of p-tau181 is not correlated with amyloid pathology.

In some embodiments, a provided method of determining Alzheimer's Disease activity in a subject comprises: obtaining or having obtained a biological sample from the subject; measuring or having measured a level of p-tau231; generating a prediction of AD progression, wherein a high level of p-tau231 is indicative of a progression of AD. In some embodiments, the level of p-tau231 is not correlated with amyloid pathology.

### Measurement of Phosphorylated Tau Proteins

Levels of phosphorylated tau proteins (e.g., p-tau217, p-tau243, p-tau181, and/or p-tau231) can be measured by any suitable method known in the art. In some embodiments, a level of phosphorylated tau proteins (e.g., p-tau217, p-tau243, p-tau181, and/or p-tau231) is measured in a biological sample selected from the group consisting of a plasma sample, a serum sample, a whole blood sample, or a cerebrospinal fluid sample. In some embodiments, the biological sample is a plasma sample. In some embodiments, the biological sample is a serum sample. In some embodiments, the biological sample is a whole blood sample. In some embodiments, the biological sample is a cerebrospinal fluid sample.

In some embodiments, levels of phosphorylated tau proteins (e.g., p-tau217, p-tau243, p-tau181, and/or p-tau231) are measured as a proportion relative to total tau protein. In some embodiments, level of phosphorylated tau proteins (e.g., p-tau217, p-tau243, p-tau181, and/or p-tau231) is measured as an absolute value (e.g., a concentration).

In some embodiments, levels of phosphorylated tau proteins (e.g., p-tau217, p-tau243, p-tau181, and/or p-tau231) are measured by a method comprising Lumipulse, Elecsys, Simoa, ELISA, immunoblot, immunoprecipitation, electrochemiluminescence (e.g., a Meso Scale Discovery assay or an S-PLEX assay), magnetic pull-down, or liquid chromatography-mass spectrometry. In some embodiments, levels of phosphorylated tau proteins (e.g., p-tau217, p-tau243, p-tau181, and/or p-tau231) are measured by a method comprising Lumipulse. In some embodiments, levels of phosphorylated tau proteins (e.g., p-tau217, p-tau243, p-tau181, and/or p-tau231) are measured by a method comprising Elecsys. In some embodiments, levels of phosphorylated tau proteins (e.g., p-tau217, p-tau243, p-tau181, and/or p-tau231) are measured by a method comprising Simoa. In some embodiments, levels of phosphorylated tau proteins (e.g., p-tau217, p-tau243, p-tau181, and/or p-tau231) are measured by a method comprising ELISA. In some embodiments, levels of phosphorylated tau proteins (e.g., p-tau217, p-tau243, p-tau181, and/or p-tau231) are measured by a method comprising immunoblot. In some embodiments, levels of phosphorylated tau proteins (e.g., p-tau217, p-tau243, p-tau181, and/or p-tau231) are measured by a method comprising immunoprecipitation. In some embodiments, levels of phosphorylated tau proteins (e.g., p-tau217, p-tau243, p-tau181, and/or p-tau231) are measured by a method comprising electrochemiluminescence (e.g., a Meso Scale Discovery assay or an S-PLEX assay). In some embodiments, levels of phosphorylated tau proteins (e.g., p-tau217, p-tau243, p-tau181, and/or p-tau231) are measured by a method comprising magnetic pull-down. In some embodiments, levels of phosphorylated tau proteins (e.g., p-tau217, p-tau243, p-tau181, and/or p-tau231) are measured by a method comprising liquid chromatography-mass spectrometry. Additional details on exemplary methods for measuring levels of phosphorylated tau proteins (e.g., p-tau217, p-tau243, p-tau181, and/or p-tau231) can be found in Meyer et al. Alzheimers Dement. 2024 May; 20(5):3179-3192; Quanterix Granted Breakthrough Device Designation from U.S. FDA for Blood-Based p-Tau 217 Test for Alzheimer's Disease. Quanterix. Published Jan. 30, 2024 (found at: quanterix.com/press-releases/quanterix-granted-breakthrough-device-designation-from-u-s-fda-for-blood-based-p-tau-217-test-for-alzheimers-disease/); and alzheimersnewstoday.com/news/biomarker-test-diagnose-alzheimers-wins-fda-breakthrough-designation).

A “level of plasma phosphorylated tau protein (e.g., p-tau217, p-tau243, p-tau181, or p-tau231) that is indicative or the presence of Alzheimer's Disease pathology in the brain and/or of likely responsiveness to a p75^{NTR} modulator” or a “high” level of phosphorylated tau protein (e.g., p-tau217, p-tau243, p-tau181, or p-tau231) refers to a level of phosphorylated tau protein (e.g., p-tau217, p-tau243, p-tau181, or p-tau231) at or above a cutoff level that is or has been previously associated with Alzheimer's Disease pathology in the brain and/or of likely responsiveness to a p75^{NTR} modulator (e.g., indicative the subject would benefit from treatment with the p75^{NTR} modulator). In some embodiments, the Alzheimer's Disease pathology in the brain is determined by amyloid PET scan and/or CSF sample.

Cutoff levels for plasma p-tau217 can depend, e.g., on the measurement method and whether the measurement is relative to total tau protein or is an absolute value. In some embodiments, a level of p-tau217 (e.g., a plasma level) of at least 4.2% p-tau217 relative to total tau or an analogous level in blood or CSF is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a level (e.g., a plasma level) of p-tau217 of at least 4.2% p-tau217 relative to total tau, e.g., as measured by mass spectrometry, or an analogous level in blood or CSF is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator. In some embodiments, a level (e.g., a plasma level) of p-tau217 of at least 7.3 pg/mL, at least 0.27 pg/mL, at least 0.273 pg/mL, at least 0.399 pg/mL, at least 0.180 pg/mL, e.g., as measured by an electrochemiluminescence immunoassay (e.g., an S-PLEX assay), or an analogous level in blood or CSF is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator. In some embodiments, a level of p-tau217 (e.g., a plasma level) of at least 7.3 pg/mL, as measured by an electrochemiluminescence immunoassay (e.g., an S-PLEX assay), or an analogous level in blood or CSF is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator. In some embodiments, a level (e.g., a plasma level) of p-tau217 of at least 0.27 pg/mL, as measured by an electrochemiluminescence immunoassay (e.g., a Meso Scale Discovery assay), or an analogous level in blood or CSF is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator. In some embodiments, a level (e.g., a plasma level) of p-tau217 of at least 0.273 pg/mL, as measured by an electrochemiluminescence immunoassay (e.g., a Meso Scale Discovery assay), or an analogous level in blood or CSF is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator. In some embodiments, a level (e.g., a plasma level) of p-tau217 of at least 0.399 pg/mL, as measured by an electrochemiluminescence immunoassay (e.g., a Meso Scale Discovery assay), or an analogous level in blood or CSF is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator. In some embodiments, a level of p-tau217 (e.g., a plasma level) that is indicative of the presence of Alzheimer's Disease pathology in the brain and/or of likely responsiveness to a p75^{NTR} modulator (e.g., indicative the subject would benefit from treatment with the p75^{NTR} modulator) is at least 0.180 pg/mL (e.g., as measured by Lumipulse) or an analogous level in blood or CSF. In some embodiments, a decrease in this level can indicate responsiveness to a p75^{NTR} modulator. In certain embodiments, a plasma ratio of p-tau217 to plasma β-amyloid (1-42 (“Aβ42”) of at least 0.00738, e.g., as measured by Lumipulse G pTau 217/0-Amyloid 1-42 Plasma Ratio assay, or an analogous level in blood or CSF is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator. In certain embodiments, a plasma ratio of p-tau217 to plasma β-amyloid (1-42 (“Aβ42”) of at least 0.00371, e.g., as measured by Lumipulse G pTau 217/0-Amyloid 1-42 Plasma Ratio assay, or an analogous level in blood or CSF is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator. Further details on the level of p-tau217 that is indicative or the presence of Alzheimer's Disease pathology in the brain can be found, e.g., in Meyer et al. Alzheimers Dement. 2024 May; 20(5):3179-3192; Kivisakk et al. Sci Rep. 2024 Jan. 5; 14(1):629; and
Howe et al. Alzheimers Res Ther. 2024 Jul. 6; 16(1):154.

In some embodiments, a plasma level of p-tau243 of at least 0.2 pg/mL, e.g., as measured by Quanterix Simoa® immunoassay, or an analogous level in blood or CSF is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator.

In some embodiments, a CSF ratio of p-tau181 to CSF Aβ42″ of at least 0.037 to at least 0.09 (e.g., at least 0.037, 0.038, 0.039, 0.040, 0.041, 0.042, 0.043, 0.044, 0.045, 0.046, 0.047, 0.048, 0.049, 0.050, 0.051, 0.052, 0.053, 0.054, 0.055, 0.056, 0.057, 0.058, 0.059, 0.060, 0.061, 0.062, 0.063, 0.064, 0.065, 0.066, 0.067, 0.068, 0.069, 0.070, 0.071, 0.072, 0.073, 0.074, 0.075, 0.076, 0.077, 0.078, 0.079, 0.080, 0.081, 0.082, 0.083, 0.084, 0.085, 0.086, 0.087, 0.088, 0.089, or 0.090), e.g., as measured by Elecsys® Phospho-Tau (181P) CSF assay, or an analogous level in blood or plasma is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator. In certain embodiments, a CSF ratio of p-tau181 to CSF Aβ42 of at least 0.037, e.g., as measured by Elecsys® Phospho-Tau (181P) CSF assay, or an analogous level in blood or plasma is indicative of the presence of Alzheimer's Disease pathology in the brain and/or of likely responsiveness to a p75^{NTR} modulator. In some embodiments, a decrease in this level can indicate responsiveness to a p75^{NTR} modulator. In certain embodiments, a CSF ratio of p-tau181 to CSF Aβ42 of at least 0.09, e.g., as measured by Elecsys® Phospho-Tau (181P) CSF assay, or an analogous level in blood or plasma is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator. In some embodiments, a plasma level of p-tau181 of at least 0.934 pg/mL, e.g., as measured by Elecsys® Phospho-Tau (181P) Plasma assay, or an analogous level in blood or CSF is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator.

In some embodiments, a plasma level of p-tau231 of at least 0.2 pg/mL, e.g., as measured by Quanterix Simoa® immunoassay, or an analogous level in blood or CSF is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator.

In some embodiments, a plasma level of p-tau243 that is above a cutoff value, e.g., as measured using established methods in the art, or an analogous level in blood or CSF is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator. In some embodiments, a CSF level of p-tau243 that is above a cutoff value, e.g., as measured using established methods in the art, or an analogous level in blood or plasma is indicative of the presence of Alzheimer's Disease pathology in the brain and/or of likely responsiveness to a p75^{NTR} modulator (e.g., indicative the subject would benefit from treatment with the p75^{NTR} modulator). In some embodiments, a decrease in this level can indicate responsiveness to a p75^{NTR} modulator.

### Diseases

A primary focus of the present disclosure is treatment of Alzheimer's Disease (e.g., identification of subjects suffering from or susceptible to Alzheimer's Disease who might be responsive to therapy with a p75^{NTR} modulator and/or monitoring impact of such therapy).

In some embodiments, the disease is Alzheimer's Disease in a subject in need thereof. (e.g., mild to moderate, or preclinical stages such as Mild Cognitive Impairment (MCI) due to underlying Alzheimer's Disease pathology). Compounds and salts described herein can be used to modulate p75^{NTR} or for the treatment of diseases or conditions that would benefit, at least in part, from modulation of p75^{NTR} activity. In addition, a method for treating any of the diseases or conditions described herein in a subject in need of such treatment, may involve (e.g., oral) administration of pharmaceutical compositions that contain or deliver at least one compound described herein, or a prodrug or active metabolite thereof, any of which may be utilized in a pharmaceutically acceptable salt and/or solvate form. The subject may exhibit or be determined to exhibit mild cognitive impairment due to underlying Alzheimer's Disease pathology, or mild to moderate Alzheimer's Disease according to McKhann (McKhann et al., Alzheimers Dement. 7(3):263-9 (2011)) criteria, e.g., within six months prior to the administration. In other cases, the subject may exhibit or be determined to be in preclinical or earlier clinical stages of the disease. In general, provided pharmaceutical compositions (e.g., that comprise and/or deliver a compound of one of Formulas (III), (I), (Ia), or (II), a prodrug or active metabolite thereof, any of which may be utilized in a pharmaceutically acceptable salt or solvate form can be administered for prophylactic and/or therapeutic treatments.

In therapeutic applications, pharmaceutical compositions suitable for use with the disclosed methods are administered to a patient already suffering from a disease or condition, in an amount effective to treat or at least partially arrest the symptoms or underlying degeneration and progression of the disease or condition. Amounts effective for this use will depend on the severity and course of the disease or condition, previous therapy, the patient's health status, weight, and response to the drugs, and the judgment of the treating physician.

In prophylactic applications, including those intended to delay onset of clinical symptoms such as loss of cognitive function, pharmaceutical compositions suitable for use with the disclosed methods are administered to a patient susceptible to or otherwise at risk of a particular disease, disorder or condition. An amount sufficient to delay onset of and/or reduce severity of one or more features of a relevant disease, disorder, or condition is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician.

In some embodiments of any one of the methods described herein, the method described may be used to treat or prevent Alzheimer's Disease in subjects with particular characteristics. For example, methods of the disclosure may be used to treat subjects who exhibit mild to moderate Alzheimer's Disease according to McKhann (2011) criteria within six months prior to said administration. Other subjects might be in earlier stages such as preclinical or prodromal stages. Methods of the disclosure may be used to treat, delay onset or prevent Alzheimer's Disease in subjects from 50 to 100 years old, such as from 50 to 90 years old, such as from 65 to 90 years old. Methods of the disclosure may be used to treat, delay onset, manage, or prevent Alzheimer's Disease in subjects exhibiting or determined to exhibit an P4 allele of apolipoprotein E (ApoE) gene (ApoE4). Methods of the disclosure may be used to treat, manage, or prevent Alzheimer's Disease in subjects not exhibiting or determined to not exhibit an P4 allele of apolipoprotein E (ApoE) gene (ApoE4).

In some embodiments of any one of the methods described herein, the method described may provide or result in a slowing of deterioration of, in an improvement of, a value of one or more Alzheimer's Disease metrics relative to a baseline value measured at or within six months (e.g., within three months) preceding said administration. The Alzheimer's Disease metrics may be selected from anatomical or statistical regional or voxel-based brain glucose metabolism (¹⁸F-FDG-PET) rate, magnetic resonance imaging (MRI) structural or voxel-based or volumetric imaging, cerebrospinal Alzheimer's Disease-relevant biomarker levels, blood or plasma biomarkers and performance on the cognitive testing methods such as ADAS-cog, MMSE and other cognitive testing approaches.

In some embodiments of any one of the methods described herein, methods of the disclosure reduce or prevent loss of certain brain region volumes in said subject. In some embodiments, the brain region volume comprises one or more of a hippocampus volume, basal forebrain volume, lingual gyrus volume, parahippocampal gyrus, and orbitofrontal cortex, parietal cortex and cingulate cortex in said subject. In some embodiments volume of lateral ventricles in measured as an indicator of general brain volume; increased size of lateral ventricular volume is an indicator of diffuse loss of brain parenchymal volume. In some embodiments, the brain region volume comprises other brain region volumes in said subject. Such volumes could be derived from whole brain or targeted region voxel wise or voxel-based analyses.

In some embodiments of any one of the methods described herein, methods of the disclosure reduce or prevent loss of hippocampus volume or loss or changes in other brain region or voxel-based volumes in said subject. In certain embodiments, methods of the disclosure reduce or prevent or mitigate an increase in certain species of amyloid beta (Aβ) level such as Aβ42 (e.g., in a bodily fluid, such as a cerebrospinal spinal fluid or blood). In certain embodiments, methods of the disclosure lead to a reduction in fluid levels of Aβ42, 40 or other species. In certain embodiments, methods of the disclosure reduce or prevent or mitigate an increase in tau or modified versions of tau level (e.g., in a bodily fluid, such as a cerebrospinal spinal fluid or blood or plasma).

In certain embodiments, methods of the disclosure reduce or prevent or mitigate an increase in biomarkers of neuronal or synaptic degeneration such as neurofilament light chain, SNAP-25, synaptotagmin-1, neurogranin; or in biomarkers of inflammation or gliosis such as sTREM or YKL-40.

In some embodiments, the disclosure of the present application provides a method for modulating one or more neurodegeneration biomarkers or indicators in a subject in need thereof, the method comprising: administering to said subject an effective amount of a p75^{NTR} modulator or a pharmaceutically acceptable salt thereof (such as described herein) to provide one or more of the following in said subject: (i) an amyloid beta (Aβ) level (e.g., in a bodily fluid) that is lower than a corresponding reference; (ii) a change (e.g., increase) or a rate of change (e.g., a rate of increase) in an AP level (e.g., in a bodily fluid) over a duration that is lower than a corresponding reference over the same duration; (iii) a change (e.g., increase) or a rate of change (e.g., a rate of increase) in a tau (τ) level (e.g., in a bodily fluid) over a duration that is lower than a corresponding reference over the same duration; (iv) a change (e.g., increase) or a rate of change (e.g., a rate of increase) in a presynaptic biomarker level (e.g., in a bodily fluid) over a duration that is lower than a corresponding reference over the same duration; (v) a change (e.g., increase) or a rate of change (e.g., a rate of increase) in a postsynaptic biomarker level (e.g., in a bodily fluid) over a duration that is lower than a corresponding reference over the same duration; (vi) a change (e.g., increase) or a rate of change (e.g., a rate of increase) in a glial marker level (e.g., in a bodily fluid) over a duration that is lower than a corresponding reference over the same duration; (vii) a rate of volume change of a brain region over a duration that is lower than a corresponding reference rate (e.g., in an untreated control) over the same duration as determined by magnetic resonance imaging (MRI) imaging; (viii) a brain glucose metabolism (¹⁸F-FDG-PET) rate that is lower than a corresponding reference rate (e.g., in an untreated control); and (ix) a change in Alzheimer's Disease Assessment Scale-Cognitive (ADAS-Cog) score over a duration that is less than a corresponding change (e.g., in an untreated control) over the same duration.

In some embodiments, the disclosure of the present application provides a method for modulating one or more neurodegeneration biomarkers or indicators in a subject in need thereof, the method comprising: administering to said subject an effective amount of a p75^{NTR} modulator or a pharmaceutically acceptable salt thereof to provide an amyloid beta (Aβ) level (e.g., in a bodily fluid) in said subject that is lower than a corresponding reference. In some embodiments, the Aβ is Aβ40 or Aβ42. In some embodiments, the Aβ is Aβ40. In some embodiments, the Aβ is Aβ42. In some embodiments, the method provides an AP level (e.g., in said bodily fluid) in said subject that is lower by at least about 1%, at least about 2%, at least about 3%, at least about 4%, or at least about 5% than said corresponding reference. In some embodiments, the corresponding reference is a corresponding pretreatment level said subject (e.g., in said bodily fluid). In some embodiments, the corresponding reference is determined from an untreated control.

In some embodiments, the disclosure of the present application provides a method for modulating one or more neurodegeneration biomarkers or indicators in a subject in need thereof, the method comprising: administering to said subject an effective amount of a p75^{NTR} modulator or a pharmaceutically acceptable salt thereof to provide a change (e.g., increase) or a rate of change (e.g., a rate of increase) in an AP level (e.g., in a bodily fluid) over a duration that is lower than a corresponding reference over the same duration. In some embodiments, the Aβ is Aβ40 or Aβ42. In some embodiments, the Aβ is Aβ40. In some embodiments, the Aβ is Aβ42. In some embodiments, the method provides a change (e.g., increase) or a rate of change (e.g., a rate of increase) in an AP level (e.g., in a bodily fluid) over a duration that is lower by at least about 1%, at least about 2%, at least about 3%, at least about 4%, or at least about 5% than a corresponding reference over the same duration. In some embodiments, the corresponding reference is a corresponding pretreatment level said subject (e.g., in said bodily fluid). In some embodiments, the corresponding reference is determined from an untreated control.

In some embodiments, the disclosure of the present application provides a method for modulating one or more neurodegeneration biomarkers or indicators in a subject in need thereof, the method comprising: administering to said subject an effective amount of a p75^{NTR} modulator or a pharmaceutically acceptable salt thereof to provide a change (e.g., increase) or a rate of change (e.g., a rate of increase) in a tau (τ) level (e.g., in a bodily fluid) over a duration that is lower than a corresponding reference over the same duration. In some embodiments, the tau comprises unphosphorylated tau. In some embodiments, the tau comprises phosphorylated tau. In some embodiments, the bodily fluid is a cerebrospinal spinal fluid, blood, serum, or plasma. In some embodiments, the bodily fluid is a cerebrospinal spinal fluid. In some embodiments, the bodily fluid is blood. In some embodiments, the bodily fluid is serum. In some embodiments, the bodily fluid is plasma. In some embodiments, the method provides a tau (τ) level (e.g., in said bodily fluid) in said subject that as a rate of increase lower by at least about 1, at least about 2, at least about 3, at least about 4, or at least about 5 annual percent change (APC)) than said corresponding reference. In some embodiments, the corresponding reference is a corresponding pretreatment level (e.g., in said bodily fluid). In some embodiments, the corresponding reference is determined from an untreated control.

In some embodiments, the disclosure of the present application provides a method for modulating one or more neurodegeneration biomarkers or indicators in a subject in need thereof, the method comprising: administering to said subject an effective amount of a p75^{NTR} modulator or a pharmaceutically acceptable salt thereof to provide a change (e.g., increase) or a rate of change (e.g., a rate of increase) in a presynaptic biomarker level (e.g., in a bodily fluid) over a duration that is lower than a corresponding reference over the same duration. In some embodiments, the presynaptic biomarker is selected from Synaptosome Associated Protein 25 (SNAP25), and Synaptotagmin 1 (SYT1). In some embodiments, the presynaptic biomarker is SNAP25. In some embodiments, the presynaptic biomarker is SYT1. In some embodiments, the bodily fluid is a cerebrospinal spinal fluid, blood, serum, or plasma. In some embodiments, the bodily fluid is a cerebrospinal spinal fluid. In some embodiments, the bodily fluid is blood. In some embodiments, the bodily fluid is serum. In some embodiments, the bodily fluid is plasma. In some embodiments, the method provides a presynaptic biomarker level (e.g., in said bodily fluid) in said subject that decreases at a slower rate than in an untreated control by at least about 1, at least about 2, at least about 3, at least about 4, or at least about 5 annual percent change (APC) or rendering a value that is lower than a corresponding reference. In some embodiments, the corresponding reference is a corresponding pretreatment level (e.g., in said bodily fluid). In some embodiments, the corresponding reference is determined from an untreated control.

In some embodiments, the disclosure of the present application provides a method for modulating one or more neurodegeneration biomarkers or indicators in a subject in need thereof, the method comprising: administering to said subject an effective amount of a p75^{NTR} modulator or a pharmaceutically acceptable salt thereof to provide a change (e.g., increase) or a rate of change (e.g., a rate of increase) in a postsynaptic biomarker level (e.g., in a bodily fluid) over a duration that is lower than a corresponding reference over the same duration. In some embodiments, the postsynaptic biomarker is neurogranin (NG), such as neurogranin 36 (NG36). In some embodiments, the bodily fluid is a cerebrospinal spinal fluid, blood, serum, or plasma. In some embodiments, the bodily fluid is a cerebrospinal spinal fluid. In some embodiments, the bodily fluid is blood. In some embodiments, the bodily fluid is serum. In some embodiments, the bodily fluid is plasma. In some embodiments, the method provides a postsynaptic biomarker level (e.g., in said bodily fluid) in said subject that increases at a rate lower by at least about 1, at least about 2, at least about 3, at least about 4, or at least about 5 annual percent change (APC) than the corresponding reference. In some embodiments, the corresponding reference is a corresponding pretreatment level (e.g., in said bodily fluid). In some embodiments, the corresponding reference is determined from an untreated control.

In some embodiments, the disclosure of the present application provides a method for modulating one or more neurodegeneration biomarkers or indicators in a subject in need thereof, the method comprising: administering to said subject an effective amount of a p75^{NTR} modulator or a pharmaceutically acceptable salt thereof to provide a change (e.g., increase) or a rate of change (e.g., a rate of increase) in a glial marker level (e.g., in a bodily fluid) over a duration that is lower than a corresponding reference over the same duration. In some embodiments, the glial marker is soluble Triggering Receptor Expressed on Myeloid cells 2 (sTREM2) or Chitinase 3-like 1 (CHI3L1, also called YKL40). In some embodiments, the glial marker is sTREM2. In some embodiments, the glial marker is YKL40. In some embodiments, the bodily fluid is a cerebrospinal spinal fluid, blood, serum, or plasma. In some embodiments, the bodily fluid is a cerebrospinal spinal fluid. In some embodiments, the bodily fluid is blood. In some embodiments, the bodily fluid is serum. In some embodiments, the bodily fluid is plasma. In some embodiments, the method provides a glial marker level (e.g., in said bodily fluid) in said subject that increases at a lower rate by at least about 1, at least about 2, at least about 3, at least about 4, or at least about 5 annual percent change (APC) than the corresponding reference. In some embodiments, the corresponding reference is a corresponding pretreatment level (e.g., in said bodily fluid). In some embodiments, the corresponding reference is determined from an untreated control.

In some embodiments, the disclosure of the present application provides a method for modulating one or more neurodegeneration biomarkers or indicators in a subject in need thereof, the method comprising: administering to said subject an effective amount of a p75^{NTR} modulator or a pharmaceutically acceptable salt thereof to provide in said subject a rate of volume change of a brain region over a duration that is lower than a corresponding reference rate (e.g., in an untreated control) over the same duration as determined by magnetic resonance imaging (MRI) imaging. In some embodiments, the brain region comprises hippocampus. In some embodiments, the brain region comprises an anatomical brain region. In some embodiments, the brain region comprises the whole brain of the subject. In some embodiments, the brain region is determined by a voxel-based whole brain analysis. In some embodiments, the method provides a rate of volume change of said brain region that is lower by at least 1%, at least 2%, or at least 3% than said corresponding rate. In some embodiments, the corresponding reference rate is a corresponding pretreatment rate of volume change of said brain region. In some embodiments, the corresponding reference rate is determined from an untreated control.

In some embodiments, the disclosure of the present application provides a method for modulating one or more neurodegeneration biomarkers or indicators in a subject in need thereof, the method comprising: administering to said subject an effective amount of a p75^{NTR} modulator or a pharmaceutically acceptable salt thereof to provide in said subject a brain glucose metabolism (¹⁸F-FDG-PET) rate (e.g., of decline) that is lower than a corresponding reference rate (e.g., of decline). In some embodiments, the ¹⁸F-FDG-PET rate is determined over a duration; and wherein said corresponding reference rate is determined over the same duration. In some embodiments, the corresponding reference is a corresponding pretreatment rate. In some embodiments, the corresponding reference is determined from an untreated control.

In some embodiments, the disclosure of the present application provides a method for modulating one or more neurodegeneration biomarkers or indicators in a subject in need thereof, the method comprising: administering to said subject an effective amount of a p75^{NTR} modulator or a pharmaceutically acceptable salt thereof to provide in said subject a change in a neurological testing score over a duration that is less than a corresponding change over the same duration.

In some embodiments, a method of the present disclosure delays or prevents the progression of Alzheimer's Disease in the subject. In some embodiments, the delaying or preventing progression comprises one or more of ameliorating, delaying, or preventing symptoms of Alzheimer's Disease compared to a subject not receiving the p75^{NTR} modulator. In some embodiments, a subject not receiving the p75^{NTR} modulator receives a placebo or no treatment. In some embodiments, the subject exhibits one or both of improved cognition or improved function compared to a patient receiving no therapy or a placebo therapy. In some embodiments, the symptoms are assessed by neurological testing. In some embodiments, the neurological testing is selected from the Alzheimer's Disease Assessment Scale-Cognitive (ADAS-Cog), the Mini Mental Status Exam (MMSE), the Amunet spatial navigation testing, the Clinical Global Impression (CGI) scale, the Geriatric Depression Scale (GDS), the neurological testing battery (NTB), and combinations thereof. In some embodiments, the NTB is selected from the digit span test, the category fluency test, the controlled oral word association test (COWAT), the digit symbol substitution test (DSST), Clinical Dementia Rating-Sum of Boxes (CDR-SB), Alzheimer's Disease Assessment Scale-Cognitive Subscale 13 (ADAS-Cog13), Alzheimer's Disease Cooperative Study-instrumental Activities of Daily Living for Mild Cognitive Impairment (ADCS-iADL), Neuropsychiatric Inventory (NPI), Alzheimer's Disease Composite Score (ADCOMS), and combinations thereof. In some embodiments, the neurological testing is the Alzheimer's Disease Assessment Scale-Cognitive (ADAS-Cog). In some embodiments, the ADAS-Cog score is ADAS11 or ADAS13. In some embodiments, the ADAS-Cog score is ADAS11. In some embodiments, the ADAS-Cog score is ADAS13. In some embodiments, the neurological testing is the Clinical Dementia Rating-Sum of Boxes (CDR-SB). In some embodiments, the neurological testing is the Alzheimer's Disease Assessment Scale-Cognitive Subscale 13 (ADAS-Cog13). In some embodiments, the neurological testing is the Alzheimer's Disease Cooperative Study-instrumental Activities of Daily Living for Mild Cognitive Impairment (ADCS-iADL). In some embodiments, the neurological testing is the Neuropsychiatric Inventory (NPI). In some embodiments, the neurological testing is the Alzheimer's Disease Composite Score (ADCOMS). In some embodiments, the corresponding reference is determined from an untreated control. In some embodiments, the corresponding reference change is a corresponding pretreatment change within a corresponding time period in said subject. In some embodiments, the time period is about three months. In some embodiments, the time period is about six months.

In certain embodiments, the disclosure provides for a method for modulating one or more neurodegeneration biomarkers or indicators in a subject in need thereof, the method comprising: administering to said subject an effective amount of a p75^{NTR} modulator or a pharmaceutically acceptable salt thereof (e.g., for a time period (e.g., of at least six months)) to provide one or more of the following in said subject: (i) an amyloid beta (Aβ) level in a bodily fluid (e.g., a cerebrospinal fluid) that is lower than a corresponding reference (e.g., a corresponding pretreatment level in said bodily fluid); (ii) a tau (τ) level in a bodily fluid (e.g., a cerebrospinal fluid) that is lower than a corresponding reference (e.g., a corresponding pretreatment level in said bodily fluid) or that increases at rate less than that expected in an untreated control; (iii) a presynaptic biomarker level in a bodily fluid (e.g., a cerebrospinal fluid) that is lower than a corresponding reference (e.g., a corresponding pretreatment level in said bodily fluid) or that increases at rate less than that expected in an untreated control; (iv) a postsynaptic biomarker level in a bodily fluid (e.g., a cerebrospinal fluid) that is lower than a corresponding reference (e.g., a corresponding pretreatment level in said bodily fluid) or that increases at rate less than that expected in an untreated control; (v) a glial marker level in a bodily fluid (e.g., a cerebrospinal fluid) that is lower than a corresponding reference (e.g., a corresponding pretreatment level in said bodily fluid) or that increases at rate less than that expected in an untreated control; (vi) a rate of volume change of a brain region (e.g., hippocampus) that is lower than a corresponding rate (e.g., a corresponding pretreatment rate of volume change of said brain region) or rate that would be expected to occur without treatment as determined by magnetic resonance imaging (MRI) imaging; (vii) a brain glucose metabolism (¹⁸F-FDG-PET) rate of decline that is lower than a corresponding reference (e.g., a corresponding pretreatment rate); and (viii) a change in Alzheimer's Disease Assessment Scale-Cognitive (ADAS-Cog) score within a time period (e.g., of about six months) that is less than a corresponding change (e.g., a corresponding pretreatment change within a corresponding time period in said subject). As described herein, a p75^{NTR} modulator exhibits a binding specificity for p75^{NTR} receptor. In some embodiments, the pre-treatment level may be determined one or more days prior to the initial administration described herein. In some embodiments, the pre-treatment level may be determined less than 24 hours prior to the initial administration described herein. In some embodiments, any one or more members of items (i)-(viii) may be determined at one or more time point(s) before or after an initial administering or the first dose. In some embodiments, any one or more members of items (i)-(viii) may be determined at one or more time point(s) after a subsequent administering or a subsequent dose. In some embodiments, any one or more members of items (i)-(viii) may be determined at one or more time point(s) after a set of administering or a chronic dosing after a time period (e.g., of at least about 3, at least about 4, at least about 5, or at least about 6 months). In some embodiments, the method described herein maintains, prevents a significant decrease in, or effects an increase in, the Alzheimer's Disease Assessment Scale-Cognitive (ADAS-Cog) score of the subject, e.g., as compared with a pre-treatment level. In many cases, for a given subject, rates of change of fluid biomarker or imaging measures are not specifically determined for that individual. Instead, treatment is started empirically as the assumption that the treatment will slow rates of progression of degeneration and its various measures based on previous trial data and based on the profile of the subject matching that of trial participants.

In some embodiments of any one of the relevant methods described herein, the cerebrospinal (e.g., fluid) Alzheimer's Disease-related biomarker levels include the levels (or certain ratios thereof) of one or more of tau, p-tau, Aβ40, Aβ42, AchE, neurofilaments light chain (an indicator of neuronal degeneration), SNAP-25 (an indicator of neuronal degeneration, especially pre-synaptic degeneration), neurogranin (an indicator of neuronal degeneration, especially post-synaptic degeneration), synaptotagmin-1 (an indicator of neuronal degeneration, especially pre-synaptic degeneration), sTREM (an indicator of neuroinflammation or gliosis) and YKL-40 (an indicator of neuroinflammation or gliosis; or any ratios thereof such as Ab 42/40.). In some embodiments, the cerebrospinal Alzheimer's Disease-related biomarker levels include the levels of one or more of tau, p-tau, Aβ40, Aβ42, AchE activity. In some embodiments, the cerebrospinal Alzheimer's Disease-related biomarker levels include the levels of one or more of indicators of neuronal degeneration such as neurofilaments light chain, SNAP-25, neurogranin, and synaptotagmin-1. In some embodiments, the cerebrospinal Alzheimer's Disease-related biomarker levels include the levels of one or more of indicators of neuroinflammation or gliosis such as sTREM and YKL-40. In some embodiments, the cerebrospinal Alzheimer's Disease-related biomarker is selected from any one or any subset of those set forth in Table A. Many of these biomarkers can also be assessed in blood or plasma samples.

TABLE A
List of cerebrospinal Alzheimer's Disease-related biomarkers

tau
p-tau
Aβ40
Aβ42
AchE
neurofilaments light chain
SNAP-25
Neurogranin
synaptotagmin-1
sTREM
YKL-40

In some embodiments of any one of the relevant methods described herein, the cognitive testing methods include one or more of the Alzheimer's Disease assessment scale (ADAS-cog), mini mental status exam (MMSE), Clinical Global Impression (CGI) scale, neurological testing battery (NTB), Spatial navigation Testing with Amunet, the Geriatric Depression Scale (GDS), and other tests. In some embodiments, the cognitive testing methods include one or more of ADAS-cog and MMSE. In some embodiments, the cognitive testing methods include one or more of CGI, Spatial navigation Testing with Amunet, CGI scale, and NTB. The NTB may include one or more of the digit span test, the category fluency test, the controlled oral word association test (COWAT), and the digit symbol substitution test (DSST). In some embodiments, the cognitive testing method is selected from any one or any subset of those set forth in Table B.

TABLE B
List of cognitive testing methods

```
Alzheimer's Disease assessment scale (ADAS-cog)
Mini Mental Status Exam (MMSE)
Clinical Global Impression (CGI) scale
Neurological Testing Battery (NTB)
  Digit span test
  Category fluency test
  Controlled oral word association test (COWAT)
  Digit symbol substitution test (DSST)
Clinical Dementia Rating-Sum of Boxes (CDR-SB)
Alzheimer's Disease Cooperative Study - instrumental Activities
of Daily Living for Mild Cognitive Impairment (ADCS-iADL)
Neuropsychiatric Inventory (NPI)
Alzheimer's Disease Composite Score (ADCOMS)
```

In some embodiments of any one of the relevant methods described herein, the bodily fluid is a cerebrospinal spinal fluid, blood, serum, or plasma. In some embodiments, the bodily fluid is a cerebrospinal spinal fluid. In some embodiments, the bodily fluid is blood. In some embodiments, the bodily fluid is serum. In some embodiments, the bodily fluid is plasma.

In some embodiments of any one of the relevant methods described herein, the corresponding reference (e.g., level, value, rate, etc.) is a corresponding pretreatment level, value, rate (etc.) of the subject (e.g., in said bodily fluid). In some embodiments, the corresponding reference (e.g., level, value, rate, etc.) is determined from an untreated control (e.g., a corresponding subject or a placebo cohort).

In some embodiments of any one of the relevant methods described herein, the duration or time period (over which a change or a rate of change, for example, an increase or a rate of increase, in the one or more neurodegeneration biomarkers or indicators is determined) is at least about 5 days, at least about 10 days, at least about 15 days, at least about 20 days, at least about 25 days, at least about 30 days, at least about 1 month, at least about 2 months, at least about 3 months, at least about 4 months, at least about 5 months, or at least about 6 months. In some embodiments, the duration or time period is at least about 5 days. In some embodiments, the duration or time period is at least about 10 days. In some embodiments, the duration or time period is at least about 15 days. In some embodiments, the duration or time period is at least about 20 days. In some embodiments, the duration or time period is at least about 25 days. In some embodiments, the duration or time period is at least about 30 days. In some embodiments, the duration or time period is at least about 1 month. In some embodiments, the duration or time period is at least about 2 months. In some embodiments, the duration or time period is at least about 3 months. In some embodiments, the duration or time period is at least about 4 months. In some embodiments, the duration or time period is at least about 5 months. In some embodiments, the duration or time period is at least about 6 months.

In some embodiments of any one of the relevant methods described herein, the duration or time period (over which a change or a rate of change, for example, an increase or a rate of increase, in the one or more neurodegeneration biomarkers or indicators is determined) is from about 5 days to about 6 months, from about 10 days to about 6 months, from about 15 days to about 6 months, from about 20 days to about 6 months, from about 25 days to about 6 months, from about 30 days to about 6 months, from about 1 month to about 6 months, from about 2 months to about 6 months, from about 3 months to about 6 months, from about 4 months to about 6 months, or from about 5 months to about 6 months. In some embodiments, the duration or time period is from about 5 days to about 6 months. In some embodiments, the duration or time period is from about 10 days to about 6 months. In some embodiments, the duration or time period is from about 15 days to about 6 months. In some embodiments, the duration or time period is from about 20 days to about 6 months. In some embodiments, the duration or time period is from about 25 days to about 6 months. In some embodiments, the duration or time period is from about 30 days to about 6 months. In some embodiments, the duration or time period is from about 1 month to about 6 months. In some embodiments, the duration or time period is from about 2 months to about 6 months. In some embodiments, the duration or time period is from about 3 months to about 6 months. In some embodiments, the duration or time period is from about 4 months to about 6 months. In some embodiments, the duration or time period is from about 5 months to about 6 months.

In some embodiments of any one of the relevant methods described herein, the subject exhibits or is determined to exhibit mild to moderate Alzheimer's Disease according to McKhann (2011) criteria within six months prior to said administration. In some embodiments, the subject exhibits or is determined to exhibit mild to moderate Alzheimer's Disease according to McKhann (2011) criteria within three months prior to said administration. In some embodiments, the subject exhibits or is determined to be at high risk or in a pre-clinical or prodromal state of Alzheimer's Disease as determined by genetic risk and/or biomarkers within six months prior to said administration. In some embodiments, the subject exhibits or is determined to be at high risk or in a pre-clinical or prodromal state of Alzheimer's Disease as determined by genetic risk and/or biomarkers within three months prior to said administration. In some embodiments, the subject exhibits or is determined to exhibit an F4 allele of apolipoprotein E (ApoE) gene. In some embodiments, the subject exhibits or is determined to exhibit no F4 allele of apolipoprotein E (ApoE) gene. In some embodiments, the subject is between the ages of 50 and 90. In some embodiments, the subject is younger than about 72 years of age. In some embodiments, the subject is of about 72 years of age or older.

### Proteomic Modules

In various embodiments, methods of the present disclosure delay or prevent progression of Alzheimer's Disease in a subject. In some embodiments, proteomic modules are used to characterize Alzheimer's Disease status or monitor progression of Alzheimer's Disease in a subject. A “proteomic module” is a group of co-expressed proteins that relate to specific functions. In some embodiments, a proteomic module is measured from a cerebrospinal fluid (CSF) sample from a subject (e.g., a subject with Alzheimer's Disease). In some embodiments, proteomic modules can be compared between subjects with Alzheimer's Disease over the course of their disease. In some embodiments, changes in one or more proteomic modules indicates progression of Alzheimer's Disease in the subject. In some embodiments, delaying or preventing progression comprises reduced levels of one or more of a ubiquitination proteomic module, a glycolysis proteomic module, a postsynaptic proteomic module, and an axonogenesis proteomic module compared to a subject not receiving the p75^{NTR} modulator, a subject receiving a placebo therapy, or a subject receiving therapy other than a p75^{NTR} modulator. In some embodiments, delaying or preventing progression comprises increased levels of one or both of a blood brain barrier (BBB) proteomic module and a collagen proteomic module compared to one or more of a subject not receiving the p75^{NTR} modulator, a subject receiving a placebo therapy, or a patient receiving therapy other than a p75^{NTR} modulator.

In some embodiments, a proteomic module is a “ubiqutination proteomic module.” In some embodiments, a ubiqutination proteomic module is an “M4 ubiqutination proteomic module.” A ubiquitination proteomic module can include one or more proteins selected from the group consisting of UCHL1, YWHAZ, YWHAG, PGK1, YWHAE, GDI1, PPIA, PGAM1, UBE2N, GLO1, NME1, YWHAB, PTPA, HSP90AA1, ENO1, GLOD4, MIF, FABP3, HAGH, LDHA, PEA15, GSR, TBCA, CFL1, SNCB, SH3BGRL3, TPT1, SCRN1, UBE2L3, PPP3CA, LASP1, PSMB7, CAND1, TXNRD1, SNCA, MAT2A, GPI, YWHAH, ST13, PRDX1, PPP1R7, FKBP1A, PSMB6, DDT, CKB, NEFM, OTUB1, MAPK1, PSMA3, TAGLN3, GAPDH, ATOX1, FAM49B, GLRX, NEDD8, PSMA4, PSMB4, PRDX3, NAXD, PSMA1, ITGB2, PSMA6, CZIB, GSTO1, FSCN1, ENOPH1, MAT2B, AKR1B1, RAD23A, DPYSL2, HSPA1A, TWF2, SNCG, PPP3R1, PSMB1, PSMA7, GABARAP, MAP1B, PLEC, CALB2, UFM1, CHI3L1, TBCB, AQP4, APOE4, CORO1C, NEFL, COTL1, FABP7, YKT6, ESD, THOP1, ATP6V1E1, PSMA5, ME1, MARCKSL1, BPNT1, CACYBP, ADD1, FDPS, CHIT1, MAP2, GSTP1, BIN1, TXNL1, FABP5, UBQLN2, IMPA1, ANGPTL4, CAP2, HEBP1, and AAMDC.

In some embodiments, a proteomic module is a “glycolysis proteomic module.” In some embodiments, a glycolysis proteomic module is an “M5 glycolysis proteomic module.” A glycolysis proteomic module can include one or more proteins selected from the group consisting of MAPT, NRGN, GAP43, PKM, GOT1, ALDOA, MDH1, UBB, CALM1, BASP1, HSPA8, SOD1, DDAH1, PITHD1, LDHB, NPEPPS, PEBP1, DYNLL2, RAD23B, CPLX2, GDA, STMN1, GSS, PARK7, GMFB, SYN1, HPRT1, DTD1, SYT1, UBE2V1, AP2B1, SKP1, PKIA, PPP5C, TXN, NSF, SOD2, TALDO1, DLD, SMOC1, TMSB4X, PDXP, NUTF2, ALDOC, DDB1, CEND1, TOLLIP, SMOC2, PRKAR1A, CRYM, NIF3L1, GABARAPL2, PFN2, GGCT, CAMK2A, TPI1, GOT2, NAXE, PREP, NUDC, RTN1, DNM1, CYCS, SPON1, HSPE1, SPP1, ENO2, HDHD2, LAMP5, DLST, DNAJB2, SCN2B, RIDA, PSAT1, EPB41L1, BLMH, RPH3A, MARCKS, CYR61, HK1, CAMK2D, ACYP2, VSNL1, CKMT1A, TXNDC17, MDH2, S100A1, STX1B, CAMK2B, ATP6AP2, LHPP, STX8, EIF4B, TPD52, SNAP91, VAPA, PRKAR1B, TNFRSF12A, SERPINE2, SELENOW, VASN, MT2A, AP1B1, DBI, HSPA2, C1orf232, NAV1, SLC1A2, QDPR, B2M, PGM1 and CAMK2G.

In some embodiments, a proteomic module is a “postsynaptic proteomic module.” In some embodiments, a glycolysis proteomic module is an “M2 postsynaptic proteomic module.” A postsynaptic proteomic module can include one or more proteins selected from the group consisting of C1QL3, C4orf48, DPP10, ENDOD1, and SUSD5.

In some embodiments, a proteomic module is an “axonogenesis proteomic module.” In some embodiments, a glycolysis proteomic module is an “M6 axonogenesis proteomic module.” An axonogenesis proteomic module can include one or more proteins selected from the group consisting of GALNT18, PVR, SEMA4D, CNTNAP2, and RTN4RL2.

In some embodiments, a proteomic module is a “blood brain barrier (BBB) proteomic module.” In some embodiments, a BBB proteomic module is an “M1 BBB proteomic module.” A BBB proteomic module can include one or more proteins selected from the group consisting of OGN, SFRP4, PDGFRL, METRNL, and MYOC.

In some embodiments, a proteomic module is a “collagen proteomic module.” In some embodiments, a BBB proteomic module is an “M3 collagen proteomic module.” A collagen proteomic module can include one or more proteins selected from the group consisting of MATN2, PTK7, FMOD, SEMA3D, and B3GNT9.

In some embodiments, all proteins in a proteomic module are measured. In some embodiments, a subset of proteins in a proteomic module are measured.

Administration of p75^{NTR} Modulators

The amount of a given agent that will correspond to such an amount (e.g., to an amount of a reference agent) may vary depending upon factors such as the particular compound, disease or condition and its severity, the identity (e.g., weight) of the subject or host in need of treatment, but can nevertheless be determined in a manner recognized in the field according to the particular circumstances surrounding the case, including, e.g., the specific agent being administered, the route of administration, the condition being treated, and the subject or host being treated.

Those skilled in the art will appreciate that, as is typical for pharmaceutical dosing, in many embodiments, a pharmaceutical composition in accordance with the present disclosure may be administered in a dosing regimen. In many embodiments, such dosing regimen is one that has been determined, when administered to a relevant population (e.g., to a population of subjects suffering from or susceptible to AD and having a tau pathology biomarker as described herein), to achieve a particular determined desirable endpoint (e.g., a certain degree of improvement in a relevant biomarker or characteristic of AD in a meaningful percentage of such population)

Those skilled in the art will further appreciate that an appropriate dosing regimen will commonly include a plurality of doses. In some embodiments, more than one dose may be administered per day. For example, in some embodiments, a pharmaceutical composition suitable for use with the disclosed methods is administered in the morning and the evening.

A pharmaceutical composition suitable for use with the disclosed methods may be administered to a subject in a fed or fasted state. That is, in some embodiments, a pharmaceutical composition may be administered to a subject in a fed state, in some embodiments, a pharmaceutical composition may be administered to a subject in a fasted state, in some embodiments, a pharmaceutical composition may be administered to a subject independent of fed or fasted state.

In some embodiments, a pharmaceutical composition suitable for use with the disclosed methods may be administered daily, e.g., once or more than once daily, such as twice or more times daily. In some embodiments, a pharmaceutical composition may be administered for a period of two or more weeks, three or more weeks, four or more weeks, five or more weeks, six or more weeks, seven or more weeks, two or more months, three or more months, four or more months, five or more months, or six or more months, or one or more years, or one or more decades. In mouse PK-PD studies, twice per day dosing of certain p75^{NTR} modulators (e.g., of a compound of Formula (Ia), which may in some embodiments be in a pharmaceutically acceptable salt form, tends to give greater efficacy than once per day dosing.

In some embodiments, a p75^{NTR} modulator is administered orally, buccally, rectally, parenterally, ophthalmically, or via inhalation. In some embodiments, a p75^{NTR} modulator is administered orally. In some embodiments, a p75^{NTR} modulator is administered buccally. In some embodiments, a p75^{NTR} modulator is administered rectally. In some embodiments, a p75^{NTR} modulator is administered parenterally. In some embodiments, a p75^{NTR} modulator is administered ophthalmically. In some embodiments, a p75^{NTR} modulator is administered via inhalation.

One attractive feature of certain p75^{NTR} modulators, including, for example, the compound of Formula (Ia), and pharmaceutically acceptable salts thereof, is their amenability to oral administration. In general, oral is a particularly desirable route of administration. However, those skilled in the art appreciate that oral administration can be problematic for certain AD dementia patients. As noted herein, alternative useful routes, especially for such AD dementia subjects include buccal, ophthalmic, parenteral, rectal, and delivery via inhalation, Particularly useful, especially for such AD dementia subjects may be rectal and/or delivery via inhalation.

Suitable unit dosage forms for oral administration include from about 1 to about 1,000 mg active ingredient (free base weight). In some embodiments, the unit dosage is about 1 to about 800 mg, about 1 to about 600 mg, or about 1 to about 500 mg, (all weights of free base). In some embodiments, the unit dosage is about 10 to about 1,000 mg, about 10 to about 800 mg, about 10 to about 600 mg, or about 10 to about 500 mg, (all weights of free base). The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the response of imaging or cerebrospinal based biomarkers, the mode of administration, the requirements of the individual subject, the severity of the disease or condition being treated, and the judgment of the practitioner.

In some embodiments, a pharmaceutical composition used in accordance with the present disclosure comprises from about 10 milligram (mg) (free base weight) to about 1000 mg (free base weight) of the salt of the compound of Formula (I). In some embodiments, the pharmaceutical composition comprises from about 10 milligram (mg) (free base weight) to about 900 mg (free base weight) of the salt of the compound of Formula (I). In some embodiments, the pharmaceutical composition comprises from about 10 milligram (mg) (free base weight) to about 800 mg (free base weight) of the salt of the compound of Formula (I). In some embodiments, the pharmaceutical composition comprises from about 10 milligram (mg) (free base weight) to about 600 mg (free base weight) of the salt of the compound of Formula (I). In some embodiments, the pharmaceutical composition comprises from about 10 milligram (mg) (free base weight) to about 500 mg (free base weight) of the salt of the compound of Formula (I). In some embodiments, the pharmaceutical composition comprises from about 10 milligram (mg) (free base weight) to about 400 mg (free base weight) of the salt of the compound of Formula (I). In some embodiments, the pharmaceutical composition comprises from about 10 milligram (mg) (free base weight) to about 300 mg (free base weight) of the salt of the compound of Formula (I). In some embodiments, the pharmaceutical composition comprises from about 10 milligram (mg) (free base weight) to about 200 mg (free base weight) of the salt of the compound of Formula (I). In some embodiments, the pharmaceutical composition comprises from about 10 milligram (mg) (free base weight) to about 100 mg (free base weight) of the salt of the compound of Formula (I).

In some embodiments, a p75^{NTR} modulator (which may be in pharmaceutically acceptable salt form), or a pharmaceutical composition comprising the same, is formulated to deliver the P75^{NTR} modulator at a dose of 100 mg b.i.d. (bis in die, twice a day). In some embodiments, a p75^{NTR} modulator (which may be in pharmaceutically acceptable salt form), or a pharmaceutical composition, is formulated to deliver the P75^{NTR} modulator at a dose of 200 mg b.i.d. In some embodiments, a p75^{NTR} modulator (which may be in pharmaceutically acceptable salt form), or a pharmaceutical composition, is formulated to deliver the P75^{NTR} modulator at a dose of 300 mg b.i.d. In some embodiments, a p75^{NTR} modulator (which may be in pharmaceutically acceptable salt form), or a pharmaceutical composition, is formulated to deliver the P75^{NTR} modulator at a dose of 400 mg b.i.d. In some embodiments, a p75^{NTR} modulator (which may be in pharmaceutically acceptable salt form), or a pharmaceutical composition, is formulated to deliver the P75^{NTR} modulator at a dose of 100 mg b.i.d. to 800 mg b.i.d. In some embodiments, a p75^{NTR} modulator (which may be in pharmaceutically acceptable salt form), or a pharmaceutical composition, is formulated to deliver the P75^{NTR} modulator at a dose of 100 mg b.i.d. to 700 mg b.i.d. In some embodiments, a p75^{NTR} modulator (which may be in pharmaceutically acceptable salt form), or a pharmaceutical composition, is formulated to deliver the P75^{NTR} modulator at a dose of 100 mg b.i.d. to 600 mg b.i.d. In some embodiments, a p75^{NTR} modulator (which may be in pharmaceutically acceptable salt form), or a pharmaceutical composition, is formulated to deliver the P75^{NTR} modulator at a dose of 100 mg b.i.d. to 500 mg b.i.d. In some embodiments, a p75^{NTR} modulator (which may be in pharmaceutically acceptable salt form), or a pharmaceutical composition, is formulated to deliver the P75^{NTR} modulator at a dose of 100 mg b.i.d. to 400 mg b.i.d. In some embodiments, a p75^{NTR} modulator (which may be in pharmaceutically acceptable salt form), or a pharmaceutical composition, is formulated to deliver the P75^{NTR} modulator at a dose of 100 mg b.i.d. to 300 mg b.i.d. In each of the foregoing, as will be understood by those skilled in the art, such dose refers to that of the free base (or acid) of the p75^{NTR} modulator; in some embodiments, where such p75^{NTR} modulator is other than a compound of Formula (Ia), such amount will be understood to be an amount corresponding to the indicated amount of the free base form of the compound of Formula (Ia).

In some embodiments of any one of the relevant methods described herein, said administering is performed more than once daily. In some embodiments, said administering is performed twice daily. In some embodiments, said administering is performed three times daily. In some embodiments, said administering is orally. In some embodiments, said administering comprises administering to said subject said effective amount of said p75^{NTR} modulator or said pharmaceutically acceptable salt thereof for a duration of at least three months. In some embodiments, said administering comprises administering to said subject said effective amount of said p75^{NTR} modulator or said pharmaceutically acceptable salt thereof for a duration of at least six months.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### EXAMPLES

### Example 1: Drug Manufacture and Stability

A salt of Formula (Ia) (e.g., the salt of Formula (II), also known as Tris[(2S,3S)-2-amino-3-methyl-N-(2-morpholinoethyl)pentanamide]tetra(monohydrogensulfate), monosulfate, a molecular formula of C₃₆H₈₅N₉S₅O₂₀, and a molecular weight of 1220.43 gm/mole). This compound is a white solid and has aqueous solubility of greater than 30 mg/mL and very good stability under accelerated temperature and humidity conditions. GMP lots have been prepared with no impurities above the LOD following storage at ICH stress conditions, indicating sufficient stability of the drug substance. Preparation of the clinical dosage form for a Phase I and Phase II studies consisted of a dry fill of the drug substance into gelatin capsules with no excipients. For the Phase II study, capsules with a salt of Formula (Ia) (200 mg or 400 mg, free base weight) are machine-filled and packaged in blister strips with aluminum foil backing, which are stored in cardboard boxes. Matching placebo capsules are also available in the same packaging.

Data show no significant changes in the drug substance through 18 months at −20° C., 5° C., 25° C./60% RH or 40° C./75% RH other than moisture content, which decreases under all conditions monitored, particularly at the refrigerated (5° C.) and frozen (−20° C.) conditions.

Similarly, data for a drug substance lot for the Phase I trial showed no significant changes related to assay or impurities over a six-month time frame under accelerated conditions, or at 48 months when stored at 25° C./60% RH (the recommended storage condition).

A stability study of capsules filled with 200 mg of the salt of Formula (Ia) (free base weight) packed in PVC-PE-PVdC/Alu at ambient conditions (15-25° C./20-60% RH) shows good drug stability. The provisional shelf-life is set at 18 months, stored in PVC-PE-PVdC/Alu blister strips at ambient conditions.

### Example 2. Regional Brain Glucose Metabolism (18F-FDG-PET)

The specific ¹⁸F FDG-PET scan for early detection of AD is conducted at the first and final visits. Quantitative and qualitative estimates of the cerebral glucose rate may be done by using FDG-PET as evident metabolic reduction is already present in patients at early stages of Alzheimer's disease. ¹⁸F FDG-PET scanning is an imaging biomarker for synaptic function. Analyses include anatomical, statistical or voxel-based regions of interest. Patients receiving daily treatment with a pharmaceutical composition comprising a salt of Formula (Ia) show a reduction in the extent of ongoing decrease in cerebral glucose rate following administration of the composition over a course of time compared to groups of subjects receiving placebo or no treatment. At baseline and final Visit the specific 18F FDG-PET scan for early detection of AD (Mosconi et al., 2010) was conducted. Quantitative and qualitative estimates of the cerebral glucose rate may be done by using FDG-PET as evident metabolic reduction was already present in patients at early stages of Alzheimer's disease (Berti et al., 2010; Minoshima et al., 1997).

### Example 3. Brain MRI Regions of Interest Volumes (ROI) and Voxel Based Whole Brain Analyses

Volumetric MRI is conducted at the first and final visits. Volumetric analyses of the whole brain, hippocampus and other regions of interest are conducted. Measures of regional volumes as determined by MRI are regarded as standard measures of brain degeneration that are known to occur in AD. Volumes of the lateral ventricles are also assessed. Increasing ventricular volume is a marker for a diffuse increase in brain atrophy. Patients receiving daily treatment with a pharmaceutical composition comprising a salt of Formula (Ia) show a reduction in the extent of ongoing decrease in one or more regional volumes during the treatment period compared to groups of subjects receiving placebo or no treatment. They also demonstrate a reduction in the extent of ongoing increase in ventricular volume during the treatment. Longitudinal AD studies demonstrate atrophy of selected brain regions over 6-month intervals as well as increased volume of lateral ventricles, each indicative of neuronal degeneration. The following Regions of interest were investigated in the ITT population: MRI Volume of the lateral ventricles in mm³−longitudinal stream change, MRI Average volume of the hippocampus in mm³−longitudinal stream change, MRI Average Volume of the entorhinal cortex in mm³−longitudinal stream change, MRI Total brain volume in mm3 change. Baseline and final Visit MRI scan were used to measure interval changes in volumes. Various cortical regions such as the Cingulate cortex were also assessed. Volumetric MRI was also conducted using a whole brain voxel-based approach in which brain regions are identified that demonstrated volume loss during the treatment period with the extent of volume loss statistically greater in the placebo group compared to the drug treated group. The identified brain regions are similar in distribution to those having been previously identified to be particularly vulnerable to degeneration in AD. Thus the drug is demonstrated to slow volume loss in brain regions known to be particularly vulnerable to degeneration in AD.

### Example 4. CSF Biomarkers

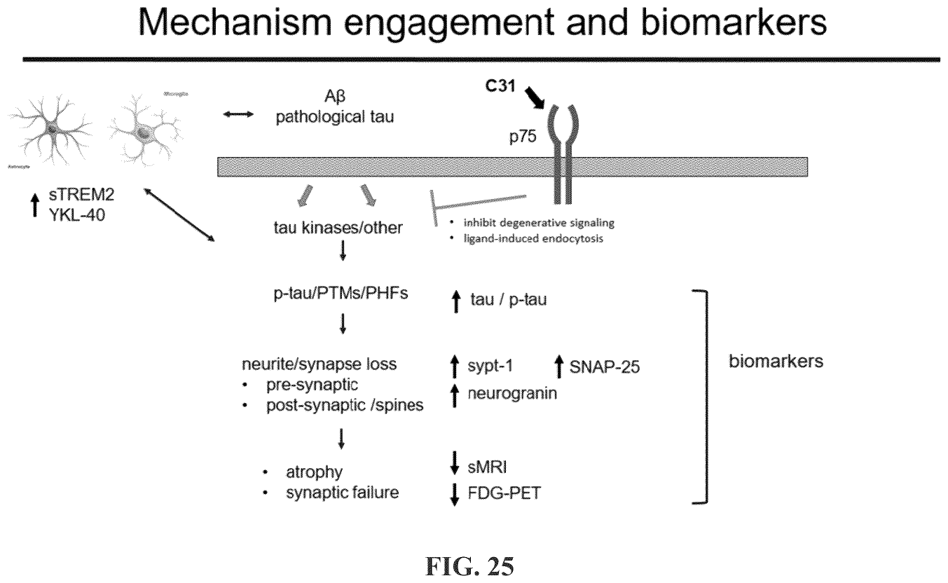

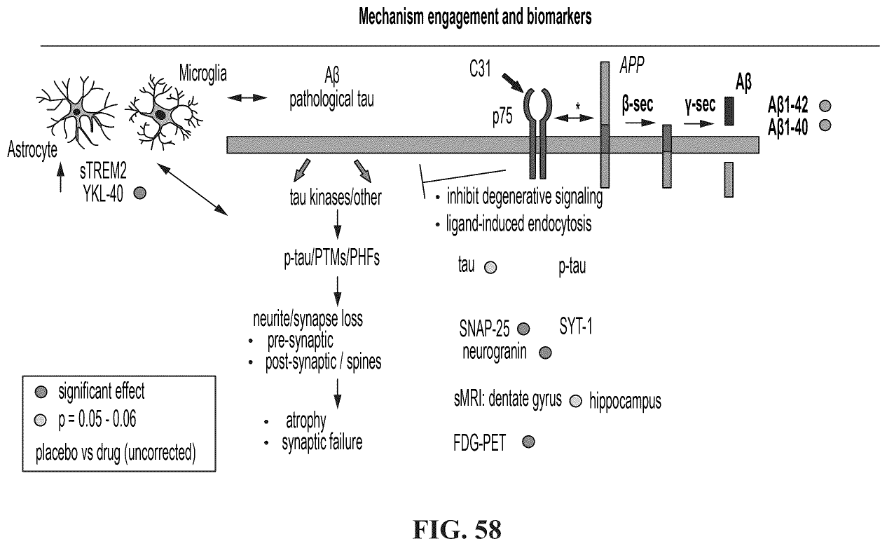

CSF samples were obtained at screening Visit and final Visit via lumbar puncture to determine levels of tau, p-tau, Aβ40, Aβ42 and AChE activity as an initial set of CSF biomarkers. Aliquots of CSF were made available for emerging CSF biomarker methods such as measurement of additional biomarkers including neurofilaments light chain, neurogranin, SNAP-25, synaptotagmin, YKL-40, TREM2, tau oligomers and for measurement of drug level in selected patients based on the time period between last dose of drug and CSF sampling (FIG. 25 and FIG. 58).

Baseline CSF samples are obtained prior to administration via lumbar puncture. These samples are used to determine levels of tau, p-tau, Aβ40, Aβ42, AChE, neurofilament light chain, SNAP-25, synaptotagmin-1, neurogranin, sTREM, and YKL-40. Aliquots of CSF are also made available for emerging CSF biomarker methods such as measurement of tau oligomers and for measurement of drug level in selected patients based on time period between last dose of the salt of Formula (Ia) and CSF sampling. Daily treatment with a salt of Formula (Ia) results in a reduction in the degree of expected increase in levels of biomarkers indicating ongoing degeneration, inflammation or gliosis such as tau, p-tau, Aβ40, Aβ42, neurofilament light chain, SNAP-25, synaptotagmin-1, neurogranin, sTREM, and YKL-40 in the CSF compared to the changes in levels found in placebo or untreated subjects. Many of these biomarkers can also be measured in blood or plasma. The change in values over the time period from pre-treatment to post-treatment are of particular importance. The drug demonstrates the ability to slow progression of increase of biomarkers indicating neuronal degeneration, gliosis and neuro-inflammation. It also demonstrated the ability to lower levels of Aβ42 and 40. Absolute levels at single time points of neurofilament light chain, SNAP-25, synaptotagmin-1, neurogranin, sTREM, and YKL-40 in CSF or blood or plasma are of less value given differences in assay techniques and wide variation in absolute levels within normal subjects and within and across AD subjects. In contrast, absolute levels of Aβ42 and 40 at baseline are useful biomarkers indicating the presence of amyloid pathology as low levels of Aβ42 and 40 at a given time point are consistent with amyloid pathology.

### Example 5. Cognitive and Clinical Function Endpoints

Exploratory outcome evaluations involved the assessment of cognitive and clinical function (Table 4). The ADAS-cog and MMSE testing demonstrated significant decline of cognitive function in the placebo group. Directionality of slowing progression of worsening in the ADAS-cog and MMSE indicated a favorable effect of drug treatment. The NTB failed to detect worsening in the placebo group. Items from the Neurological Testing Battery (NTB), together with the ADAS-Cog 13, Spatial Navigation Test (AMUNET) were performed at Baseline, Week 12 and Week 26 (Visits 2, 4 and 5). While the MMSE was used primarily as a screening and baseline disease staging tool, its repeated administration at Week 26 provided an additional exploratory cognitive outcome measure. The Geriatric Depression Scale (GDS) was assessed at each Visit (Screening, Baseline, Week 4, 12, 26: Visits 1-5) and the Clinical Global Impression test (CGI) with Severity was assessed at Screening (Visit 1) and then the Improvement was subsequently assessed at Weeks 12 and 26 (Visits 4 and 5). For each test, whether categorical or numeric, all raw scores were considered interval in nature. The change from Screening (Week −8) or Baseline (Week 0) was therefore be derived for each patient. Actual values at each Visit and Changes from Screening/Baseline were summarized.

TABLE 4
Cognitive and Clinical Function Endpoints

| | | |
|---|---|---|
| Outcome | Score Range (units) | Scale Direction |

Neurological Test Battery (NTB)

| | | |
|---|---|---|
| DST | 0-24 | High score (24-Best) |
| | (Count correct | |
| | sequences) | |
| CFT | 0-Unlimited | High score |
| | (Count acceptable | |
| | words) | |
| COWAT | Letter C = Accept* | High score |
| | Letter F = Accept* | |
| | Letter L = Accept* | |
| | Total = C + F + L | |
| | (Count correct words) | |
| DSST | 0-117 | High score (117 = Best) |
| | (Count correct | |
| | substitutions) | |

Other Cognitive Function Tests

| | | |
|---|---|---|
| AMUNET | Navigation | AMUNET analysis report |
| | AlloEgo/Ego/Allo/ | |
| | Delayed | |
| ADAS-cog | 0-85 | Low score best (0 = All |
| (13 items) | (Count incorrect items) | correct) |

Clinical Function Tests

| | | |
|---|---|---|
| GDS | 0-15 | Low score best |
| | (Score) | (0-4 = Normal/ |
| | count/classification) | 12-15 Severe depression) |
| CGI - Severity | 1-7 | Low score best |
| (At Baseline) | (Classification) | (1 = Normal/7 = |
| | | Extremely Ill) |
| CGI - Improvement | 1-7 | Low score best |
| | (Classification) | (1 = Very much improved/ |
| | | 7 = Very much worse) |

Disease Staging

| | | |
|---|---|---|
| MMSE | 0-30 | High score best |
| | (Count correct items) | (0 = Severe impairment/ |
| | | 30 = No impairment) |

*Footnote 1: Accept = Number of acceptable words beginning with ‘C, F and L’ and recorded within 60 seconds

### Example 6. Alzheimer's Disease Assessment Scale-13 Items (ADAS-Cog 13 Items)

Alzheimer's Disease Assessment Scale-13 items (total score=85) is used as a parameter for exploratory outcome analysis. ADAS-cog is a psychometric instrument designed to evaluate the severity of cognitive and non-cognitive behavioral dysfunctions characteristic of people with AD. The cognitive portion assesses memory, language and praxis functions. Most items are rated on a scale of 0 to 5, where 0 indicates no impairment and 1 to 5 indicates very mild (1), mild (2), moderate (3), moderately severe (4) or severe impairment (5) respectively. Other items are rated on the presence or absence of a characteristic number of errors or severity of errors. The total scores from the ADAS-cog sub-scales range from 0 (no impairment) to 85 (errors in all sub-tests).

A positive (i.e., increasing) change of the score indicates cognitive worsening. In a meta-analysis model of 52 Alzheimer's Disease trials involving 19,972 mild-moderate disease subjects, a spontaneous decline in performance corresponding to an increase in ADAS-cog score of 5.5 points over one year, pointing to an expected average increase in score of 2.75 points over a 6-month period.

ADAS-Cog was assessed at Visits 2, 4 and 5, as well as at the early discontinuation Visit, if applicable.

Rater: Neurologist, psychologist or other personal (listed as either Principal Investigator or Sub-Investigator) with documented training in psychometric rating.

While the MMSE was used primarily as a screening and baseline disease-staging tool, its administration at Week 26 provided an additional exploratory cognitive outcome measure. Longitudinal evaluations of MMSE performance in Alzheimer's Disease subjects demonstrate annual rates of decrease ranging from 2.8 to 3.4 points, pointing to an expected decline of approximately 1.6 points over a 6-month period. Daily treatment with a salt of Formula (Ia) results in a reduction in the extent of expected decline in MMSE scores compared to the decline in scores occurring in subjects not treated or treated with placebo.

Daily treatment with a compound Formula (Ia) (e.g., in a pharmaceutically acceptable salt form) results in a reduction in the extent of expected decline in ADAS-Cog scores relative to previous visits or a baseline value prior to administration. For example, over the course of treatment of 6 months, subjects receiving daily treatment with a salt of Formula (Ia) may show a reduction in the extent of declining performance in ADAS-Cog scores, or may show a reduction in the extent of declining performance in ADAS-Cog scores by 1, 2, 3, 4, 5, 6, 7, 8, 9, 10, 15, 20, 25, 30, 35, 40, 50, 60, 70 or 85 points. Subjects may show an ADAS-Cog increase of score (decrease in performance) of less than an increase in score observed in a six-month span prior to administration indicating a slowing in the rate of loss of performance. For example, a subject may have an increase in ADAS-Cog of 6 points (decline in performance) in a six month span prior to administration and an increase in ADAS-Cog of less than 6 points, such as an increase in 4, 3, 2 or 1 points in a six month span (indicating a slowing of cognitive decline) that includes treatment with a salt of Formula (Ia). Daily treatment with a salt of Formula (Ia) results in a reduction in the extent of expected decline in ADAS-cog performance (increased score) compared to the decline in performance occurring in subjects not treated or treated with placebo.

ADAS-cog. In the placebo group, ADAS-cog-13 score increased by 2.44 points. Using a linear model, this change of over the 26-week period would project to 4.88 at one year and 7.32 at 18 months, a range of cognitive decline as measured by ADAS-cog-13 in mild-moderate AD expected from prior reports. In the drug group (intention to treat data, ITT), the progression compared to placebo in the LS mean ADAS-cog-13 score increased by 7.1% in the 200 mg group and decreased by 35.3% in the 400 mg group over the 26-week treatment period. For ADAS-cog-11, the progression compared to placebo of the LS mean score decreased by 9.8% in the 200 mg group and decreased by 42.7% in the 400 mg group. For ADAS-cog-6 score, progression increased by 7.1% in the 200 mg group and decreased by 31.4% in the 400 mg group. Consistent with the low clinical assessment power of the study with the relatively small treatment groups in the context of clinical measures of approximately 80 subject each, and assessment of a 6-month rather than 18-month time point, measures of slowing of progression demonstrated notable trends in a favorable direction but were not statistically significant.

### Example 7. Mini Mental Status Exam (MMSE)

Mini Mental Status Exam (MMSE). The MMSE can be used by a physician to evaluate a patient's condition over the course of administration. A baseline value prior to administration of a salt of Formula (Ia) can serve as a basis for baseline disease stage and also for the assessment of the effect of treatment on slowing loss of cognitive function. The expected decline in MMSE over a six month period generally ranges 1-3 points out of the total of 30 points possible in the MMSE test.

Subjects may show a decrease in the extent of expected decline in MMSE over a six month period following or in association with treatment with a salt of Formula (Ia).

In the placebo group, the MMSE score (LS mean, ITT) decreased by 1.65 points. Using a linear model, this progression over the 26-week period would project to 3.3 points at one year and 4.95 at 18 months, a range of cognitive decline in mild-moderate AD expected from prior reports. In the drug group, degree of progression relative to placebo was decreased by 15% in the 200 mg group and by 30.2% in the 400 mg group. Consistent with the low clinical assessment power of the study with the relatively small treatment groups in the context of clinical measures of approximately 80 subject each, and assessment of a 6-month rather than 18-month time point, measures of slowing of progression demonstrated notable trends in a favorable direction but were not statistically significant.

### Example 8. Clinical Global Impression Scale

The Clinical Global Impressions Scale-Severity (CGI-S) and -Improvement (CGI-I) can be used by a physician to evaluate a patient's condition over the course of administration. A baseline value prior to administration of a salt of Formula (Ia) can serve as a basis for the assessment. The subjective categorical values of the CGI-I are as follows: 1=very much improved since the initiation of treatment; 2=much improved; 3=minimally improved; 4=no change from baseline; 5=minimally worse; 6=much worse; and 7=very much worse since the initiation of treatment. The CGI-S was assessed at Visit 1, and CGI-I for evaluation of improvement of subject's condition at Visit 4, 5 and early discontinuation Visit, if applicable. Rater: Neurologist, psychologist or other personal (listed as either Principal Investigator or Sub-Investigator) with documented training in psychometric rating.

Daily treatment with a salt of Formula (Ia) results in a mitigation of worsening of scores (lower scores) in Clinical Global Impression scores occurring during the treatment period compared to a baseline value.

### Example 9. Neurological Testing Battery (NTB)

Daily treatment with a salt of Formula (Ia) results in a decreased in the degree of decline of at least one element of the NTB compared to a pretreatment baseline measurement. The NTB comprises tests of digit span, category fluency, controlled oral word association and digit symbol substitution. These tests are performed approximately three months prior to treatment, after 10-14 weeks of treatment and after completion of treatment.

### Digit Span

Study participants were read sequences of numbers and required to repeat them as heard in the first phase of the test (Digits Forward). In the second phase of the test, Digits Backwards, study participants were required to repeat the sequence in the reverse order. Two trials were administered for each sequence length and 1-point was awarded for each sequence correctly repeated. Testing was performed at Visits 2, 4 and 5, as well as at the early discontinuation Visit, if applicable. The rater was a trained psychologist or physician

### Category Fluency Test

In this test, study participants were required to generate words from a specific category (usually animals) in one minute. Performance across the minute was scored according to acceptable rules to yield the total number of correct responses. This test measures working memory and other aspects of executive function, including planning, strategy and aspects of language and especially fluency. Testing was performed at Visits 2, 4 and 5, as well as at the early discontinuation Visit, if applicable. The rater was a trained psychologist or physician.

### Controlled Oral Word Association Test (COWAT)

The COWAT measures a person's ability to make verbal associations to specified letters (i.e., C, F, and L), evaluates the spontaneous production of words beginning with a given letter and is able to detect changes in word association fluency often found with various disorders. COWAT testing was performed at Visits 2, 4 and 5, as well as at the early discontinuation Visit, if applicable. The rater was a trained psychologist or physician.

### Digit Symbol Substitution Test

In the Digit Symbol Substitution Test (DSST), the patient was required to match symbols with their corresponding digit. The test consists of 9 digit symbols, which had to be matched with their corresponding numerical digit. The patients had limited amount of time to enter the correct symbol for each digit. DSST was performed at Visits 2, 4 and 5, as well as at the early discontinuation Visit, if applicable. The rater was a trained psychologist or physician.

### Example 10. Spatial Navigation with AMUNET

Impaired orientation in space is a frequently reported symptom in AD patients. Spatial navigation impairment occurs early in the development of AD and can be used for monitoring of the disease progression or for evaluation of presymptomatic AD.

The two modes of spatial navigation include egocentric and allocentric navigation. Egocentric navigation uses information about distances and angles from the subject positions processing proprioceptive information, whilst allocentric navigation is hippocampus dependent and uses a flexible representation of a distal landmarks ensemble independent of actual subject positions. The parietal cortex including precuneus, and especially the hippocampus, is involved in spatial navigation performance. Impairments are particularly found in patients suffering from memory deficits related to the hippocampal area—correlating to prodromal AD pathologic findings, presumably a signal for preclinical AD.

Memory paradigms used with human study participants suffering from AD typically feature tests of episodic verbal memory, paired associative learning or visual recognition memory. These tasks are very different to the memory paradigms in rodents, where the Morris water maze (MWM) is employed in preclinical studies for the development of new medicinal products for AD.

AMUNET, the computer-based simulation of MWM, tests the two basic types of navigation: world-centered—allocentric (hippocampus-dependent) and body-centered—egocentric (parietal cortex-dependent). Both of these paradigms are controlled by the structures involved in early Alzheimer's Disease pathology. Allocentric navigation is independent of an individual's position and distal cues are used for navigation, while egocentric navigation depends on an individual's position and the start position is used for navigation. AMUNET computer simulation is a map view of the arena projected on computer screen where the participant uses a touch screen to identify the target position. The arena in the computerized version of the MWM was shown as a large white circle with the start position (medium-sized red circle) and 2 orientation cues (yellow and green lines) on its perimeter. A small red circle inside the arena represents the goal.

AMUNET was conducted at Visits 2, 4 and 5, as well as at the early discontinuation Visit, if applicable. The rater was a trained psychologist or physician. The Results from the AMUNET testing after week 12 and week 26 were compared to the baseline tests. Four different result-subsets were compared for ITT and PP population as well as two subsets of subjects within the groups showing mild or moderate symptoms of AD. The four result subsets tested consisted of:
AlloEgoNavigation: The capability of spatial navigation both with the aid of visible orientation cues that are not placed near the goal (allocentricnavigation) and with the aid of information about the direction and the distance from the start that is needed to find the goal(egocentricnavigation).
EgoNavigation: Focused on egocentric navigation.
AlloNavigation: Focused on allocentric navigation
DelayedNavigation: Focused on delayed allocentric navigation.

Daily treatment with a composition comprising a compound of Formula (Ia) in salt form results in a decrease in the decline in performance in testing of either allocentric or egocentric spatial navigation with Amunet as compared to a baseline assessment prior to administration of the composition. Amunet is conducted approximately three months prior to administration of the composition, after 10-14 weeks of treatment and after completion of treatment.

### Example 11: Geriatric Depression Scale (GDS)

The Geriatric Depression Scale (GDS) is a useful validated screening tool to facilitate assessment of depression in older adults especially when baseline measurements are compared to subsequent scores. The GDS Short Form consists of 15 Items and takes about 5 to 7 minutes to be completed. Of the 15 items 10 indicate the presence of depression when answered positively, while the rest indicate depression when answered negatively. A score of 0 to 4 is normal, depending on age, education, and complaints; a score of 5 to 8 indicates a mild depression, 9 to 11 reflects a moderate depression, 12-15 a severe depression. Evaluation of the GDS was performed at screening and only patients with GDS score<5 were enrolled in the study.

The GDS was assessed at Visits 1, 2, 3, 4, 5 and early discontinuation Visit, if applicable. The rater was a trained psychologist or physician.

### Example 12. Phase IIa, Prospective, Multi-Center, Double-Blind, Placebo-Controlled, Randomized, Adaptive-Trial-Design Study to Evaluate Safety, Tolerability, Pharmacokinetics, and Exploratory Endpoints of Either Placebo or Two Different Oral Doses of a Salt of Formula (Ia) in Patients with Mild to Moderate Probable Alzheimer's Disease

### Overall Study Design and Plan Description

The study was a Phase IIa, multi-center, randomized, placebo-controlled, parallel group clinical study with a 26-week double-blind treatment duration and three treatment arms with two consisting of two doses of LM11A-31 (a salt of Formula (Ia)) (200 mg bid and 400 mg bid of free base—and one comprising placebo bid).

The primary goal of the Phase IIa clinical trial was the evaluation of safety and tolerability of two doses of LM11A-31-BHS administered orally twice daily for 26 weeks versus matched placebo. Safety monitoring included the full extent of Phase II clinical and laboratory testing. The p75 receptor had never been specifically targeted in a human trial other than the above Phase I 10 d trial, hence the critical important of determining of targeting this receptor beyond 10 days is safe. Given the fundamental and complex roles of the p75 receptor, it was not known if targeting or modulating this receptor for 26 weeks would be tolerable.

### Treatments Administered

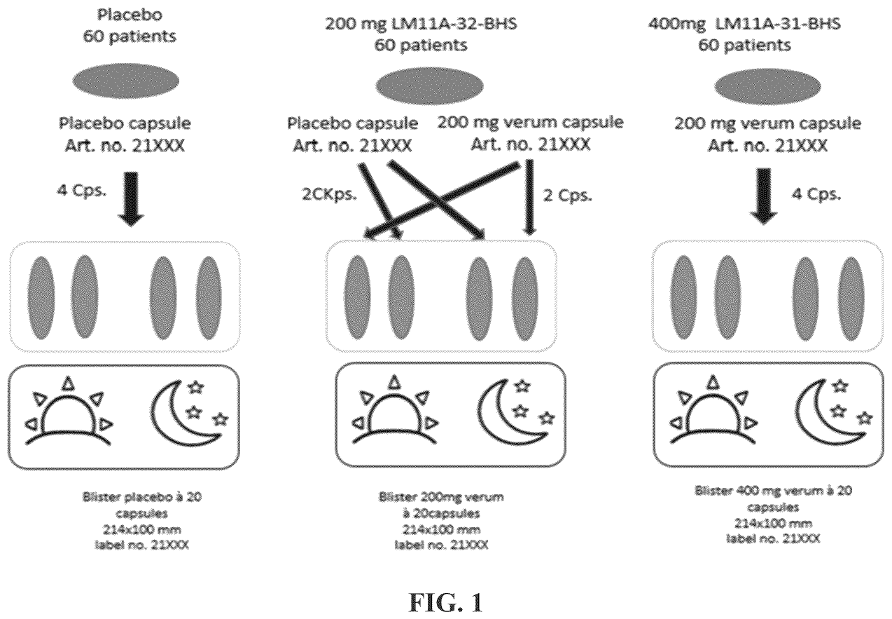

LM11A-31-BHS or the matching placebo were administered twice daily as 2 capsules of identical appearance in a blinded manner (FIG. 1).

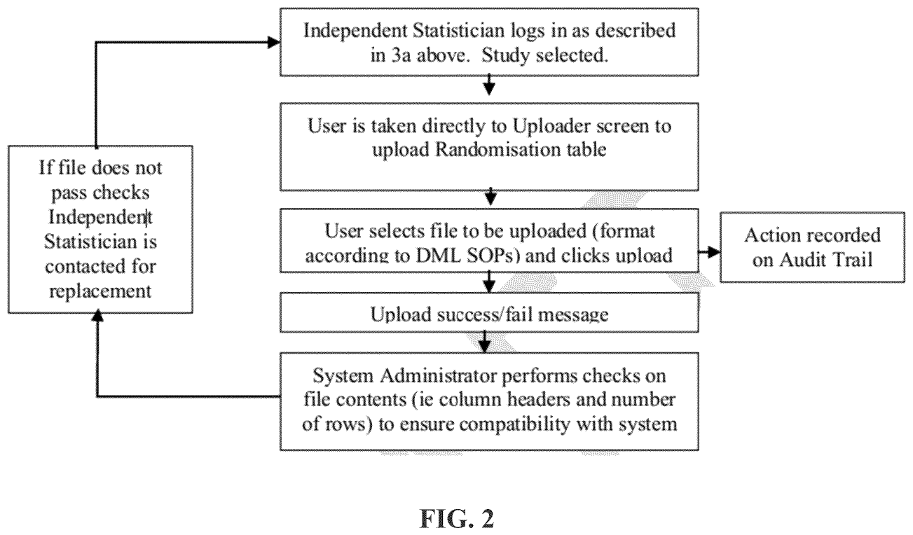

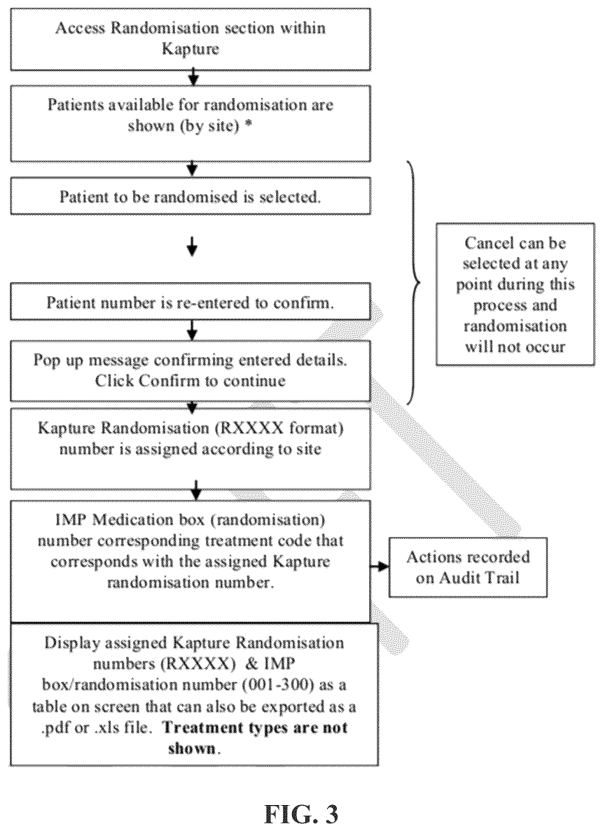

On the first treatment day, the patients were randomized to either placebo (n=80) or 200 mg bid (n=80) or 400 mg bid (n=80) LM11A-31-BHS following the procedure of FIGS. 2 and 3.

### Drug Concentration Measurements

Plasma samples were collected from all subjects who received study medication to investigate the plasma concentration of LM11A-31 and its aminoethyl morpholine metabolite. The time between PK sampling and last intake of study drug was recorded. All available samples including samples from the placebo group were analyzed. Levels of LM11A-31 and its aminoethyl morpholine metabolite were measured in selected CSF samples that were otherwise obtained for studies of CSF biomarkers.

CSF was collected from all subjects who received study medication. However, levels of LM11A-31 and its aminoethyl morpholine metabolite were measured only in CSF samples from subjects who had the last dose of study drug on the day of the CSF collection and who were based on the plasma PK results available at Quintain one of the verum arms. In addition CSF samples of 3 patients from the placebo group were analyzed as QC check.

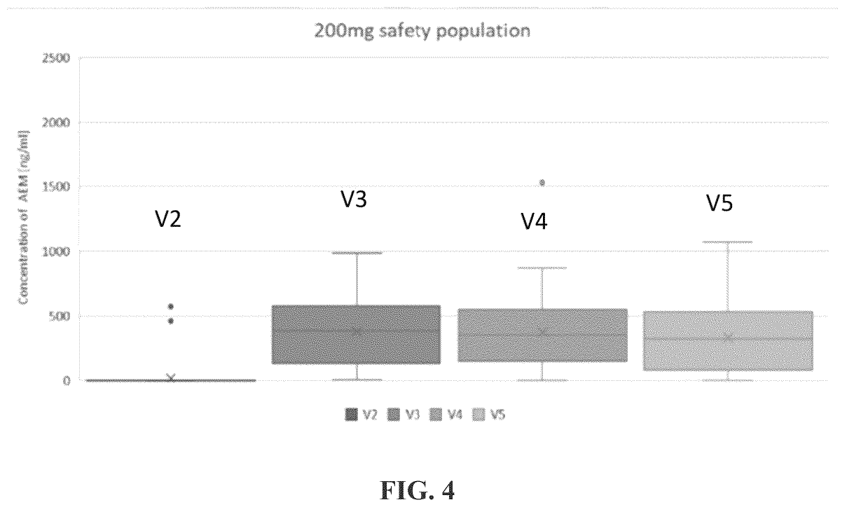

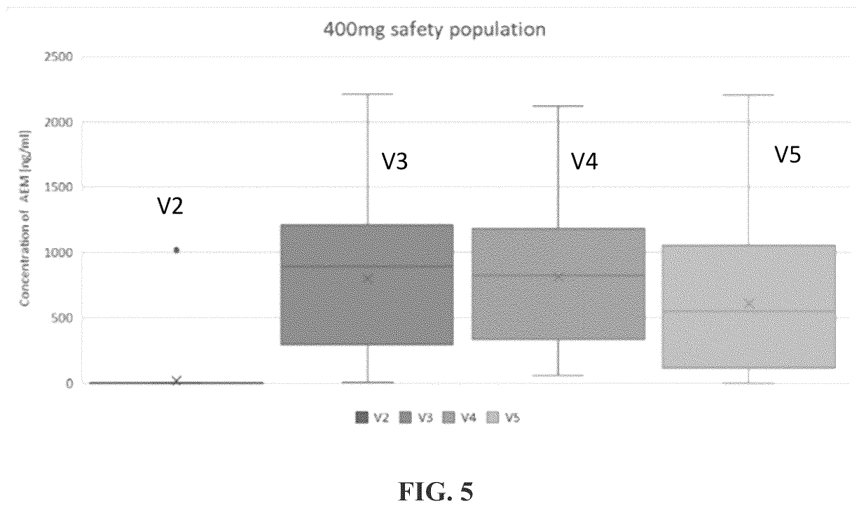

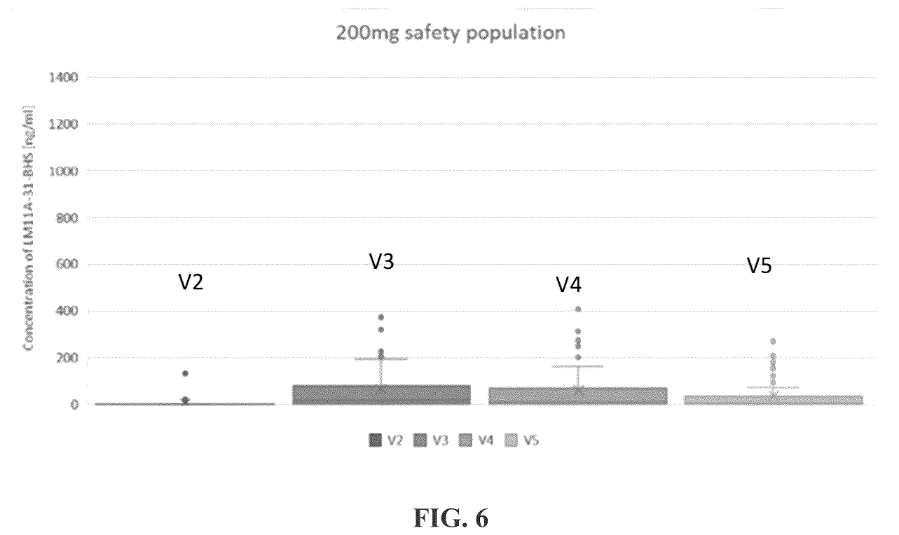

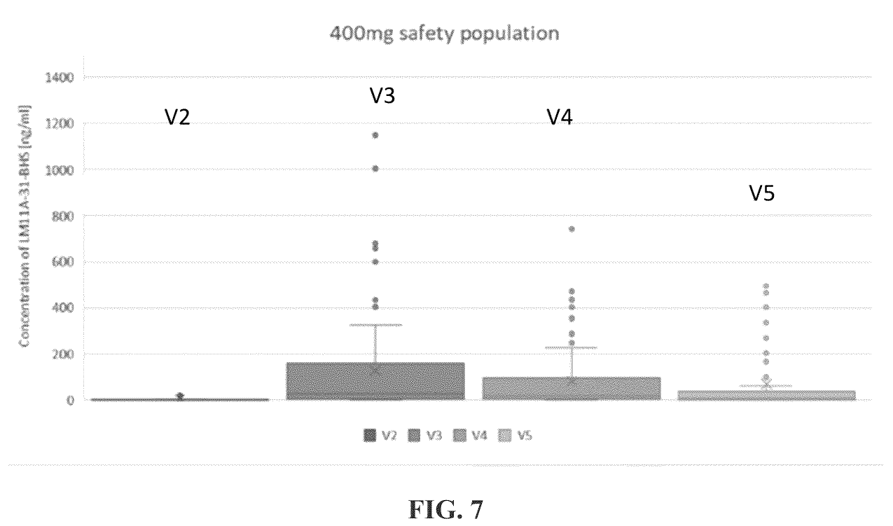

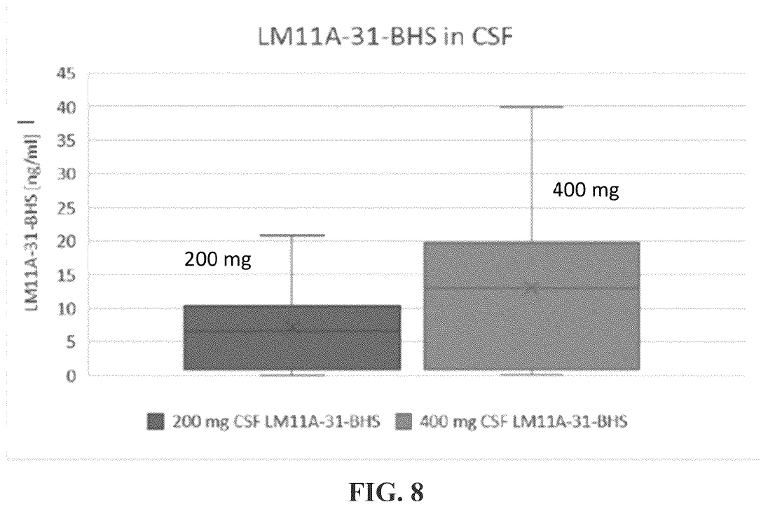

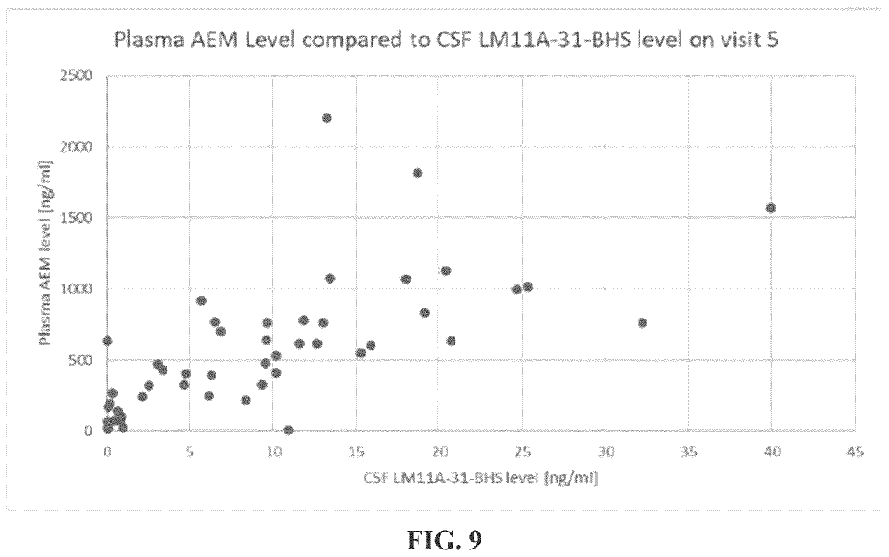

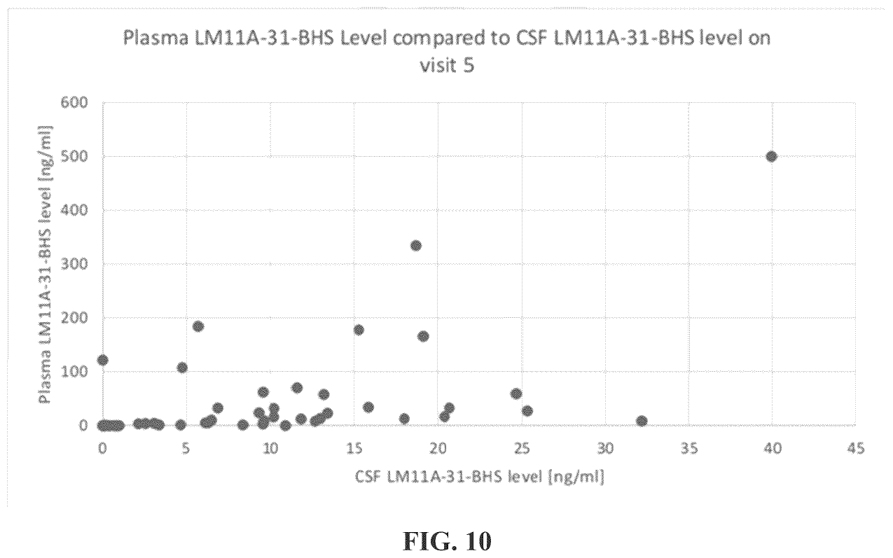

Measurements of the LM11A-31 level and AEM level in plasma (FIGS. 4-7) and, if CSF samples from Visit 5 were available, the LM11A-31 level in CSF (FIG. 8-10).

A 1:1:1 (dose 1: dose 2: control) allocation ratio was used throughout the central randomization process and was structured to allow for a total of at least 240 evaluable patients (80 per group) with center stratification, as only stratification variable, and appropriate block size. During the trial 242 patients were finally randomized, treated and accounted for in the ITT defined population (LM11A-31 200 mg bid 78, LM11A-31 400 mg bid 83, Placebo control 81 patients). Subsequently, 220 and 211 patients for the Per Protocol and Completers analysis subsets respectively were defined. For all safety evaluations, an overall safety population was defined which included all patients who received at least one dose of medication.

### Patient Disposition

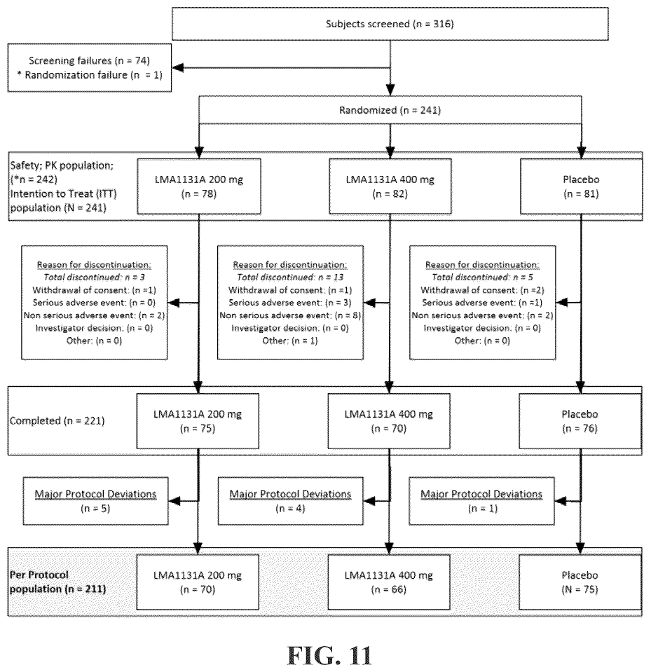

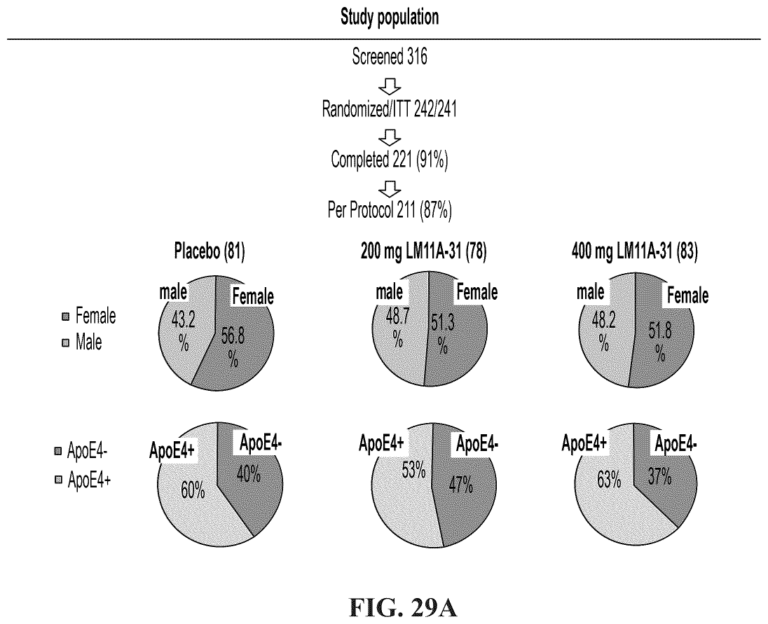

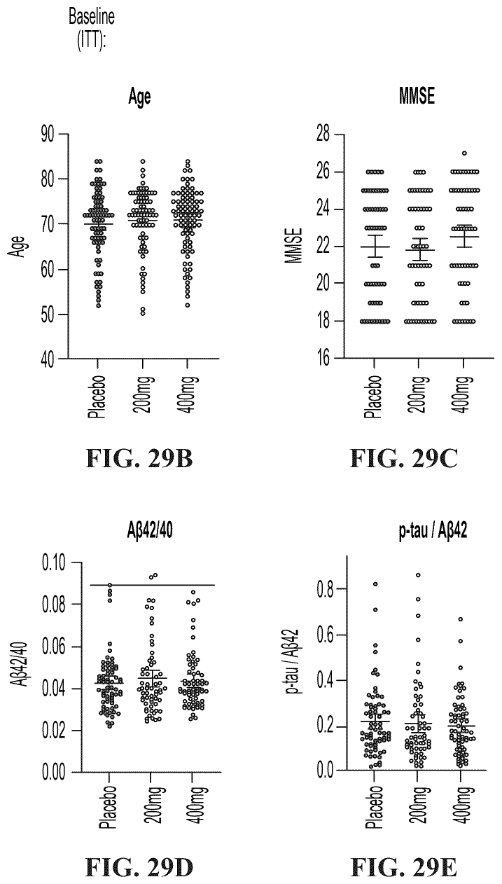

Overall, 316 patients underwent screening and 242 patients were randomized, treated and accounted for in the Safety/PK defined population (LM11A-31 200 mg bid 78, LM11A-31 400 mg bid 83, Placebo control 81 patients). One subject (LM11A-31 400 mg bid) was not properly randomized via the 1WRS system and was therefore not included in the ITT population. From the subsets of 221 and 211 patients for the Per Protocol and Completers analysis subsets were subsequently defined. (Table 5, FIGS. 11, 12, and FIG. 29A-29E). In FIGS. 29A-29E, the screening process is summarized to reach the 242 subjects that were enrolled into the trial. 221 subjects completed the trial. 20 subjects left the trial for various reasons and 211 subjects completed the trial per protocol. The distribution of enrolled subjects in terms of sex and ApoE genetic status is shown. These distributions across the 3 studies groups are not significantly different, hence the drug effects cannot be ascribed to sex or ApoE differences between the placebo versus drug study groups. Individuals with ApoE4 genetic background have fast rates of decline with Alzheimer's, thus this balance is important. The groups also did not differ with respect to age, MMSE (mini mental status exam—used to define the stage at baseline of the subject), Ab 42/40 ratio and p-tau/Ab42 ratio, another indicator of disease severity at baseline.

TABLE 5
Disposition of patients of the different sites and total

| | | Screening | | |
|---|---|---|---|---|
| Site | Complete | Failure | Withdrawn | TOTAL |
| 101 | 1 | 2 | 1 | 4 |
| 102 | 5 | 2 | 1 | 8 |
| 201 | 33 | 6 | 1 | 40 |
| 202 | 22 | 13 | 4 | 39 |
| 203 | 25 | 6 | 1 | 32 |
| 204 | 18 | 13 | 0 | 31 |
| 301 | 9 | 3 | 1 | 13 |
| 302 | 0 | 0 | 0 | 0 |
| 304 | 15 | 8 | 1 | 24 |
| 305 | 3 | 4 | 0 | 7 |
| 306 | 0 | 0 | 0 | 0 |
| 307 | 0 | 2 | 1 | 3 |
| 308 | 1 | 0 | 0 | 1 |
| 309 | 4 | 2 | 0 | 6 |
| 310 | 12 | 6 | 1 | 19 |
| 401 | 15 | 2 | 4 | 21 |
| 501 | 20 | 1 | 0 | 21 |
| 502 | 3 | 0 | 2 | 5 |
| 503 | 0 | 0 | 0 | 0 |
| 504 | 13 | 1 | 1 | 15 |
| 505 | 22 | 3 | 2 | 27 |
| TOTAL | 221 | 74 | 21 | 316 |

### Protocol Deviations

From the disposition details it appears that the study was conducted to a high standard with over 91% (221/242) patients completing and only 21 patients considered as protocol violations. For analysis purposes the participating sites were pooled according to country strata (Spain 62, Czech Republic 104 and Others (Germany, Sweden, Austria) 75 patients) and the demographics across the three treatment groups appeared comparable.

Most of the protocol deviations were related to missing or incomplete tests or missing samples followed by deviations from the visit windows. Due to AD and the high age of the patients the patients were often not able to perform all test required by the protocol. In addition, the COVID-19 pandemic was contributing to the deviations from the visit windows. An overview about major protocol violations is provided in Table 7.

TABLE 6
Protocol deviations listed by country and categorized and normalized per subject

| | | | | | Visit | | |
|---|---|---|---|---|---|---|---|
| | Eligibility | Prohibited | Treatment | Compliance | window | With- | |
| | deviations | medications | deviations | deviations | deviations | drawal | Unblinding |
| Austria | 12.5% | 0.00% | 12.5% | 0.00% | 0.00% | 25.00% | 0.00% |
| Czech | 0.96% | 0.00% | 0.96% | 1.92% | 0.96% | 5.77% | 0.96% |
| Republic | | | | | | | |
| Germany | 4.17% | 6.25% | 0.00% | 4.17% | 0.00% | 8.33% | 0.00% |
| Sweden | 5.26% | 0/00% | 0.00% | 15.79% | 0.00% | 21.05% | 0.00% |
| Spain | 1.59% | 0.00% | 1.59% | 7.94% | 1.59% | 7.94% | 0.00% |

TABLE 7
Major protocol violations (Safety Population)

| | | | | | | | | | Total | Number | No of |
|---|---|---|---|---|---|---|---|---|---|---|---|
| | | | | | | Visit | | | Number | of | Patients |
| | Site | Eligibility | Prohibited | Treatment | Compliance | Window | With- | | of | subjects | Enrolled |
| Country | No. | Deviations | Medication | Deviation | Deviation | Deviation | drawals | Unblinding | Events | involved | per site |
| Austria | 101 | 1 | | 1 | | | 1 | | 2 | 1 | 2 |
| | 102 | | | | | | 1 | | 2 | 1 | 6 |
| Total | | 1 | 0 | 1 | 0 | 0 | 2 | 0 | 4 | 2 | 8 |
| Czech | 201 | | | 1 | 1 | | 1 | | 3 | 2 | 34 |
| Republic | 202 | 1 | | | 1 | 1 | 4 | 1 | 4 | 4 | 26 |
| | 203 | | | | | | 1 | | 5 | 2 | 26 |
| | 204 | | | | | | | | 0 | 0 | 18 |
| Total | | 1 | 0 | 1 | 2 | 1 | 6 | 1 | 12 | 8 | 104 |
| Germany | 301 | 2 | 2 | | 1 | | 1 | | 1 | 1 | 10 |
| | 304 | | | | | | 1 | | 6 | 2 | 16 |
| | 305 | | | | | | | | 0 | 0 | 3 |
| | 307 | | | | 1 | | 1 | | 2 | 1 | 1 |
| | 308 | | | | | | | | 0 | 0 | 1 |
| | 309 | | | | | | | | 0 | 0 | 4 |
| | 310 | | 1 | | | | 1 | | 2 | 1 | 13 |
| Total | | 2 | 3 | 0 | 2 | 0 | 4 | 0 | 11 | 5 | 48 |
| Sweden | 401 | 1 | | | 3 | | 4 | | 8 | 4 | 19 |
| Total | | 1 | 0 | 0 | 3 | 0 | 4 | 0 | 8 | 4 | 19 |
| Spain | 501 | 1 | | | 2 | | 2 | | 3 | 2 | 20 |
| | 502 | | | 1 | 1 | 1 | 1 | | 3 | 2 | 5 |
| | 504 | | | | 2 | | 2 | | 3 | 2 | 14 |
| | 505 | | | | | | | | 4 | 2 | 24 |
| Total | | 1 | 0 | 1 | 5 | 1 | 5 | 0 | 13 | 8 | 63 |

| | | | | | | | | | | |
|---|---|---|---|---|---|---|---|---|---|---|
| Total | 6 | 3 | 3 | 12 | 2 | 21 | 1 | 48 | 27 | 242 |

### Eligibility Deviations

Overall, 6 eligibility deviations occurred within the complete trial. Single deviations occurred in every country.

### Compliance Deviations

Overall drug compliance was excellent with 93.4% of patients compliant (>=80% prescribed drug taken).

### Withdrawal

Only 21 patients in total within the safety population (n=242) were discontinued. These were, AEs (LM11A-31 200 mg bd 2, LM11A-31 400 mg bd 8, Placebo control 2 patients), serious AE (LM11A-31 200 mg bd 0, LM11A-31 400 mg bd 3, Placebo control 1 patients) and Withdrawal of consent (LM11A-31 200 mg bd 1, LM11A-31 400 mg bd 1, Placebo control 2 patients).

### Rationale and Estimate of Numbers of Subject Required Per Treatment Arm in a Phase 3 Trial of LM11A-31-BHS Conducted for Product Approval and Labeling.

The key factor in determining treatment group size is the measure of clinical effect given that CSF and imaging biomarkers generally exhibit less variation and detect treatment effects at lower n values. There are at least two approaches for estimation of a group size necessary to demonstrate a significant therapeutic effect on the primary clinical outcome measure, in this case ADAS-cog-13.

In the first approach, one can evaluate recent large Phase II or phase 3 efficacy trials with the major caveat that these studies are largely focused on 18-month endpoints and our Phase IIa data is limited to a 26-week endpoint. Another difference is that these recent and current studies generally include prodromal and mild subjects while our trial will include mild-moderate subjects. The Emerge and Trailblazer 2 trials demonstrated slowing of progression by 27% and approximately 35%, respectively, in ADAS-cog-13 scores. In general, within the recent large amyloid antibody trials, differences between placebo and treatment group clinical outcome measures derived at the 18-month time point are 2-3 fold larger than those obtained at the 6-month time points (while the degree of variation at 6- and 18-months is in a similar range). Thus the 30-45% slowing trend noted at 6 months in our Phase IIa study points to a potential 18-month effect size similar to, or perhaps greater than, those found in the Emerge and Trailblazer 2 trials. These and some of the other completed or ongoing phase 3 AD trials that include mild stage subjects have treatment group sizes for a given dose in the 500-subject range.

As a second approach, one can perform a power estimation based on the Phase IIa study. For ADAS-cog scores, our statistics group calculated (80% power, 5% two-tailed significance), based on the Phase IIa ADAS-cog data, that 802 and 616 (ADAS-cog-13 and -11) subjects per treatment arm would be required to demonstrate a significant effect at the 6-month time point. Given the pattern that effect sizes at 18 months tend to be 2-3 time larger than those observed at 18 months, it is estimated that a treatment group size of 500 subjects would be sufficient to detect a significant effect on ADAS-cog-13 with the effect size estimated to be present in the Phase IIa trial. It is also anticipated that lowering the maximum age inclusion criteria from 85 that was used in our Phase IIa trial to 75 or 80 given that in the mild-moderate population, the higher ages tend to progress at slower rates and hence age range can affect trial design and outcome.

Availability of FDA-approved amyloid antibody treatment may create a need to have additional study subjects. Given that one might estimate that up to a quarter of eligible subjects might be on amyloid antibodies when the phase 3 trial is executed, another approach would be to include 700 subjects per arm with the goal of having approximately 500 subjects per arm that are not on amyloid antibodies and thus ensuring statistical comparisons of drug versus placebo without confounding by antibody treatment. This approach will also provide statistical power to address the secondary question of whether adding LM11A-31-BHS to amyloid antibody treatment might provide additive or synergistic combination therapy effect given the separate mechanisms of action.

### Rationale for Two Drug Doses in a Phase 3 Efficacy Trial Conducted for Product Approval and Labeling.

In the Phase IIa trial, in CSF biomarker analyses, for the majority of biomarkers, similar effect sizes were found for both the 200 and 400 mg doses. Analyses for structural MRI remain ongoing. For FDG-PET sROI a greater trend is observed at 400 compared to the 200 mg dose (effect at 400 mg and the difference between 200 and 400 mg doses are not statistically significant). For FDG-PET voxel analysis, 400 mg effect is significant and significantly greater than the 200 mg effect which is significant and significantly greater than the placebo group. In cognitive assessments, trends for slower progression of ADAS-cog and MMSE scores were greater at the 400 compared to 200 mg doses but effects at neither dose reached statistical significance. In the area of adverse events, the rate for several of the adverse events was higher for the 400 mg group and for some events this increase was statistically significant. Of the 16 subjects who discontinued the Phase IIa trial due to AEs or SAEs, 11 were from the 400 mg group, 2 from the 200 mg group and 3 from the placebo group. For SAEs, 13 occurred after dosing: 4 placebo, 2 in 200 mg group, 7 in 400 mg group.

For Treatment emergent adverse events (TEAEs): 80 moderate TEAEs occurred with 21, 15 and 61% in the placebo, 200 mg and 400 mg groups. 10 severe TEAEs occurred with 30, 20 and 50% in the placebo, 200 mg and 400 mg groups. For the most common AE of GI side effects (diarrhea in most cases), approximately 35% of subjects in the 400 mg group experienced these side effects while approximately 8% in the 200 mg group.

Overall, it appears that the 400 mg dose has greater efficacy than the 200 mg dose. Given the significantly greater rate of side effects at the high dose it will be advisable to include the 200 mg dose in the event that an excessive number of subjects experience side effects at the high dose. In addition, while the 400 mg dose is also likely to have greater efficacy at the 18-month time point, it will be a high priority to confirm, with the statistical power of a phase 3 trial, their relative efficacies given the safety profiles.

### Data Quality Assurance (Phase IIa Trial)

The study design, conduct, recording, reporting and archiving of all relevant documents/data was done in accordance with GCP.

An electronic CRF (eCRF, Data Magik Limited, Salisbury, UK) was completed for each patient having entered Visit 1 and thus being screened.

The Kapture software was used for capturing and processing the data generated. The system generated an audit trail tracking every action (e.g., data entry, data modification, query raising, allocating, resolving) taken within the system. All individuals using the Kapture software were accountable and responsible for all actions initiated by their electronic signature. Access attempts to the Kapture software were monitored and appropriate actions were taken in the event of any unauthorized access attempts, or user problems with electronic signatures and logging in. User groups (e.g., Investigators, Site Staff, CRA) received appropriate, documented user training provided by the project manager. Personnel authorized for data entry (e.g., Investigator and Site Staff) were responsible for completing the eCRF in English and had to enter all clinical trial data for every patient in the indicated order. The principal investigator signed and dated the patients' eCRF after completion.

The information collected and entered in the eCRFs had to match the source documents. Once the database was frozen, completed eCRFs were saved on a DVD in pdf format and archived together with the ISF from each site.

### Monitoring

Monitoring Visits were performed by a monitor of the CRO. The trial site was monitored by means of on-site Visits and regular inspection of CRF with sufficient frequency to perform source data verification and ensure compliance with protocol procedures. The monitor advised the investigator regarding the practical conduct of the clinical trial and assisted him/her in working according to the protocol, GCP and regulatory requirements. The trial site/investigator guaranteed direct access to the source documents. Verification of data against original source documents and query resolution was conducted for 100% for the first two patients randomized at the site. For 10% of patients subsequently randomized also 100% source data verification (SDV) had to be performed, however, if data quality would have been not acceptable, extended SDV should have been done until an acceptable level of data quality was achieved. For screening failures, only capability of giving IC and correct completion of informed consent forms (ICF) were be reviewed by the CRA. For all patients who discontinued trial medication 100% SDV on study termination Visit should have been done. Standard monitoring reports were produced after each monitoring Visit. Additional internal study checks were performed by the QA personnel of the CRO.

### Audit and Inspection

Regulatory Authority may carry out an inspection during the clinical trial and/or after trial completion. Also trial-related audits by auditors mandated by PharmatrophiX Inc. are possible. Therefore, direct access to source data/documentation will be provided for audits, for review by the Ethical Committee and for any regulatory inspection.

### Confidentiality and Data Protection

The investigator assures that only authorized persons have access to the patient's personal data. Patients can only be identified by the enrolment log in correspondence with the Subject ID on CRFs. All data contained in the patients' medical history are considered confidential.

### Datasets Analyzed

Four main patient populations provide the basis for all statistical analyses, data evaluations and summaries included in the investigation.

The primary patient population of interest is the ‘Intention to Treat’ population (ITT). This includes all patients who received at least one dose of medication and who subsequently provide any post baseline information. All patients were analyzed according to the treatment they were scheduled to receive.

A secondary supporting population is also investigated. This is defined as the ‘Per-Protocol’ completers population (PPc) and this includes all patients who satisfactorily completed the 26-week treatment period and fully comply with requirements of the protocol regarding the exploratory outcome evaluations. Any major protocol violators were excluded. All patients in this group are analyzed according to the treatment they actually receive.

Furthermore, a sensitivity analysis was carried out using the ‘Per Protocol’ population but including patients who have withdrawn early. This is defined as the PP non-completers population set (PPn).

For all safety evaluations, only an overall ‘Safety’ population is described. This includes all patients who received at least one dose of medication and consider all patients according to the treatment they actually receive.

Additionally, a PK population will be described for only patients receiving active study IMP.

### Demographic and Other Baseline Characteristics

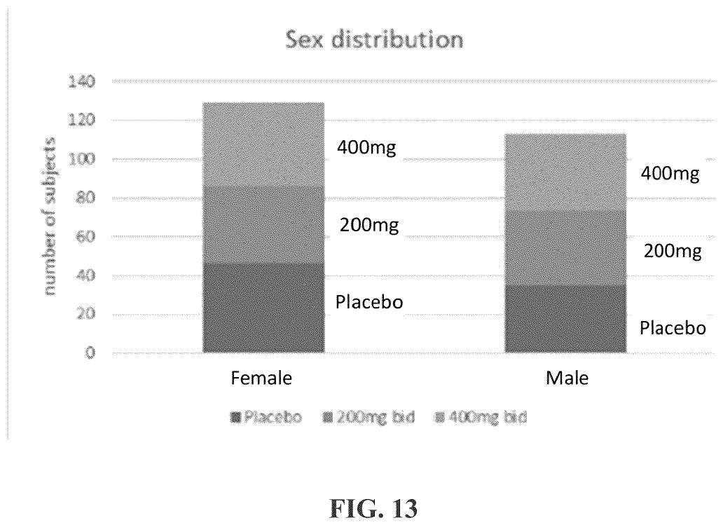

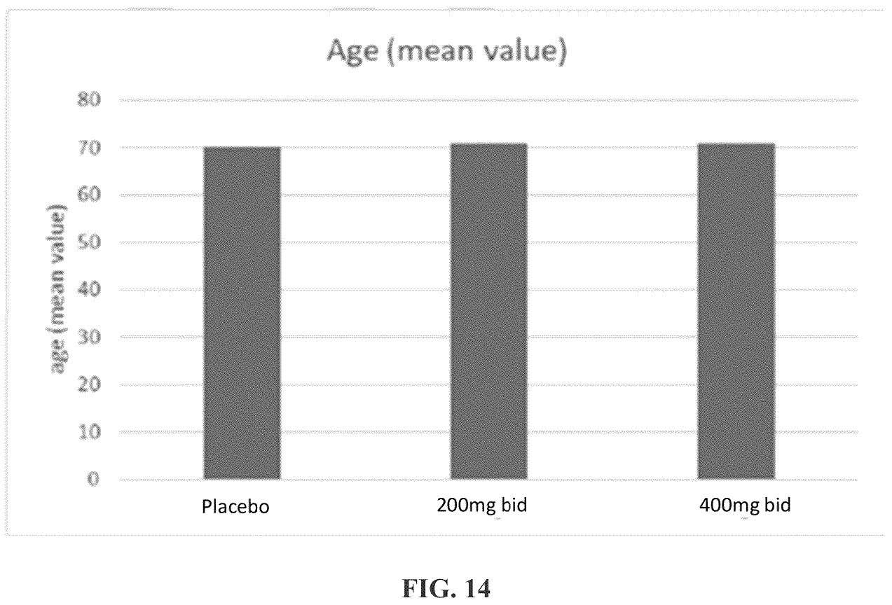

Demographics and baseline characteristics recorded at screening (Visit 1) were summarized using descriptive statistics. The uniform distribution of sex and age within the three treatment groups is visible on FIG. 13 and FIG. 14. Further demographic information is show in Table 8. The race distribution is not shown due to 100% white within this trial.

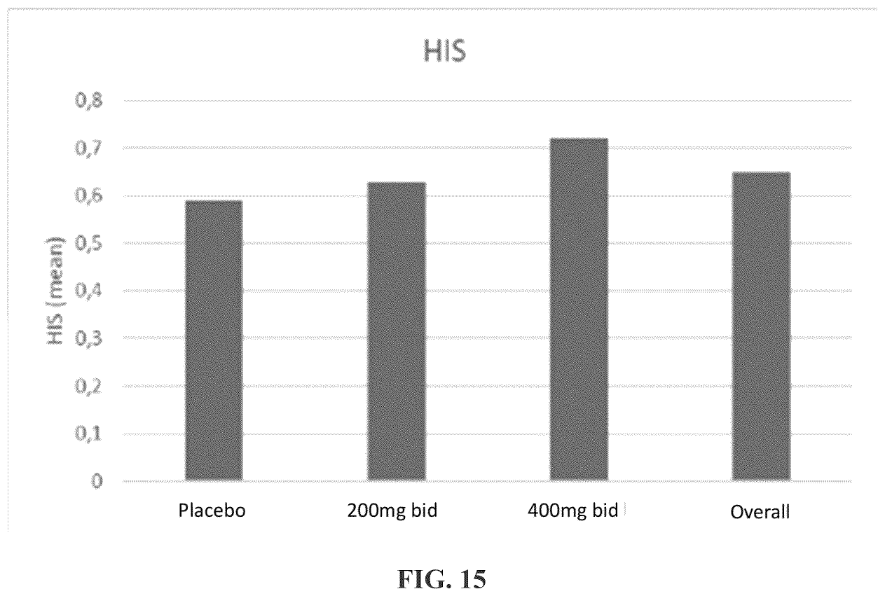

The Hachinski Ischemic Scale (Moroney et al., 1997) (HIS) is increasing within the three groups within the safety population leading to a slight skewness lightly discriminating the dosage groups over the placebo group. (FIG. 15)

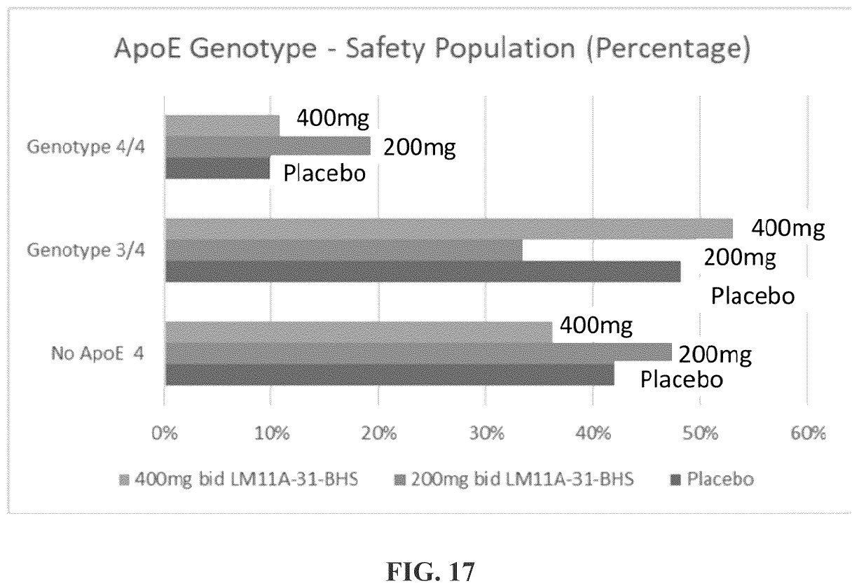

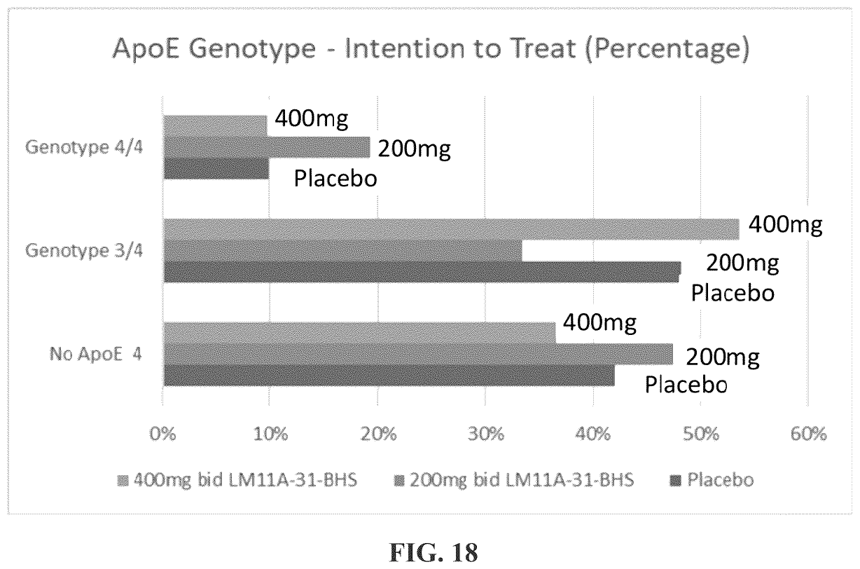

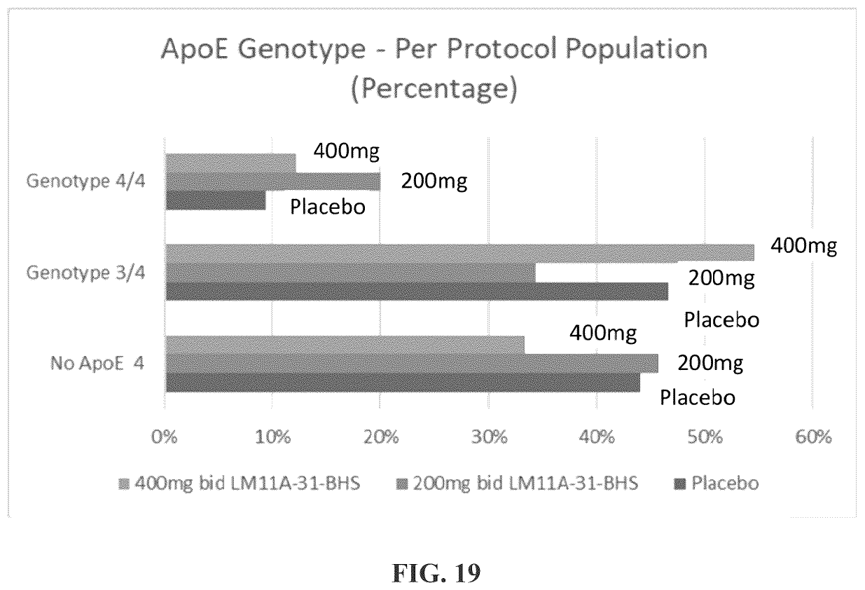

The distribution of the ApoE genotype within the groups reveals a slightly higher percentage of ApoE4 carriers within the highest dosage group and a slight decrease of the ApoE4 carrier percentage within the lower dosage group in the Safety Population. The placebo group has almost the intermediate distribution of the ApoE4 genotype within the ITT Population. Separated to the different genotypes of the ApoE4 carriers the lowest dosage group has the highest number of ApoE 4/4 carriers with almost doubled ratio compared to the residual groups. (See FIG. 17 and Table 9). For the ITT and PP population the percentage of the ApoE 4/4 carriers remains elevated and even increases for the lowest dosage group. (FIGS. 18 and 19, Table 9, and Table 10)

TABLE 8
Summary of demographic characteristics of participants in Phase IIa trial

| | | | | | LM11A | | LM11A | | | |
|---|---|---|---|---|---|---|---|---|---|---|
| | | | Placebo | | 200 mg bid | | 400 mg bid | | Overall | |
| Observation | | Statistic | (N = 81) | | (N = 78) | | (N = 83) | | (N = 242) | |
| Sex | Female | n (%) | 46 | (56.8) | 40 | (51.3) | 43 | (51.8) | 129 | (53.3) |
| | Male | n (%) | 35 | (43.2) | 38 | (48.7) | 40 | (48.2) | 113 | (46.7) |
| Race | White | n (%) | 81 | (100.0) | 78 | (100.0) | 83 | (100.0) | 242 | (100.0 |
| Domicilliary | Own | n (%) | 81 | (100.0) | 78 | (100.0) | 83 | (100.0) | 242 | (100.0 |

| | | | | | | |
|---|---|---|---|---|---|---|
| Status | Home/ | | | | | |
| | Relative | | | | | |
| Age at | | N | 81 | 78 | 83 | 242 |
| Informed | | | | | | |
| Consent | | | | | | |
| | | MEAN | 70.19 | 70.92 | 70.90 | 70.67 |
| | | SD | 7.49 | 6.93 | 7.14 | 7.17 |
| | | MEDIAN | 72.00 | 72.00 | 72.00 | 72.00 |
| | | LQ | 67.00 | 68.00 | 67.00 | 67.00 |
| | | UQ | 75.00 | 76.00 | 76.00 | 76.00 |
| | | MINIMUM | 52.00 | 50.00 | 52.00 | 50.00 |
| | | MAXIMUM | 84.00 | 84.00 | 84.00 | 84.00 |

TABLE 9
Percentual distribution of baseline characteristics (ITT)

| | | 200 mg bid | 400 mg bid |
|---|---|---|---|
| | | LM11A- | LM11A- |
| | Placebo | 31-BHS | 31-BHS |
| MMSE severity - mild | 61.73% | 51.28% | 62.20% |
| MMSE severity - moderate | 38.27% | 48.72% | 37.80% |
| ApoE 2/4 | 0.00% | 2.56% | 2.44% |
| ApoE 3/4 | 48.15% | 30.77% | 51.22% |
| ApoE 4/4 | 9.88% | 19.23% | 9.76% |
| No ApoE 4 | 41.98% | 47.44% | 36.59% |
| Sex - Female | 56.79% | 51.28% | 51.22% |
| Sex - Male | 43.21% | 48.72% | 48.78% |
| Age group - 50-59 | 14.67% | 11.43% | 12.12% |
| Age group - 60-69 | 29.33% | 18.57% | 27.27% |
| Age group - 70-79 | 56.00% | 77.14% | 74.24% |
| Age group - 80-89 | 8.00% | 4.29% | 10.61% |

TABLE 10
Percentual distribution of baseline characteristics (PP)

| | | 200 mg bid | 400 mg bid |
|---|---|---|---|
| | | LM11A- | LM11A- |
| | Placebo | 31-BHS | 31-BHS |
| MMSE severity - mild | 62.67% | 54.29% | 56.06% |
| MMSE severity - moderate | 37.33% | 45.71% | 43.94% |
| ApoE 2/4 | 0.00% | 2.86% | 2.89% |
| ApoE 3/4 | 46.67% | 31.43% | 52.24% |
| ApoE 4/4 | 9.33% | 20.00% | 11.94% |
| No ApoE 4 | 44.00% | 45.71% | 32.84% |
| Sex - Female | 57.33% | 50.00% | 53.03% |
| Sex - Male | 42.67% | 50.00% | 46.97% |
| Age group - 50-59 | 13.33% | 8.57% | 9.09% |
| Age group - 60-69 | 29.33% | 17.14% | 24.24% |
| Age group - 70-79 | 50.67% | 71.43% | 59.09% |
| Age group - 80-89 | 6.67% | 2.86% | 7.58% |

TABLE 11
Different ApoE genotype distribution within groups

| | | No ApoE 4 | Apo E 3/4 | Apo E 4/4 |
|---|---|---|---|---|
| Safety | Placebo | 41.98% | 48.15% | 9.88% |
| population | 200 mg bid | 47.44% | 33.33% | 19.23% |
| | LM11A-31-BHS | | | |
| | 400 mg bid | 36.14% | 53.01% | 10.84% |
| | LM11A-31-BHS | | | |
| Intention | Placebo | 41.98% | 48.15% | 9.88% |
| to treat | 200 mg bid | 47.44% | 33.33% | 19.23% |
| population | LM11A-31-BHS | | | |
| | 400 mg bid | 36.59% | 53.66% | 9.76% |
| | LM11A-31-BHS | | | |
| Per protocol | Placebo | 44.00% | 46.67% | 9.33% |
| population | 200 mg bid | 45.71% | 34.29% | 20.00% |
| | LM11A-31-BHS | | | |
| | 400 mg bid | 33.33% | 54.55% | 12.12% |
| | LM11A-31-BHS | | | |

The Hachinski Ischemic Scale (HIS) is increasing within the three groups within the safety population leading to a slight skewness lightly discriminating the dosage groups over the placebo group (FIG. 15).

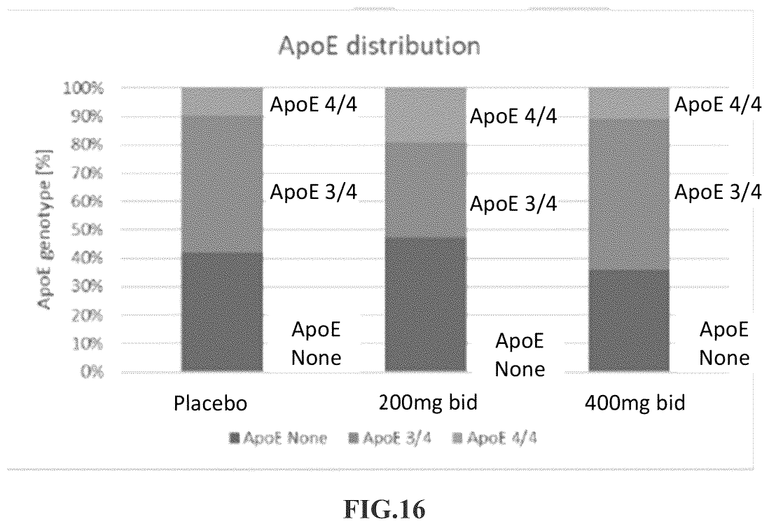

The distribution of the ApoE genotype within the groups revealed a slightly higher percentage of ApoE4 carriers within the highest dosage group and a slight decrease of the ApoE4 carrier percentage within the lower dosage group in the safety population. The placebo group had almost the intermediate distribution of the ApoE4 genotype within the safety population. Separated to the different genotypes of the ApoE4 carriers, the lowest dosage group had the highest number of ApoE 4/4 carriers with almost doubled ratio compared to the residual groups (FIG. 16).

### Measurement of Treatment Compliance

At each assessment Visit the number of capsules dispensed and returned was recorded together with the dates of issue and return. At Visits 3, 4 and 5 (Week 4, 12 and 26 or Early Discontinuation) the actual total number of capsules taken (capsules dispensed−capsules returned) was summed for the entire treatment period. Using the overall start date and end date of treatment the required total number of capsules (end date−start date)*4) was derived and used to express overall treatment compliance as a percentage ((Actual/Required)*100). A ‘cut off’ of >=80% drug taken was used to classify Compliant/Non-Compliant patients. The overall compliance within the different dosage groups was above 90%. The highest number of subjects below the compliance threshold was found in the highest dosage group followed by the low dosage group. Overall, 91.3% (221/242) patients satisfactorily completed the study and only 21 patients were considered protocol violations and excluded from the Per Protocol population. The main reasons for exclusion were <80% compliance or ‘Off’ treatment for more than 3 days (14/242 patients: 5.8%).

### Statistical/Analytical Observations

All data will be described and analyzed according to Endpoint or Assessment Visit (Screening, Week 0, 4, 12, 26), treatment group (LM11A-31 200 mg bid/400 mg bid/placebo) and Overall for each patient population (Safety, ITT, PP).

### Handling of Dropouts or Missing Data

All possible efforts were be made to ensure that patients completed the required assessments at each Visit and in general there was no imputation of any missing data for any of the assessments performed.

To allow for patient attrition or completely missing patient assessments a ‘Last observation performed’ assessment was derived for all exploratory outcome and safety variables. This approach ensured use of the last available none missing post baseline value for all patients and is consistent with the randomization and principal of Intention-To-Treat.

### Analysis of Safety and Exploratory Endpoints

With respect to vital signs, no relevant changes could be identified within the safety population. For heart rate and body temperature no clinically significant changes were measured during the visits.

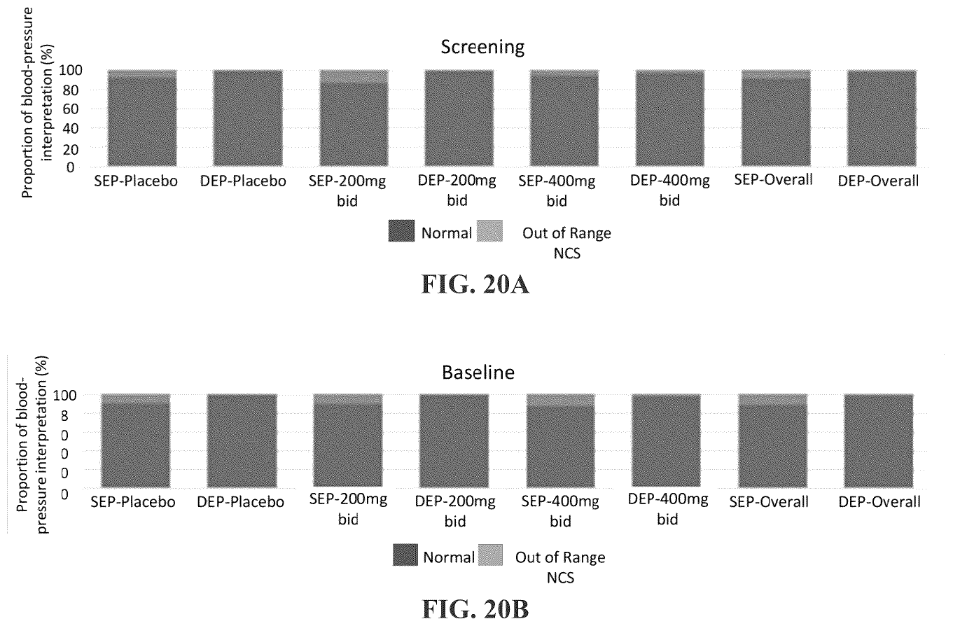

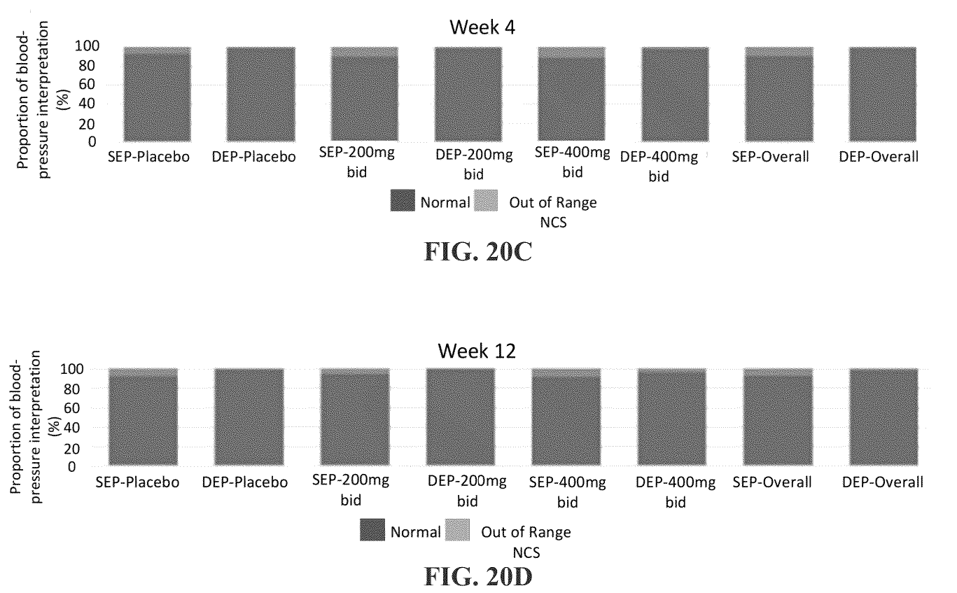

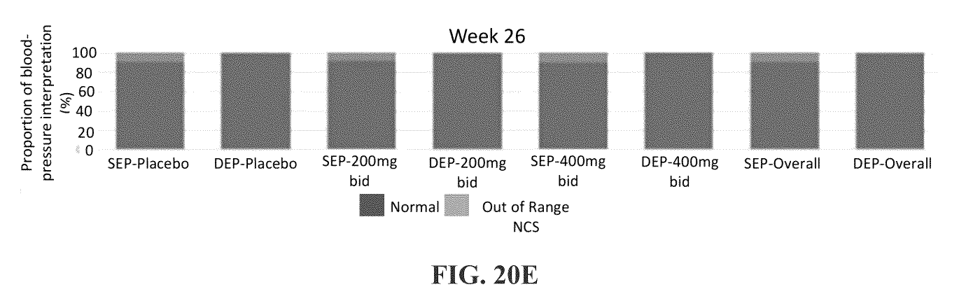

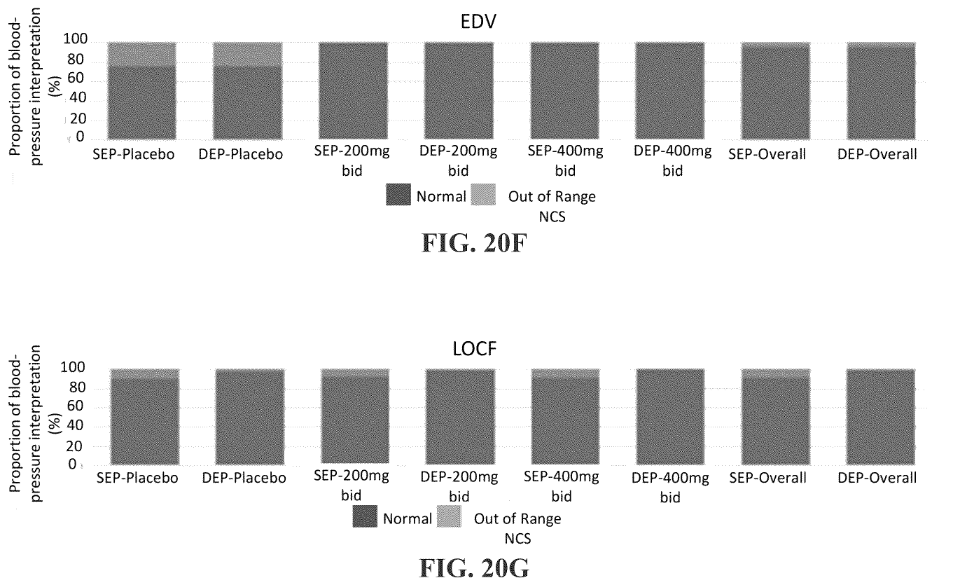

With respect to blood pressure, no relevant changes could be identified within the safety population as seen in FIGS. 20A-20G.

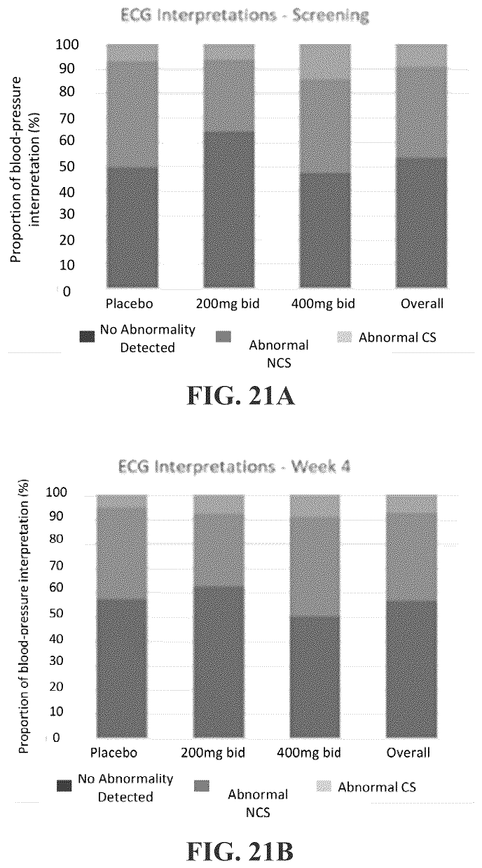

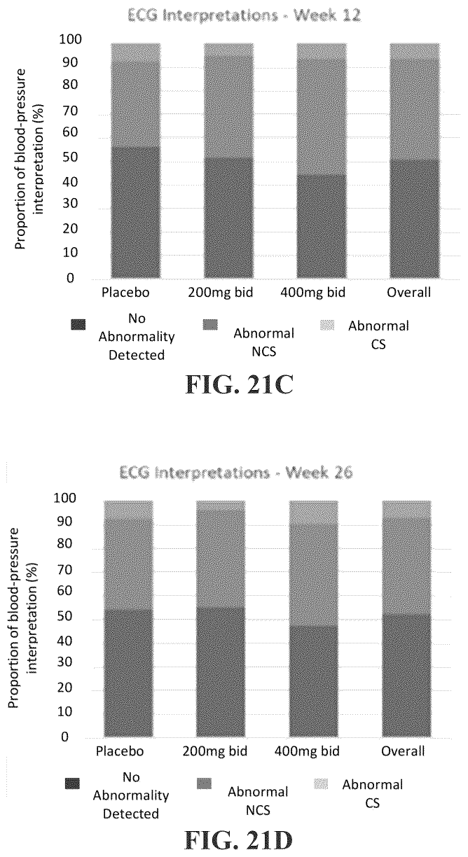

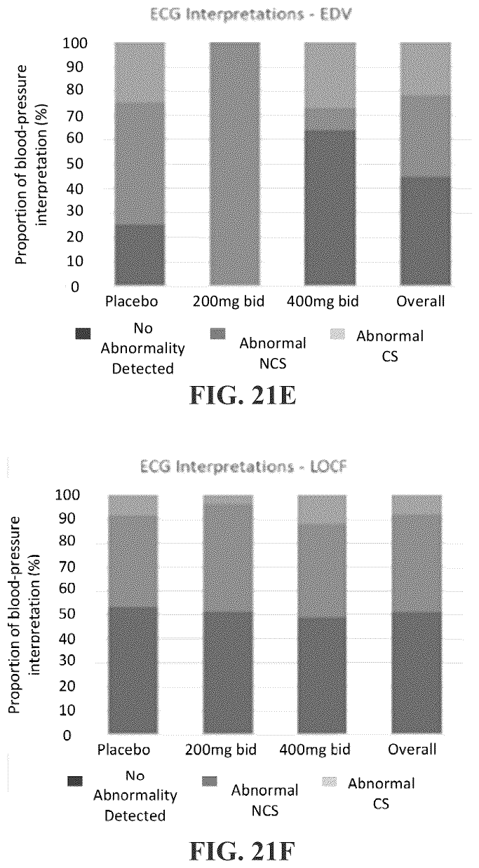

With respect to 12-lead ECG, no relevant changes could be identified within the safety population as seen in FIGS. 21A-21E.

MIRI-based safety analysis found no drug related adverse outcomes including hemorrhage or edema.

The C-SSRS scans were performed at the screening and the baseline visit and the results are listed within Table 12.

TABLE 12
C-SSRS scans performed during Phase IIa trials

| | | LM11A | LM11A | |
|---|---|---|---|---|
| | | 200 mg | 400 mg | |
| | Placebo | bid | bid | Overall |
| | (N = 81) | (N = 78) | (N = 83) | (N = 242) |
| SCREENING | | | | |
| Suicidal Ideation | | 1 (1.3) | 2 (2.4) | 3 (1.2) |
| Wish to be Dead | | 1 (1.3) | 2 (2.4) | 3 (1.2) |
| Non-specific Active Suicidal Thoughts | | 1 (1.3) | 1 (1.2) | 2 (0.8) |
| Active suicidal ideation with Any Methods (Not | | | | |
| Plan) without Intent to Act | | | | |
| Active Suicidal Ideation with Some Intent to Act | | | | |
| without Specific Plan | | | | |
| Active Suicidal Ideation with Specific Plan and | | | | |
| Intent | | | | |
| Suicidal Behavior | | | | |
| Preparatory Acts or Behavior | | | | |
| Aborted Attempt | | | | |
| Interrupted Attempt | | | | |
| Non-fatal Suicide Attempt | | | | |
| Self-injurious Behavior without Suicidal Intent | | | | |
| BASELINE (VISIT 2) | | | | |
| Suicidal Ideation | 1 (1.2) | 1 (1.3) | 2 (2.4) | 4 (1.7) |
| Wish to be Dead | 1 (1.2) | 1 (1.3) | 2 (2.4) | 4 (1.7) |
| Non-specific Active Suicidal Thoughts | | 1 (1.3) | | 1 (0.4) |
| Active suicidal ideation with Any Methods (Not | | | | |
| Plan) without Intent to Act | | | | |
| Active Suicidal Ideation with Some Intent to Act | | | | |
| without Specific Plan | | | | |
| Active Suicidal Ideation with Specific Plan and | | | | |
| Intent | | | | |
| Suicidal Behavior | | | | |
| Preparatory Acts or Behavior | | | | |
| Aborted Attempt | | | | |
| Interrupted Attempt | | | | |
| Non-fatal Suicide Attempt | | | | |
| Self-injurious Behavior without Suicidal Intent | | | | |

### Adverse Events

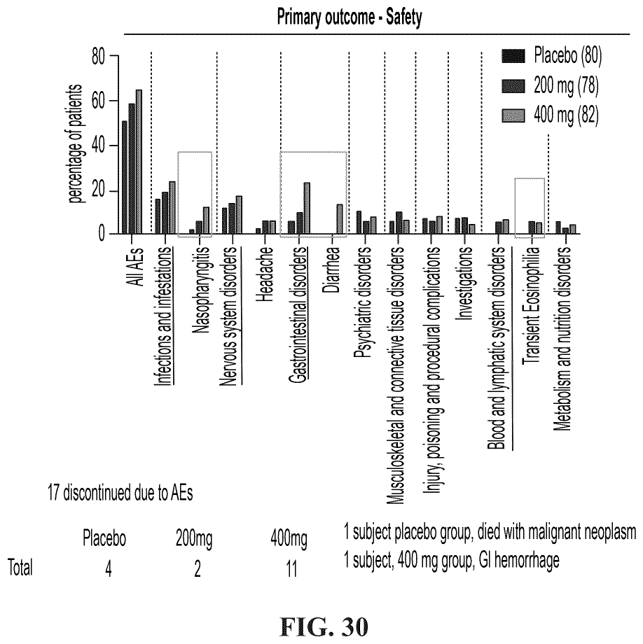

A total of 33 patients (140%) had adverse events considered to be related to LM11A-31. Twelve patients (15%) received LM11A-31 200 mg, 13 patients (15%) received LM11A-31 400 mg and 8 patients (10%) received placebo control. A total of 118 patients (49%) had mild (Grade 1) adverse events, of which 40 patients (51%) received LM11A-31 200 mg, 46 patients (550%) received LM11A-31 400 mg and 32 patients (400%) received placebo control. A total of 46 patients (190%) had moderate (Grade 2) adverse events, of which 11 patients (140%) received LM11A-31 200 mg, 24 patients (290%) received LM11A-31 400 mg and 11 patients (140%) received placebo control. A total of 10 patients (40%) had severe (Grade 3) adverse events, of which 2 patients (30%) received LM11A-31 200 mg, 5 patients (60%) received LM11A-31 400 mg and 3 patients (40%) received placebo control. There were no life-threatening (Grade 4) adverse events reported in any group, and there was one fatal (Grade 5) adverse event reported in the placebo control group. Adverse events are summarized in Table 13 and primary outcome related with AEs is shown in FIG. 30. For the safety results of FIG. 30, 17 subjects left the trial because of adverse events (AEs). AEs were similar between placebo and drug groups except for higher rates in the drug group of nasopharyngitis, GI symptoms including diarrhea and transient, asymptomatic blood eosinophilia.

TABLE 13
Summary of adverse events observed during Phase IIa trial

| | | | | | LM11A | | | LM11A | | | | | |
|---|---|---|---|---|---|---|---|---|---|---|---|---|---|
| | | Placebo | | | 200 mg bid | | | 400 mg bid | | | Overall | | |
| Adverse event | | (N = 81) | | | (N = 78) | | | (N = 83) | | | (N = 242) | | |
| category | | n | % | freq | n | % | freq | n | % | freq | n | % | Freq |
| All Adverse Events | | 42 | 51.9 | 100 | 47 | 60.3 | 109 | 55 | 66.3 | 185 | 144 | 59.5 | 394 |
| Pre-Treatment Signs | | 7 | 8.6 | 9 | 8 | 10.3 | 9 | 8 | 9.6 | 12 | 23 | 9.5 | 30 |
| and Symptoms (PTSS) | | | | | | | | | | | | | |
| Treatment Emergent | | 41 | 50.6 | 91 | 46 | 59.0 | 100 | 54 | 65.1 | 173 | 141 | 58.3 | 364 |
| Adverse Events | | | | | | | | | | | | | |
| (TEAEs) | | | | | | | | | | | | | |
| Drug | Related | 8 | 9.9 | 11 | 12 | 15.4 | 15 | 13 | 15.7 | 23 | 33 | 13.6 | 49 |
| Relationship | Not | 36 | 44.4 | 80 | 41 | 52.6 | 85 | 48 | 57.8 | 150 | 125 | 51.7 | 315 |
| | Related | | | | | | | | | | | | |
| Intensity | Mild | 32 | 39.5 | 69 | 40 | 51.3 | 86 | 46 | 55.4 | 119 | 118 | 48.8 | 274 |
| | Moderate | 11 | 13.6 | 19 | 11 | 14.1 | 12 | 24 | 28.9 | 49 | 46 | 19.0 | 80 |
| | Severe | 3 | 3.7 | 3 | 2 | 2.6 | 2 | 5 | 6.0 | 5 | 10 | 4.1 | 10 |

| | | | | | | | | | | | | |
|---|---|---|---|---|---|---|---|---|---|---|---|---|
| AEs Leading To | 4 | 4.9 | 9 | 5 | 6.4 | 5 | 15 | 18.1 | 20 | 24 | 9.9 | 34 |
| Discontinuation of | | | | | | | | | | | | |
| Study IMP | | | | | | | | | | | | |
| Serious AEs | 4 | 4.9 | 4 | 2 | 2.6 | 2 | 7 | 8.4 | 7 | 13 | 5.4 | 13 |
| AEs Leading To Death | 1 | 1.2 | 1 | | | | | | | 1 | 0.4 | 1 |

The TEAEs reported by frequency show the most frequently reported were Nasopharyngitis (17 patients), Diarrhea (13 patients), Headache (12 patients) and Eosinophilia (10 patients).

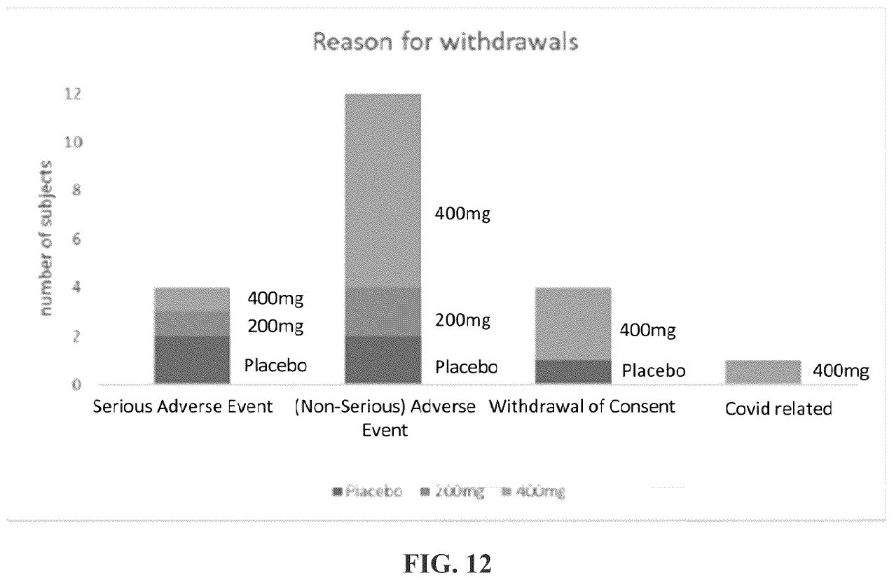

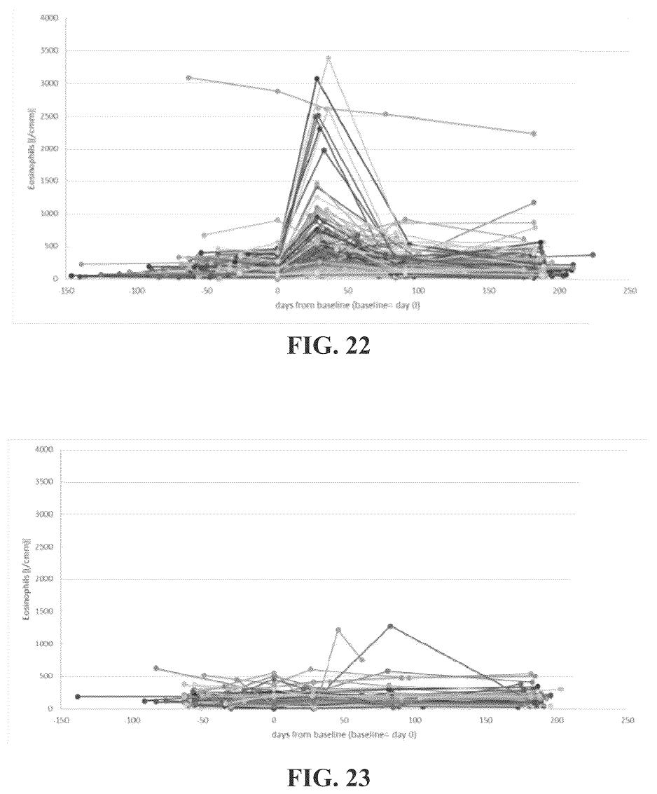

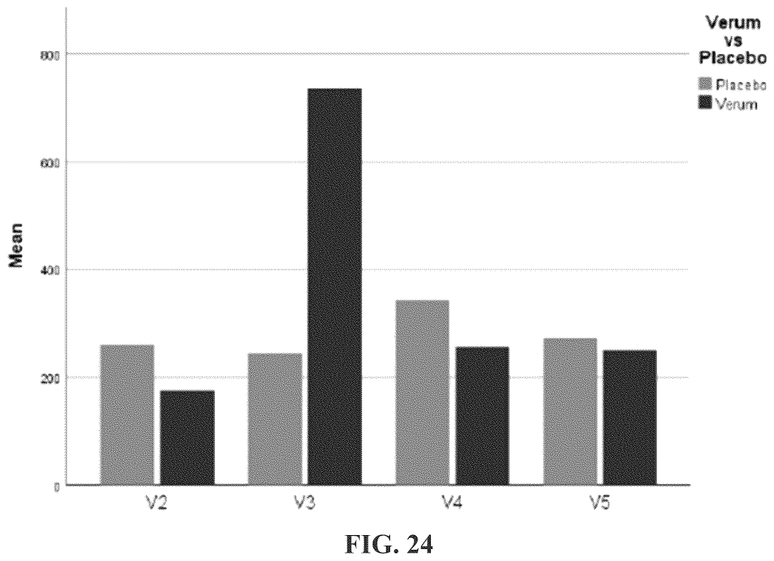

Within the trial, no increase of eosinophils of the treatment group was classified as a serious adverse event, the eosinophil level never reached a value over 5000 per μl that could have been classified as a severe increase, and no combined symptoms gave any indication of a drug induced hypersensitivity syndrome. In total, 10 subjects receiving treatment had eosinophilia reported as an adverse event. In none of these cases was the increase in eosinophil levels thought to be associated with related clinical symptoms. In cases in which treatment was continued, the increase in eosinophil level was found to be transient. Overall, 73 subjects within the verum group had eosinophil counts transiently above 500 per μl. In comparison to the subjects receiving a treatment with the salt of Formula (Ia), subjects treated with placebo showed no changes of eosinophil values (FIGS. 12-24).

This example illustrates the safety and tolerability of the compound of Formula (Ia), utilized in a salt form. Overall, no serious adverse events occurred that raised concerns about the tolerability of the drug.

### Example 13. Observations of Data

No significant changes within the different groups and analyzed populations could be identified for the vital signs, the blood pressure, the 12 lead ECG.

The analyzed MRI data shows no events that raised any concern about the drug safety. The safety laboratory and the urine analysis did not show any patterns of concern. The only statistically significant difference for the treatment groups was an increased eosinophil value that reached values that were considered as clinically significant (11 subjects at week 4) but never reached any severe state and was not associated with any assignable symptoms. Furthermore, all increased eosinophil values either considered as clinically significant or non-clinically significant declined to normal levels again in the following Visits independently from stopping the dosage of the IMP or not. Nevertheless, future trials need to pay special attention to the eosinophils values to uphold and guarantee the safety of the subjects. Overall, no results raised any concerns about the safety of LM11A-31-BHS.

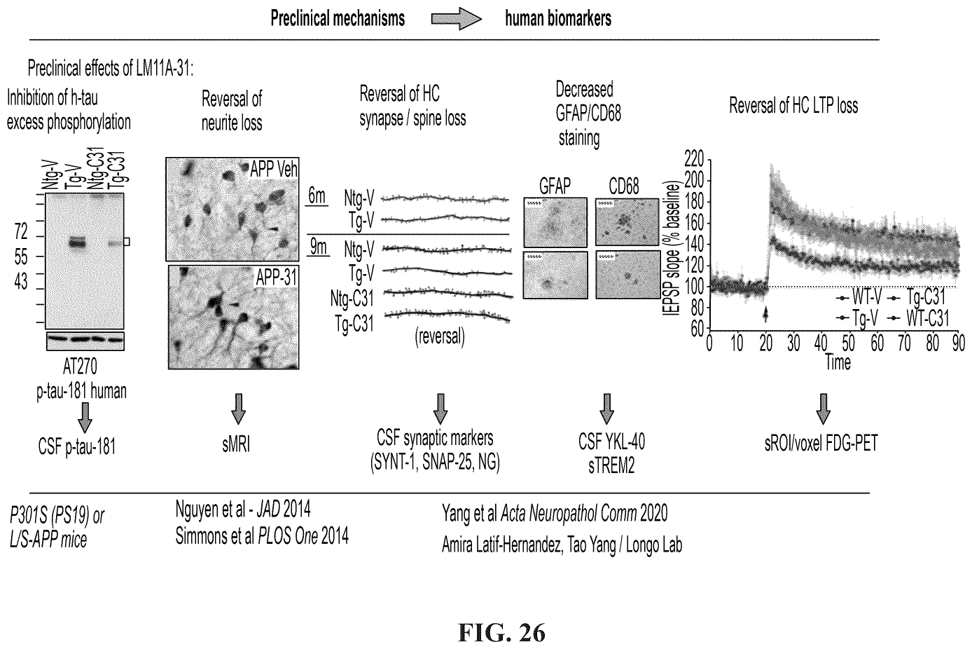

Preclinical studies in mice point to degenerative mechanisms that occur in Alzheimer's Disease (AD) and in mouse models of AD and the human biomarkers that can be used to assess these mechanisms as shown in FIG. 26. In published mouse studies involving amyloid based mice (L/S-APP) or tau based mice (PS19), LM11A-31 inhibits levels of pathological tau, loss of neurites, loss of synapses and spines, activation of astrocytes (GFAP staining is less) and activation of microglia (CD68 staining is less) and corrects synaptic function and hence the loss of LTP signal and measured by EPSP slope in hippocampal slices. Human CSF levels of p-tau indicate degree of pathological tau. Human structural MRI (sMRI) indicates degree of gray matter brain atrophy which occurs in the setting of loss of neurites and synapses. Human CSF levels of synaptotagmin-1 (SYNT-1) and SNAP-25 indicate degree of degeneration of presynaptic elements of synapses; and CSF levels of neurogranin (NG) indicate degree of degeneration of post-synaptic elements, especially dendritic spines. Elevations in CSF levels of YKL-40 and sTREM2 indicate increased activation of astrocytes and microglia, respectively. The term “glia” refers to both astrocytes and microglia. sROI and voxel based FDG-PET is used to measure synaptic function in humans.

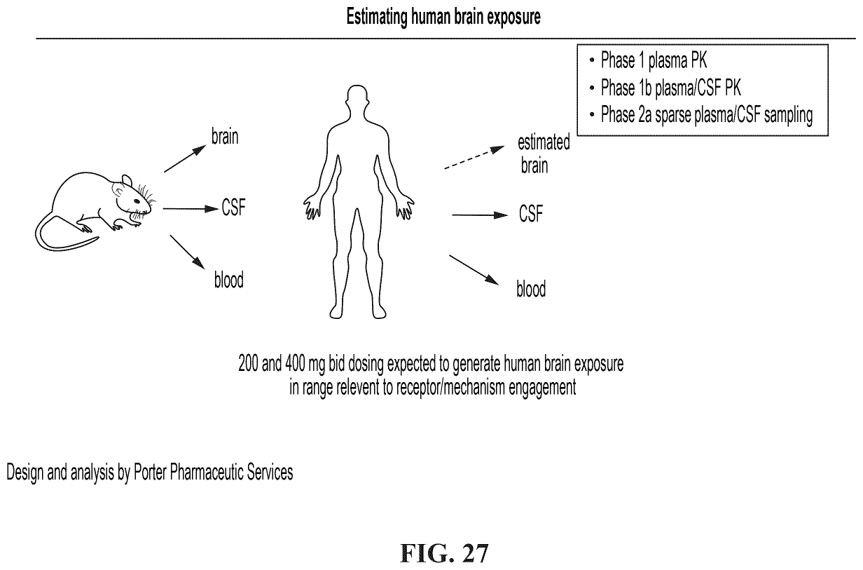

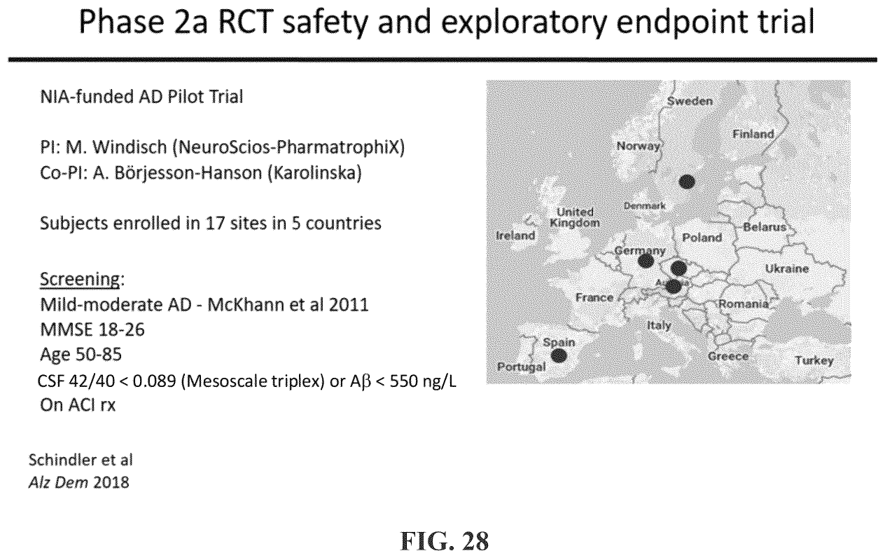

FIG. 27 shows pharmacokinetics studies in mice including measurement of drug levels in blood, CSF and brain tissue and studies done in the 3 listed human studies in which drug levels were measured in plasma in phase 1 and in plasma and CSF in the 1b and 2a study allow the first establishment that oral dosing at 200 mg bid and 400 mg bid lead to estimated human brain levels of drug that are in a range found to engage target mechanisms and derive therapeutic benefit in mouse models. Key inclusion criteria for the 2a trial include a diagnosis of mild-moderate AD by McKhann criteria and a CSF measure of Ab 42/40 ratio under 0.089 OR a Ab 42 level<550 ng/L (FIG. 28). These criteria are discussed in Schindler et al. 2018.

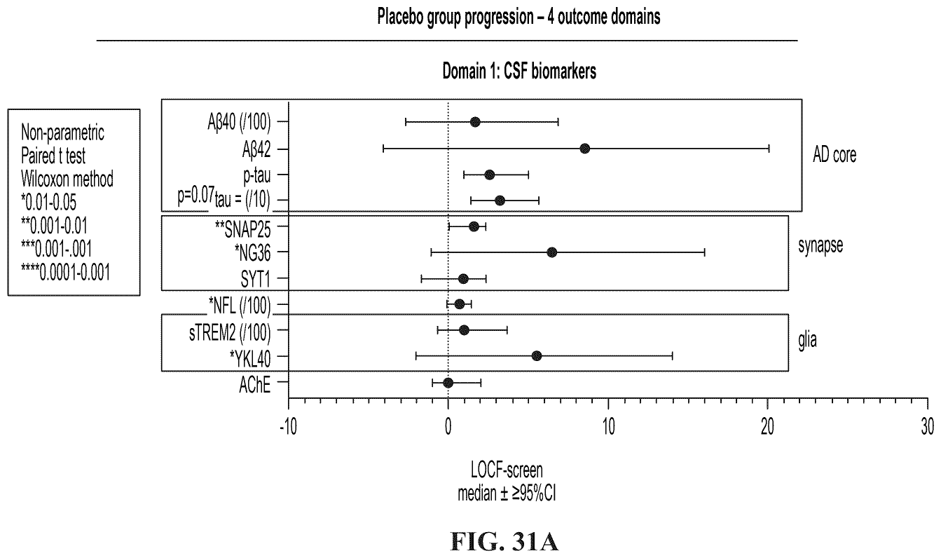

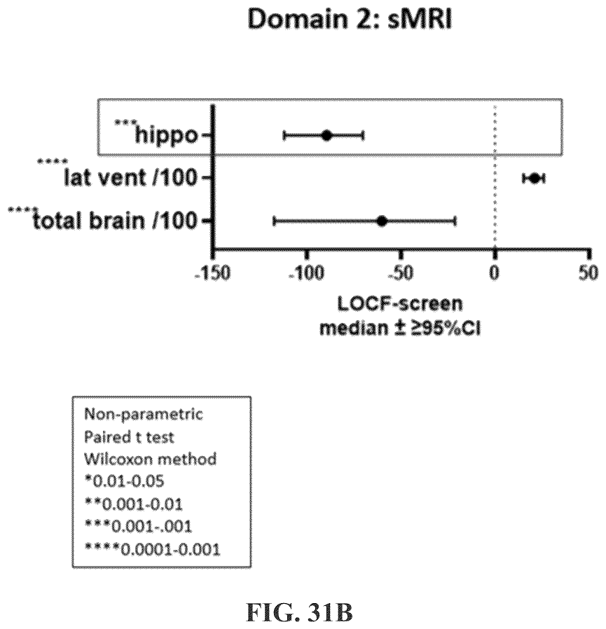

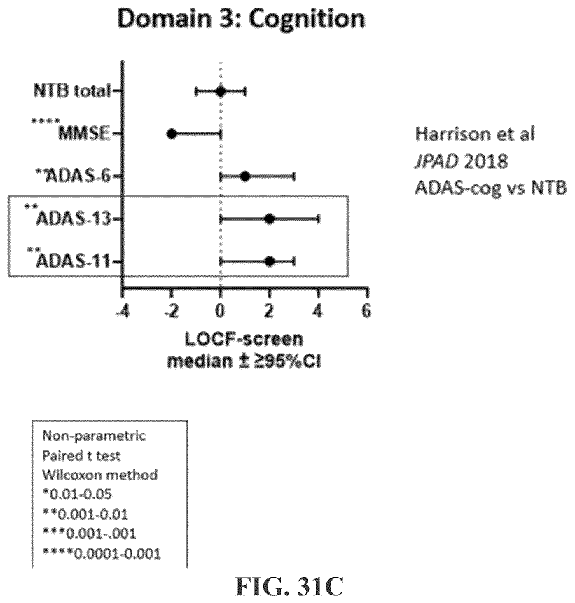

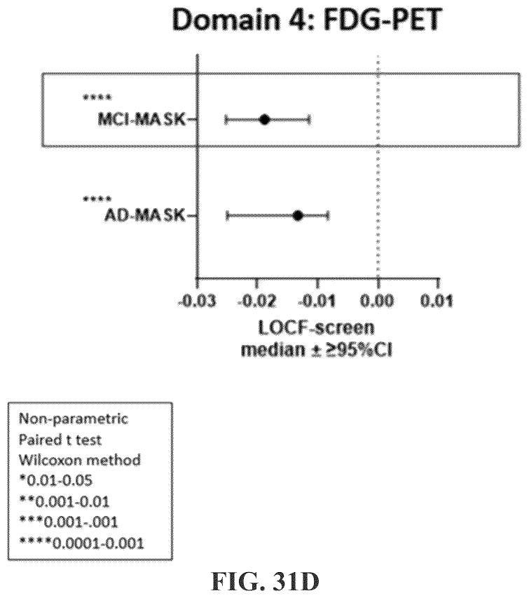

FIGS. 31A-31D demonstrate the progression of the placebo group in the population of mild-moderate AD over the 26-week study period. Many of the outcome measures showed statistically significant progression. Longitudinal progression of CSF biomarkers in the patient population over a 6-month period has not previously been studied well. This information is not obvious but is critical in terms of knowing what to evaluate in the drug groups. Bars moving to the right indicated an increase and for the measures other than Ab, indicating increased degeneration. For MRI, it is shown that the expected decreased volume (i.e., atrophy) of the hippocampus and total brain, for lateral ventricles and increased volume which goes along with brain atrophy. Similarly, for the FDG-PET measures, progression over a 6-month period is not well studied; the shift to the left is the expected loss of metabolic function which indicates loss of synaptic function. Progression in terms of cognition and sMRI is better studied but results are not consistent across studies. Therefore, assessment within this specific group of subjects is critical and would not be able to be predicted. For ADAS cognition, it is shown that the shift to the right of an increased score which is a worsening. For MMSE, the shift to the left is also a worsening. NTB did not change and in the population was not useful.

Exploratory Outcomes for Domain 1 Related with CSF AD Core Biomarkers

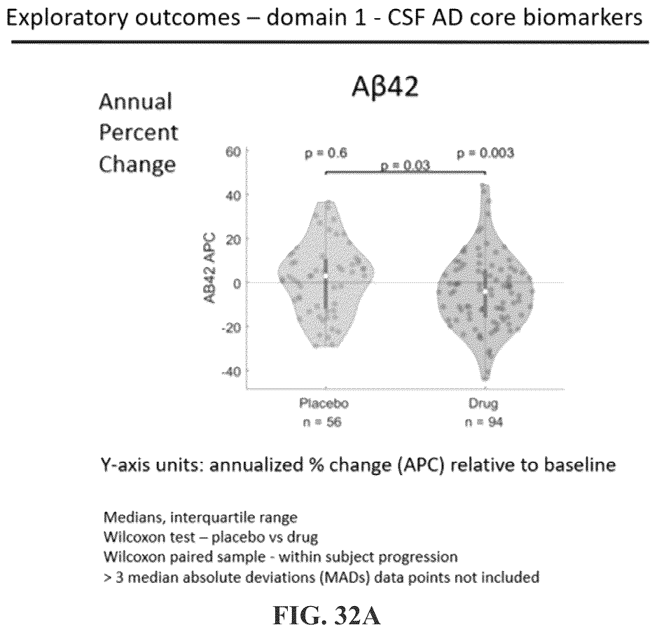

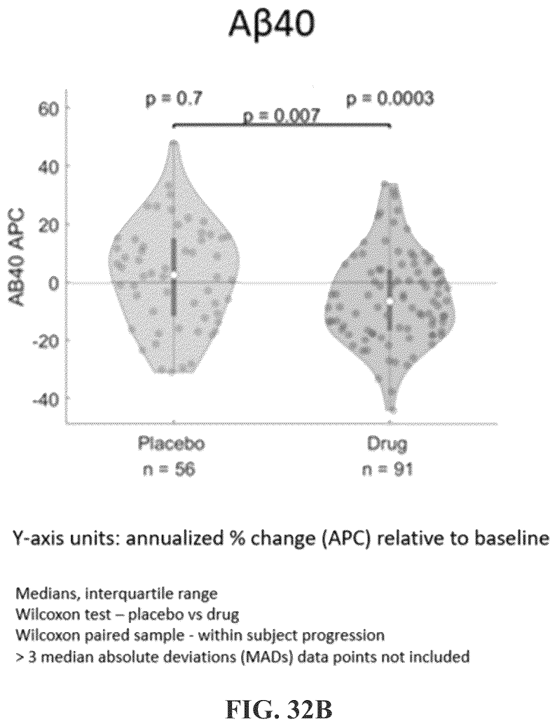

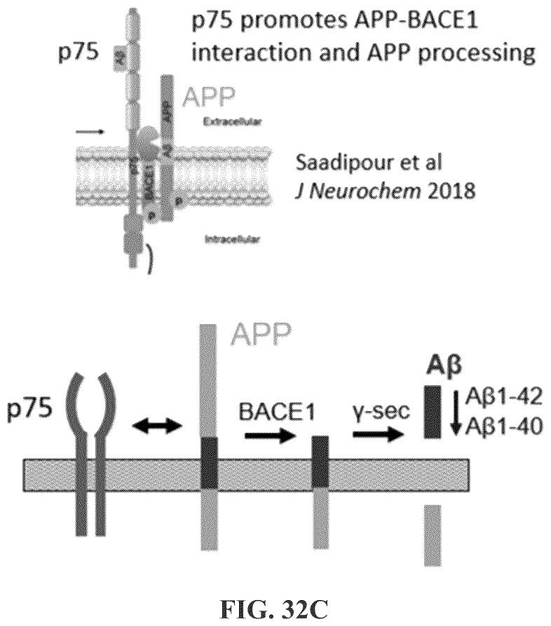

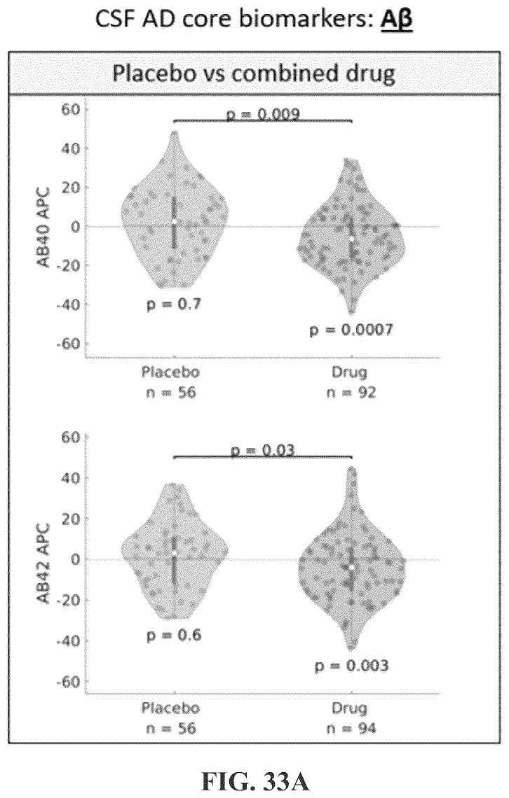

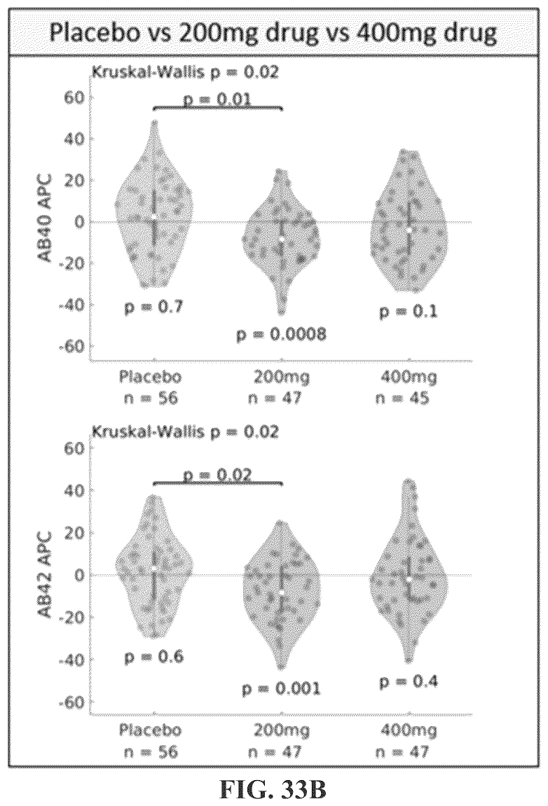

In the placebo group, Ab 42 went up slightly (not significant, p=0.6) but in the drug group it went down by a significant amount (p=0.003) as shown in FIGS. 32A-32C. The difference between placebo and drug is significant (p=0.03). The ability of drug to decrease CSF Ab 42 levels was not expected. The decrease however is consistent with the drug engaging its p75 receptor target. The 2018 study on the right found that if p75 is removed away from the amyloid precursor protein (APP) which it normally associates with, the ability of BACE1 to process APP into Ab42 is decreased. Since LM11A-31 causes endocytosis of the p75 receptor, it would be expected to move it away from APP when it engages p75 and thus LM11A-31 when engaging its p75 receptor target would be expected to lead to decreased CSF Ab levels. This shows, for the first time, that small molecule modulation of p75 is able to affect Ab 42 and Ab 40 levels. Another class of AD treatment, the BACE inhibitors, also has a goal of inhibiting APP processing to Ab 42. Thus LM11A-31 achieves a desired effect in humans that is similar to the BACE inhibitors. The drug also promotes increased volume of the dentate gyrus which is a part of the hippocampus and is vulnerable to degeneration in Alzheimer's and other neurodegenerative diseases. Drug dose analysis of Aβ42 and 40 in CSF in subjects treated with placebo, 200 mg bid or 400 mg bid of LM11A-31 reveals that both doses demonstrate a trend for reduction of Aβ42 and 40 levels relative to placebo with the 200 mg dose reaching statistical significance (FIGS. 33A and 33B). The 200 mg dose also leads to a significant reduction in Aβ42 and 40 relative to pre-treatment baseline values. These findings support target engagement of LM111A-31-BHS with the p75 receptor given that p75 mediates the production of Aβ42 and 40 from the amyloid protein precursor (FIGS. 33A and 33B). As described herein, the dataset indicated as “a combined drug” are derived from corresponding analyses that combine both datasets indicated as “200 mg” and “400 mg” for the purpose of statistical tests.

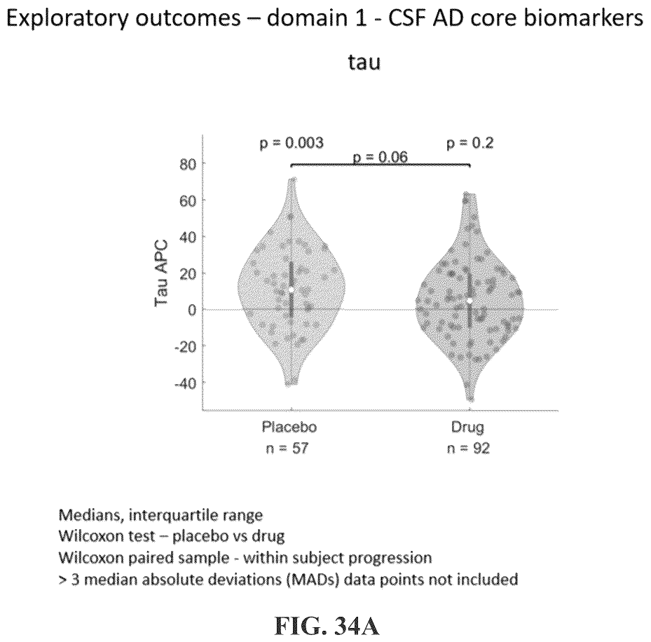

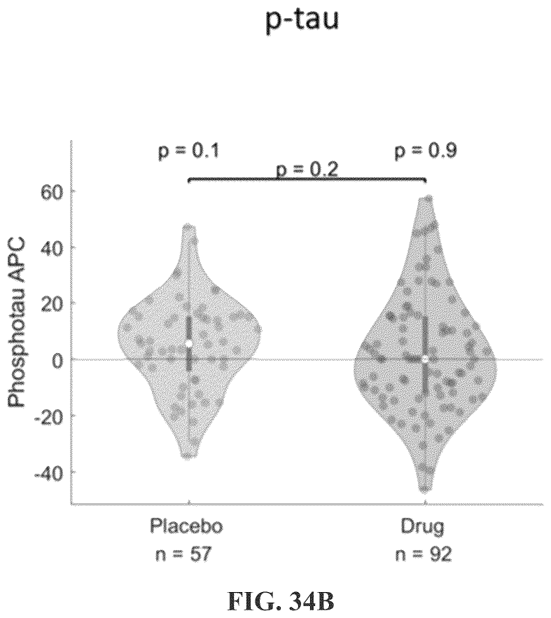

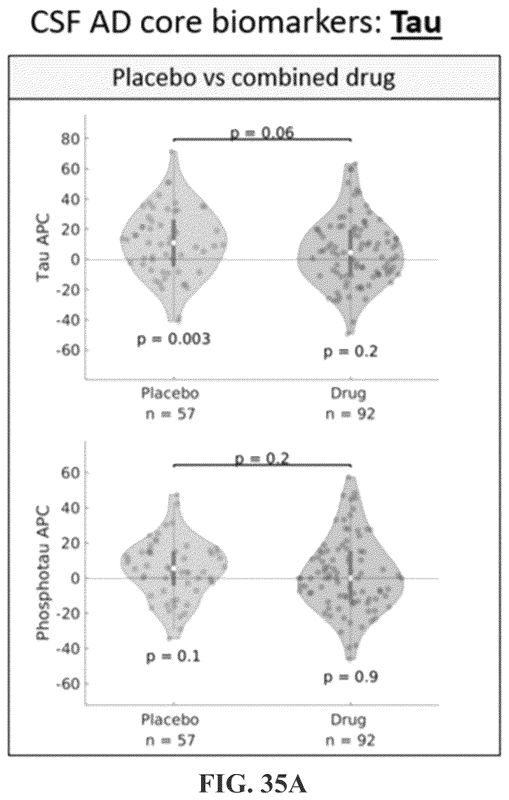

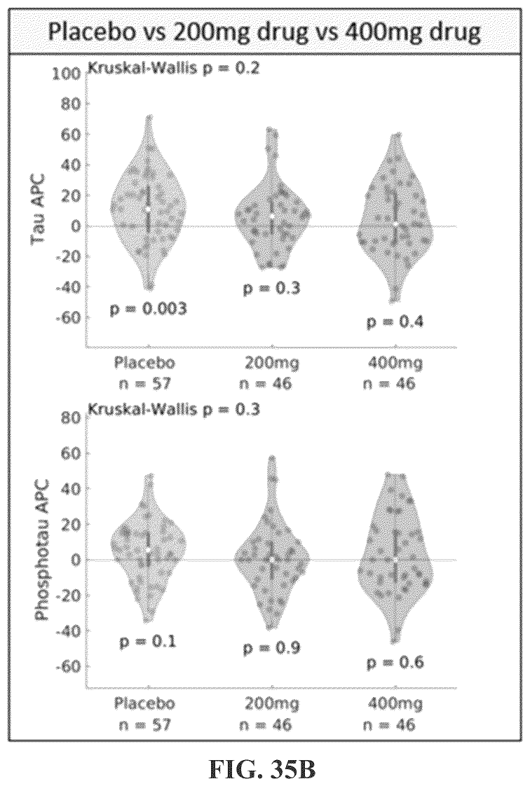

In the placebo group, CSF tau levels increase significantly (p=0.003). In the drug group, this increase is no longer detected at a significant level (p=0.2). When compared to placebo, the drug has a near-significant effect compared to placebo in decreasing the elevation of tau that is occurring over the 6 month treatment period (p=0.06). For p-tau, there is a trend for the drug to inhibit the trend to rise in p-tau in the placebo group (FIGS. 34A and 334B). Drug dose analysis of total tau and phospho-tau CSF levels in CSF in subjects treated with placebo, 200 mg bid or 400 mg bid of LM11A-31 reveals trends for lowering of total tau reduction, especially at the 400 mg dose (FIGS. 35A and 35B). Analysis of p-tau indicates similar trends for reduction at both doses. Total tau analysis demonstrates a significant increase in the placebo group which is no longer present at the 200 and 400 mg doses. The total tau findings suggest the possibility that LM11A-31-BHS, at over the 200 and 400 mg dose range, and possibly at lower and higher doses, reduces neuronal injury and the p-tau findings indicated a drug effect in reducing pathological tau phosphorylation events occurring in Alzheimer's Disease (FIGS. 35A and 35B).

Exploratory Outcomes for Domain 1 Related with CSF Pre-Synaptic Biomarkers

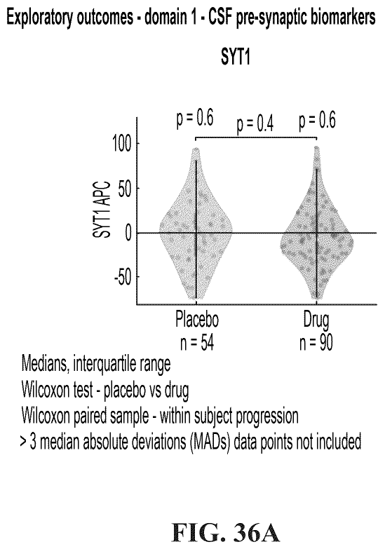

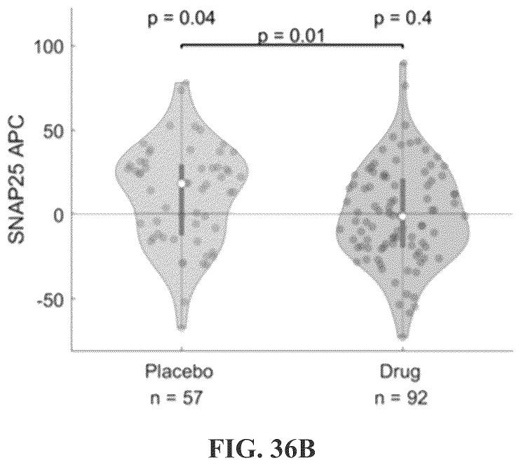

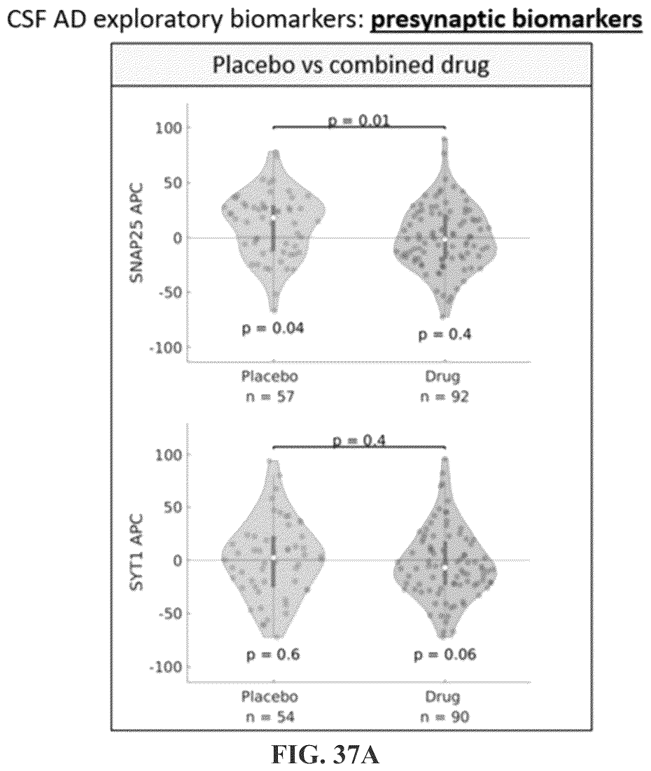

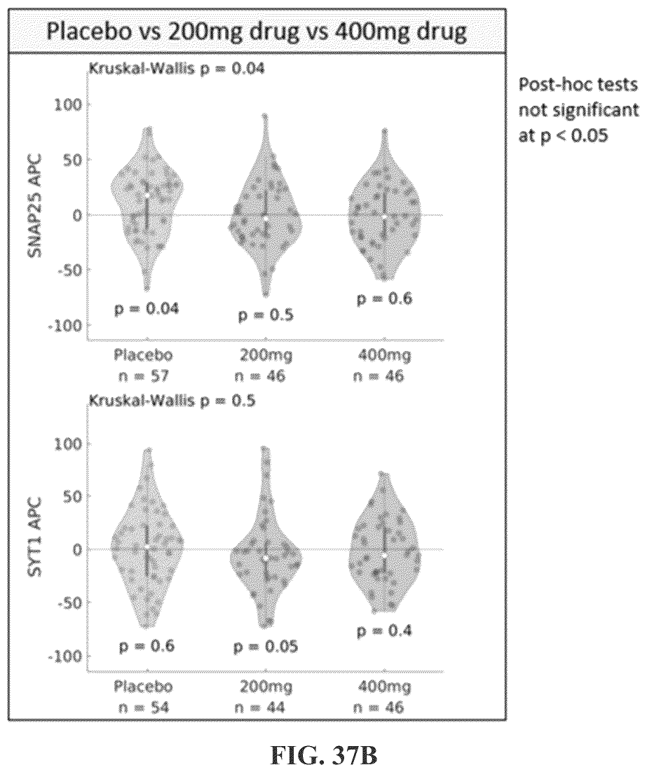

In the placebo group the levels of SYT1 remain about the same during the 6 months of the study (FIGS. 36A and 36B). There is a trend for a decrease in the drug group (p=0.06) indicating a beneficial effect on pre-synaptic components. For the SNAP-25 pre-synaptic marker, there was a significant elevation during the 6-month period indicating significant degeneration of pre-synaptic elements. In the drug group no increase was detected indicating a significant (p=0.01) drug effect in blocking progression of degeneration in presynaptic elements. To our knowledge this is the first demonstration of a therapy preventing degeneration of pre-synaptic terminals as indicated by this pre-synaptic marker. In FIGS. 37A and 37B, the SNAP-25 Kruskall Wallis test detects a difference in the 3 overall group distributions, but the post hoc testing with Dunn's test does not reach significance. Drug dose analysis of the SNAP-25 and SYT1 pre-synaptic markers in CSF in subjects treated with placebo, 200 mg bid or 400 mg bid of LM11A-31 reveals trends for prevention of SNAP-25 increases that are found in the placebo group with a significant main effect of drug dose which appears to be driven by lowering of SNAP-25 at the 200 and 400 mg doses (FIGS. 37A and 37B). SNAP-25 analysis demonstrates a significant increase in the placebo group which is no longer present at the 200 and 400 mg doses. Analysis of SYT1 levels demonstrates only nominal reductions below baseline at both doses. The SNAP-25 data indicates that drug treatment prevents degeneration of pre-synaptic components of synapses.

Exploratory Outcomes for Domain 1 Related with CSF Post-Synaptic Biomarkers

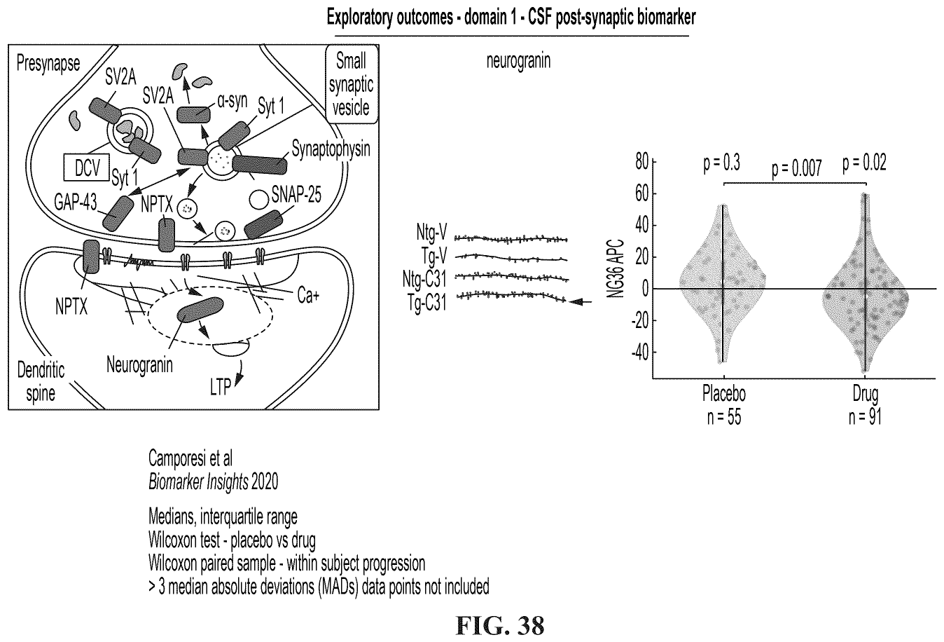

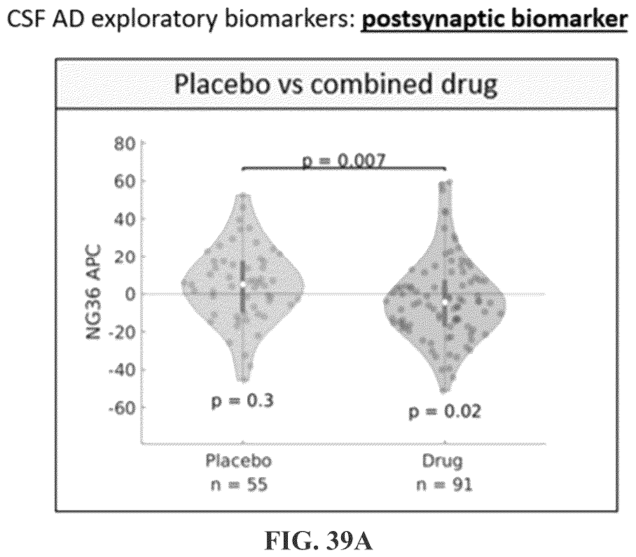

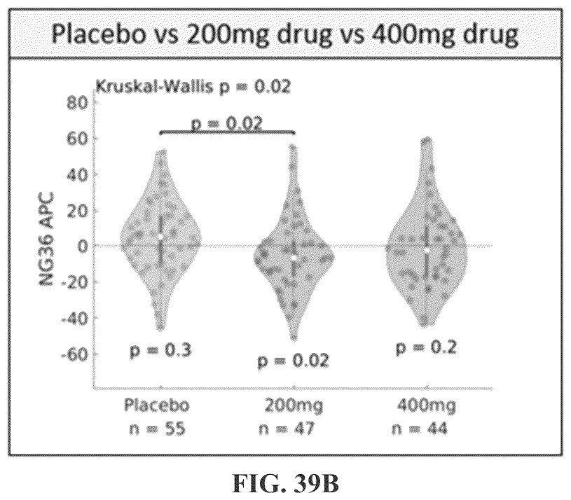

Neurogranin is present in post synaptic elements known as dendritic spines. In the placebo group there was a trend to increase in neurogranin levels during the 6-months (FIG. 38). Notably, in the drug group, there was a significant decrease (p=0.02) in neurogranin levels indicating not only a block of increased degeneration, but a reversal of spine degeneration as was found in the mouse studies (middle panel). Drug dose analysis of the neurogranin-36 post-synaptic marker in CSF in subjects treated with placebo, 200 mg bid or 400 mg bid of LM11A-31 reveals significant lowering of NG-36 at the 200 mg dose and a trend for reduction at the 400 mg dose (FIGS. 39A and 39B). The 200 mg dose also demonstrates a significant lowering of NG-36 relative to the pre-treatment baseline value. This NG-36 data indicates that drug treatment prevents degeneration of post-synaptic components of synapses.

Exploratory Outcomes for Domain 1 Related with CSF Glial Biomarkers

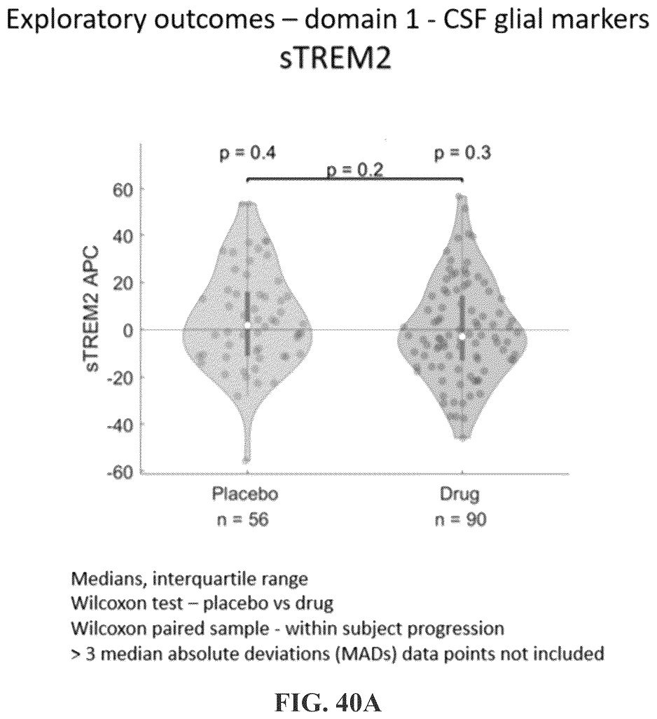

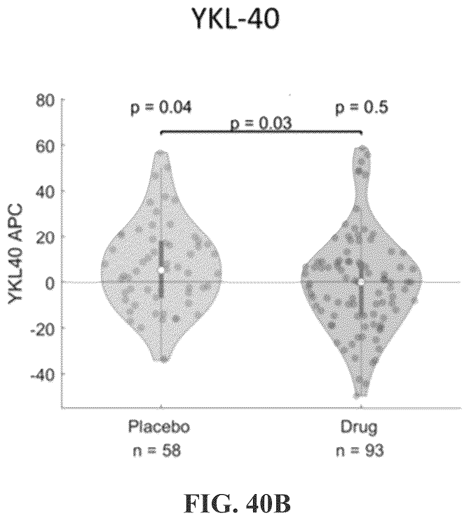

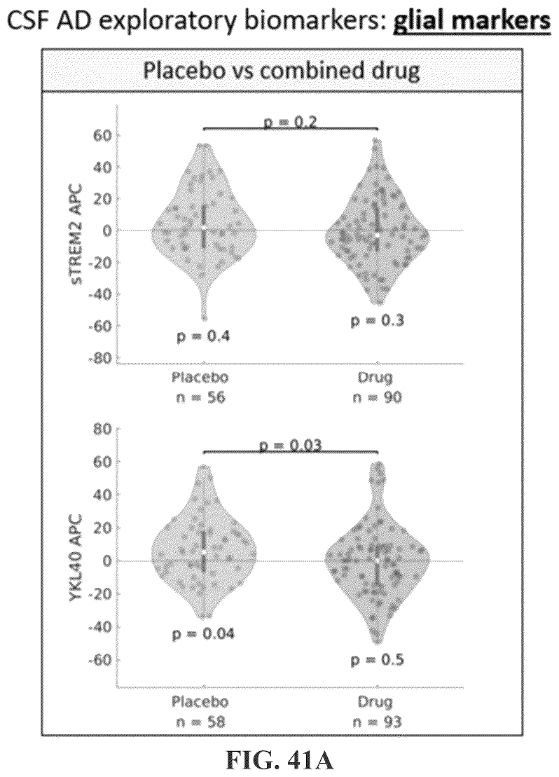

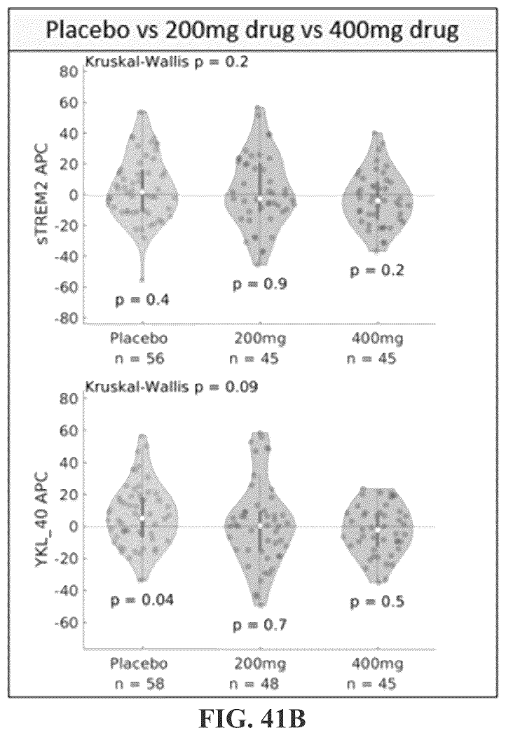

CSF levels of sTREM2 did not change during the 6-month period and no drug effect was detected (FIGS. 40A and 40B). Levels of CSF YKL-40 increase significantly (p=0.04) indicating increased gliosis and neuroinflammation during this period. This increase was not detected in the drug group and the difference between placebo and drug groups was significant (p=0.03) indicting that treatment reduces progression of neuroinflammatory processes. FIGS. 41A and 41B show drug dose analyses of the sTREM2 and YKL40 glial activation markers. sTREM2 analysis reveals minimal change for sTREM2 in the placebo group with a nominal reduction at the 200 and 400 mg doses. YKL40 analysis demonstrates a significant increase in the placebo group which is no longer present at the 200 and 400 mg doses.

### Exploratory Outcomes for Domain 2 Using Structural MRI

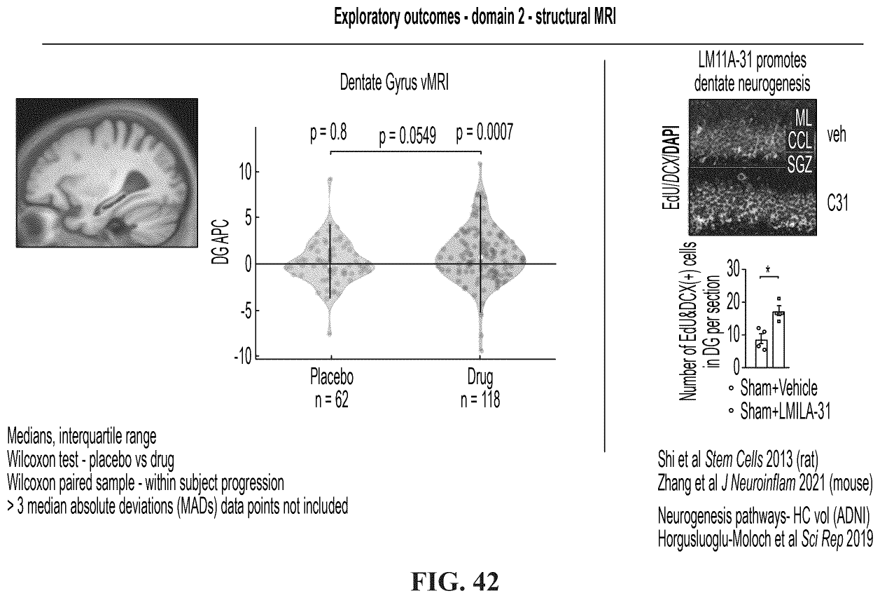

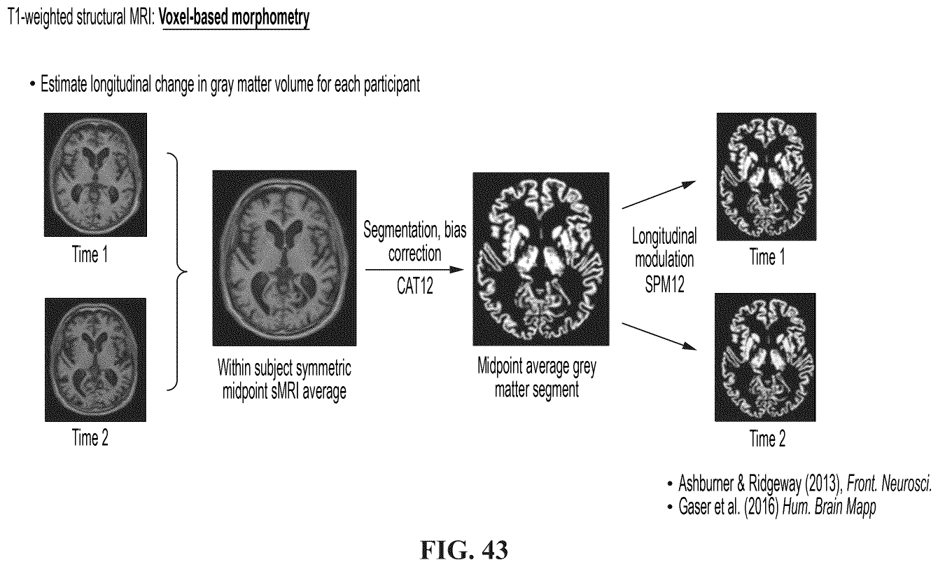

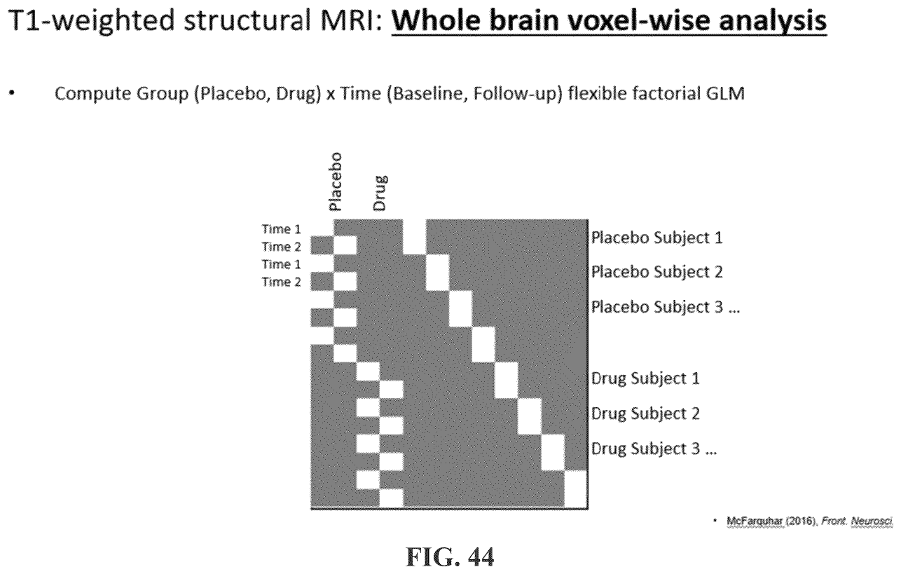

There was no change detected in HC volume during the 6-month period (FIG. 42). Notably, there was a significant increase in HC volume in the drug group (p=0.006). This finding is consistent with the known effects in preclinical models of the drug in promoting hippocampal neurogenesis and promoting neurite growth and complexity. The dentate gyrus is the part of the hippocampus in which neurogenesis occurs (formation of new neurons on the adult). Promotion of neurogenesis is a candidate mechanism for leading to increased volume of the dentate gyrus. One cannot directly measure neurogenesis in humans. The drug was shown to lead to a significant increase in the volume of the dentate gyrus in humans and this effect is consistent with demonstrations that the p75 receptor is present on neuroprogenitor cels and that the drug promotes dentate neurogenesis in mice and rat studies.

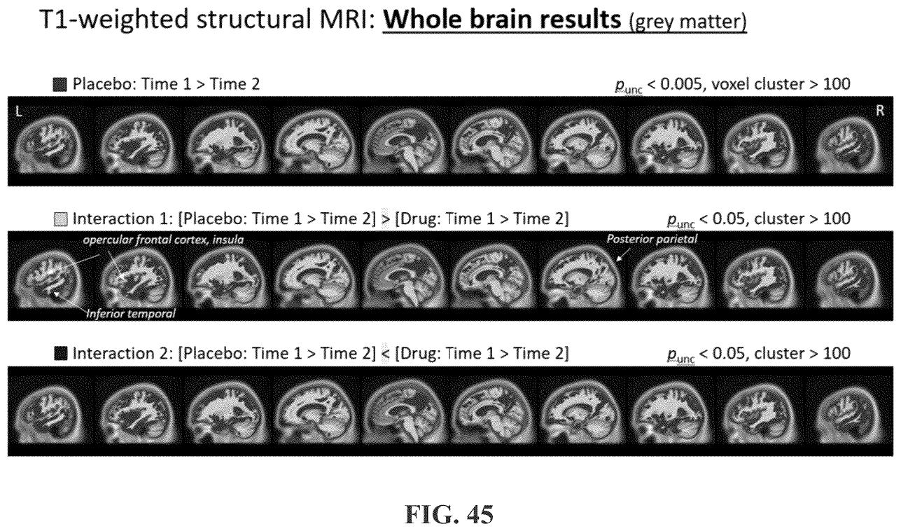

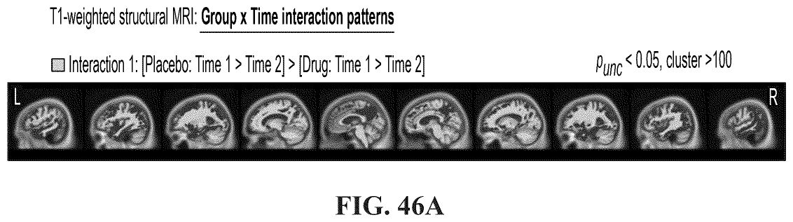

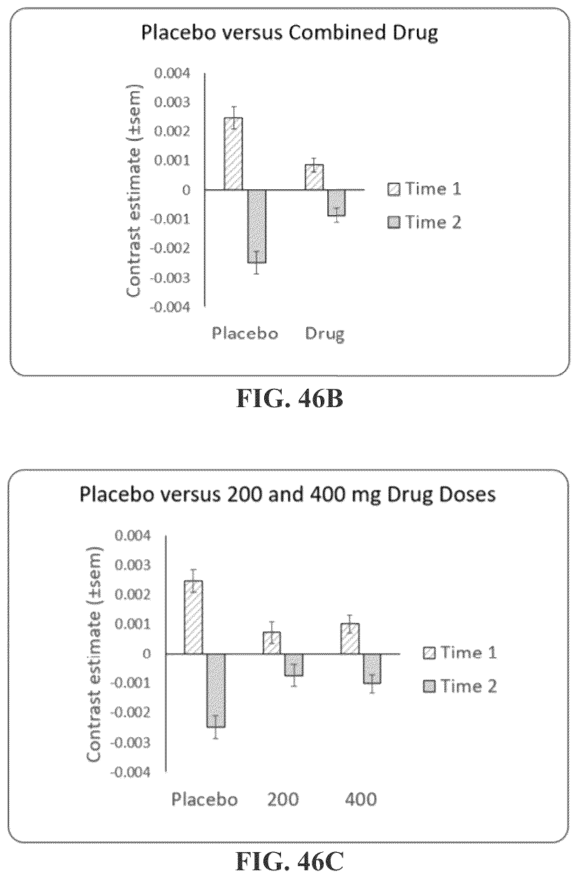

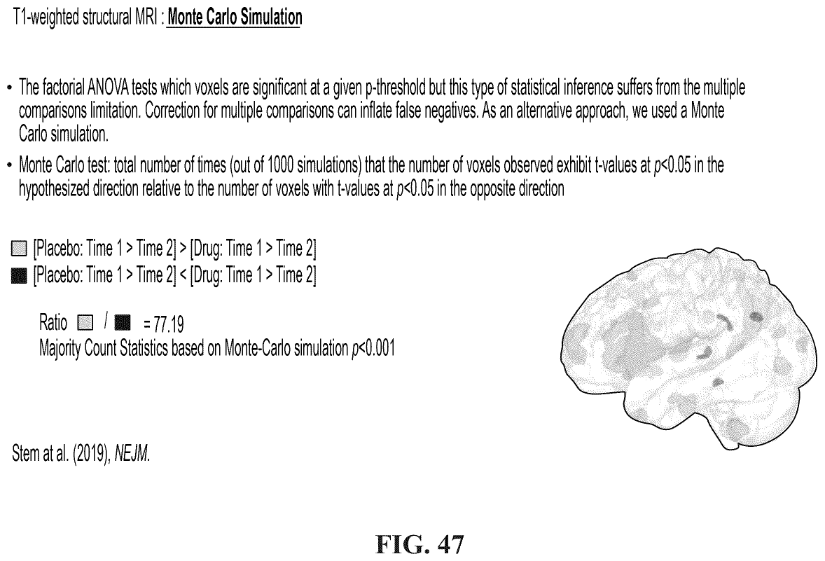

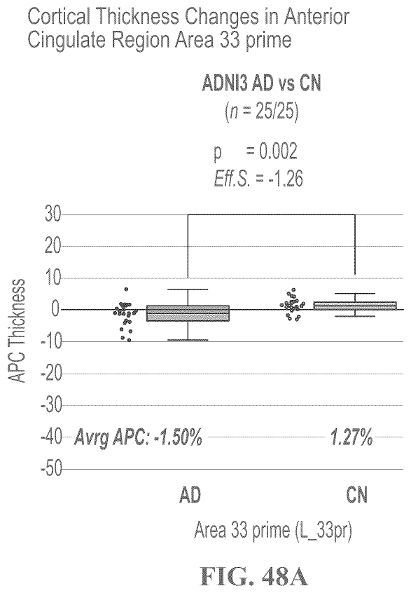

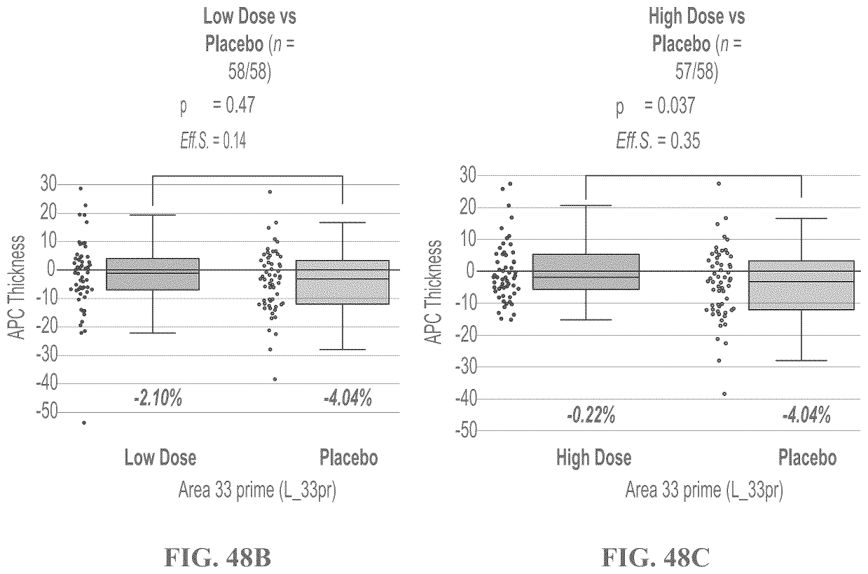

FIG. 45 shows whole brain results of T1-weighted structural MRI. In FIG. 45, upper row indicates (red) regions demonstrating loss of volume in the placebo treatment group between baseline (Time 1) and post-treatment (Time 2). Middle row demonstrates regions of loss of volume as shown in the upper row (red) and areas in green indicate regions in which volume loss in the placebo group was significantly greater than that found in drug-treated subjects. For this analysis, the drug treatment group contains subject treated with either 200 or 400 mg doses. The lower row demonstrates regions of loss of volume as shown in the upper row (red) and areas in blue indicate regions in which volume loss in the placebo group was significantly less than that found in drug-treated subjects. Overall, these findings indicate multiple areas within the degenerating region that demonstrate slowed degeneration in the drug group and rare areas demonstrating increased degeneration in the drug group. FIGS. 46A-46C show group×time interaction patterns using T1-weighted structural MRI. Contrast estimate plots show the magnitude and direction of the interaction effects observed in the flexible factorial GLM. Hence, the bars in green show the magnitude of volume change over Time at each level of treatment Group, averaged over all voxels in the green overlay of the map above, i.e., voxels which show a pattern of [Placebo: Time 1>Time 2] loss of volume greater than drug loss of volume [Drug: Time 1>Time 2] at a p<0.05 and cluster size>100. Dose analysis indicates a significant effect at both the 200 and 400 mg doses in slowing loss of volume. FIG. 47 shows T1-weighted structural MRI, Monte Carlo Simulation (MCS). The ratio and p-value output of the MCS is derived from the total number of times (out of 1000 simulations) that the number of voxels observed in the input map (at a given p and k threshold) exhibit t-values in the hypothesized direction (slowing loss of volume with drug treatment) relative to the number of voxels with t-values in the opposite direction. Therefore, if the actual observed ratio of t-values is close to equivalent in the input map at a given p and k threshold, the MCS ratio and p-value will tend to support the null hypothesis of no drug effect. If the actual observed ratio is skewed heavily in favor of one direction over the other (as demonstrated here with a ratio of 77.19), the MCS ratio and p-value will tend to reject the null hypothesis. These findings demonstrate a significant drug effect in slowing loss of brain volume and is consistent the GLM results. FIGS. 48A-48C show a drug effect on improving cortical thickness or decreasing gyrification in a broad sense as both are indicators of cortical atrophy/degeneration. Increased thickness is similar to increased volume and means less degeneration. Here increased gyrification means increased atrophy.

### Exploratory Outcomes for Domain 3: Cognition

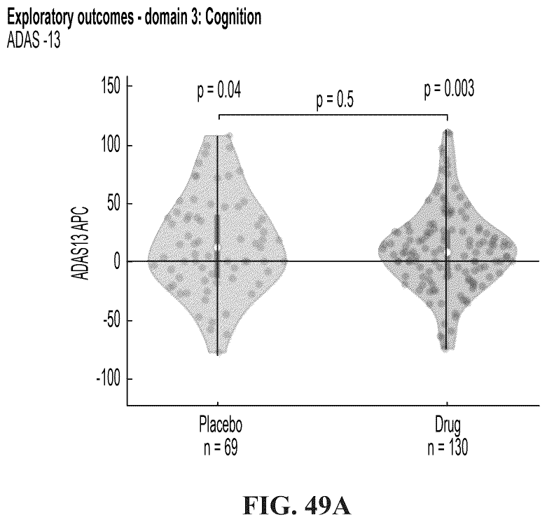

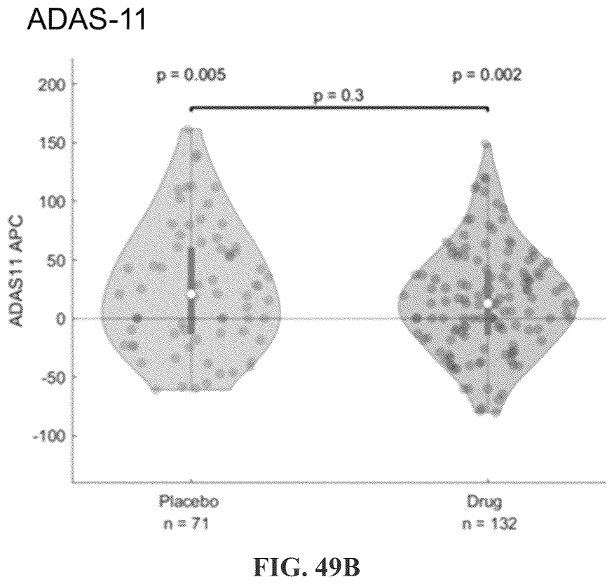

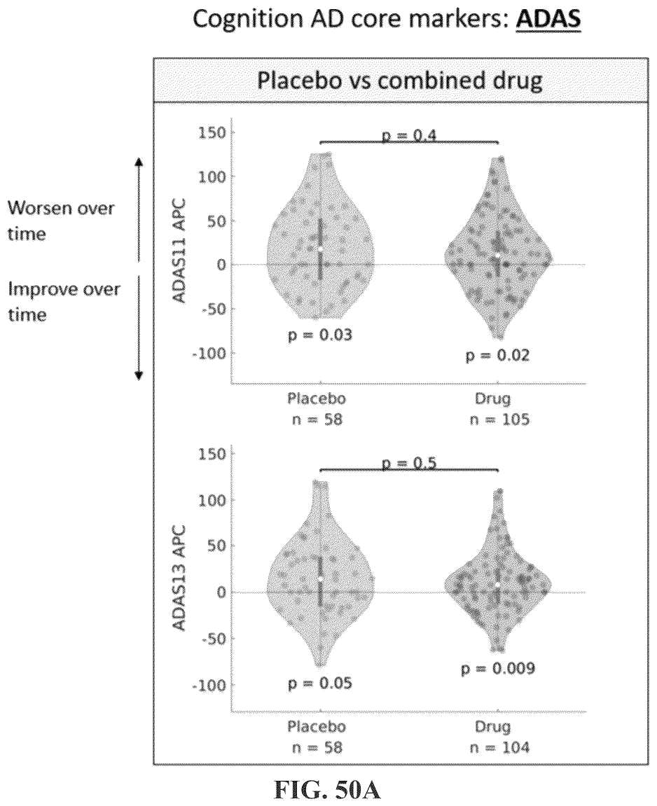

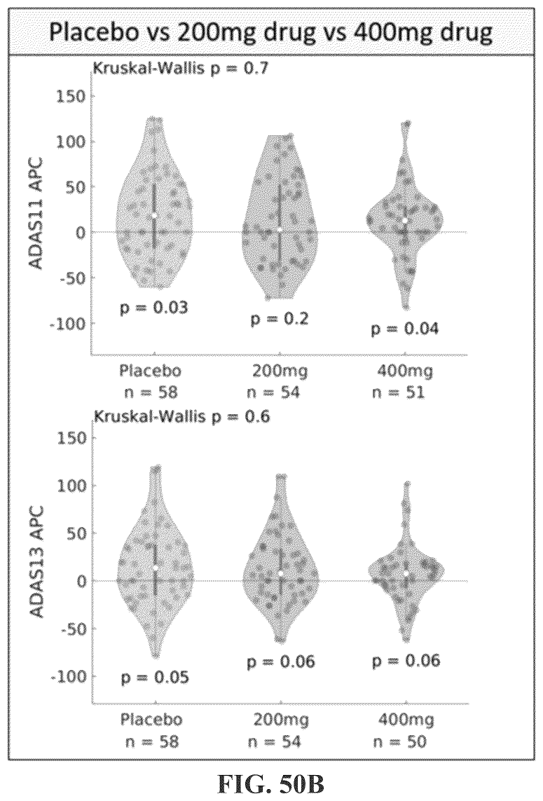

There is significant worsening of cognitive function but both the ADAS-13 and -11 scores (FIGS. 49A and 49B). The drug is associated with a trend in slowing the worsening, but this effect does not reach statistical significance in the study which as an exploratory trial was not powered sufficiently to derive statistically significant cognitive data. FIGS. 50A and 50B show drug dose analysis of ADAS-cog-11 and -13 cognition scores in subjects treated with placebo, 200 mg bid or 400 mg bid of LM11A-31. In the placebo group, there was a significant increase in −11 and −13 scores indicating a decline in cognition. Analysis for ADAS-cog-11 measures indicate a trend for decreased worsening of cognitive scores at both doses with a loss of the significant increase in score in the 200 mg group. Analysis for ADAS-cog-13 demonstrates a nominal decrease in the increase in score found in the placebo group in the 200 and 400 mg groups.

### Exploratory Outcomes for Domain 4: FDG-PET

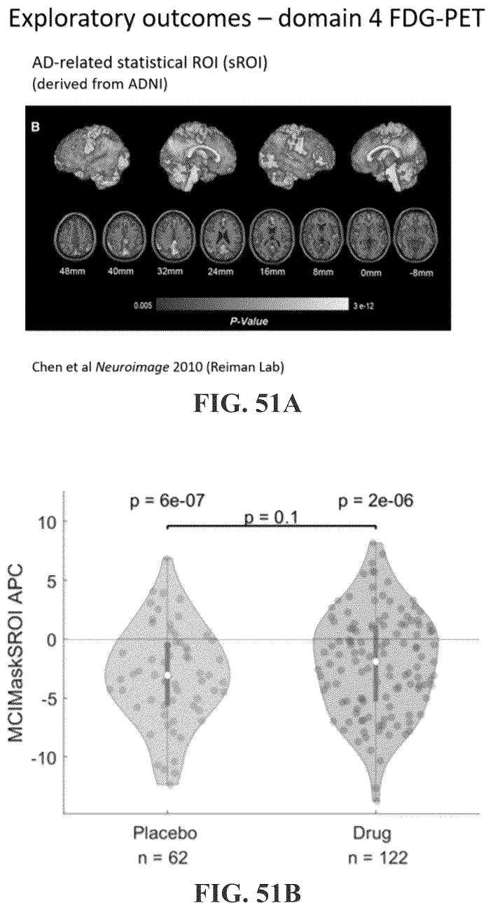

FDG-PET using statistical ROI measures derived from ADNI as described in the Chen et al. paper. This measure indicted significant decline in the placebo group with a trend to less decline over the 6 month period in the drug group (FIGS. 51A and 51B).

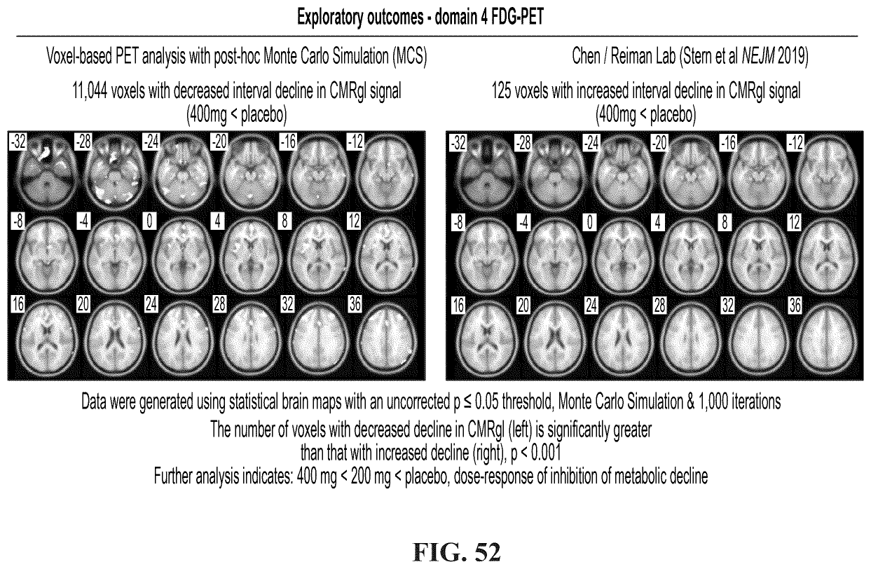

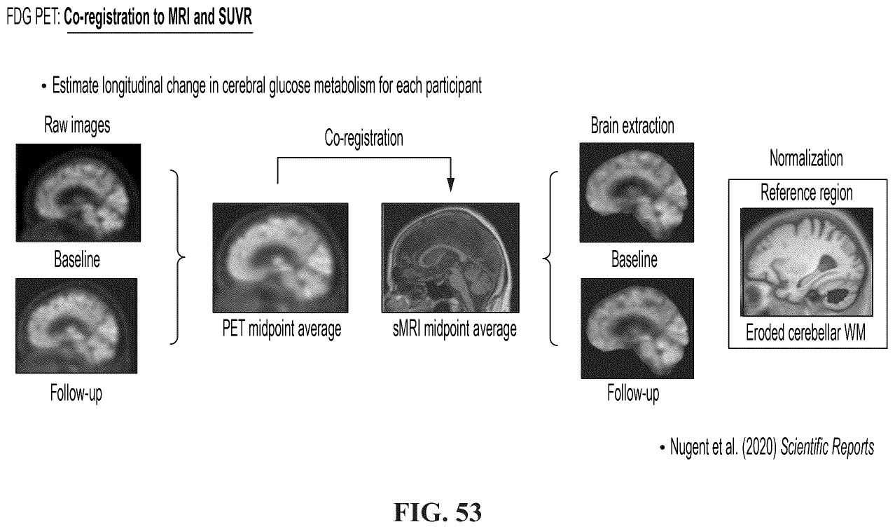

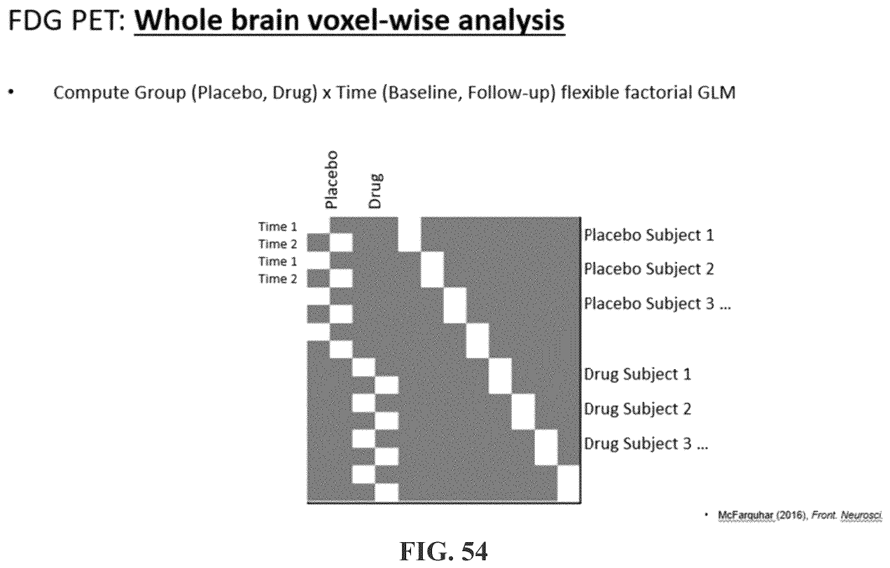

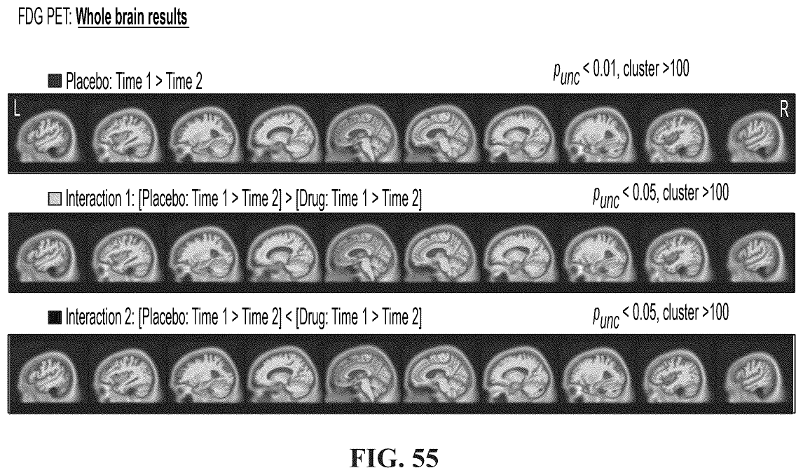

FDG-PET using the MCS (Monte Carlo Simulation) analysis approach is described in Stern et al. The number of voxels showed less decline (left) in metabolic function (a surrogate measure of synaptic function) in the drug while the placebo group was significantly much higher than the number of voxels showing increased decline (right) (FIG. 52). Areas showing less decline are highlighted in yellow. It is notable that these areas are relevant to areas that degeneration in AD. The effect at the 400 mg dose is higher than that at the 200 mg dose which is higher than that found in placebo (no effect found in placebo). Thus, a dose response effect is demonstrated. FIG. 53 shows three-dimensional co-registration of structural MRI and FDG-PET as a strategy for analysis of FDG-PET metabolic signal (SUVR—Standardized update value ratio) changes over time. This method makes possible analysis of FDG-PET signal in a common three-dimensional sampling space with the MRI analysis for each subject thereby enhancing accuracy of FDG-PET analysis and allowing the combination of the two modalities for further analyses and the assessment of the spatial relationship between structural and functional degeneration over time. FIG. 54 shows FDG-PET using whole brain voxel-wise analysis. It demonstrates flexible factorial generalized linear model (GLM) analysis matrix for brain voxel-wise FDG-PET metabolic analysis. FIG. 55 shows FDG-PET whole brain SUVR analysis. In FIG. 55, upper row indicates (red) regions demonstrating loss of SUVR signal in the placebo treatment group between baseline (Time 1) and post-treatment (Time 2). Middle row demonstrates regions of loss of SUVR signal as shown in the upper row (red) and areas in green indicate regions in which SUVR signal loss in the placebo group was significantly greater than that found in drug-treated subjects. For this analysis, the drug treatment group contains subjects treated with either 200 or 400 mg doses. The lower row demonstrates regions of loss of SUVR signal as shown in the upper row (red) and areas in blue indicate regions in which SUVR signal loss in the placebo group was significantly less than that found in drug-treated subjects. Overall, these findings indicate multiple areas within the degenerating region that demonstrate slowed degeneration in the drug group and rare areas demonstrating increased degeneration in the drug group.

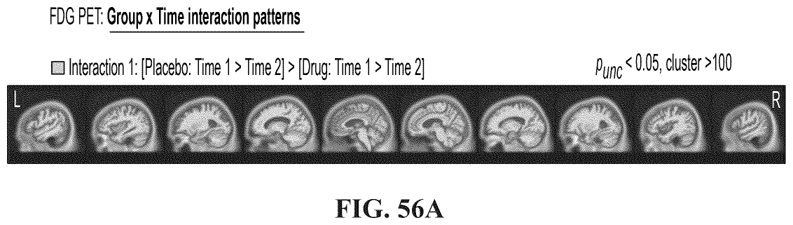

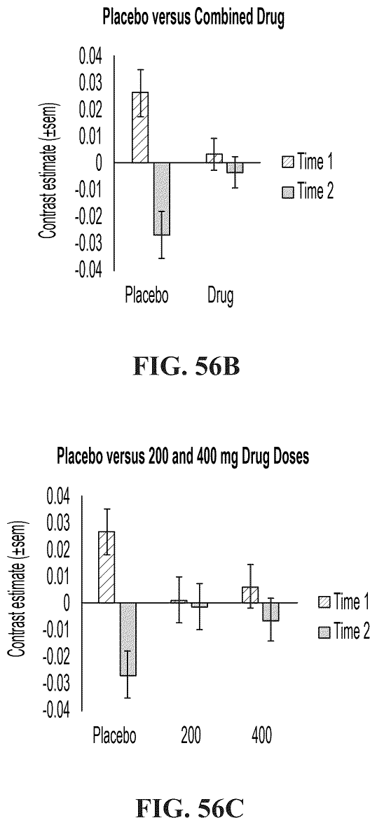

FIGS. 56A-56C show FDG PET using group×time interaction patterns. In FIGS. 56A-56C, contrast estimate plots show the magnitude and direction of the interaction effects observed in the flexible factorial GLM. Hence, the bars in green show the magnitude of SUVR change over Time at each level of treatment Group, averaged over all voxels in the green overlay of the map above, i.e., voxels which show a pattern of [Placebo: Time 1>Time 2] loss of SUVR signal greater than drug loss of SUVR signal [Drug: Time 1>Time 2] at a p<0.05 and cluster size>100. Dose analysis indicates a significant effect at both the 200 and 400 mg doses in slowing loss of SUVR signal.

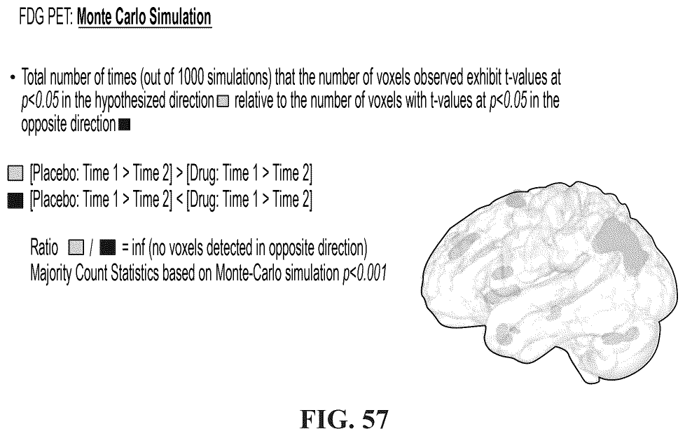

FIG. 57 shows FDG PET using MCS analysis. In FIG. 57, the ratio and p-value output of the MCS is derived from the total number of times (out of 1000 simulations) that the number of voxels observed in the input map (at a given p and k threshold) exhibit t-values in the hypothesized direction (slowing loss of SUVR signal with drug treatment) relative to the number of voxels with t-values in the opposite direction. Therefore, if the actual observed ratio of t-values is close to equivalent in the input map at a given p and k threshold, the MCS ratio and p-value will tend to support the null hypothesis of no drug effect. If the actual observed ratio is skewed heavily in favor of one direction over the other (as demonstrated here with a ratio of infinity), the MCS ratio and p-value will tend to reject the null hypothesis. These findings demonstrate a significant drug effect in slowing loss of brain metabolic function and is consistent the GLM results.

In general, for the range of biomarkers, effects at both doses are similar in many cases and in some cases, effects are greater at the higher dose. Given the smaller numbers available for analysis in the separate dose groups, many of these analyses do not reach statistical significance. The optimal design for a phase 3 trial would continue to include both doses when considering: dose-related side effects; likely efficacy at both doses; and some loss of statistical power for a given number of total subjects by including both doses versus one dose.

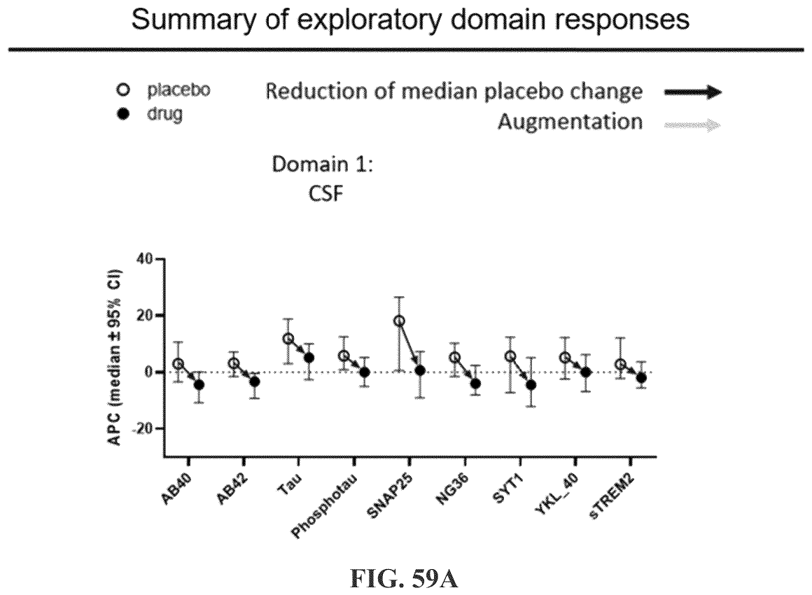

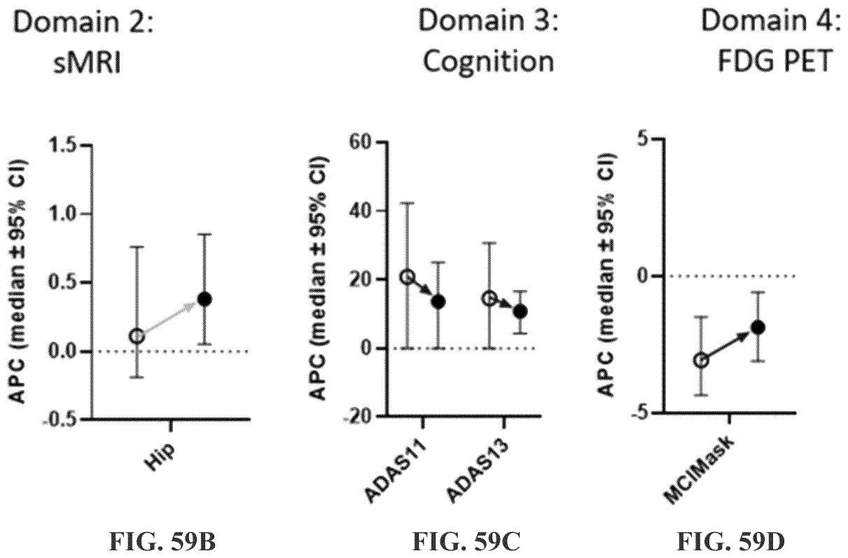

FIGS. 59A-59D show summary of the placebo progression measured by the measures in the 4 outcome domains and the effect and trend of the drug. The relatively remarkable consistency of the trend across all 4 outcome domains of the drug to correct measures of progression occurring in the placebo group is strongly suggestive of an underlying effect of the drug to slow degeneration in AD.

### Example 14: Assessment of p-Tau217 in Patients Treated with LM11A-31

This Example details the effects of LM11A-31 on a novel set of exploratory biomarkers collected and analyzed at a later stage of the Phase IIa trial. Moreover, this example describes how these plasma and CSF proteomic biomarkers relate with ELISA-based CSF biomarkers, neuroimaging, and cognitive data from the study.

### Methods

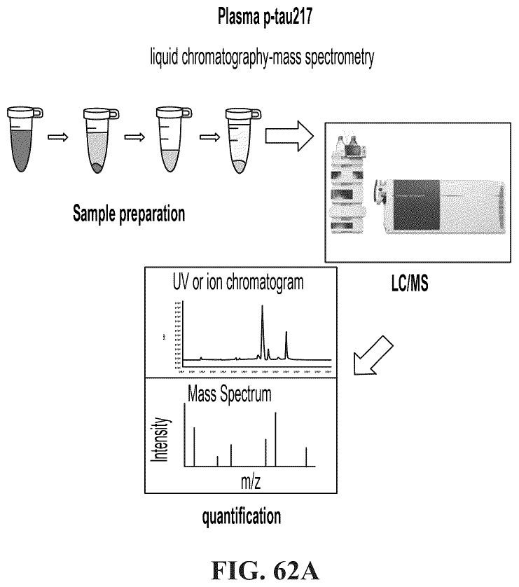

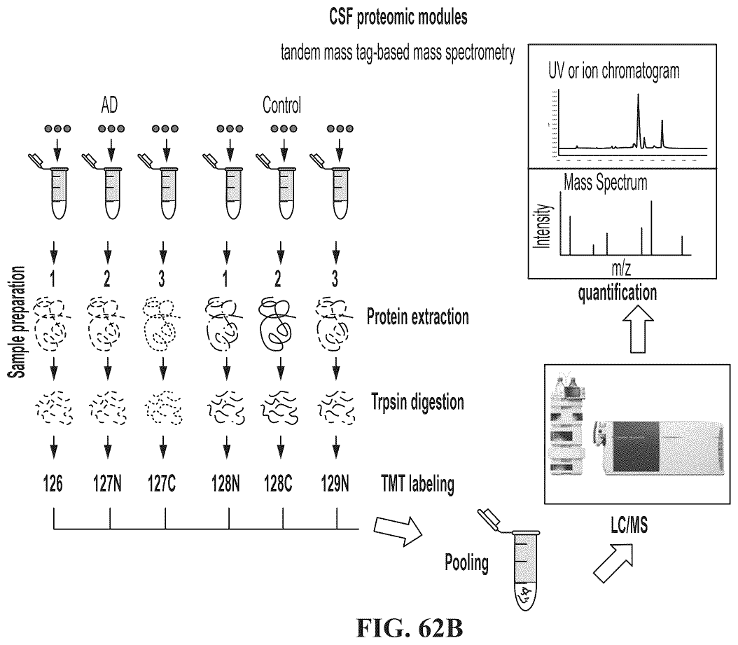

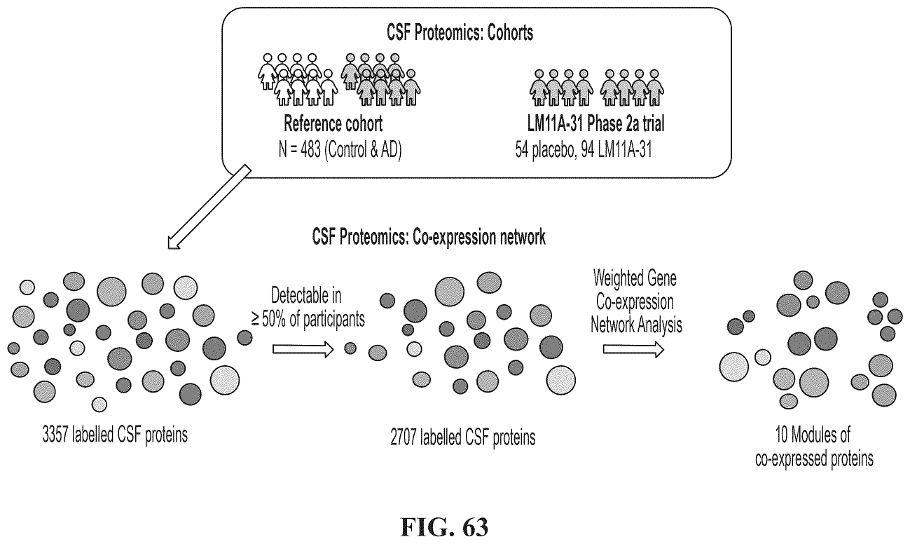

Data were collected as part of the randomized, double-blinded, placebo-controlled 26-week Phase IIa safety and exploratory endpoint trial of LM11A-31 detailed in Example 12. Briefly, participants with mild to moderate AD dementia (n=241) were treated twice daily with placebo, 200 mg of LM11A-31 or 400 mg of LM11A-31. Exploratory endpoints included plasma p-tau217 as measured by liquid chromatography-mass spectrometry (LC-MS/MS) (FIG. 62A) and cerebrospinal fluid (CSF) proteomic modules (FIG. 62B) as measured by tandem mass tag-based mass spectrometry (TMT-MS; Emtherapro, Atlanta). For the CSF proteomics, an independent cohort of participants was used for module definition and to inform on how the expression of proteins in the module changes with AD (FIG. 63). Weighted gene co-expression network analysis created a protein network based on similar patterns of expression across samples, and these patterns are usually indicative of shared biology. Additionally, relationships with CSF AD (AP40, Aβ42, p-tau181, t-tau), synaptic (SNAP25, SYT1, neurogranin), and glial markers (sTREM2, YKL40), as well as neuroimaging (structural MRI, FDG PET) and cognitive test data (MMSE, ADAS-Cog13, NTB) were evaluated. Additional details of the clinical trial are described in Shanks et al. Nat Med. 2024 June; 30(6):1761-1770.

### Results

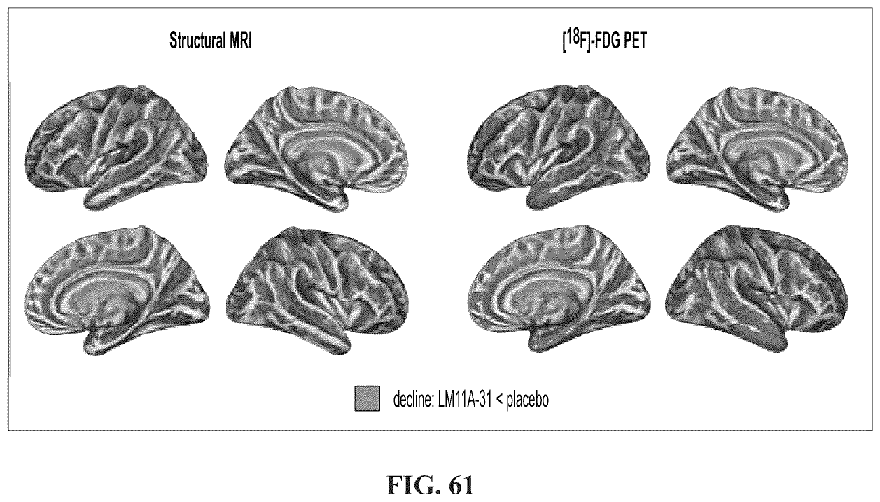

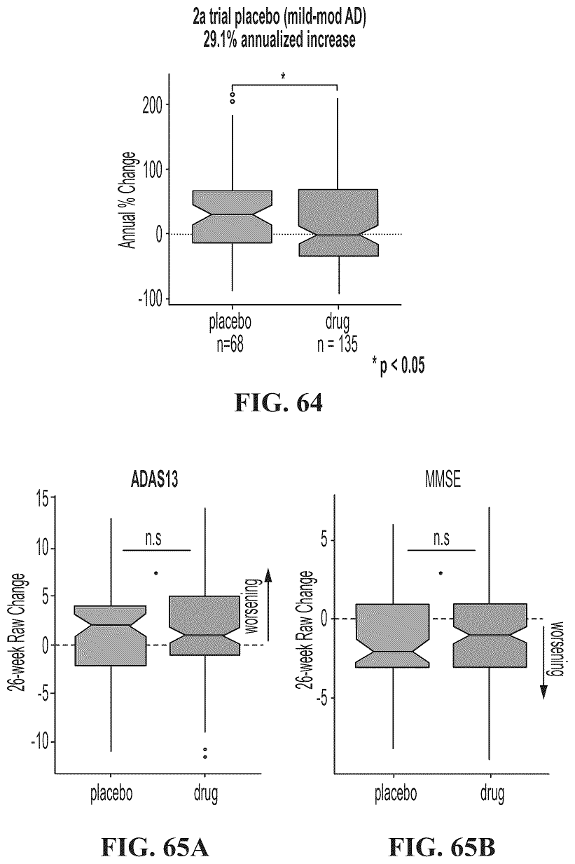

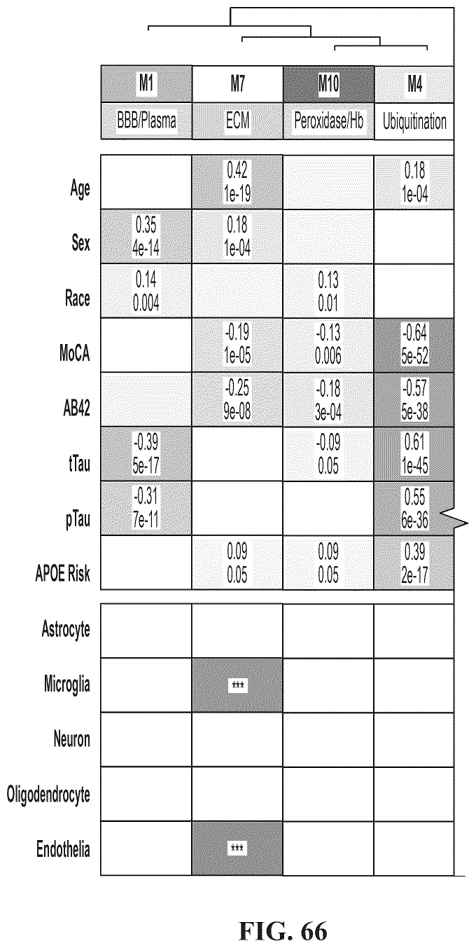

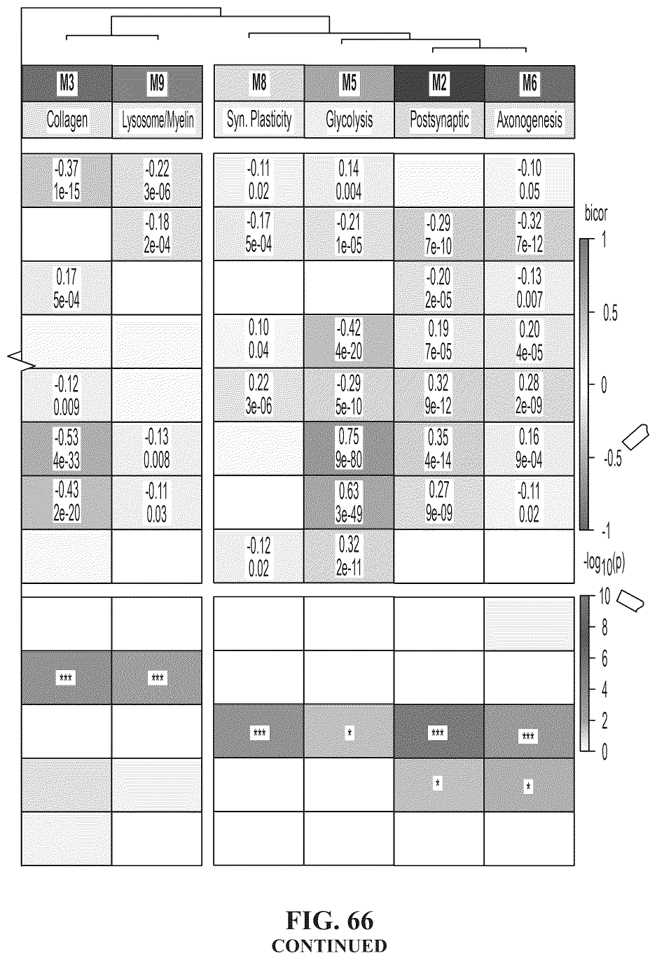

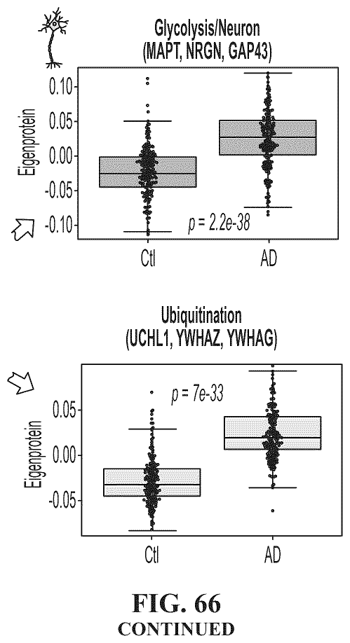

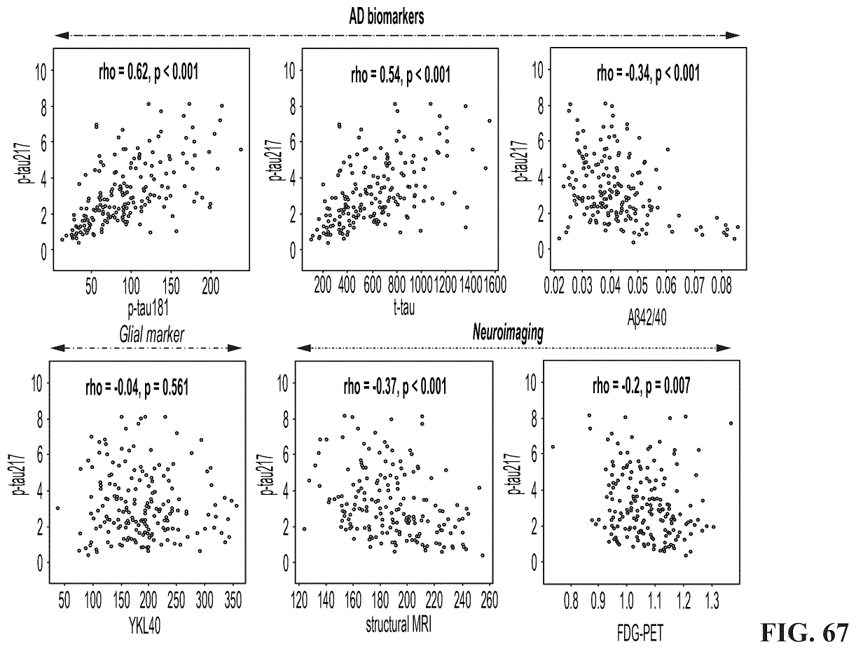

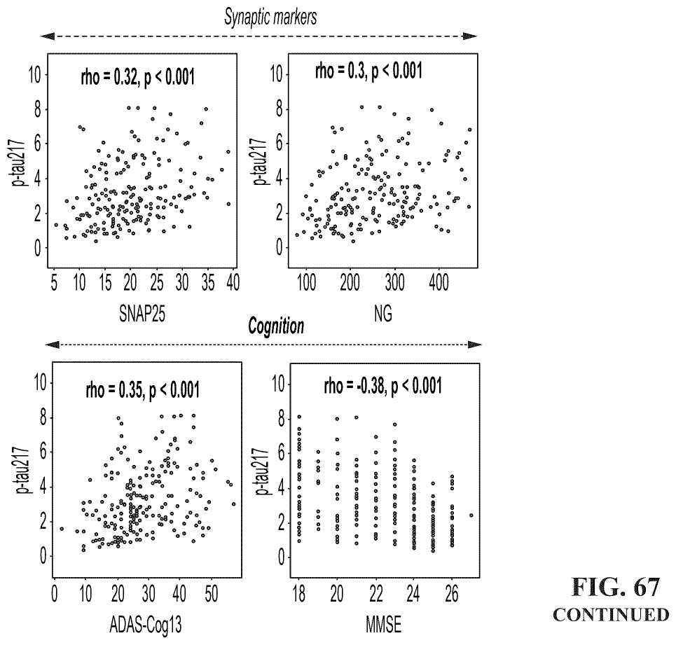

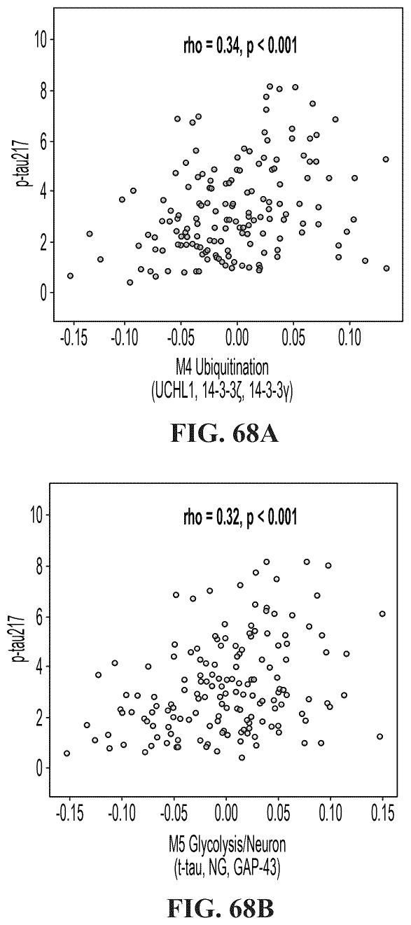

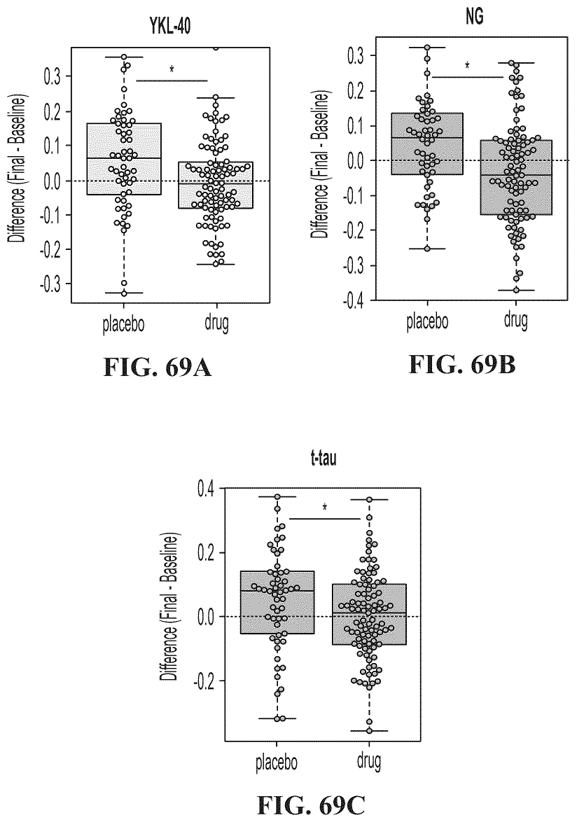

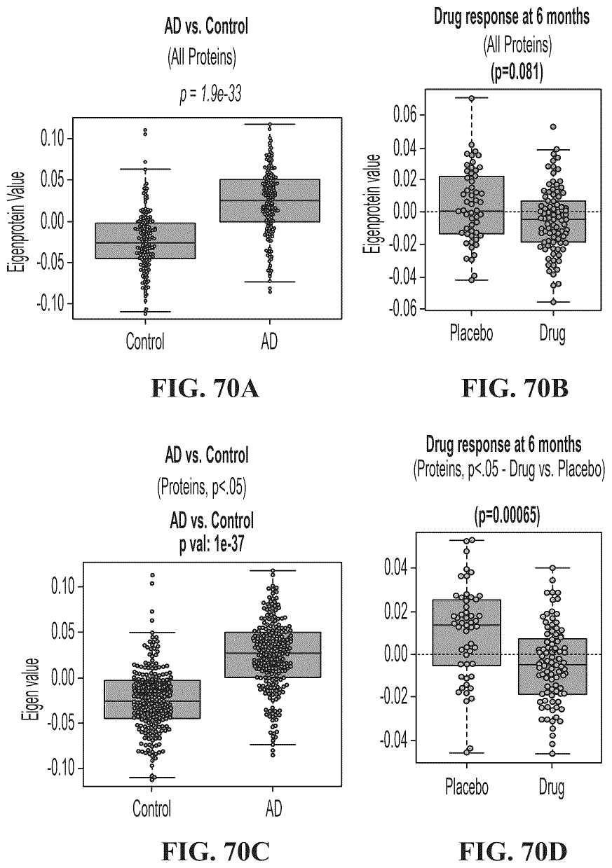

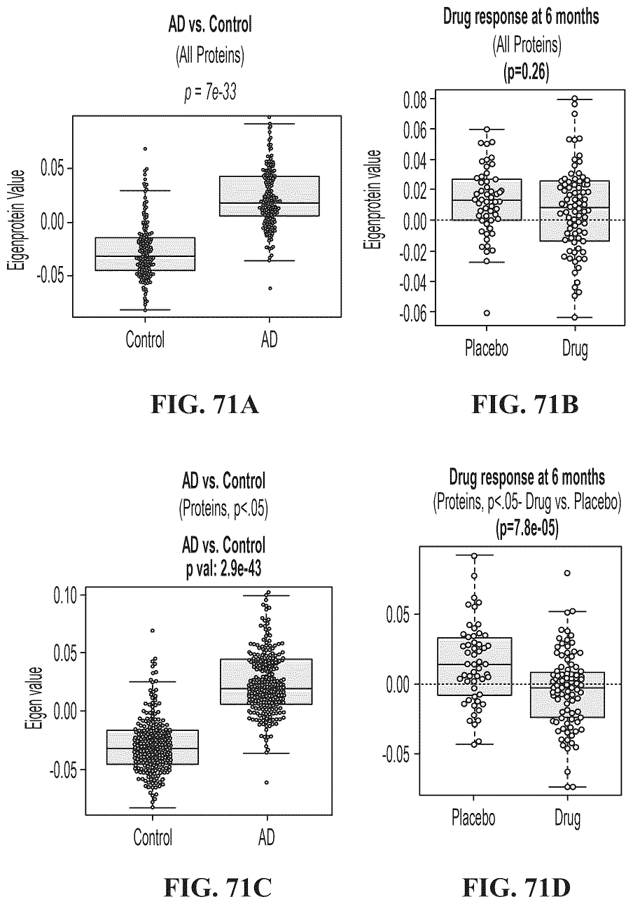

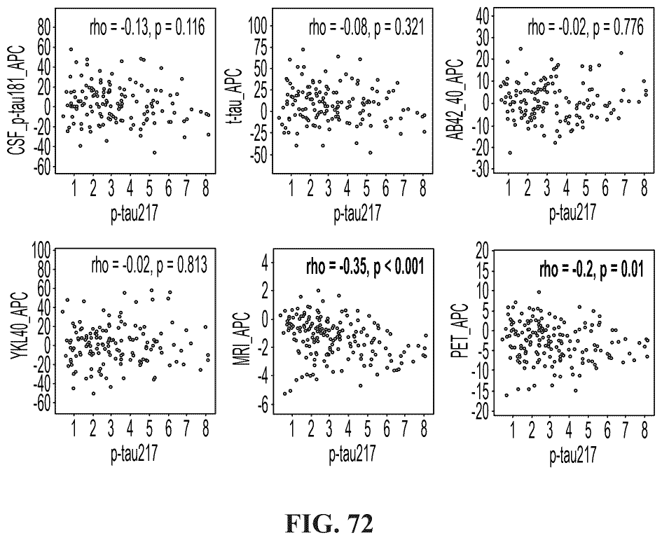

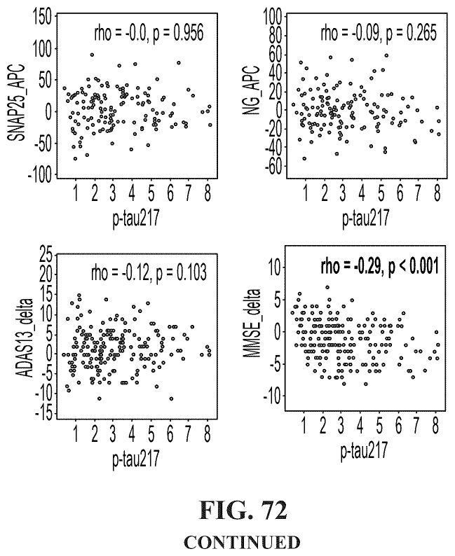

Treatment with LM11A-31 over the 26-week trial period slowed longitudinal progression on neuroimaging and CSF glial and synaptic markers (FIG. 61), significantly attenuated longitudinal increases in plasma p-tau217 relative to placebo (FIG. 64), and slowed disease progression based on analysis of cognitive markers (FIGS. 65A and 65B). Of the 10 preserved modules from a consensus AD CSF proteomic network, LM11A-31 treatment reduced the levels of AD-relevant modules associated with ubiquitination (module M4) and glycolysis (module M5), which included tau and neurogranin (FIG. 66). Notably, plasma p-tau217 levels showed significant correlations at baseline with degenerative measures, including sMRI, FDG-PET and CSF synaptic biomarkers (FIG. 67). Moreover, plasma p-tau217 correlated with CSF markers of glial activation, as well as with the CSF ubiquitination and glycolysis proteomic modules (FIGS. 68A-68B). It was further shown that that LM11A-31 slowed progression on YKL40, NG and t-tau, this time as measured by mass spec (FIGS. 69A-69C). LM11A-31 treatment was also shown to slow longitudinal progression on CSF proteomic modules (FIGS. 70A-70D and 71A-71D). Finally, it was shown that baseline plasma p-tau217 correlated with longitudinal changes in neuroimaging and cognition markers (FIG. 72).

Overall, 26-week treatment with LM11A-31 slowed progression of AD biomarkers, as measured by plasma p-tau217 and CSF proteomic modules, in addition to sMRI, FDG-PET and CSF synaptic and glial biomarkers. The relevance of plasma p-tau217 and the CSF proteomic modules to AD clinical status was highlighted by significant correlations with cognitive function.

### Example 15: a Randomized, Placebo-Controlled, 18 Month, Double-Blind Study with an Open-Label Extension Phase to Confirm Safety and Efficacy of LM11A-31 in Subjects with Early Alzheimer's Disease

### Background & Rationale

The objective of this Phase IIb/3, double-blind, placebo-controlled study is to evaluate the safety and efficacy of LM11A-31 (in this example, the compound of Formula (Ia)) in subjects with prodromal (MCI) and mild dementia (stage 3 and 4) due to AD with the presence of amyloid pathology. The study assesses whether LM11A-31 slows the progression of the disease as assessed by clinical outcomes for cognition and function, and by imaging, plasma, and CSF biomarker measures of disease pathology and neurodegeneration over 18 months of treatment and observation.

The total duration of study participation for each participant, including screening and the post-treatment follow-up periods, is up to 39 months:
Complete Screening: up to 2 months
Double-Blind Treatment: 18 months
Open-Label Extension Period: 18 months+/−30 days

### Study Objectives

The primary clinical objective of the study is to evaluate the efficacy of LM11A-31 versus placebo on clinical progression as measured by iADRS change from baseline at 18 months of treatment.

The secondary clinical objective of the study is to assess the effect of LM11A-31 versus placebo on clinical progression as measured by the following cognitive, functional, and behavioral scales from baseline through 18 months of treatment:
Clinical Dementia Rating-Sum of Boxes (CDR-SB)
Alzheimer's Disease Assessment Scale-Cognitive Subscale 13 (ADAS-Cog13)
Alzheimer's Disease Cooperative Study-instrumental Activities of Daily Living for Mild Cognitive Impairment (ADCS-iADL)
Mini Mental State Examination (MMSE)
Neuropsychiatric Inventory (NPI)
Alzheimer's Disease Composite Score (ADCOMS)

The primary biomarker objective of the study is to assess the effect of LM11A-31 versus placebo on AD progression as measured by plasma p-tau217 change from baseline at 18 months of treatment. The secondary biomarker objective of the study is to assess the correlations between the biomarker of plasma p-tau217 with clinical outcomes.

Other biomarker objectives of the study are to assess the relationships between the plasma, CSF subgroup, and imaging biomarkers and clinical measures (iADRS, CDR-SB, ADAS-Cog13, ADCS-iADL, NPI, MMSE) and to assess the effects of LM11A-31 versus placebo on biomarkers and imaging progression as measured by change from baseline through 18 months of treatment for following blood/plasma biomarkers, imaging, and cerebrospinal fluid (CSF) biomarkers (in a subset of patients):
Blood/plasma biomarkers
   BD-tau
   GFAP
   Proteomics
   other biomarkers
Imaging
   FDG-PET
   Brain region volumes as measured by volumetric MRI
   Tau-PET (in the tau PET subgroup)
   CSF (in the CSF subgroup)
   Aβ42-40
   p-tau181
   p-tau217
   brain-derived (BD)-tau
   total tau
   SNAP-25
   neurogranin, YKL-40
   neurofilament light chain (NfL)
   glial fibrillary acidic protein (GFAP)
   proteomics change
   other biomarkers

The safety objectives of the study are to evaluate the safety and tolerability of LM11A-31 as measured through the following standard safety assessments:
Adverse Events
Clinical laboratory tests
Vital signs and body weight
12-lead ECGs
Physical and neurological examinations
MRI
Columbia-Suicide Severity Rating Scale (C-SSRS)

Other exploratory objectives of the study are to evaluate the effects of LM11A-31 compared to placebo on overall health-related quality of life (QoL) as measured by European Quality of Life-5 Dimensions (EQ-5D; 5 Level version (EQ-5D-5L)) change from baseline through 18 months and to evaluate the population pharmacokinetics (PK) of LM11A-31 and its major metabolite AEM including correlations of plasma drug levels as a measure of exposure with clinical measures as well as plasma, CSF and imaging biomarker outcomes.

The objectives of the open-label 18-month extension phase are: (1) to evaluate the longer-term safety and tolerability of LM11A-31 in subjects in the Extension Phase; (2) to evaluate the longer-term effects of LM11A-31 on clinical outcome measures including iADRS, CDR-SB, ADAS-Cog13, ADCS-iADL, MMSE, NPI at each visit assessed in subjects in the Extension Phase; (3) to evaluate the longer-term effects of LM11A-31 across plasma and imaging biomarkers at each visit in subjects in the Extension Phase; and (4) to evaluate the safety and efficacy of LM11A-31 in subjects who roll over from placebo to active treatment.

### Overall Design

This study is a multicenter, randomized, double-blind, placebo-controlled, Phase IIb/3 study of LM11A-31 in subjects with early Alzheimer's Disease (mild cognitive impairment (MCI) or mild dementia due to AD) with MMSE of 18-28 and diagnosis of AD via clinical criteria and plasma p-tau217.

A total of 1600 participants who meet entry criteria are randomized in a 1:1 ratio to one of the following treatment groups:
LM11A-31: 400 mg BID oral tablets.
Placebo

Randomization in a 1:1 ratio to LM11A-31 and placebo is stratified by clinical subgroup, MCI vs mild dementia due to AD (with no more than 60% of MCI or mild) and past or current anti-amyloid treatment (with no more than 10% of patients with past or current anti-amyloid treatment). All patients enrolled in the study follow the same schedule of assessments. Treatment in the double-blinded placebo-controlled core study is administered for 18 months. After 18 months of treatment, an additional 18-month open-label extension phase is available for all subjects who complete the full 18 months of placebo-controlled treatment in the core study. During the open-label study period, treatment status of blinded period for each subject is maintained blinded.

The Phase 3 portion of the study includes additional patients enrolled plus all patients enrolled in the Phase IIb portion. After completion of enrollment and when all enrolled patients have completed the 18-month treatment, or discontinued, a final analysis is conducted for the placebo-controlled period.

### Number of Subjects

Approximately 1600 participants are randomized in the trial with 800 per arm (LM11A-31 vs. placebo)

### Screening

Primary screening includes history and examination consistent with MCI or early dementia, such as clinical criteria (NIA-AA) for MCI and mild dementia due to AD and MMSE 18-28+Delayed Paragraph Recall test≤4.

Secondary Screening includes clinical blood, urinalysis, ECG, as well as plasma screening of p-tau217 for Alzheimer's Disease pathology biomarker. Tertiary Screening includes MRI safety studies (per structural MRI protocol to be used for baseline and endpoint measures).

The inclusion criteria used for the study include the following:
Age 50-85
Meets the 2018 National Institute of Aging-Alzheimer's Association (NIA-AA) core clinical criteria for MCI due to AD-intermediate likelihood or probable Mild AD Dementia (primary screening).
Report a history of subjective memory decline with gradual onset and slow progression over the last 1 year before screening; must be corroborated by an informant (primary screening).
Evidence of memory impairment as indicated by the delayed Paragraph Recall Test score≤4 (primary screening)
MMSE score greater than or equal to 18 and less than or equal to 28 at Screening
Positive biomarker for brain amyloid pathology as indicated by the following:
   Plasma p-tau217 above “normal” cutoff for selected assay.
   NOTE: ONLY for patients who are currently on or have a history of treatment with approved anti-amyloid therapies (lecanemab or donanemab) prior evidence of brain amyloid pathology can be accepted. Prior evidence includes any one of the following:
      PET assessment of imaging agent uptake into brain
      CSF assessment of t-tau/AB[1-42]
      Plasma p-tau217 above “normal” cutoff
Treatment with concurrent or historic anti-amyloid treatment is allowed (not required) if meeting the following dosing requirements:
   Receiving therapy for >6 months and has had final label required MRI (i.e. before 14th dose of lecanemab or 7th dose of donanemab) prior to screening
   Has been treated according to label recommendations with respect to ARIA screening and management:
      Had all required MRI evaluations (for lecanemab had MRIs prior to 5th, 7th, and 14th infusion; for donanemab had MRIs prior to 2nd, 3rd, 4th, and 7th infusion)
      No requirement for suspension of dosing as per section 2.3 of lecanemab/donanemab prescribing information
      Final required MRI (i.e. prior to 14th infusion of lecanemab or 7th infusion of donanemab) or most recent MRI (whichever is later) with no evidence of moderate or severe ARIA-E or ARIA-H as described in section 5.1 of lecanemab/donanemab prescribing information
   Anticipated to continue therapy for at least 3 months following randomization (stopping therapy at any time for safety concerns is allowed, stopping therapy after 3 months for amyloid levels is allowed).
   For historic use discontinuation at least 3 months prior to randomization and did not require dosing suspension or discontinuation due to ARIA symptoms or MRI findings or other serious or significant complications.
Treatment with concurrent or historic acetylcholinesterase inhibitor or memantine is allowed (not required) if meeting the following requirements:
   Stable dose for at least 3 months prior to randomization
   Anticipated to continue therapy throughout the 18 month blinded portion of the study (stopping therapy at any time for safety concerns is allowed).
   For historic use, discontinuation at least one month prior to randomization

The exclusion criteria used for the study include the following:
Treatment with a tau antibody or other tau-based therapy
Anti-amyloid treatment<6 months or with prior ARIA (per MRI) or other serious or significant complications
Ongoing treatment for anticoagulation therapy (only applicable to CSF subgroup or patients taking anti-amyloid therapies).
Subjects who were dosed in a clinical study involving any new chemical entities for AD within 6 months
Have any clinically important abnormality at screening, as determined by investigator, in physical or neurological examination, vital signs, ECG, or clinical laboratory test results that could be detrimental to the participant, could compromise the study, or show evidence of other etiologies for dementia.
Screening MRI which shows evidence of significant abnormality that would suggest another potential etiology for progressive dementia or a clinically significant finding that may impact the participant's ability to safely participate in the study.
Significant neurological disease affecting the central nervous system other than AD, that may affect cognition or ability to complete the study, including but not limited to, other dementias, significant cerebrovascular disease (such as major infarct, one strategic or multiple lacunar infarcts or extensive white matter lesions as determined by Fazekas scale), Parkinson's disease, serious infection of the brain, multiple concussions, or epilepsy or recurrent seizures (except febrile childhood seizures)

The study treatments are 400 mg (two 200 mg) oral tablets BID of LM11A-31. All subjects receive 18 months of treatment. For subjects who complete the core study an extension phase continues for up to 18 months.

For subjects who enter the study on AChEIs or memantine they must remain on the same dose for the duration of the study unless discontinuation is medically necessary. For subjects who enter the study on anti-amyloid therapy they must remain on the same dose for at least 3 months after randomization unless discontinuation is medically necessary (e.g. event of ARIA while on anti-amyloid therapy). Subjects are not allowed to initiate new AD therapies or non-AD drugs approved for cognitive impairment during the 18 month placebo controlled period of the study.

### Assessments

The following assessments are performed throughout the study and as specified in Table 14.

TABLE 14
Schedule of Assessments

| | | | | | | | | | | Open-Label Extension | | | | | | |
|---|---|---|---|---|---|---|---|---|---|---|---|---|---|---|---|---|
| | | | | | | | | | | | | | | | | V16 |
| | | | | Blinded Treatment | | | | | | | | | | | | EOT/ |
| | Screening | Baseline | | | | | | | V9 | | | | | | | ED/ |
| | V1 | | V3 | V4 | V5 | V6 | V7 | V8 | EOT | V10 | V11 | V12 | V13 | V14 | V15 | UNS |
| | D-60 | V2 | D | M | M | M | M | M | M | D | M | M | M | M | M | M |
| Procedure or | to | D | 7 ± | 1 ± | 2 ± | 3 ± | 6 ± | 12 ± | 18 ± | 7 ± | 19 ± | 20 ± | 21 ± | 24 ± | 30 ± | 36 ± |
| Assessment ^{a} | D-1 | 0 | 1 d | 7 d | 7 d | 7 d | 7 d | 7 d | 7 d | 1 d | 7 d | 7 d | 7 d | 7 d | 7 d | 7 d |

Screening/History

| | | |
|---|---|---|
| Informed | X | |
| Consent | | |
| Demographics | X | |
| Medical & | X | |
| Disease | | |
| History | | |
| Eligibility | X | X |
| Assessment | | |
| MMSE + Delayed | X | |
| Paragraph | | |
| Recall Test | | |

Safety Assessments

| | | | | | | | | | | | | | | | | |
|---|---|---|---|---|---|---|---|---|---|---|---|---|---|---|---|---|
| Vital Signs | X | X | X | X | X | X | X | X | X | X | X | X | X | X | X | X |
| Height & | X | | | | | | | | | | | | | | | |
| Weight | | | | | | | | | | | | | | | | |
| Physical & | X | | | | | X | X | X | X | | X | | X | X | X | X |
| Neurological | | | | | | | | | | | | | | | | |
| Examination ^{c} | | | | | | | | | | | | | | | | |
| ECG ^{h} | X | X ^{h} | | | | X | X | X | X | | X | | X | X | X | X |
| Concomitant | X | X | X | X | X | X | X | X | X | X | X | X | X | X | X | X |
| Medications & | | | | | | | | | | | | | | | | |
| Procedures | | | | | | | | | | | | | | | | |
| AE Assessment | X | X | X | X | X | X | X | X | X | X | X | X | X | X | X | X |
| CSSR-S^{g} | X | X | X | X | X | X | X | X | X | X | X | X | X | X | X | X |
| Self-Harm | X | X | X | X | X | X | X | X | X | X | X | X | X | X | X | X |
| Supplement Forms^{g} | | | | | | | | | | | | | | | | |
| Self-Harm | X | X | X | X | X | X | X | X | X | X | X | X | X | X | X | X |
| Follow-up Form^{g} | | | | | | | | | | | | | | | | |

Clinical Laboratory Assessments

| | | | | | | | | | | | | | | |
|---|---|---|---|---|---|---|---|---|---|---|---|---|---|---|
| Blood Chemistry & | X | | X | X | X | X | X | X | X | X | X | X | X | X |
| Hematology | | | | | | | | | | | | | | |
| Urinalysis | X | | | | | X | X | X | | | | X | X | X |
| Blood Plasma - | X | | | | | | | | | | | | | |
| Screening (ApoE, p- | | | | | | | | | | | | | | |
| tau217, total tau) | | | | | | | | | | | | | | |
| Blood Plasma | | X ^{b} | | | | X | X | X | | | | X | X | X |
| Biomarker Samples - | | | | | | | | | | | | | | |
| Baseline (BD tau, | | | | | | | | | | | | | | |
| p-tau217, total | | | | | | | | | | | | | | |
| tau, GFAP) ^{b} | | | | | | | | | | | | | | |
| CSF (Spinal Tap) | | X ^{b} | | | | | | X | | | | | | X |
| Biomarker Samples | | | | | | | | | | | | | | |
| (subgroup only) | | | | | | | | | | | | | | |
| (p-tau181, p-tau217, | | | | | | | | | | | | | | |
| BD-tau, tau, | | | | | | | | | | | | | | |
| SNAP-25, neurogranin, | | | | | | | | | | | | | | |
| YKL-40, NfL, GFAP, | | | | | | | | | | | | | | |
| Proteomics) ^{b} | | | | | | | | | | | | | | |
| Pharmacokinetic | | X ⁱ | | | X | X | X | X | | | | X | X | X |
| Samples | | | | | | | | | | | | | | |

Imaging

| | | | | | |
|---|---|---|---|---|---|
| Structural | X ^{b} | X | X | X | X |
| MRI/DTI ^{b} | | | | | |
| FDG PET | X ^{b} | X | | X | X |
| Tau-PET | X ^{d} | | | X ^{d} | X |
| (subgroup only) ^{d} | | | | | |

Efficacy Measures

| | | | | | | | |
|---|---|---|---|---|---|---|---|
| iADRS | X | X | X | X | X | X | X |
| CDR-SB | X | X | X | X | X | X | X |
| ADAS-Cog-13 | X | X | X | X | X | X | X |
| ADCS-iADL | | X | X | X | X | X | X |
| MMSE ^{b} | X ^{b} | X | X | X | X | X | X |
| NPI | X | X | X | X | X | X | X |
| QoL-AD | X | X | X | X | X | X | X |

Study Drug Administration

| | | | | | | | | | | | | | | | |
|---|---|---|---|---|---|---|---|---|---|---|---|---|---|---|---|
| LM11A-31/Placebo | X | X | X | X | X | X | X | X | X | X | X | X | X | X | X |
| Dosing ^{f} | | | | | | | | | | | | | | | |

Abbreviations:
Aβ = amyloid beta;
AD—Alzheimer's Disease;
ADAS-Cog = Alzheimer's Disease assessment scale-cognitive;
ADCS-iADL = Alzheimer's Disease Cooperative Study-Instrumental Activities of Daily Living scale;
AE = adverse event;
ApoE = apolipoprotein E;
CSF = cerebrospinal fluid;
C-SSRS = Columbia Suicide Severity Rating Scale;
D = study date;
d = days;
DTI = diffusion tensor imaging;
ECG = electrocardiogram;
ED = early discontinuation;
EOT = end of treatment;
FDG = F-fluorodeoxyglucose;
GFAP = glial fibrillary acidic protein;
iADRS = integrated Alzheimer's Disease rating scale;
M = Study Month;
MCI = mild cognitive impairment;
MMSE = mini-mental state examination;
MRI = magnetic resonance imaging;
NfL = neurofilament light chain;
NPI = neuropsychiatric inventory;
PET = positron emission tomography;
P-tau = phosphorylated tau;
QoL = quality of life;
Tau—tauopathies;
UNS = unscheduled visit;
V = visit
^{a} Full details and descriptions of assessments are detailed in the full protocol.
^{b} Assessments completed within the prior 8 weeks during screening may be used for Screening/Baseline
assessments. Blood PK samples and ECG to be taken 1.5 hours post-first dose.
^{c} Any clinically significant changes from baseline on physical/neurological examinations should be noted on the AE CRF.
^{d} Tau-PET is offered to 50% of the study-eligible subjects who enroll at sites and are able to participate (based on site's geographical location or proximity to tau PET). Participation in the tau PET substudy is optional and does not affect enrollment or treatment in the study.
e. CSF is offered to 50% of the study-eligible subjects who enroll at sites and are able to participate. Participation in the CSF sub study is optional and does not affect enrollment or treatment in the study.
^{f} Twice daily study drug administration.
^{g}Required if triggered by the Self-Harm Supplement Form per instructions. If visit not conducted on site, may collect by phone.
^{h} 12-Lead ECGs are collected in triplicate. In addition to baseline ECG (pre-dose), ECG is collected post-dose 1.5 hrs (90-120 minute range is allowed).
ⁱ In addition to baseline PKs (pre-dose), PK samples are collected post-dose 1.5 hrs (90-120 minute range is allowed).

Efficacy Assessments the study include the iADRS, CDR-SB, ADAS-Cog 13, ADCS-iADL, MMSE, and NPI, which are well-established clinical tools for use in assessment of AD and are used for assessment of clinical outcomes including cognition and functional assessment. These are assessed at baseline and every 6 months during participation in the study.

For biomarker assessments, blood plasma levels of p-tau217, BD tau, and GFAP are collected from all subjects every 6 months during participation in the study. CSF is performed on subjects who qualify and consent to participate in the CSF sub study at baseline, 18, and 36 months to assess the levels of Aβ42-40, p-tau181, p-tau217, total tau, SNAP-25, neurogranin, YKL-40, NfL, GFAP, proteomics and other exploratory biomarkers using validated and commercially available kits (anticipated to be approximately 30-40% of subjects). Structural MRI/DTI is performed on all subjects at baseline (screening MRI is used for baseline), 6, 12, 18, and 36 months. FDG-PET is performed on all subjects at baseline, 6, 18, and 36 months. Tau-PET is performed on subjects who consent to participate in the Tau-PET sub study at baseline, 18 and 36 months (anticipated to be approximately 20-25% of subjects). Health-Related Quality of Life is measured using the EQ-5D-5L at baseline and every 6 months during participation in the study.

Safety is monitored via AEs, hematology, blood chemistry, and measurement of vital signs during the treatment period at baseline, 1, 2, 3, 6, 12, 18, 19, 20, 21, 24, 30, and 36 months. Urinalysis is collected at screening and every 6 months throughout the study. ECGs are performed for safety assessments at baseline, 3, 6, 12, 18, 19, 21, 24, 30, and 36 months. Additional ECGs are performed at anticipated Tmax for LM11A-31 and its primary metabolite AEM after first dose 1.5 hr. Physical and neurological examinations are performed for safety assessments at Screening, 3, 6, 12, 18, 19, 21, 24, 30, and 36 months.

### Pharmacokinetic Assessments

Blood samples are collected from all subjects for determination of plasma LM11A-31 and its primary metabolite AEM at baseline pre-dose, after first dose 1.5 hr and at 3, 6, 12, and 18 months. Subjects who withdraw from the study or discontinue study drug early have blood samples collected at the early termination visit. Population PK approach is used to characterize PK and PK/biomarker relationships of LM11A-31.

### Pharmacodynamic, Pharmacogenomic, and Other Assessments

APOE4 genotyping is conducted at baseline (APOE4 positive and negative). APOE4 homozygous and heterozygous status are used in statistical analysis to determine effects on treatment response and safety. Blood samples obtained from participants are analyzed for global proteomic measures at baseline, 18, and 36 months.

### Stratification

For each treatment arm, stratification is based on past/current amyloid antibody treatment (yes or no) and disease stage (MCI (CDR global of <=0.5) vs mild AD (CDR global of >=1)).

### Randomization

Simple randomization with 1:1 ratio (with block sizes of 4 or 6) within each strata defined by past/current amyloid antibody treatment (yes or no) and disease stage (MCI vs mild AD). These randomization codes and the associated treatment assignments are kept by the unblinded statistician. The randomization codes are blinded and shared only as needed, such as with the unblinded DMC. Access to the randomization code prior to the final data lock is documented as described in the study unblinding plan. Blinding for Investigators, subjects, site staff, and other personnel involved with the conduct of the trial is maintained after the final database lock for the core placebo-controlled study through the end of the open-label extension.

### Sample Size Rationale

To determine sample size for the primary outcome, historical data were collected and mean to standard deviation ratios (MSDRs) were calculated. An assumed 25% slowing of disease progression was used to estimate a Cohen's D effect size of 0.20 for the iADRS, based on an 18-month MSDR of 0.80 (0.8×25% slowing=0.20). With a dropout rate of 30%, a total sample size of approximately 1600 with 1120 completers (560 per group) is required to achieve at least 90% power. For the go/no go internal decision-making interim analysis, assuming a true treatment effect of Cohen's D=0.30 on p-tau217, 250 patients per treatment arm yields 96% power using a two-sided alpha=0.10.

### Study Endpoints

For the final analysis, the primary clinical endpoint is change in iADRS from baseline to Month 18. The key secondary clinical endpoint is change in CDR-SB from baseline to month 18. Additional secondary clinical endpoints are:
Change from baseline for cognition as measured by ADAS-cog-13 score, MMSE score, ADCOMS score at 6, 12, and 18 months;
Change from baseline for function as measured by ADCS-iADL score at 6, 12, and 18 months; and
Change from baseline for behavior/neuropsychiatric symptoms as measured by Neuropsychiatric Inventory (NPI) score at 6, 12, and 18 months;
The primary biomarker endpoint is change from baseline to Month 18 of plasma p-tau217. Secondary Biomarker Endpoints are correlations between the biomarker of plasma p-tau217 with clinical outcomes.

Safety endpoints of the study are:
The incidence of AEs, out of normal range laboratory tests, abnormal ECG findings, out of range vital signs, CSSR-S, and safety assessments from physical and neurological examinations; and
The relationship between exposure of LM11A-31 and its primary metabolite AEM and most frequent AEs are explored, as appropriate.

Biomarker, imaging, and CSF endpoints of the study are:
change from baseline in FDG-PET at 6, 12, and 18 months;
change from baseline in volumetric structural MRI measures at 6, 12, and 18 months;
change from baseline in tau PET signal at 18 months (study subgroup);
change from baseline in CSF tau, synaptic, inflammatory, and exploratory biomarkers at 18 months (study subgroup); and
change from baseline in plasma biomarkers of p-tau217/tau, BD tau, and GFAP at 6, 12, and 18 months.

The relationships between biomarker and clinical endpoints are:
correlations between changes from baseline in iADRS and change in biomarker of plasma p-tau217; and
correlations between blood, imaging, and CSF biomarkers (subgroup) and clinical outcomes.

Other endpoints of the study are:
change from baseline for Health-Related QoL as measured by EQ-5D-5L at 18 months; and
correlations between drug exposure and responses with clinical, biomarker, and safety endpoints and any emergent relationships are explored through population PK/PD modeling.

### Statistical Methods

The primary estimand for the final analysis is as follows:
population: Patients with stage 3 or 4 (MCI/Mild) AD as defined by the inclusion/exclusion criteria;
treatment: LM11A-31 400 bid or placebo;
primary Endpoint: iADRS;
summary statistic: Difference between treatment groups in mean change to month 18, which is also converted to % slowing and time saved.
Inter-current events (ICE): Deviations from the intended treatment regimens, including concomitant medication for non-AD indications usage are handled using a treatment policy strategy. Early discontinuation of the study or concomitant medications for AD indications are handled using a hypothetical strategy where data collected after initiation of AD concomitant medication are censored to estimate what would have been observed if the patient had not discontinued or been treated with AD concomitant medications. To account for potential imbalance of AD concomitant medication initiation between treatment arms, a sensitivity analysis follows a composite strategy of worst case imputation, where data collected after initiation of AD concomitant medication are imputed as the worst score observed by all patients within the visit being imputed.
In order to understand clinical meaningfulness of the iADRS results, the results are also described as % slowing and time savings.

The key primary and secondary objectives and estimands are based on changes from baseline. These objectives and their associated estimands are evaluated using a restricted maximum likelihood-based repeated measures analysis (MMRM). The dependent variable is change from baseline. The model includes the fixed, categorical effects of treatment, visit, and investigative site along with the treatment-by-visit interaction. Continuous fixed covariates in the model include baseline score of the dependent variable and its interaction with visit. Within-subject errors are modeled using an unstructured covariance matrix. If this analysis fails to converge, heterogeneous Toeplitz, heterogeneous first-order auto-regressive, and heterogeneous compound symmetric structures are tested. The first structure to yield convergence is the primary analysis. If none of these analyses converge, site is deleted from the model and the testing sequence is repeated. If something other than unstructured is used as the primary analysis, the sandwich estimator is used to account for potential misspecification of the correlation structure in determining standard errors. The Kenward-Roger approximation is used to estimate denominator degrees of freedom. Outputs include the contrasts between treatment group LSMEANS at each visit.

Correlation analyses relating the biomarker absolute values and changes from baseline to the clinical outcome absolute values and changes from baseline are performed to assess how well the biomarker outcomes predicted clinical outcomes.

Subgroups are analyzed using the MMRM model as described above, with the following modification. Baseline severity and its interaction with visit are deleted from the model. Subgroup and its interactions with treatment and visit are added to the model along with the 3-way subgroup-by-treatment-by-Visit interaction. Other subgroup variables include age, gender, APOE4 status, MCI vs mild AD, and past or current anti-amyloid treatment.

### Analyses

An interim analysis for internal go/no go decision-making is conducted when 500 subjects (250 subjects in each group) have completed 6-months of treatment. This interim analysis evaluates mean changes from baseline to month 6 in the biomarkers of plasma p-tau217, FDG-PET and a global statistical test combining these two endpoints. If the two-sided p value is less than 0.10 for any of the 3 endpoints the study continues as planned. If the two-sided p value is not less than 0.10 for all of the 3 endpoints the data monitoring committee may suggest that the study be stopped for futility. If the true drug effect is as assumed to be a Cohen's D=0.30, the chance of a false negative result is 4% for each of the 3 endpoints. Therefore, the probability of falsely stopping the study for futility because all 3 endpoints are negative is between 1 and 4% depending on the correlation between the endpoints.

A final analysis is conducted after enrollment is complete and all enrolled patients have completed the 18-months placebo control period or discontinued.

The design of this study is commensurate with expectation for a confirmatory (pivotal) trial. The family wise type 1 error is controlled using a multiplicity adjustment specified in the SAP. Type I error is controlled through a standard gatekeeping approach, where each clinical endpoint is tested according to a pre-specified hierarchy and each endpoint can be declared significant only if all preceding endpoints have been declared significant. The biomarker results at the interim analysis is reported to confirm whether biomarker results are associated with clinical outcomes.

Analysis of the open label study phase is used to assess the evidence of a disease modification. This is done by assessing whether a treatment effect is established during the randomized phase and then whether the treatment arms continue to maintain separation after 18-months on treatment in the open label extension.

### Example 16: Modulation of the p75 Neurotrophin Receptor Attenuates Tau Phosphorylation and Promotes Synaptic Resilience in Alzheimer's Disease

### Introduction

At the earliest stages of Alzheimer's Disease (AD), accumulation of amyloid beta (Aβ) initiates a broad pathological cascade that includes the formation of toxic tau species and glial activation. These interacting pathological processes converge on eventual synaptic degeneration, which is the strongest neuropathological predictor of cognitive impairment in AD. Thus, synaptic failure is a critical therapeutic target to treat AD progression.

Given the diverse pathways driving AD-related synaptic dysfunction, therapies targeting shared cellular mechanisms that regulate synapses, tau metabolism, and glial responses may more effectively shift multiple disease processes and slow progression than agents aimed at a single pathology. The p75 neurotrophin receptor (p75^{NTR}) is a compelling therapeutic target in this context, governing synaptic integrity and cell fate. Specifically, p75^{NTR} regulates broad intracellular signaling networks impacted in AD, affecting tau pathology by influencing multiple tau kinases such as CDK5, Fyn, INK, and GSK30, as well as influencing synaptic integrity and function by modulating core synaptic regulators including RhoA and cofilin. Moreover, preclinical studies have shown that p75^{NTR} modulation reduces tau pathology, preserves synapses, and improves cognition across multiple Aβ precursor protein- and tau-based mutant mouse models.

Based on this rationale, a randomized, double-blinded, placebo-controlled Phase IIa trial of the first-in-class p75^{NTR} modulator LM11A-31 in mild to moderate AD was previously conducted (see Example 15). The study met its primary safety endpoint and showed drug-placebo differences across prespecified exploratory biomarker domains (CSF biomarkers, structural MRI, and [¹⁸F]-fluorodeoxyglucose (FDG) PET), consistent with slowed pathophysiology.

Here, three critical extensions not examined in the original report are provided. First, because a ‘deep biology’ receptor is expected to modulate several signaling pathways at once, targeted assays, which quantify one protein at a time, may miss pathway-level changes distributed across multiple proteins. Therefore, proteomic analyses of CSF samples were performed using unbiased tandem mass tag based liquid chromatography-mass spectrometry (TMT-MS), quantifying more than 2,700 proteins and enabling network-level estimation of treatment effects on synaptic, tau, and glial pathways beyond conventional single-analyte assays. From this dataset, the hypothesis that LM11A-31 affects the synaptic proteome was tested. Network modules of covarying proteins that were previously derived from an independent reference cohort of AD and control participants were incorporated.

Second, state-of-the-art blood-based biomarkers were examined, including the plasma phosphorylated tau (p-tau) epitopes p-tau181, p-tau217, p-tau231, and glial fibrillary acidic protein (GFAP). See, for example, Ashton et al. Differential roles of Aβ42/40, p-tau231 and p-tau217 for Alzheimer's trial selection and disease monitoring. Nat Med 28, 2555-2562 (doi.org/10.1038/s41591-022-02074-w), Dec. 1, 2022; Abu-Rumeileh et al. Phosphorylated tau 181 and 217 are elevated in serum and muscle of patients with amyotrophic lateral sclerosis. Nat Commun 16:2019 (doi.org/10.1038/s41467-025-57144-7), Mar. 5, 2025; Horie et al., Plasma MTBR-tau243 biomarker identifies tau tangle pathology in Alzheimer's disease. Nat Med. 31(6):2044-2053 (doi: 10.1038/s41591-025-03617-7), June 2025, Epub Mar. 31, 2025). These measures provide scalable readouts of tau metabolism and glial activation suitable for multisite trials and clinical practice. Notably, these plasma biomarkers were linked to CSF proteomic signatures, allowing direct anchoring of clinically deployable markers to the underlying pathway-level effects detected by proteomics.

Third, global cognitive scales can dilute domain-specific signal and are susceptible to test-level noise. To overcome these obstacles, a psychometrically validated composite scores were used to evaluate four cognitive domains (memory, language, executive function, and visuospatial ability) constructed from items in the Mini Mental State Exam (MMSE), Alzheimer's Disease Assessment Scale-Cognitive Subscale 13 (ADAS-Cog13), and the trial's neuropsychological test battery. These composites, developed and validated in independent cohorts, provide improved measurement precision and robustness to item-specific variance, thereby increasing sensitivity to treatment effects within domains.

Overall, these analyses tested whether p75^{NTR} modulation (i) alters CSF proteomic network modules enriched for synaptic structure and function, (ii) affects clinically relevant plasma biomarkers, and (iii) preserves domain-specific cognition. The findings reveal LM11A-31-induced network-level proteomic changes, and effects on translatable biomarker and cognitive outcomes.

### Results

### Participants

Of 316 participants screened for inclusion, 241 were randomized into the intention to treat (ITT) population. In the ITT population, 128 participants (53.1%) were female, and the mean (SD) age was 70.7 (7.2) years. Detailed study characteristics have been reported previously. Endpoint analyses were conducted in participants from the ITT population with available data that met quality control procedures. As with the initial trial report, participants receiving 200 mg LM11A-31 or 400 mg LM11A-31 were pooled into one group for exploratory analyses (see Materials and Methods).

### Pre-Treatment Correlations Between Study Outcome Measures

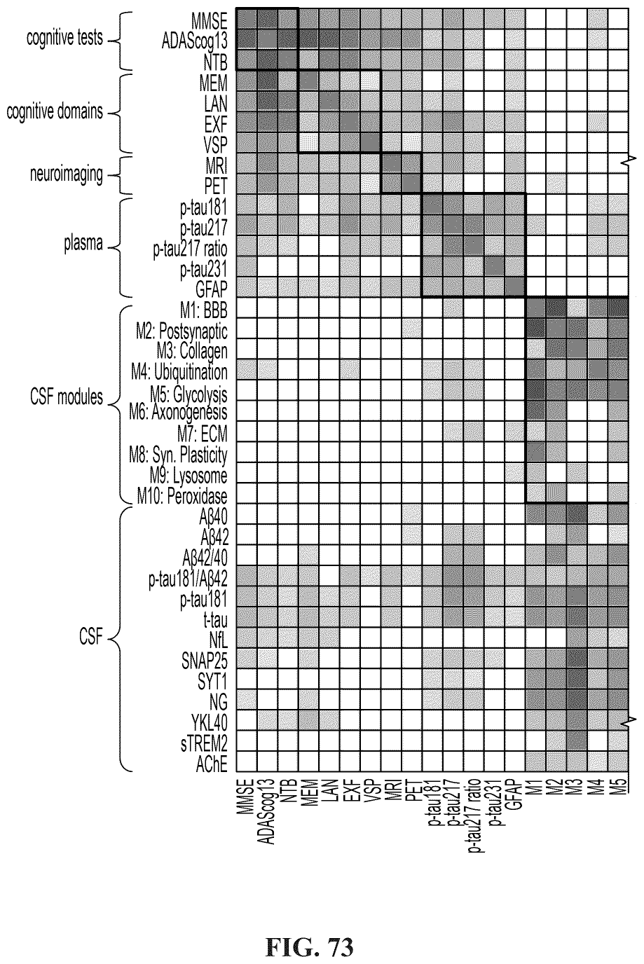

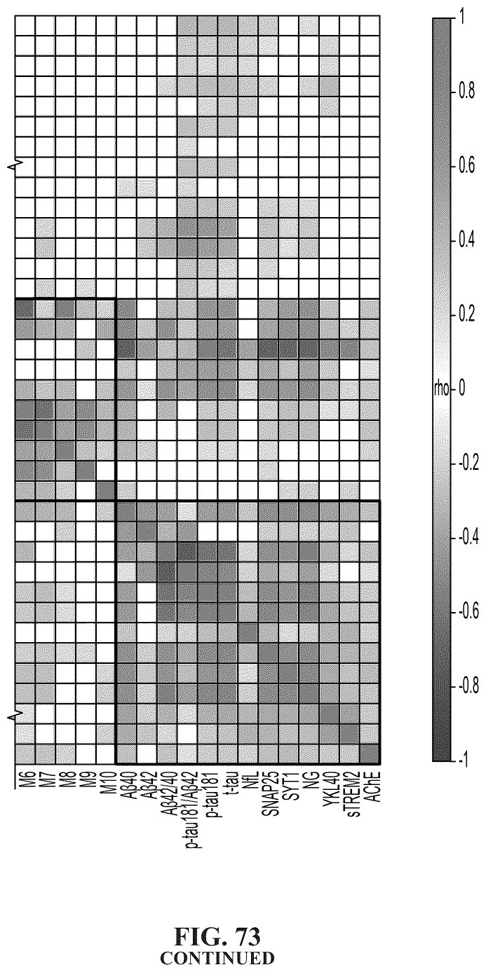

Prior to assessing treatment effects, a cross-sectional set of correlation analyses were performed to explore relationships between newly measured outcome variables at study baseline across the trial cohort (FIG. 73). The relationships among five biomarker-based outcomes were explored: structural MRI, [¹⁸F]-FDG PET, individual CSF biomarkers, plasma biomarkers of tau phosphorylation and glial status, as well as CSF proteomics data, grouped into modules of related proteins based on co-expression in an independent AD cohort. Additionally, the interrelationships of these biomarkers with individual tests of global cognitive function and composite cognitive domain scores (executive function, memory, language, and visuospatial) were examined. After correcting for multiple comparisons, significant relationships among variables broadly recapitulate prior work, and extend prior findings to show novel relationships among plasma biomarkers, previously established TMT-MS-derived CSF proteomic modules, structural MRI, [¹⁸F]-FDG PET and cognitive composite domain scores (FIG. 73).

For the TMT-MS proteomic analyses, individual proteins were assigned to clusters based on independently-defined proteomic networks that have been thoroughly characterized in terms of their relatedness, cell-type enrichment, and relevance to AD. The CSF proteins in the LM11A-31 trial cohort (n=2702 proteins) were mapped to 10 independently defined protein co-expression modules (M1-M10), which were derived from a TMT-MS CSF proteomic dataset collected from 431 individuals with or without AD at the Emory University Goizueta Alzheimer's Disease Research Center (ADRC; see Materials and Methods). Importantly, all 10 modules within the trial cohort are highly preserved in the networks defined within the ADRC cohort, highlighting the applicability of this technique for understanding proteomic changes within the trial cohort. Modules M1, M4, and M10 are generally non-specific in their cell-type enrichment. Modules M3 and M9 are enriched for microglia. M7 is enriched for microglia and endothelial cells. Modules M2, M5, M6 and M8 are primarily enriched for neuronal cell types.

Significant relationships were observed between the proteomic modules and multiple biomarkers. A priori, M4 and M5 were of particular interest based on their significant associations with AD pathological markers (CSF Aβ, p-tau181, total tau), APOE risk, and cognition in the ADRC cohort. M4, a module linked to ubiquitination and agnostic to biological sex in the ADRC cohort, correlated with plasma p-tau217, multiple CSF markers, and cognition as measured by the MMSE, ADAS-cog13, and executive function composite (FIG. 73). These latter associations with cognition are consistent with prior work in independent AD cohorts. In addition to being related to AD markers in the ADRC cohort, M5 also showed sex differences, with higher levels observed in women. M5 contains several AD- and synaptic-relevant proteins including total tau (MAPT) and neurogranin. M5 exhibited broad profiles of significant associations with core CSF AD, synaptic, and inflammatory biomarkers, as well as plasma p-tau181 and p-tau217 (FIG. 73).

Plasma p-tau217 was significantly and inversely corelated with grey matter volume as quantified by structural MRI and cerebral glucose metabolism as quantified by [¹⁸F]-FDG PET. For the TMT-MS-derived CSF proteomic modules, plasma p-tau217 was significantly positively correlated with M4 and M5 and significantly negatively correlated with M1 and M7. Additionally, significant negative associations of plasma p-tau217 were observed with all four subdomains of the cognitive composite scores (memory, language, executive function, and visuospatial), along with each of the three global cognitive scores (MMSE, ADAS-cog, NTB). Consistent with the observed significant relationships of p-tau217 with cognition, it was also observed that p-tau217 significantly correlated with all three synaptic biomarkers (SNAP-25, SYT1, NG; FIG. 73).

### CSF Proteins Affected by LM11A-31 are Enriched for Synaptic Structure and Function

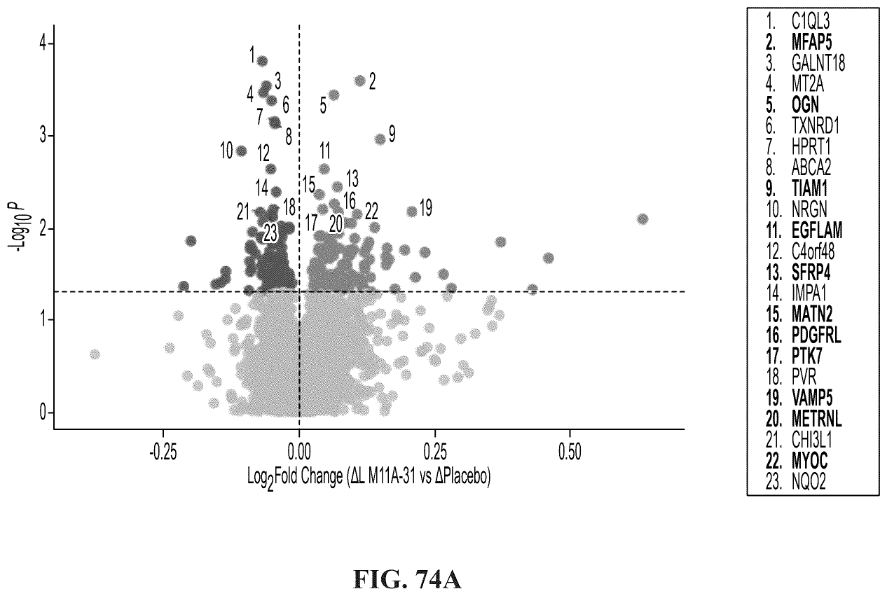

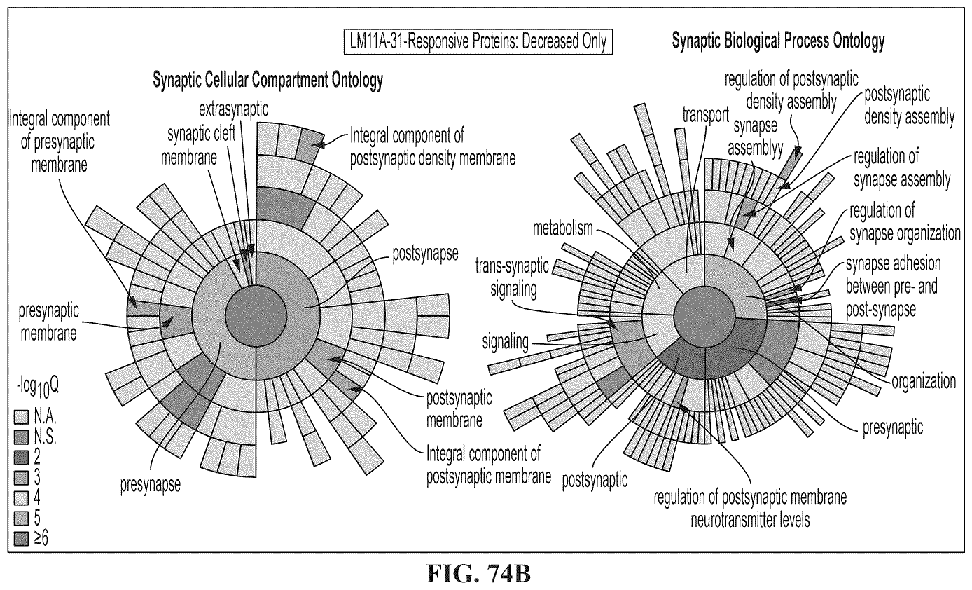

CSF proteins derived from the TMT-MS dataset were analyzed individually to screen for proteome-wide effects of LM11A-31. Of the 2702 proteins detected in >50% of the samples (Materials and Methods), 241 were significantly altered by 26-week LM11A-31 treatment at an uncorrected P<0.05 (FIG. 74A). Compared to placebo, treatment with LM11A-31 significantly decreased the abundance of 131 proteins from baseline to follow-up, and significantly increased the abundance of 110 proteins from baseline to follow-up. Proteins above the 90^{th} percentile, ordered by P value, are labelled in FIG. 74A.

The TMT-MS proteomic dataset included three proteins previously measured by immunoassay in this cohort: the postsynaptic protein NRGN (neurogranin), the glial protein CHI3L1 (YKL40) and MAPT (t-tau). Neurogranin, YKL40, and t-tau levels measured by immunoassay were highly correlated with those obtained by TMT-MS (neurogranin r=0.83, P=2.4E-81; YKL40 r=0.7; P=1.1E-47; t-tau r=0.82, P=7.8E-78), demonstrating consistency across platforms. In line with prior work, all three proteins measured by TMT-MS also showed significantly reduced abundance with LM11A-31 treatment compared to placebo, with neurogranin (log₂ FC=−0.11; P=0.001) and YKL40 (log₂ FC=−0.073; P=0.007) both ranking among the most significantly affected proteins across the entire dataset (FIG. 74A).

Given the direct role of p75^{NTR} on regulating synaptic integrity, the 241 proteins affected by LM11A-31 were mapped to their gene symbols and tested for synaptic enrichment using an expertly curated database of 1602 unique genes with 4218 synaptic annotations (SynGO; see Materials and Methods). This strategy enabled associations of proteins which were either significantly increased or decreased in abundance by LM11A-31 to cellular compartments and biological processes associated with the synapse.

The set of 131 proteins with reduced abundance were significantly enriched for cellular compartment terms and biological processes in SynGO, with 39 (30% of the significantly decreased proteins) mapped to at least one SynGO annotation (FDR-corrected threshold of Q<0.01; Table 4). The significantly enriched cellular compartment terms included both presynaptic (e.g., presynapse, presynaptic membrane, integral component of presynaptic membrane) and postsynaptic compartments (e.g., postsynapse, postsynaptic membrane, postsynaptic density membrane), as well as general synaptic structures such as the synapse and synaptic cleft (FDR corrected P=4.17E-09; Table 15, FIG. 74B). The significantly enriched biological process terms included synapse organization, synaptic signaling, synapse and postsynaptic density assembly, and trans-synaptic signaling, among others (FDR corrected P=6.72E-11; Table 15, FIG. 74B). Of the 110 proteins that were increased in abundance by LM11A-31 treatment, only 13 were mapped to an annotation in SynGO. Enrichment for synaptic cellular compartment or biological process annotations were not detected at an FDR-corrected threshold of Q<0.01.

TABLE 15
CSF proteins which decreased in abundance with LM11A-31
treatment show enrichment of synaptic ontology terms

| | Gene | | |
|---|---|---|---|
| | Count | P-Value | Q-Value |
| Cellular Compartment Ontology | | | |
| synapse | 35 | 3.48E−10 | 4.17E−09 |
| presynapse | 18 | 3.47E−06 | 2.08E−05 |
| presynaptic membrane | 7 | 2.18E−04 | 5.23E−04 |
| integral component of presynaptic membrane | 6 | 4.58E−04 | 9.16E−04 |
| postsynapse | 18 | 1.22E−04 | 4.14E−04 |
| postsynaptic membrane | 7 | 1.38E−04 | 4.14E−04 |
| integral component of postsynaptic membrane | 5 | 2.05E−03 | 2.73E−03 |
| integral component of postsynaptic density membrane | 5 | 1.65E−03 | 2.47E−03 |
| synaptic cleft | 3 | 7.51E−04 | 1.29E−03 |
| Biological Process Ontology | | | |
| process in the synapse | 33 | 4.48E−12 | 6.72E−11 |
| synapse organization | 14 | 3.42E−06 | 2.57E−05 |
| synapse assembly | 8 | 3.39E−05 | 1.69E−04 |
| regulation of synapse assembly | 5 | 1.65E−03 | 2.47E−03 |
| postsynaptic density assembly | 4 | 6.12E−05 | 1.84E−04 |
| regulation of postsynaptic density assembly | 3 | 5.84E−04 | 1.25E−03 |
| regulation of synapse organization | 3 | 1.56E−03 | 2.47E−03 |
| synapse adhesion between pre- and post-synapse | 3 | 5.00E−03 | 6.25E−03 |
| process in the presynapse | 7 | 8.58E−03 | 9.91E−03 |
| process in the postsynapse | 7 | 4.22E−03 | 5.76E−03 |
| regulation of postsynaptic membrane neurotransmitter receptor levels | 6 | 1.60E−03 | 2.47E−03 |
| synaptic signaling | 9 | 4.59E−05 | 1.72E−04 |
| trans-synaptic signaling | 8 | 2.09E−04 | 5.23E−04 |

Q-values are FDR corrected P values from the enrichment analysis, which was performed with SynGO. The corresponding visualization of the enrichment analysis is available in FIG. 74B. Only ontology terms with Q<0.01 are shown. Terms are shown nested under their closest significant parent term.

### LM11A-31 Attenuates Longitudinal Progression of CSF Proteomic Modules

In addition to examining effects on synaptic proteins, the broader effects of LM11A-31 treatment on the CSF proteome network were assessed using data from the ADRC reference cohort which inform on the expected direction of change in individual proteins and proteomic network modules (i.e., clusters of co-expressed proteins) in AD. The proportion of proteins whose changes under LM11A-31 treatment were in the opposite direction to these priors was investigated. Of 204 proteins meeting an uncorrected P<0.05 in the LM11A-31 group with a counterpart in the ADRC group, 129 (63.2%) moved in the opposite direction of AD progression as defined by the ADRC cohort, with 75 in the same direction (χ² P=0.00016), suggesting that LM11A-31 may tend to broadly normalize AD-related changes in CSF protein levels.

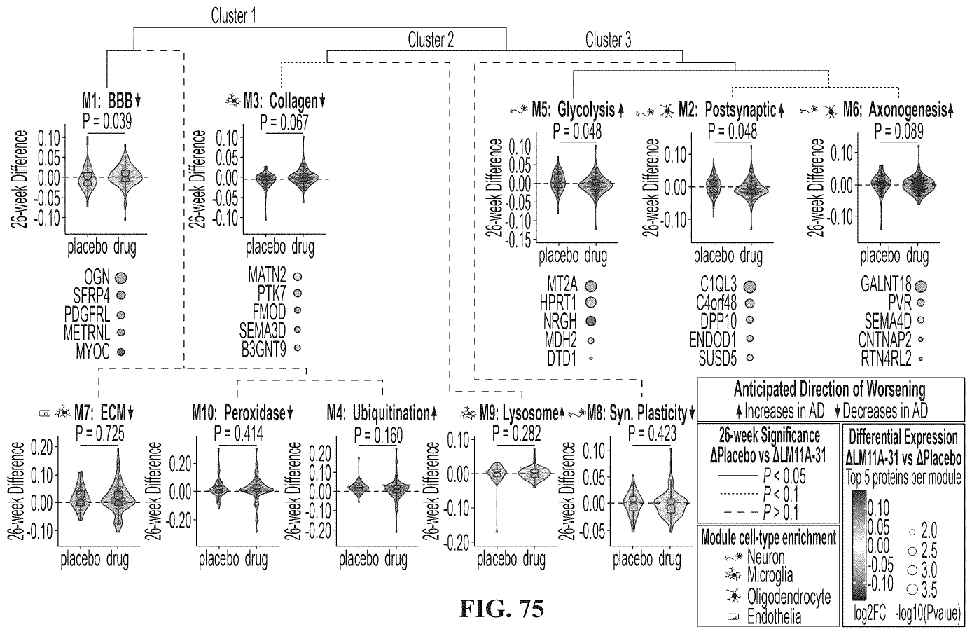

Next, the effects of LM11A-31 treatment were examined at the module level. For reference, a schematic of the original cluster dendrogram that produced the 10 proteomic modules, labelled M1 (largest module) to M10 (smallest module), is shown in FIG. 75. The dendrogram broadly distinguishes 3 clusters of modules. Arrows indicate the expected direction of worsening for each module, based on associations with p-tau and Aβ presented in Bangs et al. bioRxiv 2025.03.14.643332 (2025).

Modules in Cluster 1 are generally non-specific in their cell-type enrichment (M1, M4 and M10), indicating broad involvement of multiple cell types, though M7 is enriched in both microglial and endothelial cells. Proteins within M1 decreased over the course of the trial in the placebo group (placebo 26-week median percent change=−14.4%), consistent with progression of AD as defined within the ADRC cohort. This decrease in M1 protein abundance was attenuated with LM11A-31 treatment (LM11A-31 26-week median percent change=−1.6%). Group differences were statistically significant (placebo vs LM11A-31 P=0.039). The top gene ontology (GO) terms for M1 were plasma and blood-brain barrier. Longitudinal changes under LM11A-31 and placebo did not differ significantly for M7, M10 and M4 (FIG. 3; P>0.05 for each).

Modules in Cluster 2 are enriched for microglia (M3 and M9). In the placebo group, M3 exhibited a median percent change of −7.2%, consistent with disease progression, whereas the LM11A-31 group showed a small percent increase of 4.6% (FIG. 75). These group differences in M3 protein levels trended towards statistical significance (P=0.067). The top GO term for M3 was collagen. Significant differences for LM11A-31 and placebo were not observed for M9 (P=0.282).

Modules in Cluster 3 are enriched for neuronal cell types (M8, M5, M2, and M6) as well as oligodendrocytes (M2 and M6). Although no significant differences between placebo and LM11A-31 were observed on M8 levels (P=0.423), LM11A-31 treatment had a significant or trend-level effect on three of the four remaining modules in this cluster (FIG. 75). In the placebo group, the median 26-week percent change was 2.6% for M5, −6.2% for M2 and 0.7% for M6. In the LM11A-31 group, the median 26-week change opposed the direction of worsening as defined in the ADRC cohort for each of these modules (FIG. 75), with 26-week median percent changes in the LM11A-31 group being −15.8% for M5 (LM11A-31 vs placebo P=0.048), −25.3% for M2 (LM11A-31 vs placebo P=0.056), and −14.8% for M6 (LM11A-31 vs placebo P=0.089). Of note, the top GO enrichment terms linked to these modules were postsynaptic (M2), glycolysis (M5), and axonogenesis (M6). Thus, using an independent, unbiased, network-based approach, the results again converge to suggest an ability of LM11A-31 to preserve neuronal and synaptic integrity in AD, while also impacting glial and blood-brain-barrier related pathways.

### LM11A-31 Attenuates Longitudinal Increases in Plasma Phosphorylated Tau

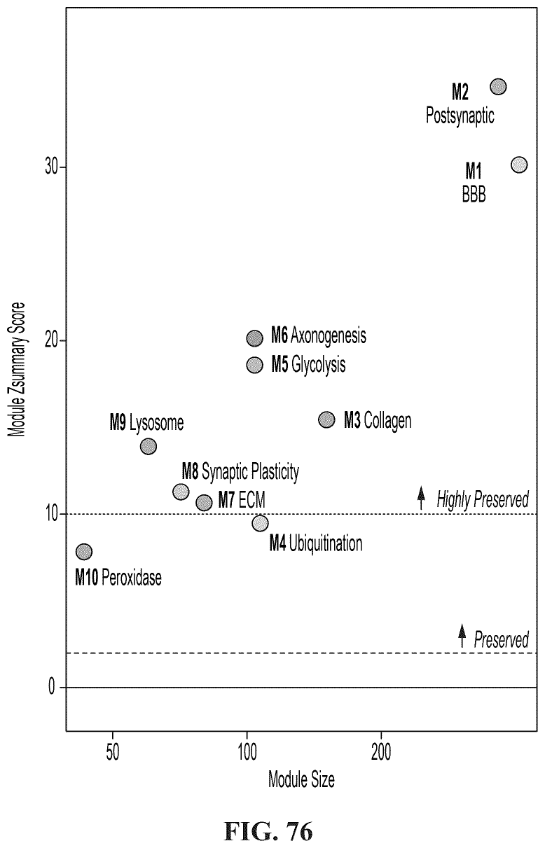

Next, three phosphorylated tau species in the plasma: p-tau181, p-tau217, and p-tau231 were measured. Consistent with prior literature examining longitudinal changes in plasma p-tau in participants on the AD continuum, each of the plasma p-tau species exhibited a longitudinal increase in the placebo group from baseline to 26 week follow-up (FIG. 76). The median annualized increase in the placebo group was 7.0% for p-tau181, 29.1% for p-tau217 and 16.1% for p-tau231.

In the present study, participants in the LM11A-31 group exhibited a negative annualized change on each of these plasma p-tau species, with median annual percent change values of −1.5% for p-tau181, −2.1% for p-tau217 and −5.0% for p-tau231. Comparisons between the LM11A-31 and placebo groups revealed non-significant but hypothesis-consistent attenuation of annual percent increases in p-tau181 and p-tau231 (p-tau181 P=0.68; p-tau231 P=0.37). The net difference in longitudinal progression on p-tau217 between LM11A-31 and placebo was −31.2% (+29.1%→2.12%) and was significantly different between groups (P=0.049; FIG. 76).

A significant positive relationship between plasma p-tau217 and plasma GFAP, a marker of glial activation, was reported in a secondary baseline analysis of the TRAILBLAZER-ALZ donenamab Phase 3 trial. Consistent with this result, it was found that plasma p-tau217 and plasma GFAP were significantly positively correlated at baseline in the Phase IIa LM11A-31 trial. Moreover, GFAP exhibited longitudinal increases in the placebo group from baseline to 26-week follow-up (annualized percent change: 3.9%). In line with initial findings from the Phase IIa trial, which showed that LM11A-31 significantly attenuated longitudinal increases in the CSF glial marker YKL40, this study found that LM11A-31 inverted the direction of the median annual percent change in plasma GFAP relative to placebo (−7.2%). The net difference in longitudinal progression on GFAP between LM11A-31 and placebo was −11.1% (+3.9%→7.2%). While this change was hypothesis consistent, it was nominally significant (LM11A-31 vs placebo P=0.15; FIG. 76).

### LM11A-31 Preserves Visuospatial Function

In the initial trial report, MMSE and ADAS-Cog13 scores demonstrated a significant decline in the placebo group, while NTB scores demonstrated no detectable change. Treatment with LM11A-31 was associated with an approximately 50% slowing of the decline detected with MMSE and ADAS-Cog13 ratings relative to placebo. Although these effects did not reach statistical significance, the observed directionality raises the possibility that there could be a cognitive effect that may be detectable with either a longer trial interval or more sensitive cognitive measures. Thus, to increase the sensitivity of the cognitive outcome measures, composite cognitive domain scores were calculated using the items from this trial's cognitive test battery, with the goal of investigating whether LM11A-31 treatment had effects on specific domains of cognition that may have been obscured in the analyses of global cognitive tests.

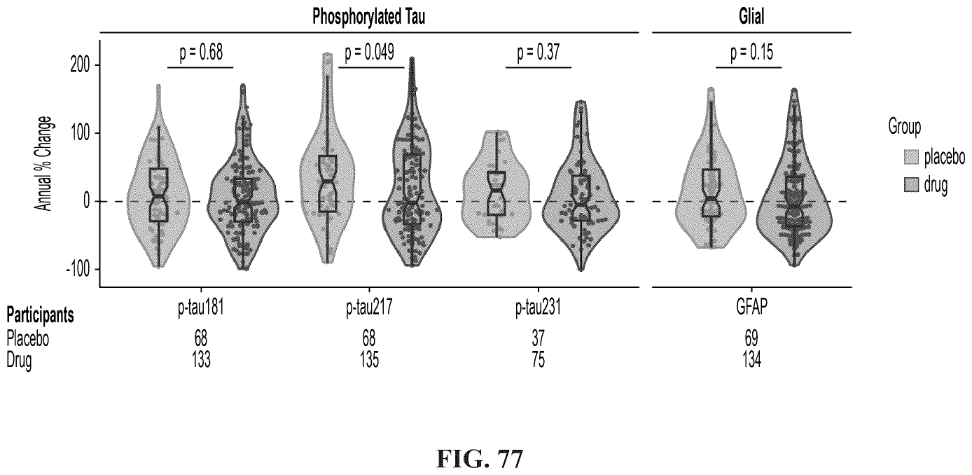

Composite scores for memory, language, executive function, and visuospatial ability were calculated using items from the MMSE, ADAS-Cog13, and NTB. Group by visit interaction effects on cognitive domains were examined using linear mixed models (FIG. 77).

Of the four composite domains, significant decline within the placebo group was detected at the 26-week visit for the memory (P<0.0001) and visuospatial composites (P=0.015). When examining the group by visit interaction effects, there was a small but significant effect of LM11A-31 treatment on the visuospatial composite domain at 12-weeks (least squares (LS) mean placebo=−0.193; LS mean LM11A-31=−0.083; P=0.032; FIG. 77), with the LM11A-31 group having significantly higher mean scores (better function) on the visuospatial domain compared to placebo. This effect remained significant at the 26-week visit (LS mean placebo=−0.263; LS mean LM11A-31=−0.118; P=0.015), suggesting that LM11A-31 treatment may preserve visuospatial function. At the 26-week timepoint, group by visit interactions for the memory, language and executive function composite scores were not statistically significant (FIG. 77).

### Discussion

In this post-hoc analysis of the LM11A-31 Phase IIa trial, converging biomarker and cognitive evidence for disease-modifying effects of p75^{NTR} modulation in mild-to-moderate AD was found. Plasma p-tau217, a sensitive marker of tau pathology, differentiated drug and placebo groups, with LM11A-31 slowing its rate of increase over 26 weeks. TMT-MS-based CSF proteomics identified 241 proteins altered by treatment, with those which decreased in abundance significantly enriched for synaptic compartments and processes. An independent, network-based module analysis confirmed effects on multiple protein co-expression clusters, including those enriched for neurons, oligodendrocytes, and microglia. Finally, a composite visuospatial score exhibited slowing of decline under LM11A-31. Together, these results demonstrate that p75^{NTR} modulation impacts tau, synaptic integrity, and cognition in parallel.

Before assessing longitudinal change, cross-sectional associations among biomarkers and cognition were examined (FIG. 73). Well-established relationships among core AD pathophysiology measures were replicated, including correlations between p-tau217, CSF synaptic markers, grey matter volume, and [¹⁸F]-FDG PET metabolism, as well as cognitive composite scores. These findings provide important internal replication of known biomarker-cognition links and reinforce the robustness of the trial dataset. Additionally, new associations between plasma p-tau217 and specific CSF proteomic modules were identified, linking this blood-based biomarker to broader CSF-derived protein networks involved in synaptic and glial biology. Such relationships support the validity of the biomarker set and strengthen the rationale for their inclusion in early-stage therapeutic trials.

Among the plasma tau biomarkers, p-tau217 showed the largest separation between groups, with a 31% net difference in progression over 26 weeks. This magnitude is notable for a short interval trial in symptomatic AD. As a fluid biomarker, p-tau217 has been suggested to reflect synaptic dysfunction stemming from Aβ pathology. In tauopathy mouse models and preclinical AD brains, p-tau217 precedes tau aggregates, increasing in vulnerable neuronal populations and co-localizing with synaptic markers. Antibody-based targeting of the p-tau217 epitope rescues neuronal and synaptic loss in mice, and monoclonal antibodies against p-tau217 are in early stage clinical testing.

Synaptic preservation is a primary goal for AD therapeutics, as synaptic loss is the strongest neuropathological predictor of cognitive decline. LM11A-31's ability to modulate p75^{NTR} directly influences signaling pathways that maintain dendritic spine structure, synaptic vesicle function, and actin cytoskeleton dynamics. Prior immunoassay-based analyses in this trial showed treatment-related decreases in CSF neurogranin and SNAP-25. The current proteomic approach extends on this work, demonstrating that proteins affected by LM11A-31 treatment are enriched for presynaptic and postsynaptic compartments and processes including synapse organization, synaptic signaling, and trans-synaptic communication (FIG. 74). These results replicate earlier targeted findings and broaden them to an unbiased proteome-wide scale, strengthening the evidence that LM11A-31 impacts synaptic biology in humans.

Because p75^{NTR} signaling extends beyond synaptic maintenance to include glial regulation, metabolism, and cytoskeletal remodeling, an analysis framework was sought that could detect such broad physiological effects. The CSF protein co-expression modules used here were defined in an independent AD reference cohort and captured coordinated protein changes across diverse cellular processes. Significant or trend-level effects of LM11A-31 were observed on neuron-enriched modules (M2, M5, M6), a microglia-enriched module (M3), and a module representative of blood-brain barrier dysfunction (M1). These findings suggest that p75^{NTR} modulation can simultaneously affect neuronal structural integrity and microglial activation states, consistent with preclinical evidence that LM11A-31 reduces neuroinflammation and promotes resilience of multiple brain cell types.

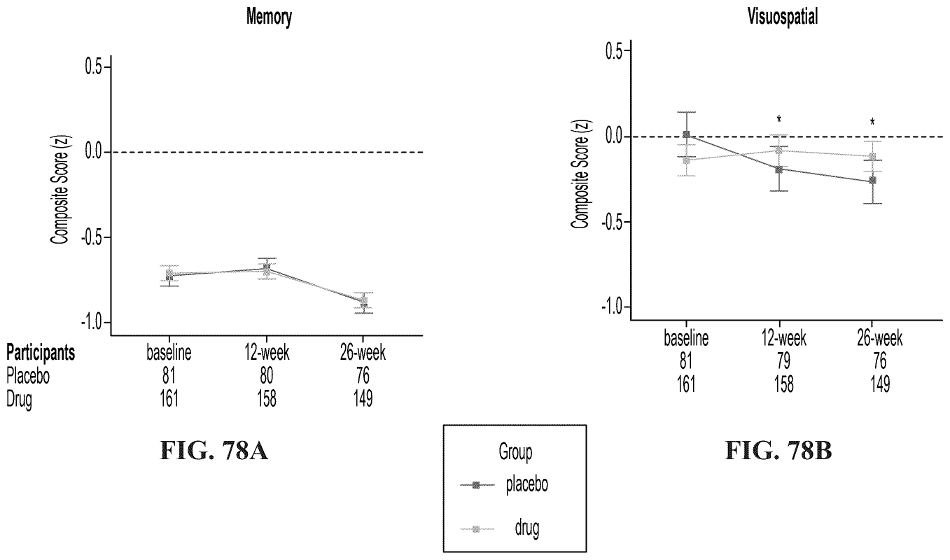

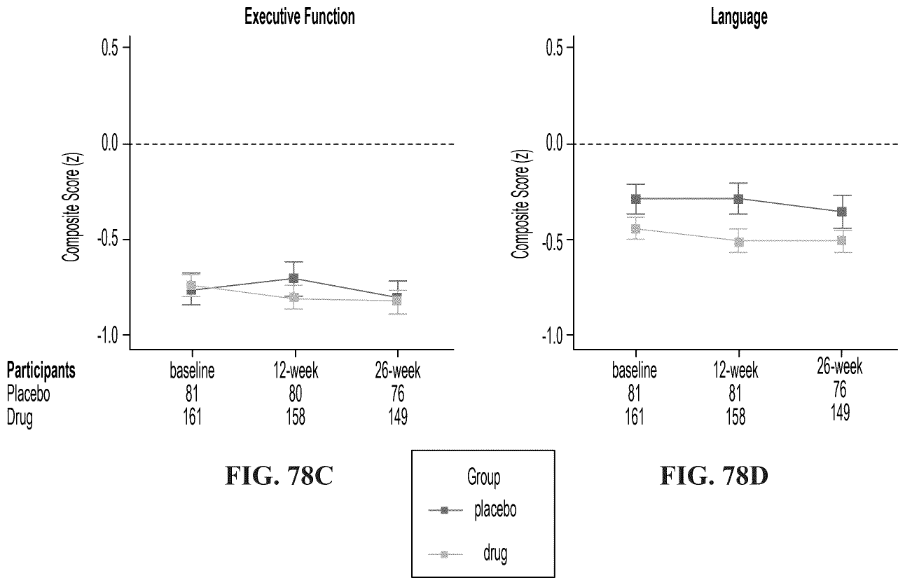

While the trial was not powered for cognitive efficacy, a consistent preservation of visuospatial ability was detected under LM11A-31, with effects of treatment emerging at 12 weeks and maintained through 26 weeks (FIG. 78B). Baseline p-tau217 was correlated with visuospatial performance (FIG. 73), potentially linking tau-associated synaptic disruption to this domain. Visuospatial function may be particularly sensitive to short-term therapeutic effects because it relies on rapid integration of perceptual information and spatial working memory, processes that depend heavily on synaptic efficiency in parietal and occipital association cortices.

The breadth of p75^{NTR} modulation is evident in its impact on individual CSF proteins with known or emerging biomarker or therapeutic roles in AD. These include osteopontin (SPP1), a pro-inflammatory cytokine linked to progression from MCI to AD; semaphorin-4D (SEMA4D), implicated in astrocyte activation and barrier dysfunction and CAP2, involved in tau and actin cytoskeleton regulation. The modulation of such diverse targets suggests that p75^{NTR} signaling intersects with multiple pathogenic pathways, raising the possibility of combination therapeutic strategies that exploit these nodes.

Overall, across biomarkers and cognition, LM11A-31 slowed progression of key molecular and cognitive markers in mild-to-moderate AD, with converging effects on tau phosphorylation, synaptic proteins, glial-linked networks, and visuospatial function. This convergence aligns with p75^{NTR}'s role as a ‘deep biology’ receptor that integrates neuronal and glial signaling pathways. Because promoting synaptic resilience may be most effective earlier in AD, longer trials or prodromal interventions may yield broader cognitive effects. Collectively, these effects support continued development of p75^{NTR} modulators, including LM11A-31, as multi-mechanism AD therapeutics, and highlight the value of integrating blood-based p-tau assays, CSF proteomics, and domain-specific cognitive composites to sensitively detect treatment effects in AD trials.

### Materials and Methods

The current study is a post hoc analysis of the LM11A-31 Phase IIa clinical trial. Three additional endpoints (CSF proteomic modules, plasma biomarkers, cognitive domain scores) which were not included in the original trial protocol and statistical analysis plan are described below.

### Participants and Study Design

Data was collected as part of a 26-week randomized, double-blinded, placebo-controlled, Phase IIa safety and exploratory endpoint trial of LM11A-31 (LM11A-31-BHS) in individuals with mild to moderate AD dementia (EU Clinical Trials registration: 2015-005263-16; ClinicalTrials.gov registration: NCT03069014). The trial methodology and initial results have been described previously. In brief, the study was conducted between May 2017 and June 2020 at 18 active sites in five European countries: Austria, the Czech Republic, Germany, Spain, and Sweden. The trial was conducted in accordance with the Declaration of Helsinki and ICH-GCP. All required study documents were submitted to the Ethics Committees of the participating countries. Participants were aged between 50 and 85 years, had Mini Mental State Exam (MMSE) scores between 18 and 26, met criteria for CSF Aβ abnormality, and had a clinical diagnosis of AD according to the McKhann criteria (McKhann et al., Alzheimers Dement. 7(3):263-9 (2011)). A full list of trial inclusion criteria can be found in the previous report (Shanks et al., Nat. Med. 30(6):1761-70 (2024)) Participants were randomized 1:1:1 into placebo, 200 mg LM11A-31, or 400 mg LM11A-31. Participants, their caregivers, and study physicians were blinded to assigned treatment arm. Study medication was administered per oral dosing twice daily.

Previous pharmacokinetic studies have investigated CSF concentrations of LM11A-31 in rodents and humans (unpublished data). Brain exposure estimates from these studies, along with prior pre-clinical dose-response studies suggest that doses of 200 mg or 400 mg LM11A-31 twice daily are adequate to fully engage targeted p75^{NTR}-associated mechanisms. Consistent with these findings, the 200 mg and 400 mg dose groups exhibited similar change in biomarker and cognitive endpoints and were therefore pooled into one group in our previous report. Dose groups of LM11A-31 are also pooled for the post-hoc outcomes presented here.

### Previously Reported CSF and Neuroimaging Data

The baseline correlation matrix presented in FIG. 73, in addition to showing novel data (described below), includes previously published CSF, imaging, and cognitive test data which were collected in the trial participants. The CSF biomarker panel included AD biomarkers Aβ40, Aβ42, p-tau181, total tau (t-tau), the degenerative marker neurofilament light chain (NfL), synaptic markers synaptotagmin-1 (SYT1), synaptosomal-associated protein 25 (SNAP25), and neurogranin (NG), the glial markers Chitinase-3-like protein 1 (YKL40), soluble triggering receptor expressed on myeloid cells (sTREM2) and acetylcholinesterase (AChE) activity. Trial neuroimaging data consisted of T1 weighted structural MRI data and [¹⁸F]-FDG PET data. Grey matter volumes and standardized uptake value ratios (SUVr) were extracted within AD-vulnerable brain regions, defined in an independent cohort of participants who met key trial inclusion criteria. The cognitive test data are described in the cognitive composite score methods subsection.

### CSF Proteomics

Participants with CSF samples of sufficient remaining quantity after pre-specified endpoints were conducted were included in CSF proteomic analyses. Global unbiased tandem-mass tag mass spectrometry (TMT-MS) based proteomics was performed on Orbitrap Ecliples instrument by Emtherapro, Inc. (Atlanta, Georgia).

### Tandem-Mass Tag Mass Spectrometry CSF Proteomic Measurements

Samples were collected, processed, and analyzed using TMT-MS followed by unbiased quantification as described previously.

### Protein Digestion of CSF

First, 70 μl of CSF was digested with lysyl endopeptidase (LysC) and trypsin, reduced and alkylated with 1.4 μl of 0.5 M tris2(-carboxyethyl)-phosphine (Thermo Fisher) and 7 μl of 0.4 M chloroacetamide in a 90° C. water bath for 10 min, following with bath sonication for 5 min. After letting samples cool to room temperature, 78 μl of 8 M urea buffer (8 M urea, 10 mM Tris, 100 mM NaH2PO4, pH 8.5) and 3.5 g of LysC (Wako) were added to each sample, resulting in a final urea concentration of 4 M. The samples were mixed, spun down and incubated overnight at 25° C. for digestion with LysC. The following day, samples were diluted to 1 M urea with solution containing 468 μl of 50 mM ammonium bicarbonate and 7 μg of Trypsin (Thermo Fisher). The samples were subsequently incubated overnight at 25° C. for digestion with trypsin, acidified to a final concentration of 1% formic acid and 0.1% trifluoroacetic acid. This was immediately followed by desalting on 30 mg HLB columns (Waters) and then eluted with 1 ml of 50% acetonitrile (I). To normalize protein quantification across batches, 100 μl was taken from all CSF samples and then combined to generate a pooled sample. This pooled sample was then divided into global internal standards (GIS). All individual samples and the pooled standards were then dried using a speed vacuum (Labconco).

### TMT Labeling of CSF Peptides

All CSF samples were balanced for treatment group, sex, and APOE status across 4 batches and loaded on to a 16-plex TMT (TMTpro) kit (Catalog #A44520 and Lot #U1292951). Care was taken to ensure baseline and 6-month CSF samples from same participant were loaded onto the same batch. Further, one GIS pool and AD and control standard pools created by aggregating CSF from age matched AD and control cohorts from Emory Alzheimer's Disease Research Center (ADRC) was added per batch. In preparation for labeling, each CSF peptide digest was resuspended in 75 μl of 100 mM triethylammonium bicarbonate (TEAB) buffer. Meanwhile, 5 mg of TMT reagent was dissolved into 200 μl of I. Once both were in suspension, 15 μl of TMT reagent solution was subsequently added to the resuspended CSF peptide digest. After 1 h, the reaction was quenched with 4 μl of 5% hydroxylamine (Thermo Fisher Scientific) for 15 min. The peptide solutions were then combined according to the batch arrangement. Finally, each TMT batch was desalted with 60 mg HLB columns (Waters) and dried via speed vacuum (Labconco).

### High-pH Peptide Fractionation

Dried samples were re-suspended in high pH loading buffer (0.07% vol/vol NH4OH, 0.045% vol/vol FA, 2% vol/vol I) and loaded onto a Water's BEH column (2.1 mm×150 mm with 1.7 μm particles). A Vanquish UPLC system (Thermo Fisher Scientific) was used to carry out the fractionation. Solvent A consisted of 0.0175% (vol/vol) NH4OH, 0.01125% (vol/vol) FA, and 2% (vol/vol) I; solvent B consisted of 0.0175% (vol/vol) NH4OH, 0.01125% (vol/vol) FA, and 90% (vol/vol) I. The sample elution was performed over a 25 min gradient with a flow rate of 0.6 mL/min with a gradient from 0 to 50% solvent B. A total of 192 individual equal volume fractions were collected across the gradient. Fractions were concatenated to 96 fractions and dried to completeness using vacuum centrifugation.

### Mass Spectrometry Analysis and Data Acquisition

All samples (˜1 μg for each fraction) were loaded and eluted by 1200 Ultimate U3000 RSLCnano (Thermo Fischer Scientific) with an in-house packed 15 cm, 150 μm i.d. capillary column with 1.7 μm CSH (Water's) over a 35 min gradient. MS was performed with a high-field asymmetric waveform ion mobility spectrometry (FAIMS) Pro front-end equipped Orbitrap Eclipse (Thermo Fisher) in positive ion mode using data-dependent acquisition with 1 s top speed cycles for each FAIMS compensative voltage. Each cycle consisted of one full MS scan followed by as many MS/MS events that could ft within the given 1 s cycle time limit. MS scans were collected at a resolution of 120,000 (410-1600 m/z range, 4×10⁵ AGC, 50 ms maximum ion injection time, FAIMS compensative voltage of −45 and −65). Only precursors with charge states between 2+ and 6+ were selected for MS/MS. All higher energy collision-induced dissociation (HCD) MS/MS spectra were acquired at a resolution of 30,000 (0.7 m/z isolation width, 35% collision energy, 1.25×10⁵ AGC target, 54 ms maximum ion time). Dynamic exclusion was set to exclude previously sequenced peaks for 20 s within a 10-ppm isolation window.

### Database Search and Protein Quantification

All raw files were analyzed using the Proteome Discoverer Suite (v.2.5.0.400, Thermo Fisher). MS/MS spectra were searched against the UniProtKB human proteome database (downloaded in February 2019 with 20,340 total sequences). The MSfragger search node was used to search the RAW files, with search parameters specified as follows: semi tryptic specificity, maximum of one missed cleavages, minimum peptide length of seven, fixed modifications for TMTPro tags on lysine residues and peptide N-termini (+304.2071 Da) and carbamidomethylation of cysteine residues (+57.02146 Da), variable modifications for oxidation of methionine residues (+15.99492 Da), serine, threonine and histidine TMTPro (+304.2071 Da) precursor mass tolerance of 20 ppm and a fragment mass tolerance of 0.05 Da. Percolator was used to filter peptide spectral matches and peptides to a False Discovery rate (FDR)<1%. Following spectral assignment, peptides were assembled into proteins and were further filtered based on the combined probabilities of their constituent peptides to a final FDR of 1%. Peptides were grouped into proteins following strict parsimony principle. Following human database search, 3357 proteins were detected. This dataset was filtered to exclude any proteins not detectable in at least half of the participants with CSF data available, leaving 2702 proteins. Proteomic data was normalized and log transformed using known methods. Age, sex, trial site, and APOE status were regressed out prior to further analysis.

### Analysis of CSF Synaptic Proteins

To test that hypothesis that CSF proteins affected by LM11A-31 treatment were located in synapses or had synaptic function, enrichment analysis was conducted using the SynGO synaptic database. The SynGO database is a global partnership between the GO consortium and expert laboratories to provide systematic, accurate annotations of synaptic genes. SynGO categorizes gene annotations into cellular compartments (e.g. post-synapse, pre-synapse, synaptic membrane), and biological processes (e.g. synaptic signaling, synaptic organization, transport). Prior research demonstrates that performing enrichment analysis of all differentially expressed genes pooled together may reduce power to detect disease-associated changes relative to analyzing up- and down-regulated gene sets separately. With this in mind, the enrichment analysis with SynGO was performed separately for proteins increased or decreased in abundance by LM11A-31.

Additionally, the CSF proteins were assigned to modules based on an independently-defined network created with Weighted Gene Co-Expression Network Analysis (WGCNA). Module definition with WGCNA was previously performed in an independent reference cohort of 431 older adults from the Goizueta Alzheimer's Disease Research Center (ADRC) at Emory University, and proteins detected in the trial cohort were mapped to their corresponding module. All 10 modules in the clinical trial TMT-MS data are highly preserved in the ADRC-defined network. There were 758 proteins in the trial cohort which were not detectable in at least half of the ADRC cohort and were not assigned to a module. Principal components analysis was used to compute an eigenprotein for each module that reflects each participant's expression of the proteins within that module.

In addition to module definition, the ADRC cohort served as an independent reference cohort to determine the directionality of AD-related progression for each module, based on correlations with known cognitive and AD biomarkers (FIG. 74A). Module cell type enrichment, as depicted in FIG. 75, was also determined in the ADRC cohort.

Outlier analyses were conducted using network adjacency methods. Seven samples were flagged as outliers (>3 standard deviations from the mean) and excluded from further analysis.

### Plasma Biomarkers

Participants with plasma samples of sufficient remaining quantity after pre-specified endpoints were conducted were included in the plasma biomarker analyses. Plasma samples were sent to C2N Diagnostics (St. Louis, Missouri) and the University of Gothenburg for plasma biomarker quantification. C2N Diagnostics used liquid chromatography-tandem mass spectrometry (LC-MS/MS) to measure p-tau181 and p-tau217. Plasma p-tau231 and plasma GFAP were quantified using antibody-based assays using known methods.

### Annual Percent Change Calculation

Prior to statistical analysis, the annual percent change of each plasma biomarker was calculated using formula (1). This formula controls for differences in total plasma volume, starting concentration, and length of follow-up interval.

$$Annual percent change = [ {\frac{final concentration}{baseline concentration}}^{\frac{365}{interval ( days )}} - 1 ] * 100$$

### Outlier Analysis

For each plasma biomarker, outliers were defined as data points that exceeded three median absolute deviations from the median. There were between 6 and 9 outliers excluded across the entire sample. Outliers were primarily excluded to facilitate visualization of the data; statistical analyses were performed with and without outliers to verify that exclusion of outliers did not alter conclusions drawn from Wilcoxon rank sum tests at a threshold of P<0.05.

### Cognitive Composite Scores

Cognitive tests collected in the trial included the MMSE, the AD assessment scale-cognitive subscale 13 (ADAS-Cog13) and a custom neuropsychological test battery (NTB) consisting of a digit span task, a digit symbol substitution task, a category fluency task and a controlled oral word association test (COWAT). The MMSE was collected at the screening and 26-week visit. The ADAS-Cog13 and NTB were collected at baseline, 12-week and 26-week visits. Participants who discontinued the study prior to 26-weeks underwent the 26-week visit cognitive test battery at their early discontinuation visit, if possible.

Composite scores for memory, language, executive function, and visuospatial domains of cognition were calculated using items from the ADAS-Cog13, NTB, and MMSE. Items from cognitive tests were assigned to a cognitive domain by expert raters. The following number of items were included in the calculation of each composite: memory, 17;
language, 10; executive function, 4; visuospatial, 2.

A pre-treatment composite score was derived for each participant using their MMSE collected at screening, and their ADAS-Cog13 and NTB, which were collected at study baseline. For all participants, the baseline visit occurred within 8 weeks of the screening visit. The composite score calculated at the 12-week visit was derived from the ADAS-Cog13 and NTB, as MMSE was not collected at this visit. The final 26-week composite scores were calculated using MMSE, ADAS-Cog13 and NTB.

The measurement precision of each cognitive domain depends on the number of items available that correspond to that domain, and the appropriateness of the difficulty level of the items relative to the ability level of the participant. For each participant, their composite score for each domain at each time is accompanied by a standard error of measurement (SEM) value, where higher values indicate a less precise measure of the cognitive domain. During statistical analyses, linear mixed models were weighted by the inverse of the SEM to down-weight datapoints with greater measurement imprecision. Given that statistical models were weighted based on their measurement precision, outlier analysis was not performed on the longitudinal composite data and all participants with data for a given cognitive domain at a given timepoint were included in statistical analyses.

### Statistical Analysis

Spearman's rank correlation was used to test for monotonic associations between biomarkers and/or cognition at baseline. P values were corrected for multiple comparisons using the FDR correction.

For CSF module eigengenes and plasma biomarkers, Wilcoxon rank sum tests were used to assess differences between LM11A-31 and placebo. Following statistical analysis, the percent change of CSF module eigengenes was calculated using formula (2) to facilitate interpretation of the longitudinal changes within each group.

$$Percent change = ( \frac{Final - Baseline}{\vert Baseline \vert} ) * 100$$

For the analysis of individual CSF proteins in the TMT-MS dataset (FIG. 74A), the log₂ fold change for each protein was calculated using Formula (3) below:

$$log2 FC = median ( log2 ( Δ drug ) ) - median ( log2 ( Δ placebo ) )$$

P values were calculated for each individual TMT-MS protein using a Wilcoxon rank sum tests on the 26-week change under LM11A-31 or placebo. Significant CSF proteins, using an uncorrected threshold of P<0.05, were mapped to their corresponding gene symbols and entered into SynGO v1.2 (syngoportal.org/). The default settings for enrichment analysis were used. These included a background gene set of all brain expressed genes, a one-sided, greater than Fisher's exact test for each synaptic ontology term, and FDR correction for multiple comparisons across synaptic terms at a threshold of Q<0.01.

Linear mixed models were used to assess longitudinal changes in memory, language, executive function and visuospatial composite scores. Fixed effects were drug group (placebo, LM11A-31) and visit (baseline, 12-week, and 26-week). Random intercepts were included for each subject. Analyses covaried for total number of days between the first and final visit. Observations were weighted by the inverse of the SEM to decrease the weight of observations with high measurement imprecision (see Cognitive Composite Scores section).

All statistics were conducted two-tailed, and an a of 0.05 was used as the threshold for statistical significance unless otherwise stated. Due to the exploratory nature of the trial, longitudinal comparisons between the LM11A-31 and placebo group were not corrected for multiple comparisons.

### Code Availability

All statistical analyses were performed in R version 4.2.2. Custom code was not created for this publication.

### Example 17: Use of p-Tau243 for Monitoring Therapeutic Efficacy of a p75^{NTR} Modulator in a Subject Having Alzheimer's Disease

The methods of the present disclosure can be used to predict the responsiveness of a subject (e.g., a subject with Alzheimer's Disease) to treatment with a p75^{NTR} modulator, such as a compound of Formula (Ia). A biological sample (e.g., a plasma or cerebrospinal fluid (CSF) sample) may be obtained from the subject through methods well known in the art. The sample may be processed and analyzed using standard techniques. Levels of p-tau243 can be measured from the sample, e.g., using electrochemiluminescence immunoassay, Lumipulse, mass spectrometry, or other methods described herein. For example, the plasma level of p-tau243 can be determined and compared to established cutoff values. An elevated level of p-tau243 (e.g., above normal cutoff levels) can indicate the presence of Alzheimer's Disease pathology in the brain and likely responsiveness of the subject to treatment with a p75^{NTR} modulator (e.g., indicative the subject would benefit from treatment with the p75^{NTR} modulator). For example, an elevated level of p-tau243 in the subject, relative to a control (e.g., a healthy subject) can indicate that the subject has Alzheimer's Disease pathology and is likely to respond to p75^{NTR} modulation therapy. Furthermore, the baseline level of p-tau243 can serve as a reference point for monitoring therapeutic efficacy during treatment. If a subject is determined to have elevated p-tau243 levels indicative of Alzheimer's Disease pathology, then treatment of the subject can be initiated with a p75^{NTR} modulator, such as a compound of Formula (Ia), administered at an effective amount (e.g., about 200 mg to about 400 mg twice daily). During treatment, p-tau243 levels can be monitored at regular intervals (e.g., every 3 months, 6 months, or 12 months) to assess therapeutic efficacy. A decrease in p-tau243 levels from baseline can indicate therapeutic engagement and effectiveness of the p75^{NTR} modulator. Conversely, if p-tau243 levels remain elevated or continue to increase during treatment, then subsequent treatment of the subject having Alzheimer's can be altered, e.g., to increase dosage, frequency of administration, and/or length of treatment with the p75^{NTR} modulator, or to add additional therapeutic agents. Furthermore, sustained reduction in p-tau243 levels over time can indicate continued therapeutic benefit and disease modification, supporting continuation of p75^{NTR} modulation therapy.

### Example 18: Use of p-Tau181 for Monitoring Therapeutic Efficacy of a p75^{NTR} Modulator in a Subject Having Alzheimer's Disease

The methods of the present disclosure can be used to predict the responsiveness of a subject (e.g., a subject with Alzheimer's Disease) to treatment with a p75^{NTR} modulator, such as a compound of Formula (Ia). A biological sample (e.g., a plasma or cerebrospinal fluid (CSF) sample) may be obtained from the subject through methods well known in the art. The sample may be processed and analyzed using standard techniques. Levels of p-tau181 can be measured from the sample, e.g., using electrochemiluminescence immunoassay, Lumipulse, mass spectrometry, or other methods described herein. For example, the plasma level of p-tau181 can be determined and compared to established cutoff values. An elevated level of p-tau181 (e.g., above normal cutoff levels) can indicate the presence of Alzheimer's Disease pathology in the brain and likely responsiveness of the subject to treatment with a p75^{NTR} modulator (e.g., indicative the subject would benefit from treatment with the p75^{NTR} modulator). For example, an elevated level of p-tau181 in the subject, relative to a control (e.g., a healthy subject) can indicate that the subject has Alzheimer's Disease pathology and is likely to respond to p75^{NTR} modulation therapy. Furthermore, the baseline level of p-tau181 can serve as a reference point for monitoring therapeutic efficacy during treatment. If a subject is determined to have elevated p-tau181 levels indicative of Alzheimer's Disease pathology, then treatment of the subject can be initiated with a p75^{NTR} modulator, such as a compound of Formula (Ia), administered at an effective amount (e.g., about 200 mg to about 400 mg twice daily). During treatment, p-tau181 levels can be monitored at regular intervals (e.g., every 3 months, 6 months, or 12 months) to assess therapeutic efficacy. A decrease in p-tau181 levels from baseline can indicate therapeutic engagement and effectiveness of the p75^{NTR} modulator. Conversely, if p-tau181 levels remain elevated or continue to increase during treatment, then subsequent treatment of the subject having Alzheimer's can be altered, e.g., to increase dosage, frequency of administration, and/or length of treatment with the p75^{NTR} modulator, or to add additional therapeutic agents. Furthermore, sustained reduction in p-tau181 levels over time can indicate continued therapeutic benefit and disease modification, supporting continuation of p75^{NTR} modulation therapy.

### Example 19: Use of p-Tau231 for Monitoring Therapeutic Efficacy of a p75^{NTR} Modulator in a Subject Having Alzheimer's Disease

The methods of the present disclosure can be used to predict the responsiveness of a subject (e.g., a subject with Alzheimer's Disease) to treatment with a p75^{NTR} modulator, such as a compound of Formula (Ia). A biological sample (e.g., a plasma or cerebrospinal fluid (CSF) sample) may be obtained from the subject through methods well known in the art. The sample may be processed and analyzed using standard techniques. Levels of p-tau231 can be measured from the sample, e.g., using electrochemiluminescence immunoassay, Lumipulse, mass spectrometry, or other methods described herein. For example, the plasma level of p-tau231 can be determined and compared to established cutoff values. An elevated level of p-tau231 (e.g., above normal cutoff levels) can indicate the presence of Alzheimer's Disease pathology in the brain and likely responsiveness of the subject to treatment with a p75^{NTR} modulator (e.g., indicative the subject would benefit from treatment with the p75^{NTR} modulator). For example, an elevated level of p-tau231 in the subject, relative to a control (e.g., a healthy subject) can indicate that the subject has Alzheimer's Disease pathology and is likely to respond to p75^{NTR} modulation therapy. Furthermore, the baseline level of p-tau231 can serve as a reference point for monitoring therapeutic efficacy during treatment. If a subject is determined to have elevated p-tau231 levels indicative of Alzheimer's Disease pathology, then treatment of the subject can be initiated with a p75^{NTR} modulator, such as a compound of Formula (Ia), administered at an effective amount (e.g., about 200 mg to about 400 mg twice daily). During treatment, p-tau231 levels can be monitored at regular intervals (e.g., every 3 months, 6 months, or 12 months) to assess therapeutic efficacy. A decrease in p-tau231 levels from baseline can indicate therapeutic engagement and effectiveness of the p75^{NTR} modulator. Conversely, if p-tau231 levels remain elevated or continue to increase during treatment, then subsequent treatment of the subject having Alzheimer's can be altered, e.g., to increase dosage, frequency of administration, and/or length of treatment with the p75^{NTR} modulator, or to add additional therapeutic agents. Furthermore, sustained reduction in p-tau231 levels over time can indicate continued therapeutic benefit and disease modification, supporting continuation of p75^{NTR} modulation therapy.

### Example 20: Preparation and Characterization of the Compound of Formula (I)

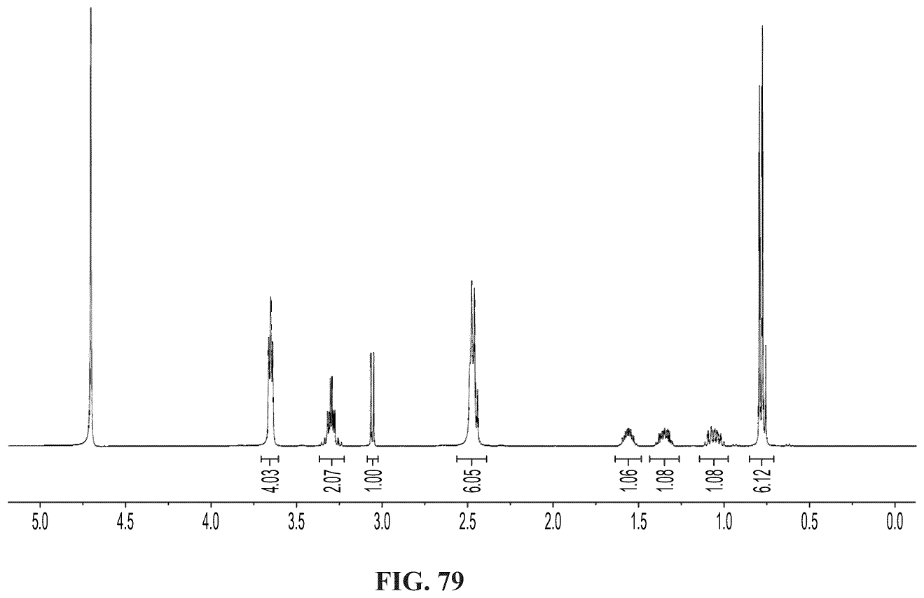

The BHS salt of the compound of Formula (I) (1 g) was suspended in DCM (20 V, 20 mL/g). An aqueous solution of NaOH 1M (9.2 mL, 4 equivalents) was added forming two layers. The mixture was transferred to a separatory funnel, was shaken, the phases were allowed to separate and the organic layer (lower) was drained. The aqueous layer (upper) was extracted with DCM (3×20 V, 20 mL) and the organic layers were combined and dried over sodium sulfate. The solution was concentrated to dryness in the rotary evaporator to give an oil (555 mg, 98%). The oil was analyzed by ¹H NMR and it is consistent with the structure (see FIG. 79).

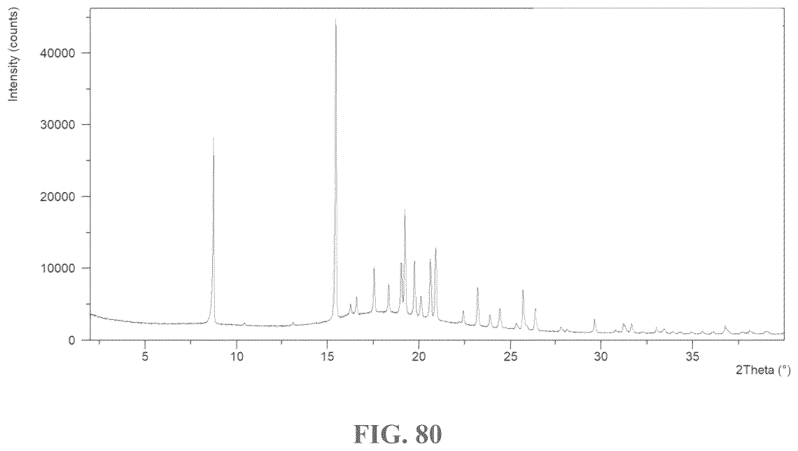

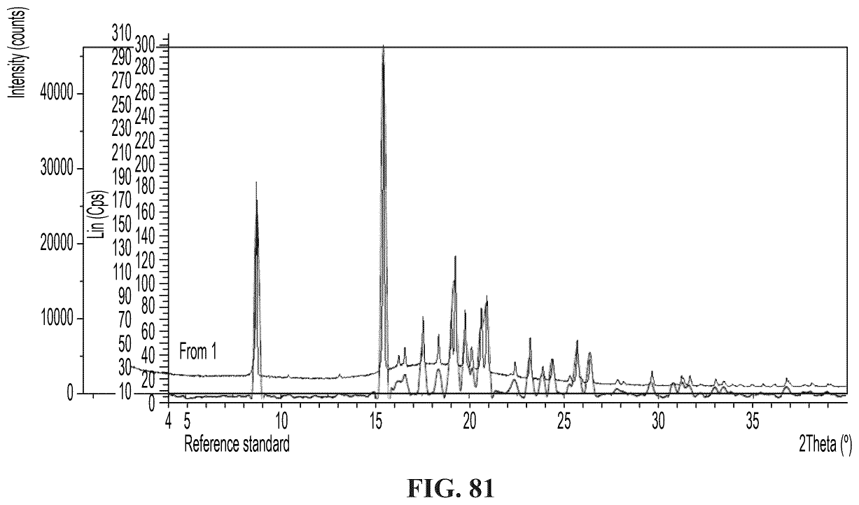

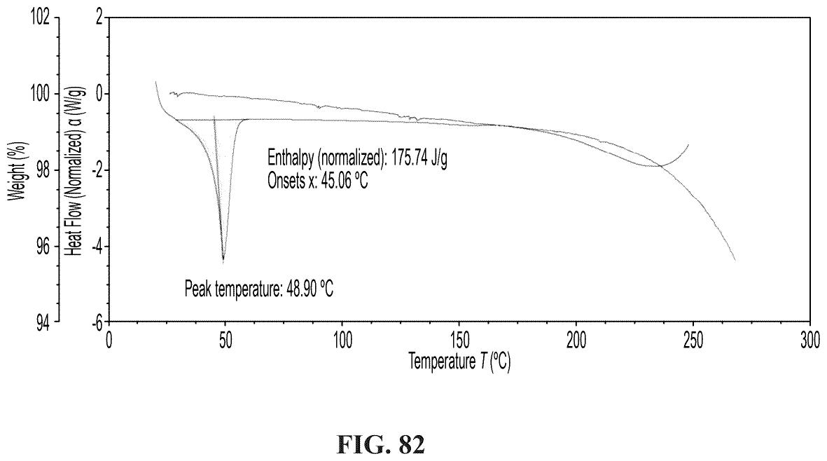

The oil, 1 g was dissolved in IPA (10 mL) and evaporated in the rotary evaporator to dryness. Residual solvent was evaporated with nitrogen flow and a white solid was obtained in the round-bottomed flask. The resulting solid was analyzed by XRPD, DSC and TGA (see FIG. 80 and FIG. 82) and an XRPD comparison is shown in FIG. 81. The crystalline free base is anhydrous (no significant weight loss in TGA), with low melting point (onset 45° C. by DSC). The results obtained for the crystalline form are consistent with a reference standard for the BHS salt of the compound of Formula (I).

### Example 21: Salt/Cocrystal Screening

A series of salt screens were performed on the compound of Formula (I). The gentisate and hemi-adipate salts provide favorable stability and hygroscopicity characteristics in comparison with the sulfate salt as benchmark. These three salts showed high solubility (>10 mg/mL) over the pH range 1.2 to 7.4. Polymorphism assessments showed that the gentisate salt exhibits polymorphism, while only one form was seen for the hemi-adipate salt.

Process development, solubility modelling and scale-up were performed for the gentisate and hemi-adipate salts. Three batches of 80-100 g of gentisate and two batches of 140-150 g of the hemi-adipate salt were prepared and characterized.

The compound of Formula (I) contains two basic sites with pKas of 6.9 and 8.4. The molecule also contains both hydrogen bond donors (NH) and acceptors (amine) and may form cocrystals with suitable coformers.

Solid forms identified in the screening experiments were analyzed by XRPD before and after drying under vacuum at RT overnight. The resulting patterns were compared with the corresponding coformer. Those samples showing a distinct XRPD pattern were further characterized by some, or all, of the following techniques, depending on material availability: ¹H NMR, DSC and TGA. Then, they were stored in XRPD sample holders in a humidity chamber at 40° C./75% RH for one week. Upon storage, samples were monitored by XRPD over time.

An additional stability study was performed at 25° C./96% RH for one week with the salts considered most promising for stability reasons. Samples were also monitored by XRPD over time.

The results are provided in Table 16, Table 17, Table 18, and Table 30.

### Heat-Cool Cycling

Dried compound of Formula (I) (approx. 20 mg) and 1.1 equivalents of the corresponding acid were weighed into 2 mL HPLC vials. The corresponding solvent (100 μL) was added and the mixtures were subjected to heat-cool cycles between 25° C. and 50° C. (30 min at each temperature, with heating and cooling at 1° C./min) for 1 to 4 days. The suspensions were centrifuged, decanted and solids were analyzed by XRPD before and after drying under vacuum at RT.

Slurries at Low Temperature after Heat-Cool Cycling

Oils and solutions from the heat-cool cycling experiments were stirred at 0° C. for 24 h. However, no solids were observed in any of the experiments.

### Slow Evaporation of Samples at Low Temperature

Solutions of samples at low temperature were allowed to slowly evaporate until dryness at room temperature; via a needle in a septum, or directly opening the vial for the low volatile solvents. However, oils were observed in all the experiments.

### Wet Grinding

The compound of Formula (I) Form 1 (20 mg), 0.5-2.01 equivalents of the corresponding coformer and the corresponding solvent (5-10 μL) were weighed into 2 mL HPLC vials, each one equipped with 3 stainless steel 3 mm ø balls and were ground in a Fritsch Pulverisette planetary mill at 400 rpm for a period of 16 cycles of 1 h with 5 minutes resting between cycles. The resulting solids were analyzed by XRPD before and after drying under vacuum at RT unless oils or solutions were obtained.

### Results—Screening

Results from the salt/cocrystal screening study are provided in Table 16 below.

TABLE 16
All Salt/Cocrystal Screen Experiments

| | | | | Heat-Cool Cycling | | Wet Grinding* | |
|---|---|---|---|---|---|---|---|
| Ref. | Coformer | Eq. | Solvent | Wet | Dry | Wet | Dry |
| 1 | Gentisic acid | 1.1 | IPA | Solution | NP | NP | NP |
| 2 | Gentisic acid | 2 | IPA | Solution | NP | NP | NP |
| 3 | Gentisic acid | 1.1 | EtOAc | GEN Form A | GEN Form A | NP | NP |
| 4 | Gentisic acid | 2 | EtOAc | Amorphous | NP | NP | NP |
| 5 | Gentisic acid | 1 | EtOAc | NP | NP | GEN Form A | GEN Form A |
| 6 | Adipic acid | 0.5 | EtOAc | NP | NP | ADI Form A | ADI Form A |
| 7 | Adipic acid | 0.5 | EtOAc | ADI Form A | NP | NP | NP |
| 8 | Adipic acid | 1 | EtOAc | NP | NP | ADI Form A | ADI Form A** |
| 9 | Adipic acid | 1 | EtOAc | Solution | Solution | NP | NP |
| 10 | Adipic acid | 2 | EtOAc | NP | NP | ADI Form | Coformer |
| | | | | | | B + coformer | |

*Experiments performed with the compound of Formula (I) Form 1.
**Solids not stable at ambient conditions (25 ± 1° C./70 ± 5% RH). Deliquescence was observed.

Results from the salt/cocrystal screening study are summarized in Table 17 below.

TABLE 17
Summary of Salt/Cocrystal Screen Experiments

| | | Acid | | Heat-Cool Cycling | | Wet Grinding* | |
|---|---|---|---|---|---|---|---|
| Reference | Coformer | equivalents | Solvent | Wet | Dry | Wet | Dry |
| 1 | Gentisic acid | 1.0 | EtOAc | NP | NP | GEN Form A | GEN Form A |
| 2 | Adipic acid | 1.0 | EtOAc | NP | NP | ADI Form A | ADI Form A** |
| 3 | Adipic acid | 2.0 | EtOAc | NP | NP | ADI Form | Coformer |
| | | | | | | B + coformer | |
| 4 | Adipic acid | 0.5 | EtOAc | NP | NP | ADI Form A | ADI Form A |
| 5 | Adipic acid | 0.5 | EtOAc | ADI Form A | NP | NP | NP |

*Experiments performed with the compound of Formula (I) Form 1.
**Solids not stable at ambient conditions (25 ± 1° C./70 ± 5% RH). Deliquescence was observed.

Stability tests in the XRPD sample holders at 40° C./7500 RH were performed with the crystalline forms that were stable after drying the solid under vacuum at RT overnight (see Table 6) and they were compared with the sulfate salt of the compound of Formula (I). The results are shown in Table 18.

TABLE 18
Stability Study at 40° C./75% RH of Salts in XRPD Sample Holder

| Reference | Acid | XRPD t0 | XRPD 1 d | XRPD 2 d | XRPD 3 d | XRPD 6 d | XRPD 7 d |
|---|---|---|---|---|---|---|---|
| 1 | Gentisic acid | GEN | GEN | GEN | GEN | GEN | GEN |
| | | Form A | Form A | Form A | Form A | Form A | Form A |
| 2 | Adipic acid | ADI | ADI | ADI | NP | NP | ADI |
| | | Form A | Form A | Form A | | | Form A |

Two of the crystalline salts (GEN Form A and ADI Form A) stored under accelerated conditions at 40° C./7500 RH were stable for at least 1 week.

Additionally, the salts that were stable at 40° C./7500 RH were stored at 25° C./9600 RH in an XRPD sample holder. The results are shown in Table 19.

TABLE 19
Stability Study at 25° C./96% RH of Salts in XRPD Sample Holders

| Reference | Acid | XRPD t0 | XRPD 1 d | XRPD 2 d | XRPD 3 d | XRPD 6 d | XRPD 7 d |
|---|---|---|---|---|---|---|---|
| 3 | Gentisic acid | GEN | GEN | GEN | GEN | GEN | GEN |
| | | Form A | Form A | Form A | Form A | Form A | Form A |
| 5 | Adipic acid | ADI | ADI | Deliquescent | NP | NP | NP |
| | | Form A | Form A | | | | |

GEN Form A stored under accelerated conditions at 25° C./96% RH was stable for at least 1 week.

### Summary of Example 2

The crystalline salt with gentisic acid (GEN Form A) was stable for at least 1 week at 40° C./75% RH. At 25° C./96% RH, GEN Form A was stable for at least 1 week. ADI Form A was stable under ambient conditions. ADI Form A was stable for at least 1 week at 40° C./75% RH. At 25° C./96% RH, ADI Form A was deliquescent after 2 to 7 days. The crystalline salt with adipic acid (ADI Form A) was stable for at least 1 week at 40° C./75% RH while at 25° C./96% RH deliquescence was observed after 2 days.

### Example 22: Scale Up and Additional Characterization

### Preparation at 50-250 mg Scale

GEN Form A and ADI Form A were prepared at 50-250 mg scale for a further characterization using the compound of Formula (I) Form 1. Heat-cool cycles between 25° C. to 50° C. (30 min at each temperature, with heating and cooling at 1° C./min) for 1 day were performed. The procedures were not optimized. The experiments are described in Table 20.

TABLE 20
Summary of Experiments Performed at 100-250 mg Scale

| | | | Scale | | | |
|---|---|---|---|---|---|---|
| Reference | Acid | Equivalents | (mg) | Solvent | XRPD wet | XRPD dry |
| 1 | Gentisic acid | 1 | 100 | EtOAc | GEN Form A | GEN Form A |
| 2* | Adipic acid | 1 | 250 | EtOAc | ADI Form A | ADI Form A |

*Sticky solid recovered after the filtration.

GEN Form A and ADI Form A were reproduced successfully.

### Preparation at 500 mg or 1 g Scale

GEN Form A and ADI Form A were prepared at 1 g scale to complete the characterization and perform a polymorphism assessment and a solubility study in water at different pHs. A stability study at different humidity and temperature conditions was also performed. In these scale up experiments the compound of Formula (I) was added as a solid and the processes used were based on the screening procedures. The experiments are described in Table 21.

TABLE 21
Summary of Experiments Performed at 500 mg or 1 g Scale

| | | | | Vol | | |
|---|---|---|---|---|---|---|
| Reference | Acid | Equivalents | Solvent | (V) | XRPD wet | XRPD dry |
| 1 | Gentisic | 1 | EtOAc | 5 + 15 | GEN Form A | GEN Form A |
| 2 | Adipic acid | 1 | EtOAc | 1 | ADI Form A* | ADI Form A |
| 3 | Adipic acid | 1 | THF | 1 | ADI Form A | ADI Form A |

*Sticky solid obtained.

### Scale Up of Gentisate Salt

After some initial small-scale trials, the process (starting from the sulfate salt) was scaled up first to 4 g and then to 80-100 g batches in a 1-L jacketed vessel (see Table 22). Approximately 260 g of gentisate salt was prepared from the sulfate in 3 batches, first cracking the sulfate with NaOH and partitioning in DCM-water, concentrating the organic and crystallizing the gentisate in ethanol (4 vol) with seeding at 68° C., followed by cooling to 5° C. and antisolvent addition (EtOAc, 7 vol).

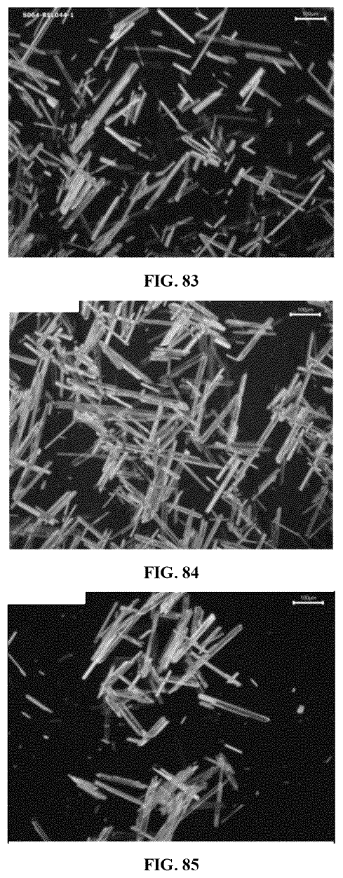

TABLE 22
Scale-up Batches, Gentisate Salt

| Reference | Yield (g, %) | XRPD | ¹H NMR | DSC | TGA | Microscopy |
|---|---|---|---|---|---|---|
| 1 | 4 g, 84.1% | GEN Form A | conforms | NP | NP | NP |
| 2 | 78 g, 85.9% | GEN Form A | conforms | Onset 168.2° C. | 0.80% | FIG. 83 |
| | | | | (133 J/g) | | |
| 3 | 82 g, 89.5% | GEN Form A | conforms | Onset 168.6° C. | 0.60% | FIG. 84 |
| | | | | (134 J/g) | | |
| 4 | 99 g, 88.4% | GEN Form A | conforms | Onset 168.6° C. | 0.70% | FIG. 85 |
| | | | | (135 J/g) | | |

### Experimental Procedures—Scale Up

Preparation of ADI Form A in EtOAc, 1 g Scale. The compound of Formula (I) (1 g) and adipic acid (622.9 mg, 1.0 eq.) were suspended in EtOAc (10 mL, 10 vol). The resulting mixture was subjected to heat-cool cycles between 25° C. and 50° C. with 30 minutes at each temperature with heating and cooling of 1° C./min for 1 day. A thick suspension was obtained and EtOAc (2 mL, 2 vol) were added. The resulting suspension was subjected to heat-cool cycles between 25° C. and 50° C. with 30 minutes at each temperature with heating and cooling of 1° C./min for another day. The mixture was filtered, washed with EtOAc (2×2 mL, 2 vol) and analyzed wet by XRPD affording ADI Form A. Then, the solid was dried under vacuum at 25° C. overnight. An off-white solid 1.247 g (79.6%) was obtained which corresponded to ADI Form A by XRPD. ADI Form A was also characterized by ¹H NMR, DSC, TGA, DVS and HPLC.

Preparation of ADI Form A in THF, 1 g Scale. The compound of Formula (I) (1 g) and adipic acid (618.4 mg, 1.0 eq.) were suspended in THF (10 mL, 10 vol). After 30 minutes a thick suspension was obtained. THF (15 mL, 15 vol) were added and the mixture was stirred at RT for 2 h. The mixture was filtered, washed with THF (2×2 mL, 2 vol) and analyzed wet by XRPD affording ADI Form A. Then, the solid was dried with a nitrogen flux. An off-white solid 1.088 g (82.2%) was obtained which corresponded to ADI Form A. ADI Form A was also characterized by 1H NMR, DSC, TGA, DVS, and KF.

Preparation of GEN Form A in EtOAC. The compound of Formula (I) (1 g) and 2,5-dihydroxybenzoic acid (647.9 mg, 1.0 eq.) were suspended in EtOAc (5 mL, 5 vol). The resulting mixture was subjected to heat-cool cycles between 25° C. and 50° C. with 30 minutes at each temperature with heating and cooling of 1° C./min for 1 day. A thick suspension was obtained and EtOAc (15 mL, 15 vol) were added. After 1 h, the mixture was filtered, washed with EtOAc (2×2 mL, 2 vol) and analyzed wet by XRPD affording GEN Form A. Then, the solid was dried under vacuum at 25° C. overnight. An off-white solid 1.496 g (91.6%) was obtained which corresponded to GEN Form A by XRPD. GEN Form A was also characterized by ¹H NMR, DSC, TGA, DVS and HPLC.

Preparation of GEN Form A in EtOAc, 2 g Scale. The compound of Formula (I) (2.013 g) and 2,5-dihydroxybenzoic acid (1.303 g, 1.0 eq.) were suspended in EtOAc (40 mL, 10 vol). The resulting mixture was stirred at 25° C. for 22 hours. The mixture was filtered, washed with EtOAc (2×2 vol, 4 mL) and dried under vacuum at 25° C. for 24 h. An off-white solid 3.044 g (92.5%) was obtained which corresponded to GEN Form A by XRPD.

Preparation of GEN Form A in EtOAc, 100 g Scale. The BHS salt of the compound of Formula (I) (100.5 g), sodium hydroxide (41.4 g) and water (400 mL) were introduced into a 1 L jacketed vessel and stirred for 15 minutes. The mixture was allowed to settle, and the layers were separated. The lower aqueous phase was reintroduced to the reactor with dichloromethane (200 mL) and stirred for 5 minutes. The mixture was allowed to settle, the layers separated, and the organic phases combined were separated giving a biphasic mixture. The layers were separated, and the aqueous phase extracted with dichloromethane (3×100 mL). The organic phases were combined and washed with water (100 mL) and then evaporated to dryness on a rotary evaporator. To the residue ethanol (365 mL) was added, charged to an oversized 1 L jacketed vessel, heated to 75° C. and 2,5-dihydroxybenzoic acid (36.074 g, 1.0 eq.) were added together with an additional 8 mL of ethanol washing the funnel. The solution was cooled to 68° C. and seeded with GEN Form A, 50 mg. The mixture was stirred for 1 hour at this temperature, then cooled at 0.5° C./min to 5° C. and maintained at 5° C. for 13 hours. EtOAc (640 mL) was added and the mixture stirred for 90 minutes. The mixture was filtered, washed with cold EtOAc (2×100 mL) and dried under vacuum at 50° C. for 24 h. An off-white solid 78.25 g (85.9%) was obtained.

### Additional Characterization of New Salt Forms

The new stable crystalline salt form (GEN Form A) were characterized by XRPD, ¹H NMR, DSC, TGA and DVS. The results are summarized in Table 23.

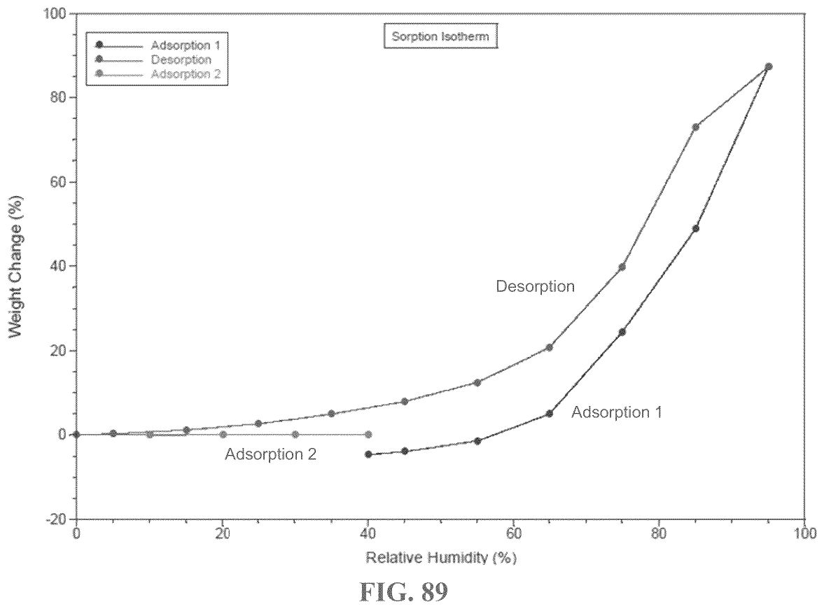

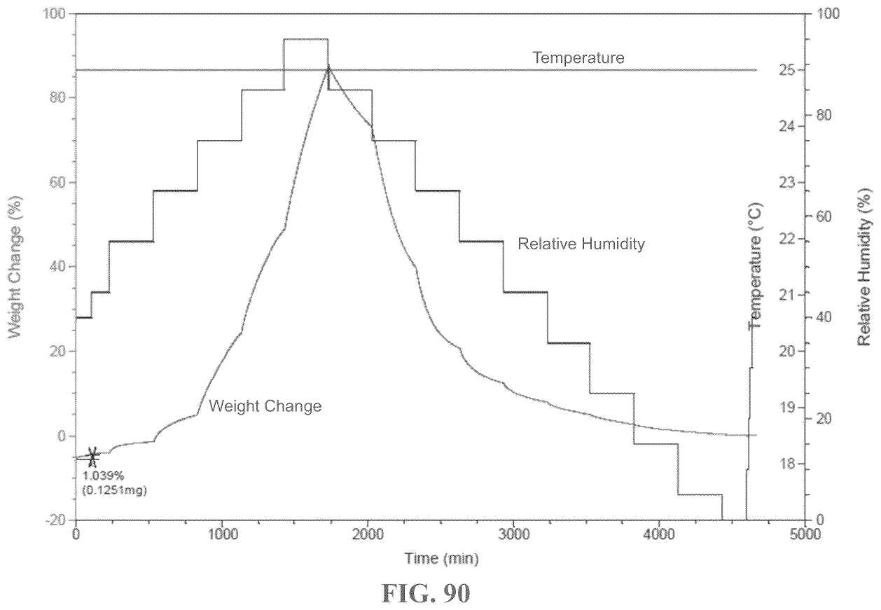

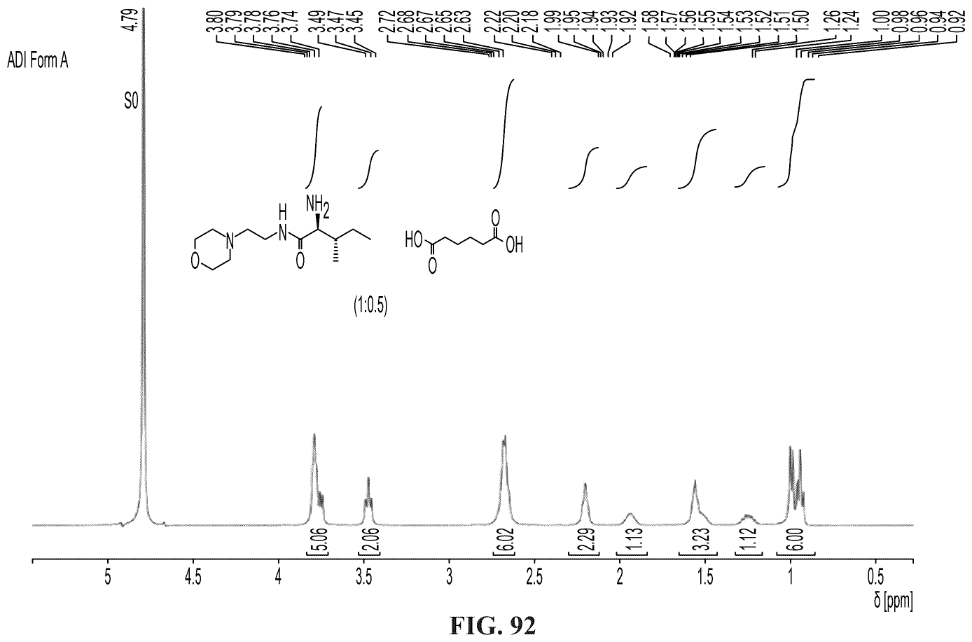

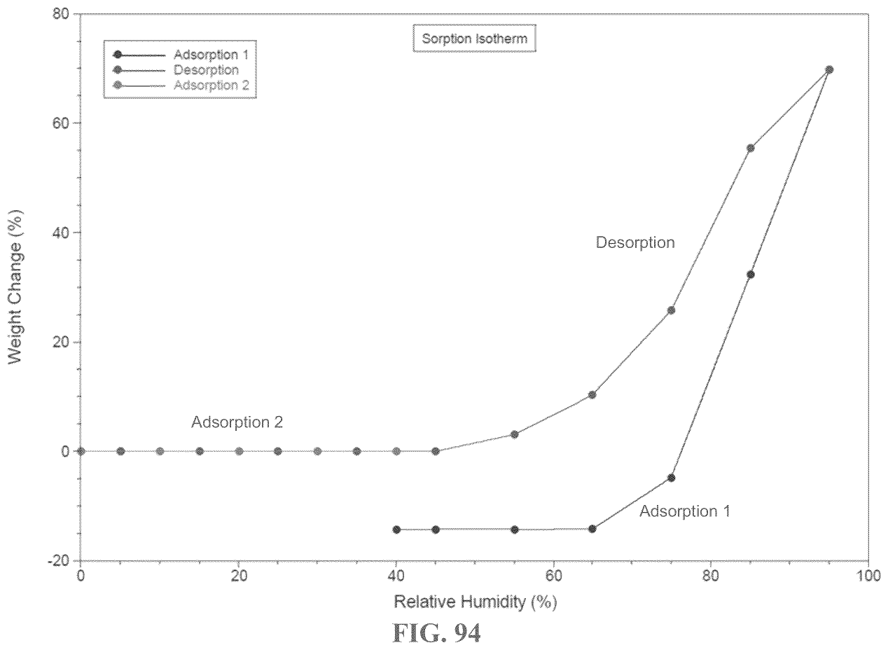

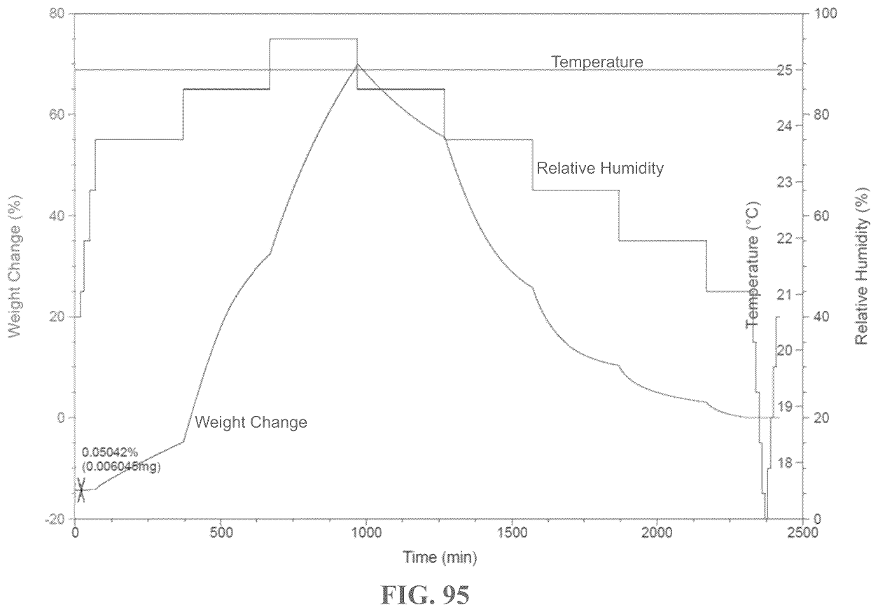

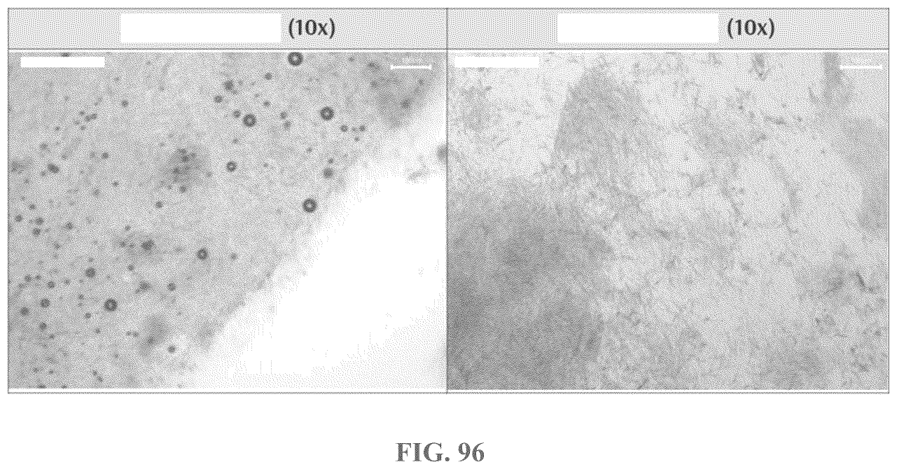

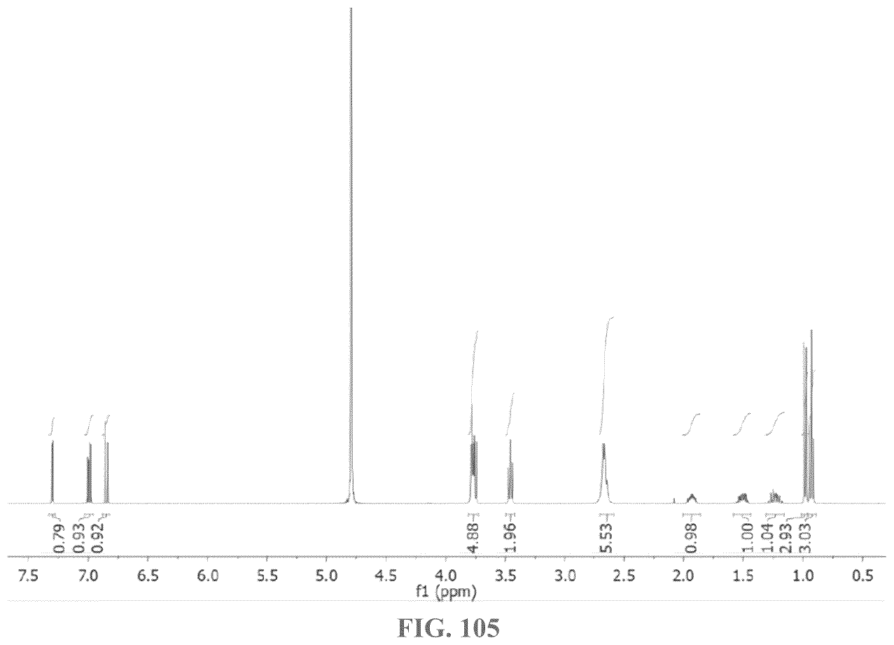

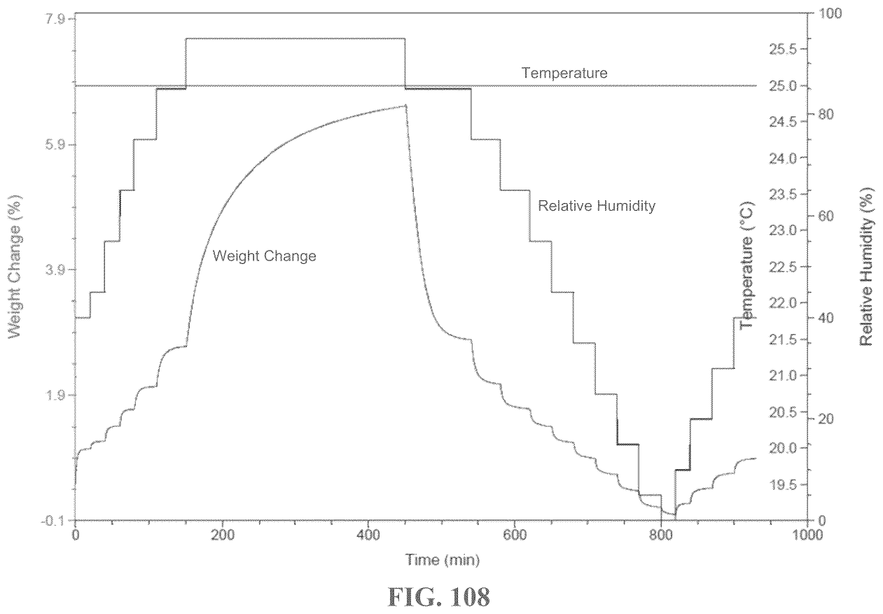

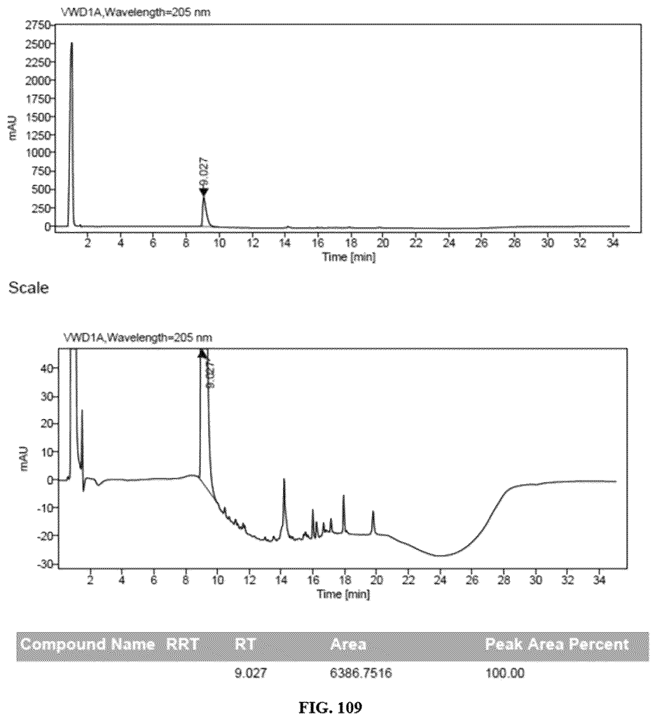

TABLE 23
Characterization Summary

| | | | | Stability at | Stability at | |
|---|---|---|---|---|---|---|
| Form | ¹H NMR (D₂O) | DSC | TGA | 40° C./75% RH | 25° C./96% RH | DVS |
| GEN Form | API:Gentisic acid | Melt onset at | 0.6% loss at 30° C. to | Stable for 1 | Stable for 1 | 6.5% weight |
| A FIG. 104 | (1:1) FIG. 105 | 167° C. FIG. | 60° C. Degradation | week | week | change GEN Form |
| | | 106 | at ~200° C. FIG. 106 | | | A by XRPD FIG. |
| | | | | | | 107 and FIG. 108 |
| ADI Form A | API:Adipic acid | Melt onset at | 0.5% loss at 30° C. to | Stable for 1 | Deliquescent | 87.3% weight |
| in EtOAc | (2:1) FIG. 87 | 104° C. FIG. | 65° C. Degradation | week | after 2 days | change FIG. 89 |
| FIG. 86 | | 88 | at ~150° C. FIG. 88 | | (sample holder) | and FIG. 90 |
| ADI Form A | API:Adipic acid | Melt onset at | Degradation at ~150° | Stable for 1 | Deliquescent | 84% weight |
| in THF FIG. | (2:1) FIG. 92 | 104° C. FIG. | C. FIG. 93 | week | after 2 days | change FIG. 94 |
| 91 | | 93 | | | (sample holder) | and FIG. 95 KF: |
| | | | | | | 0.41% |

### Summary of Example 3

Experiments at 1 g scale were performed successfully for GEN Form A. This form was further characterized by ¹H NMR, DSC, TGA, DVS and HPLC. GEN Form A was stable in open vials after 1 week of storage at 40° C./75% RH and 25° C./96% RH by XRPD and HPLC. Slightly higher amounts of water were observed by TGA in comparison with the starting materials. Experiments at 500 mg scale were performed successfully for ADI Form A. This form was further characterized by ¹H NMR, DSC, TGA and DVS. ADI Form A was prepared successfully to 1 g scale in THF. The API:adipic acid ratio was 2:1 for the same prepared in EtOAc and the sample prepared in THF. Experiments at 1 g scale were performed successfully to obtain ADI Form A in THF and EtOAc. However, in EtOAc a sticky solid was obtained while in THF a fine powder was observed.

### Example 23: Further Investigation of GEN Form A

### Polymorphism Assessment of Salts

The GEN Form A salt obtained during the salt screening and scaled up to 500 mg or 1 g scale were further studied by means of polymorphism assessment. Twelve different solvent systems were studied for the salt to provide an early insight into the propensity for polymorphism of the salts.

Procedure. The samples (20 mg±1 mg) were weighed and suspended in 5-10 volumes of the corresponding solvent. Then, mixtures were subjected to heat-cool cycles between 25° C. to 50° C. (30 min at each temperature, with heating and cooling at 1° C./min) for 2 days. Then, mixtures were centrifuged, decanted and the solids were first analyzed by XRPD as wet cakes before drying under vacuum at ambient temperature and reanalyzed by XRPD if a new form was obtained. The results for the polymorphism assessment for the salt are tabulated below.

TABLE 24
Polymorphism Assessment of GEN Form A

| Reference | Solvent | Aspect 2 days | XRPD wet | XRPD dry |
|---|---|---|---|---|
| 1 | Water | Red solution | NA | NA |
| 2 | MeCN | Suspension | GEN Form A | NP |
| 3 | EtOH | Suspension | GEN Form A | NP |
| 4 | IPA | Suspension | GEN Form A | NP |
| 5 | Acetone | Solution | NA | NA |
| 6 | MEK | Solution | NA | NA |
| 7 | EtOAc | Suspension | GEN Form A | NP |
| 8 | i-PrOAc | Suspension | GEN Form A | NP |
| 9 | MTBE | Suspension | GEN Form A | NP |
| 10 | THF | Suspension | GEN Form B | GEN Form A |
| 11 | Toluene | Suspension | GEN Form A | NP |
| 12 | Heptane | Suspension | GEN Form A | NP |

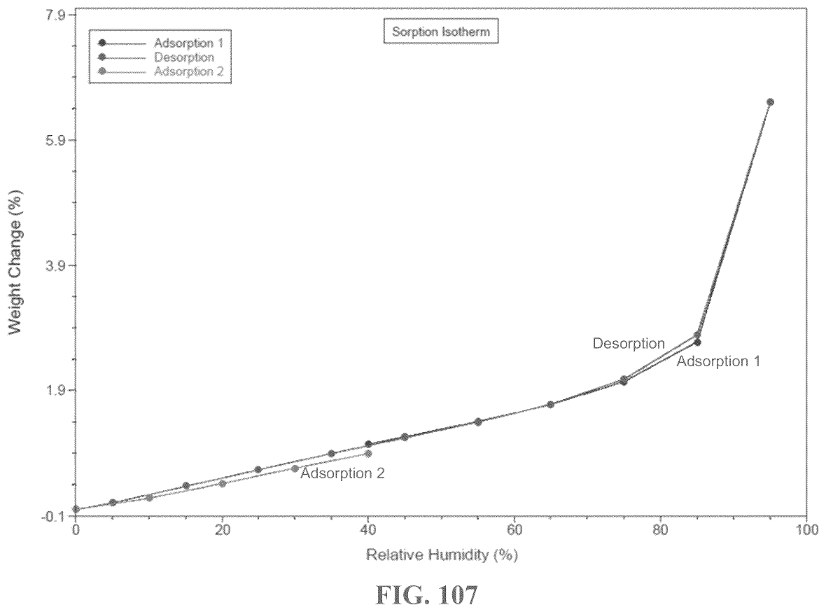

Two different polymorphs of the gentisate salt were found, GEN Form A and Form B. They appear to be related by a different degree of hydration and GEN Form A appears more stable under ambient conditions. XRPD (FIG. 104), 1H NMR (FIG. 105) DSC, TGA (FIG. 106) and DVS analyses (FIG. 107 and FIG. 108) were performed for GEN Form A. By 1H NMR the stoichiometry is 1:1 API:gentisic acid. This form showed good crystallinity by XRPD and contains some moisture (0.6%) with a melt at onset 167° C. By DVS approximately 6.5 wt % uptake was seen, mainly above 80% RH. The adsorption-desorption process was reversible with XRPD reanalysis showing unchanged GEN Form A. GEN Form A was stable for at least 1 week at both 40° C./75% RH, 25° C./96% RH. The salt showed high solubility (>10 mg/ml) over the pH range 1.7-7.2.

### Stability Study of GEN Form A

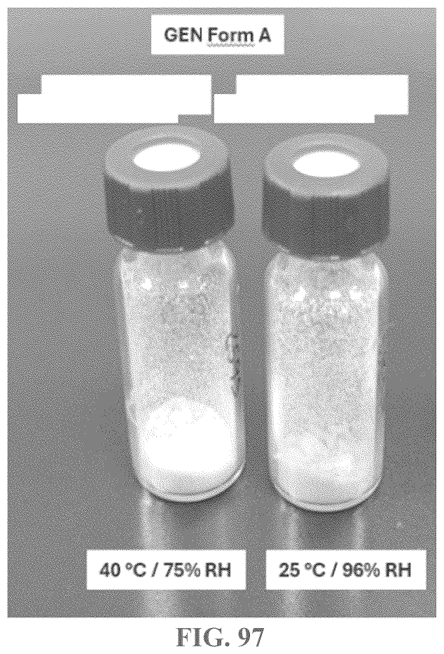

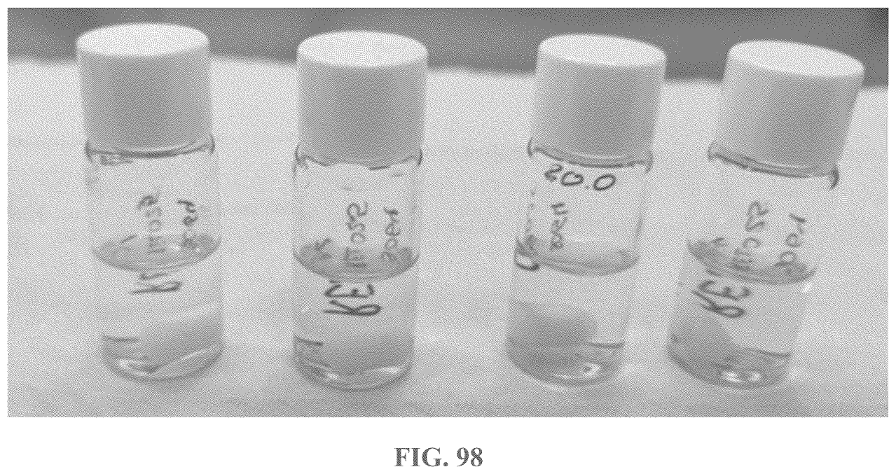

GEN Form A was stored in open vials at 25° C./96% RH and 40° C./75% RH, monitored with dataloggers, for 2 weeks and were analyzed by XRPD, TGA and HPLC every week to study its crystalline and chemical stability. A summary of the results is shown in Table 25 and Table 26. All experiments showed the same form as the starting material by XRPD after 1 week, with no evidence of chemical degradation by HPLC. Some discoloration from white or colorless to pink was seen in the gentisate salt stored at 25° C./96% RH. See FIG. 97.

TABLE 25
Stability Studies of GEN Form A in Different
Conditions by XRPD and TGA

| | | | Aspect | XRPD | TGA 1 week* |
|---|---|---|---|---|---|
| Reference | Form | Conditions | 1 week | 1 week | (% weight loss) |
| 1 | GEN | 40° C. / | Off-white | GEN | 0.9 |
| | Form A | 75% RH | solid | Form A | |
| 2 | GEN | 25° C. / | Beige | GEN | 0.7 |
| | Form A | 96% RH | solid | Form A | |

*0.6% weight loss observed by TGA for GEN Form A at time 0.

TABLE 26
Stability Studies of GEN Form A in Different Conditions by HPLC

| | | | Time | % Peak Area |
|---|---|---|---|---|
| Reference | Starting Form | Conditions | Point | 1 week |
| 1 | GEN Form A | — | Time 0 | 100 |
| 2 | GEN Form A | 25° C. / 96% RH | 1 week | 100 |
| 3 | GEN Form A | 40° C. / 75% RH | 1 week | 100 |

### Thermodynamic Solubility of GEN Form A at Different pHs

The thermodynamic solubility study at pH 1.2, 4.5, 6.5 and 7.4 were performed with GEN Form A. The salt, 20 mg, was treated with the different buffer solutions (2 mL) and stirred for 24 hours. The pH was measured after 24 hours. In case suspensions were obtained, 1 mL of each suspension was filtered with a nylon syringe filter (0.45 μm) and were diluted by a factor of 6 v/v in MeCN/water (1:1) for HPLC analysis.

TABLE 27
Thermodynamic Solubility at pH 1.2, 4.5, 6.5 and 7.4

| | | XRPD | | | | XRPD |
|---|---|---|---|---|---|---|
| Refer- | Condi- | Initial | Observa- | pH | Conc. | Recovered |
| ence | tions | Solid | tions | 24 h | (mg/mL) | Solid |
| 1 | pH 1.2 | GEN | Solution | 1.8 | >10 | NA |
| | | Form A | | | | |
| 2 | pH 4.5 | GEN | Solution | 5.2 | >10 | NA |
| | | Form A | | | | |
| 3 | pH 6.5 | GEN | Solution | 6.6 | >10 | NA |
| | | Form A | | | | |
| 4 | pH 7.4 | GEN | Solution | 7.2 | >10 | NA |
| | | Form A | | | | |

High solubility (>10 mg/mL) was observed in all the pH for GEN Form A.

### Example 24: Solubility Data of GEN Form A in Organic Solvents and Mixtures

### Evaluation

An initial evaluation of the solubility at 25° C. was conducted in different organic solvents and solvent mixtures. The solid (GEN Form A, 50 mg) was weighed in HPLC vials and 500 μL of each solvent were added. Samples were slurried at 25° C. for 24 hours, then approximately 300 μL of suspension were taken, diluted in 2 mL of MeCN/H₂O (1:1), filtered through a 0.45 μm hydrophilic syringe filter and analyzed by HPLC. Experiments where solutions were obtained were not analyzed by HPLC.

TABLE 28
Dissolution of GEN Form A in Different Organic
Solvents and Solvent Mixtures at 25° C.

| | | | Conc | Conc |
|---|---|---|---|---|
| | | Aspect | (mg/g | (mg/mL |
| Reference | Solvent | at RT | solution) | solution) |
| 1 | IPA | Suspension | 8.5 | 6.7 |
| 2 | IPA:H₂O (95:5) | Solution | >126.6 | >100 |
| 3 | IPA:H₂O (90:10) | Solution | >126.6 | >100 |
| 4 | EtOAc | Suspension | 0.17 | 0.15 |
| 5 | MeOH | Solution | NA | >100 |
| 6 | EtOH | Suspension | 43 | 34 |
| 7 | EtOH:H₂O (95:5) | Solution | >126.6 | >100 |
| 8 | EtOH:H₂O (90:10) | Solution | >126.6 | >100 |
| 9 | Heptane | Suspension | NA* | NA* |

*2 phases observed when diluted in MeCN/H₂O (1:1)

### Solubility in IPA and EtOH by Determination of Clear Points

Following from the previous solubility results, IPA and EtOH were studied in more detail. The solid (GEN Form A) was weighed in HPLC vials, 0.5 mL of solvent added, and the solvent weighed for accuracy. A heating ramp from 25-80° C. at 0.5° C./min was applied and the clear point (dissolution temperature) recorded using reflectance probes. All the experiments resulted in dissolution at high temperature and the mixtures were then cooled to ambient. In most cases, spontaneous precipitation occurred during cooling. Reanalysis of the solids by XRPD showed that GEN Form A was recovered. The results are given in Table 29.

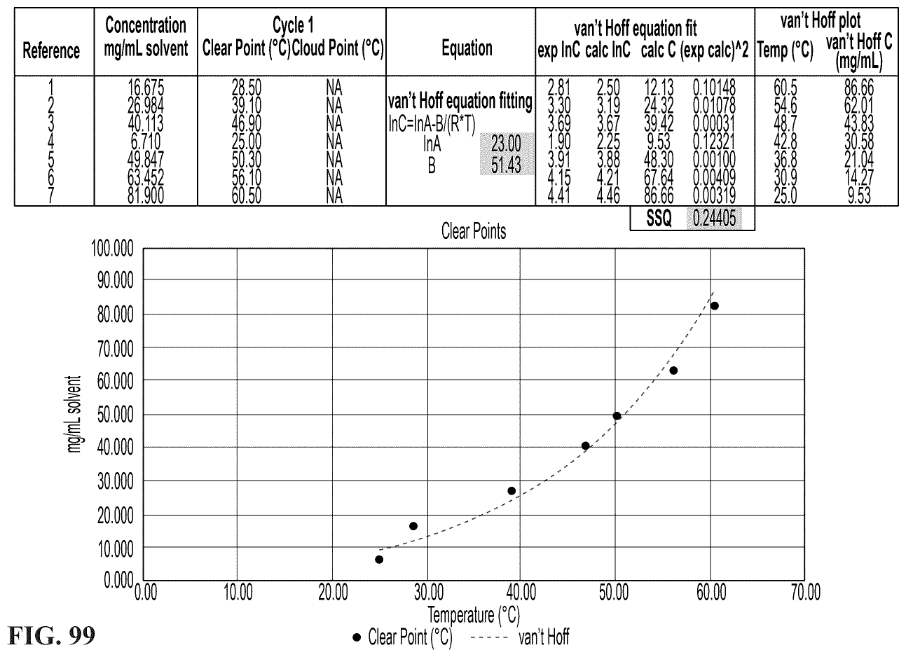

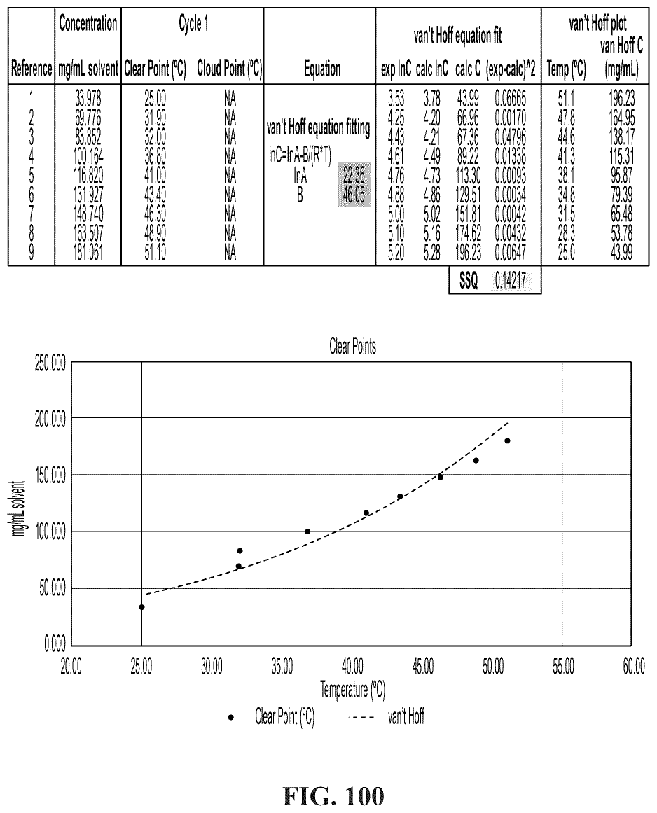

The solubility was higher in EtOH than IPA. The data was used to generate solubility curves as shown in FIG. 99 (IPA) and FIG. 100 (EtOH). The difference of solubility in IPA between high and low temperature seems promising for a cooled crystallization process. In EtOH, an antisolvent will probably be necessary to provide a high yield.

TABLE 29
Dissolution of GEN Form A in IPA and EtOH

| | | API | Solvent | Conc (mg/g | Conc (mg/g | Conc | Conc | | Clear point | Aspect RT |
|---|---|---|---|---|---|---|---|---|---|---|
| Reference | Solvent | (mg) | (mg) | solvent) | solution) | (mg/mL | (mL/mg | Aspect RT | (° C.) | after cooling |
| 1 | IPA | 5 | 381.5 | 13.1 | 12.9 | 16.7 | 60 | Suspension | 28.5 | Solution |
| 2 | IPA | 8.1 | 381.9 | 21.2 | 20.8 | 27 | 37.1 | Suspension | 39.1 | Suspension |
| 3 | IPA | 12 | 380.6 | 31.5 | 30.6 | 40.1 | 24.9 | Suspension | 46.9 | Suspension |
| 4 | IPA | 15.1 | 385.4 | 39.2 | 37.7 | 49.8 | 20.1 | Suspension | 50.3 | Suspension |
| 5 | IPA | 19.7 | 395 | 49.9 | 47.5 | 63.5 | 15.8 | Suspension | 56.1 | Suspension |
| 6 | IPA | 24.7 | 383.7 | 64.4 | 60.5 | 81.9 | 12.2 | Suspension | 60.5 | Suspension |
| 7 | EtOH | 20.1 | 365.1 | 55.1 | 52.2 | 69.8 | 14.3 | Suspension | 31.9 | Solution |
| 8 | EtOH | 25.2 | 380.9 | 66.2 | 62.1 | 83.9 | 11.9 | Suspension | 32 | Suspension |
| 9 | EtOH | 30.3 | 383.4 | 79 | 73.2 | 100.2 | 10 | Suspension | 36.8 | Suspension |
| 10 | EtOH | 35.2 | 381.9 | 92.2 | 84.4 | 116.8 | 8.6 | Suspension | 41 | Suspension |
| 11 | EtOH | 39.7 | 381.4 | 104.1 | 94.3 | 131.9 | 7.6 | Suspension | 43.4 | Suspension |
| 12 | EtOH | 45.1 | 384.3 | 117.4 | 105 | 148.7 | 6.7 | Suspension | 46.3 | Suspension |
| 13 | EtOH | 49.9 | 386.8 | 129 | 114.3 | 163.5 | 6.1 | Suspension | 48.9 | Suspension |
| 14 | EtOH | 55.1 | 385.7 | 142.9 | 125 | 181.1 | 5.5 | Suspension | 51.1 | Suspension |

### Solubility Model for GEN Form a in EtOAc-EtOH

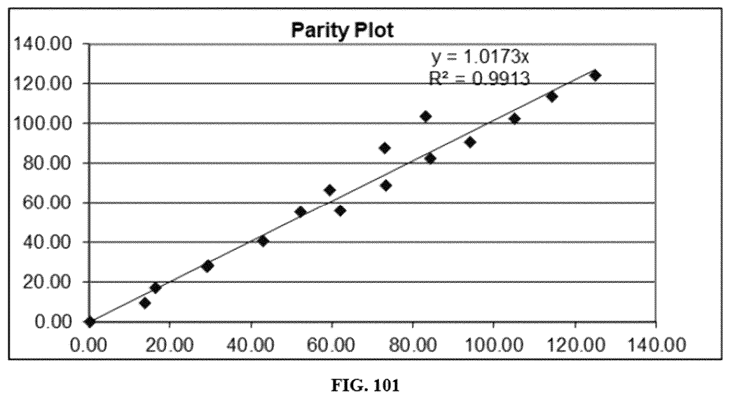

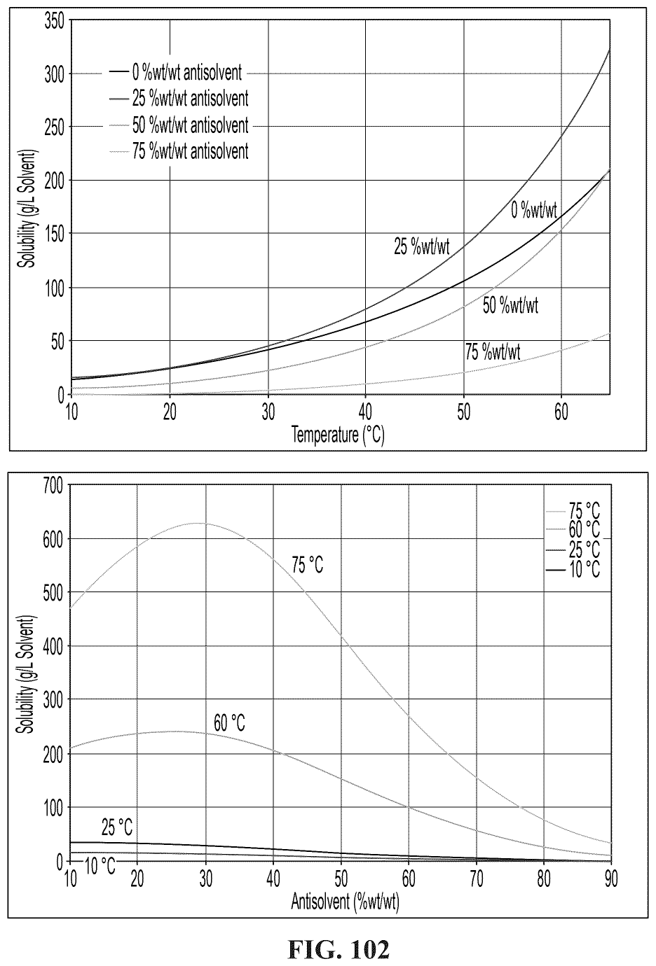

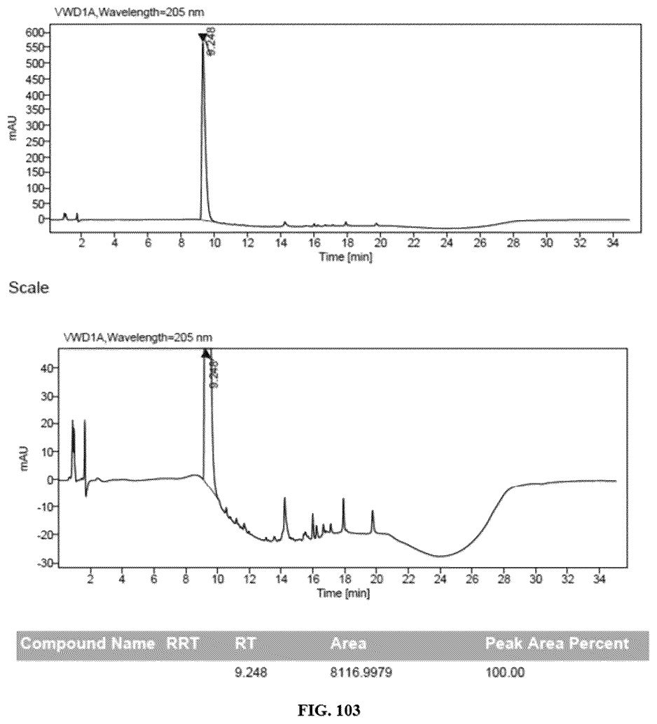

The combination of the solubility data for different experiments in EtOH, EtOAc and mixtures of both allowed the elaboration of solubility models in binary mixtures. Different models based on the van′t Hoff equation were used and the constants A, B, C and D regressed against the solubility data (see Table 30). These models require the data to be expressed as g/Kg solution and x1 is the wt % fraction of antisolvent (in this case EtOAc was defined as antisolvent). The data and predictions are given in Table 30 below. The best fit was obtained in model 1, see parity plot FIG. 101, and the solubility curves are shown in FIG. 102.

TABLE 30
Van't Hoff Solubility Models

| | | | | | | | corr | |
|---|---|---|---|---|---|---|---|---|
| A | B | C | D | Equation | WSSQ | SSQ | coeff | R2 |
| 13.07026 | 22.09897 | −44.9672 | 124.0457 | C = exp(lnA0 − B0/(R*T) = −C0*(X1) − | 0.391 | 4.582 | 0.9382 | 0.88 |
| | | | | D0*X1/(R*T)) | | | | |
| 17.59626 | 11.83111 | −10.3479 | 22.60885 | C = exp(lnA1 − B1/(R*T) = −C1*(X1) − | 0.019 | 0.288 | 0.9962 | 0.993 |
| | | | | D1*exp(X1)/(R*T)) | | | | |
| −22.2027 | −37.7474 | −37.9963 | 18.9455 | C = exp(A2*(X12) + B2*(X1)/(R*T) + C2/ | 0.115 | 1.326 | 0.9825 | 0.965 |
| | | | | (R*T) + D2*exp(x1)) | | | | |
| 33.7936 | 51.09297 | 9.77998 | −30.34575 | C = exp(lnA3 − B3/(R*T) = −C3*EXP(X1)− | 0.155 | 1.462 | 0.9807 | 0.962 |
| −7.64334 | 7.34841 | −47.1072 | 22.4669 | C = exp(A4*(X12) + B4*(X1)/(R*T) + C4/ | 0.128 | 1.292 | 0.983 | 0.966 |
| | | | | (R*T) + D4 | | | | |

TABLE 31
Solubility Data and Model Predictions in EtOAc—EtOH

| | | | Fraction | Conc (g/Kg | | | | | | |
|---|---|---|---|---|---|---|---|---|---|---|
| | Conc g/L | | antisolv | solution) | | Model Predictions, g/kg solution | | | | |
| Reference | solvent | Solvent | wt/wt % | Temp (° C.) | | model0 | model1 | model2 | model3 | model4 |
| 1 | 131.9 | EtOH | 0 | 43.4 | 94.28 | 107.06 | 90.94 | 90.75 | 99.47 | 96.32 |
| 2 | 148.7 | EtOH | 0 | 46.3 | 105.03 | 115.53 | 102.41 | 103.46 | 118.64 | 113.31 |
| 3 | 163.5 | EtOH | 0 | 48.9 | 114.27 | 123.56 | 113.71 | 116.12 | 138.58 | 130.76 |
| 4 | 181.1 | EtOH | 0 | 51.1 | 125 | 130.68 | 124.08 | 127.86 | 157.73 | 147.33 |
| 5 | 69.8 | EtOH | 0 | 31.9 | 52.18 | 78.01 | 55.52 | 52.66 | 47.85 | 49.05 |
| 6 | 83.9 | EtOH | 0 | 32 | 62.05 | 78.23 | 55.77 | 52.92 | 48.16 | 49.35 |
| 7 | 100.2 | EtOH | 0 | 36.8 | 73.24 | 89.53 | 68.82 | 66.73 | 65.79 | 65.79 |
| 8 | 116.8 | EtOH | 0 | 41 | 84.39 | 100.4 | 82.28 | 81.27 | 85.76 | 84.01 |
| 9 | 16 | EtOAc:EtOH | 77 | 39.9 | 13.82 | 12.25 | 9.56 | 7.3 | 7.52 | 7.46 |
| | | (74.6:25.4) | | | | | | | | |
| 10 | 19.1 | EtOAc:EtOH | 77 | 48.1 | 16.4 | 38.83 | 17.32 | 14.09 | 9.87 | 11.2 |
| | | (74.6:25.4) | | | | | | | | |
| 11 | 36 | EtOAc:EtOH | 53.1 | 33 | 29.48 | 10.89 | 28.13 | 23.49 | 30.54 | 28.15 |
| | | (49.4:50.6) | | | | | | | | |
| 12 | 35.7 | EtOAc:EtOH | 53.1 | 32.9 | 29.26 | 10.76 | 27.95 | 23.31 | 30.4 | 27.99 |
| | | (49.4:50.6) | | | | | | | | |
| 13 | 77.3 | EtOAc:EtOH | 27 | 34.8 | 59.35 | 33.07 | 66.78 | 79.68 | 68.47 | 72.64 |
| | | (24.4:75.6) | | | | | | | | |
| 14 | 96.7 | EtOAc:EtOH | 27 | 40.1 | 73.18 | 47.75 | 87.83 | 109.56 | 90.92 | 97.88 |
| | | (24.4:75.6) | | | | | | | | |
| 15 | 110.9 | EtOAc: EtOH | 27 | 43.4 | 83.02 | 59.65 | 103.68 | 132.87 | 107.95 | 117.25 |
| | | (24.4:75.6) | | | | | | | | |
| 16 | 0.15 | EtOAc | 100 | 25 | 0.17 | 0.4 | 0.2 | 0.28 | 0.31 | 0.3 |
| 17 | 33.98 | EtOH | 0 | 25 | 43.06 | 63.76 | 40.55 | 37.23 | 30.02 | 31.91 |

It is clear from the data and models that the EtOAc-EtOH system is not a simple solvent-antisolvent system and synergistic effect is seen with higher solubility at intermediate composition.

### Crystallization Experiments of GEN Form A

Following the solubility experiments, crystallization experiments with GEN Form A at 500 mg scale were performed in IPA and a mixture of EtOAc-EtOH. Results were described in Table 32.

TABLE 32
Crystallization Experiments

| | Scale | | Vol | T | Seed T | Seed | Final T | | Yield |
|---|---|---|---|---|---|---|---|---|---|
| Reference | (mg) | Solvent | (V) | (° C.) | (° C.) | age | (° C.) | XRPD | (%) |
| 1 | 500 | IPA | 13 | 75 | 67 | 30 | 25 | GEN | 77 |
| | | | | | | min | | Form A | |
| 2 | 500 | EtOAc:EtOH | 15 | 75 | 70 | 30 | 25 | GEN | 69 |
| | | (65:35) | | | | min | | Form A | |

### Summary for Example 5

Solubility curves in IPA and EtOH and a solubility model in EtOAc-EtOH were elaborated. Crystallization experiments were performed in IPA and EtOAc-EtOH affording 77% and 69% yields respectively.

### Summary of Salt/Cocrystal Studies

Salts were identified with different acids: gentisic (2,5-dihydroxybenzoic acid) andadipic. The salt from adipic acid was shown to be a hemi-adipate salt. All other salt forms were 1:1 API:acid. From the perspective of acceptable pharmaceutical dosing, the hemi-adipate salt may be advantageous.

GEN Form and ADI Form A stored under accelerated conditions at 40° C./75% RH were stable for at least 1 week.

The, gentisate and hemi-adipate salts were characterized by 1H NMR, XRPD, DSC, TGA and DVS. The gentisate and hemi-adipate salts were also characterized by microscopy. The hemi-adipate exhibited fine needles whereas the particles of the gentisate were larger and blade-like. A KF analysis was performed on the hemi-adipate (0.4%).

With the exception of the gentisate salt, other salt forms were hygroscopic and showed non-reversible behavior in the DVS, resulting in deliquescence. The hemi-adipate salt showed little water uptake less than 70% RH, after which approximately 80 wt % moisture was absorbed.

High solubility (>10 mg/mL) was observed for GEN Form A and ADI Form A in four different buffers (pH 1.2, 4.5, 6.5 and 7.4).

The gentisate salt appears to have better physical stability than the hemi-adipate, while the hemi-adipate may be advantageous from the perspective of acceptable pharmaceutical dosing. Care should be taken to store and process the hemi-adipate under controlled humidity to prevent deliquescence.

For the gentisate salt, process development was performed with solubility curves in IPA and EtOH and a solubility model in EtOAc-EtOH being elaborated to guide scale-up conditions. Three batches at 80-100 g scale were performed in 85-90% yield from EtOAc-EtOH.

In a similar way, solubility modelling of the hemi-adipate salt in 2-MeTHF and EtOAc provided conditions for scale-up. Two batches on 150 g scale in 2-MeTHF-heptane gave quantitative yield.

### Example 25: ADI Form A Scale Up and Additional Characterization

The hemi-adipate salt shows better stability under humidity conditions (40° C./75% RH) than the sulfate salt and crystalline and chemical degradation was not observed at these conditions. However, by DVS, irreversible moisture uptake was observed at high relative humidity resulting in deliquescence of the sample. Solubility modelling was performed to aid initial process development, and two batches were performed at approximately 140-150 g scale from 2-methyltetrahydrofuran-heptane in quantitative yield.

Preparation at 250 mg-1 g Scale

ADI Form A was prepared at 250 mg-1 g scale for a further characterization using the compound of Formula (I) Form 1. Heat-cool cycles between 25° C. to 50° C. (30 min at each temperature, with heating and cooling at 1° C./min) for 1 day was performed. The procedures were not optimized. The experiments are described in Table 33.

TABLE 33
Preparation of Hemi-Adipate at 250 mg-1 g Scale

| Reference | Acid Eq. | Scale (mg) | Solvent | Vol (V) | XRPD wet | XRPD dry | Yield (%) |
|---|---|---|---|---|---|---|---|
| 1 | 1 | 250 | EtOAc | 5 | ADI Form A* | ADI Form A | 49 |
| 2 | 1 | 1000 | EtOAc | 10 + 2 | ADI Form A* | ADI Form A | 79.6 |
| 3 | 1 | 1000 | THF | 10 + 15 | ADI Form A | ADI Form A | 82.2 |
| 4 | 0.7 | 1000 | THF | 10 + 5 | ADI Form A | ADI Form A | 87.8 |

In TIFF the procedure gave a free flowing suspension that was easily filtered. However, when EtOAc was used, the solid was sticky after the filtration.

### Additional Characterization of ADI Form A

ADI Form A obtained from EtOAc and THF were characterized by XRPD, ¹H NMR, DSC, TGA and DVS. The results are summarized in Table 34.

TABLE 34
Characterization Summary of ADI Form A

| Solvent | EtOAc | THF |
|---|---|---|
| Aspect | Sticky solid | Free flowing solid |
| XRPD | ADI Form A FIG. 86 | ADI Form A FIG. 83 |
| ¹H NMR (D₂O) | API:Adipic acid (2:1) FIG. 87 | API:Adipic acid (2:1) FIG. 92 |
| DSC | Melt onset at 104° C. FIG. 88 | Melt onset at 104° C. FIG. 93 |
| TGA | 0.5% loss at 30° C. to 65° C. | Degradation at~150° C. FIG. 93 |
| | Degradation at~150° C. FIG. 88 | |
| Stability at | Stable for 1 week | Stable for 1 week |
| 40° C. / 75% RH | | |
| Stability at | Deliquescent after 2 days | Deliquescent after 2 days |
| 25° C. / 96% RH | | |
| DVS | 87.3% weight change | 84% weight change |
| | FIG. 89 and FIG. 90 | FIG. 94 and FIG. 95 |
| KF | Not analyzed | 0.41% |

### Example 26: Further Investigation of ADI Form A

### Polymorphism Assessment

ADI Form A was further studied by means of polymorphism assessment. 13 different solvent systems were studied to provide an early insight into the propensity for polymorphism of ADI Form A.

Procedure. The sample (20 mg±1 mg) was weighed and suspended in 5-10 volumes of the corresponding solvent. Then, mixtures were subjected to heat-cool cycles between 25° C. to 50° C. (30 min at each temperature, with heating and cooling at 1° C./min) for 1 day. Then, mixtures were centrifuged, decanted and the solids were first analyzed by XRPD as wet cakes before drying under vacuum at ambient temperature and reanalyzed by XRPD if a new form was obtained.

TABLE 35
Polymorphism Assessment of ADI Form
A (from preparation in EtOAc)

| Reference | Solvent | Aspect 1 day | XRPD wet | XRPD dry |
|---|---|---|---|---|
| 1 | Water | Solution | NA | NA |
| 2 | MeCN | Suspension/Wax | ADI Form A | ADI Form A |
| 3 | EtOH | Solution | NA | NA |
| 4 | IPA | Solution | NA | NA |
| 5 | Acetone | Solution | NA | NA |
| 6 | MEK | Solution | NA | NA |
| 7 | EtOAc | Suspension/Wax | ADI Form A | ADI Form A |
| 8 | i-PrOAc | Suspension/Wax | ADI Form A | ADI Form A |
| 9 | MTBE | Suspension/Wax | ADI Form A | ADI Form A |
| 10 | THF | Suspension | ADI Form A | ADI Form A |
| 11 | Toluene | Suspension/Wax | ADI Form A | ADI Form A |
| 12 | Heptane | Suspension/Wax | ADI Form A | ADI Form A |

TABLE 36
Polymorphism Assessment of ADI Form
A (from preparation in THF)

| Reference | Solvent | Aspect 1 day | XRPD wet | XRPD dry |
|---|---|---|---|---|
| 1 | 2-MeTHF | Cotton like | ADI Form A | ADI Form A |
| 2 | MeCN | Free flowing | ADI Form A | ADI Form A |
| | | suspension | | |
| 3 | EtOH | Solution | NA | NA |
| 4 | IPA | Solution | NA | NA |
| 5 | Acetone | Solution | NA | NA |
| 6 | MEK | Solution | NA | NA |
| 7 | EtOAc | Free flowing | ADI Form A | ADI Form A |
| | | suspension | | |
| 8 | i-PrOAc | Free flowing | ADI Form A | ADI Form A |
| | | suspension | | |
| 9 | MTBE | Free flowing | ADI Form A | ADI Form A |
| | | suspension | | |
| 10 | THF | Cotton like | ADI Form A | ADI Form A |
| 11 | Toluene | Free flowing | ADI Form A | ADI Form A |
| | | suspension | | |
| 12 | Heptane | Free flowing | ADI Form A | ADI Form A |
| | | suspension | | |

Only one polymorph of the hemi-adipate salt was seen. XRPD (FIG. 91), 1 NMR (FIG. 92) DSC, TGA (FIG. 93) and DVS analyses (FIG. 94 and FIG. 95) were performed for ADI Form A. By 1H NMR the ratio of API:adipic acid is 1:0.5. This form showed good crystallinity by XRPD, is essentially non-solvated with a melt at onset 104° C. By DVS approximately 80 wt % uptake was seen, mainly above 8000 RH. The adsorption-desorption process was non-reversible with deliquescence occurring during the process. ADI Form A was stable for at least 1 week at 25° C./75% RH, but deliquesced at 25° C./96% RH after 2 days. The salt showed high solubility (>10 mg/mL) over the pH range 1.7-7.2.

### Stability Study of ADI Form A

ADI Form A was stored in open vials at 25° C./60 RH and 40° C./7500 RH, monitored with dataloggers, for 2 weeks and were analyzed by XRPD, TGA and HPLC every week to study their crystalline and chemical stability. A summary of the results is shown in Table 27. By HPLC no impurities were seen.

TABLE 37
Stability Studies of ADI Form A in Different Conditions by XRPD and TGA

| | | | | | TGA (% |
|---|---|---|---|---|---|
| Reference | Conditions | Time Point | Aspect | XRPD | weight loss) |
| 1 | — | Time 0 | Off-white solid | ADI Form A | 0 |
| 2 | 40° C. / 75% RH | 1 week | Off-white solid | ADI Form A | 1.9 |
| 3 | 40° C. / 75% RH | 2 weeks | Off-white solid | ADI Form A | 0.4 |
| 4 | 25° C. / 60% RH | 1 week | Off-white solid | ADI Form A | 0 |
| 5 | 25° C. /60% RH | 2 weeks | Off-white solid | ADI Form A | 0 |

### Thermodynamic Solubility of ADI Form A at Different pHs

The thermodynamic solubility study at pH 1.2, 4.5, 6.5 and 7.4 was performed with ADI Form A.

Procedure. The salt, 20 mg of each, was treated with the different buffer solutions (2 mL) and stirred for 24 hours at 25° C. The pH was measured after 24 hours. In case suspensions were obtained, 1 mL of each suspension was filtered with a nylon syringe filter (0.45 μm) and were diluted by a factor of 6 v/v in MeCN/water (1.1) for HPLC analysis.

TABLE 38
Thermodynamic Solubility at pH 1.2, 4.5, 6.5 and 7.4

| | | XRPD | | | | XRPD |
|---|---|---|---|---|---|---|
| Condi- | | Initial | Observa- | pH | Conc. | Recovered |
| tions | Reference | Solid | tions | 24 h | (mg/mL) | Solid |
| pH 1.2 | 1 | ADI | Solution | 1.8 | >10 | NA |
| | | Form A | | | | |
| pH 4.5 | 2 | ADI | Solution | 5.5 | >10 | NA |
| | | Form A | | | | |
| pH 6.5 | 3 | ADI | Solution | 6.7 | >10 | NA |
| | | Form A | | | | |
| pH 7.4 | 4 | ADI | Solution | 7.2 | >10 | NA |
| | | Form A | | | | |

High solubility (>10 mg/mL) was observed in all aqueous buffers, from pH 1.2 to 7.4.

### Summary of Example 6

During the polymorphism assessment in 13 different solvents no new crystalline forms were detected. High solubility (>10 mg/mL) was observed for ADI Form A in four different buffers (pH 1.2, 4.5, 6.5 and 7.4).

### Example 27: Solubility Data of ADI Form A in Organic Solvents and Mixtures

### Evaluation

An initial evaluation of the solubility at 25° C. was conducted in different organic solvents. Solvents were chosen on the basis of ICH class, looking for both high (solvent) and low (antisolvent) solubilizing potential. The solid (ADI Form A, 50 mg) was weighed in HPLC vials and 500 μL of each solvent were added. Samples were slurried at 25° C. for 24 hours, then approximately 300 μL of suspension were taken, diluted in 2 mL of MeCN/H₂O (1:1), filtered through a 0.45 μm hydrophilic syringe filter and analyzed by HPLC. Experiments where solutions were obtained were not analyzed by HPLC.

TABLE 39
Dissolution of ADI Form A in Different Organic Solvents at 25° C.

| | | | | Conc | Conc |
|---|---|---|---|---|---|
| | | ICH | | (mg/g | (mg/mL |
| Reference | Solvent | Class | Aspect at RT | solution) | solution) |
| 1 | 2-MeTHF | 3 | Suspension | 19 | 16.2 |
| 2 | MeCN | 2 | Suspension | 13.7 | 10.8 |
| 3 | EtOH | 3 | Solution | >126.6 | >100 |
| 4 | IPA | 3 | Solution | >126.6 | >100 |
| 5 | Acetone | 3 | Solution | >126.6 | >100 |
| 6 | MEK | 3 | Solution | >126.6 | >100 |
| 7 | EtOAc | 3 | Suspension | 6 | 5.3 |
| 8 | i-PrOAc | 3 | Suspension | 57.3 | 49.9 |
| 9 | MTBE | 3 | Suspension | 2.3 | 1.7 |
| 10 | THF | 2 | Suspension | 34 | 30.2 |
| 11 | Toluene | 2 | Suspension | NA* | NA* |
| 12 | Heptane | 3 | Suspension | NA* | NA* |

*2 phases observed when diluted in MeCN/H₂O (1:1)

Solubility in 2-MeTHF, iPrOAc, and EtOAc by Determination of Clear Points

Following from the previous solubility results, 2-MeTHF, iPrOAc, and EtOAc were studied in more detail. The solid (ADI Form A) was weighed in HPLC vials, 0.5 mL of solvent added, and the solvent weighed for accuracy. A heating ramp from 25-80° C. at 0.5° C./min was applied and the clear point (dissolution temperature) recorded using reflectance probes. All the experiments resulted in dissolution at high temperature and the mixtures were then cooled to 25° C. at 0.5° C./min. In all cases, spontaneous precipitation occurred during cooling. Reanalysis of the solids by XRPD showed that ADI Form A was recovered. The results are given in Table 40.

TABLE 40
Dissolution of ADI Form A in 2-MeTHF, iPrOAc, and EtOAc

| | | | | Conc | Conc | Conc | Conc | Clear | Cloud |
|---|---|---|---|---|---|---|---|---|---|
| | | API | Solvent | (mg/g | (mg/g | (mg/mL | (mL/mg | point | point |
| Reference | Solvent | (mg) | (mg) | solvent) | solution) | solvent) | solvent) | (° C.) | (° C.) |
| 1 | 2-MeTHF | 14.9 | 400.7 | 37.2 | 35.9 | 43.5 | 23 | 43.2 | 25.2 |
| 2 | 2-MeTHF | 19.4 | 421 | 46.1 | 44.1 | 54 | 18.5 | 46.1 | 39 |
| 3 | 2-MeTHF | 25.8 | 425.8 | 60.6 | 57.1 | 71 | 14.1 | 50.8 | 37.5 |
| 4 | 2-MeTHF | 35.7 | 414.3 | 86.2 | 79.3 | 100.9 | 9.9 | 53.9 | 50.4 |
| 5 | 2-MeTHF | 41.8 | 418.4 | 99.9 | 90.8 | 117 | 8.5 | 57.8 | 45.7 |
| 6 | 2-MeTHF | 44.9 | 417.9 | 107.4 | 97 | 125.8 | 7.9 | 60 | 55.2 |
| 7 | 2-MeTHF | 49.8 | 425.1 | 117.1 | 104.9 | 137.2 | 7.3 | 61.3 | 54.3 |
| 8 | 2-MeTHF | 54.3 | 425.5 | 127.6 | 113.2 | 149.4 | 6.7 | 62.7 | 44.3 |
| 9 | 2-MeTHF | 59.2 | 425.3 | 139.2 | 122.2 | 163 | 6.1 | 62.8 | 55.5 |
| 10 | iPrOAc | 30.6 | 415.8 | 73.6 | 68.5 | 84.6 | 11.8 | 64.2 | 62.8 |
| 11 | iPrOAc | 34.5 | 426.5 | 80.9 | 74.8 | 93 | 10.8 | 64.6 | 65.9 |
| 12 | iPrOAc | 40.7 | 425.8 | 95.6 | 87.2 | 109.9 | 9.1 | 64.8 | 67.5 |
| 13 | iPrOAc | 57.8 | 426.2 | 135.6 | 119.4 | 155.9 | 6.4 | 68.7 | 70.2 |
| 14 | EtOAc | 5.5 | 440.9 | 12.5 | 12.3 | 13.8 | 72.3 | 38.9 | 26.3 |
| 15 | EtOAc | 10 | 427.4 | 23.4 | 22.9 | 25.9 | 38.6 | 49.6 | 38.4 |
| 16 | EtOAc | 15.2 | 443.3 | 34.3 | 33.2 | 38 | 26.3 | 53.9 | 30.6 |
| 17 | EtOAc | 19.7 | 444.2 | 44.3 | 42.5 | 49.2 | 20.3 | 57.6 | 47.5 |
| 18 | EtOAc | 24.9 | 446.3 | 55.8 | 52.8 | 61.9 | 16.2 | 58.5 | 52.4 |
| 19 | EtOAc | 30 | 444.1 | 67.6 | 63.3 | 74.9 | 13.4 | 62 | 50 |
| 20 | EtOAc | 35.9 | 450.6 | 79.7 | 73.8 | 88.3 | 11.3 | 61.3 | 56.2 |

In 2-MeTHF, a good difference in solubility at high temperature (59° C., 163 mg/mL) vs low (25° C., 16 mg/mL) was observed while in iPrOAc the salt has a similar solubility to 2-MeTHF at high temperature (58° C., 156 mg/mL), but higher solubility at low temperature (25° C., 50 mg/mL). A wider temperature range is potentially offered by iPrOAc. However, the higher solubility at low temperatures favors 2-MeTHF for increased yield, potentially without the need for antisolvent.

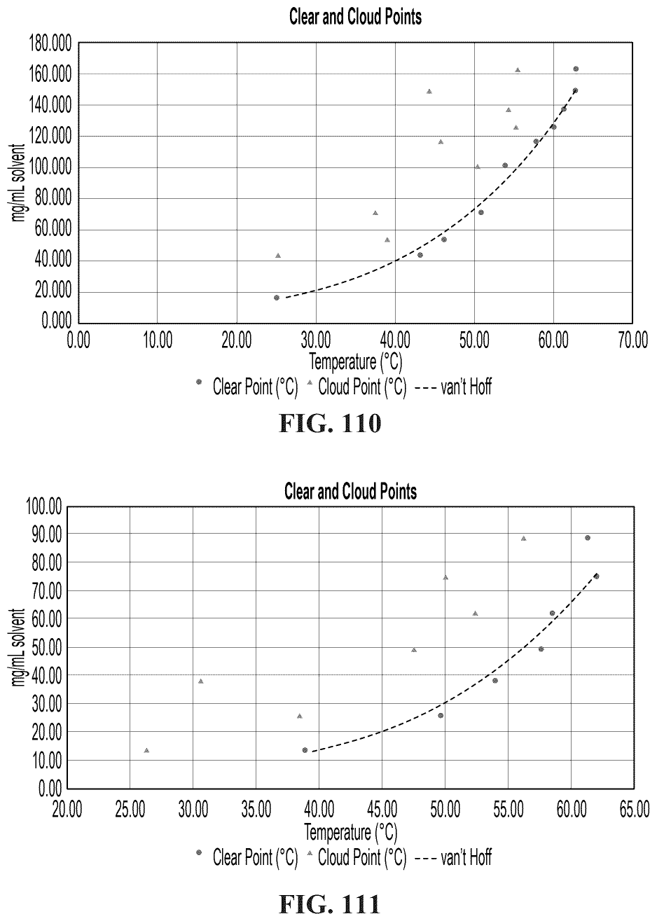

The solubility data for 2-MeTHF is represented in FIG. 110. The clear points, together with HPLC data at 25° C., provide the solubility curve. Together with the cloud points we get an idea of the meta-stable zone. Significant variability is seen in the cloud points, which is not uncommon. The MSZ width under these conditions (2-MeTHF, cooling at 5° C./min) is about 5-10′° C.

The solubility data for EtOAc is represented above in FIG. 111. The clear points, together with HPLC data at 25° C., provide the solubility curve. Together with the cloud points we get an idea of the meta-stable zone. Significant variability is seen in the cloud points, which is not uncommon. The MSZ width under these conditions (EtOAc, cooling at 5° C./min) is about 10-15° C.

### Example 28: ADI Form A Scale Up

After an initial small-scale trial at 2 g scale, the process (starting from the sulfate salt) was scaled up first to 20 g and then to 200 g batches in a 2-L jacketed vessel (see Table 41). Approximately 280 g of hemi-adipate salt will be prepared from the sulfate in 2 batches, first cracking the sulfate with NaOH and partitioning in DCM-water, concentrating the organic and crystallizing the hemi-adipate in 2-MeTHF (6 vol) with seeding at 70° C., followed by cooling to 20° C. and antisolvent addition (heptane, 2.5 vol). The experimental procedures of the two big scale-ups are described below.

TABLE 41
Scale-up Batches, Hemi-Adipate Salt

| | Yield | | 1H NMR | | | |
|---|---|---|---|---|---|---|
| Reference | (g, %) | XRPD | (DMSO) | DSC | TGA | Microscopy |
| 1 | 13.38 g, | ADI Form | NP | Melt with onset | No weight | NP |
| | 92.7% | A | | at 104° C. | loss | |
| 2 | 146.1 g, | ADI Form | 2:1 ratio | Melt with onset | No weight | Small needle |
| | 101% | A | | at 102.3° C. | loss | shape particles |
| 3 | 143.7 g, | ADI Form | 2:1 ratio | Melt with onset | No weight | Small needle |
| | 99.7% | A | | at 102.3° C. | loss | shape particles |

### Preparation of ADI Form A, 200 g Scale

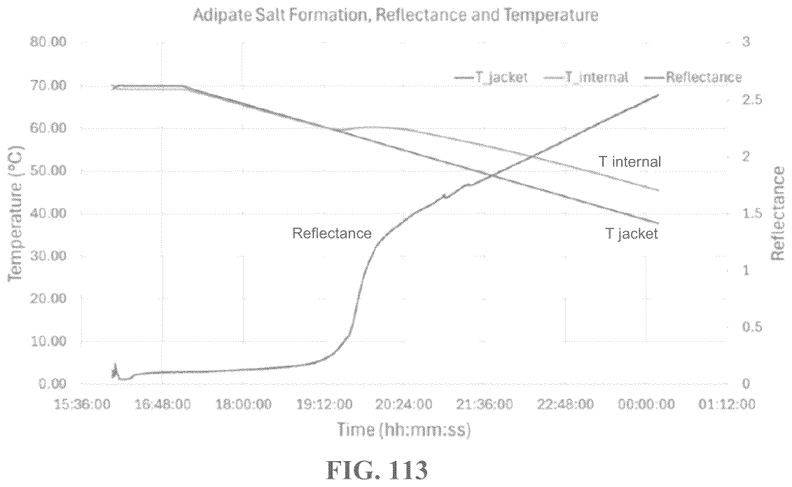

The BHS salt of the compound of Formula (I) (200.3 g), sodium hydroxide (78.8 g), water (600 mL) and dichloromethane (200 mL) were introduced into a 2 L jacketed vessel and stirred for 20 minutes. The mixture was allowed to settle, the layers were separated and the aqueous phase extracted with dichloromethane (4×100 mL). The organic phases were combined and evaporated to dryness on a rotary evaporator. To the residue 2-MeTHF (800 mL, 6 V) was added, charged to a 2 L jacketed vessel, heated to 50° C. and adipic acid (33.75 g, 0.5 eq.) were added together with an additional 64 mL of 2-MeTHF washing the funnel. A suspension was immediately observed. The mixture was heated to 75° C. and a solution was observed. The solution was cooled to 70° C. and seeded with ADI Form A, 865 mg. The mixture was stirred for 1 hour at this temperature, then cooled to 20° C. in 10 hours and maintained at 20° C. for 13 hours. Heptane (432 mL, 2.5 V) was added and the mixture stirred for 30 minutes. The mixture was filtered, washed with heptane (432 mL) and dried under vacuum at 50° C. for 24 h and 30° C. for 3 days. An off-white solid 146.1 g (101%) was obtained. The temperature and reflectance profile (FIG. 113) was obtained.

### Summary of ADI Form A Studies

Two batches of ADI Form A were obtained at 1 g scale from EtOAc and THF. The XRPDs are almost identical.

No new polymorphs were observed for ADI Form A in the different solvents tested while it shows good stability by XRPD and HPLC in the conditions tested (40° C./75% RH and 25° C./60% RH) and very high solubility in different pH buffers (>10 mg/mL).

2-MeTHF may be a useful solvent for the hemi-adipate preparation at a higher scale for several reasons: (1) ICH class 3; (2) good solubility profile; and (3) immiscible with water to potentially use for extractions of the free base precursor.

The crystalline salt with adipic acid (ADI Form A) was stable for at least 1 week at 40° C./75% RH while at 25° C./96% RH deliquescence was observed after 2 days. Experiments at 1 g scale were performed successfully to obtain ADI Form A in THF and EtOAc. However, in EtOAc a sticky solid was obtained while in THF a fine powder was observed. During the polymorphism assessment in 13 different solvents no new crystalline forms were detected. High solubility (>10 mg/mL) was observed for ADI Form A in four different buffers (pH 1.2, 4.5, 6.5 and 7.4). No crystalline or chemical degradation was detected by XRPD or HPLC at different conditions. Solubility curves in 2-MeTHF and EtOAc were elaborated. Two batches at 200 g scale were prepared by crystallization in 2-MeTHF-heptane.

### OTHER EMBODIMENTS

While the disclosure has been particularly shown and described with reference to a preferred embodiment and various alternate embodiments, it will be understood by persons skilled in the relevant art that various changes in form and details can be made therein without departing from the spirit and scope of the disclosure. While preferred embodiments of the present disclosure have been shown and described herein, it will be apparent to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

All references, issued patents and patent applications cited within the body of the instant specification are hereby incorporated by reference in their entirety, for all purposes.

1. A method of treating Alzheimer's Disease in a subject identified as having a level of tau protein phosphorylated at a threonine at amino acid 217 (p-tau217) elevated relative to a control, the method comprising administering orally to the subject a pharmaceutical composition comprising from about 200 mg to about 400 mg of a compound represented by Formula (Ia): 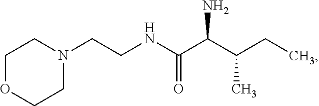 [SMILES: CC[C@H](C)[C@H](N)C(=O)NCCN1CCOCC1]
or a pharmaceutically acceptable salt thereof to the subject twice daily.

2-8. (canceled)

9. The method of claim 1, wherein the subject further has one or more of a level of tau protein phosphorylated at a threonine at amino acid 181 (p-tau181), or a level of tau protein phosphorylated at a threonine at amino acid 231 (p-tau231), that is each indicative of the presence of Alzheimer's Disease pathology in the brain.

10. The method of claim 1, wherein the method delays progression of Alzheimer's Disease in the subject.

11. The method of claim 10, wherein the delaying progression comprises reduced levels of one or more of a glycolysis proteomic module, a postsynaptic proteomic module, and an axonogenesis proteomic module compared to one or more of a subject not receiving the compound represented by Formula (Ia) or a pharmaceutically acceptable salt thereof.

12. The method of claim 10, wherein the delaying progression comprises increased levels of one or both of a blood brain barrier (BBB) proteomic module and a collagen proteomic module compared to one or more of a subject not receiving the compound represented by Formula (Ia) or a pharmaceutically acceptable salt thereof.

13. The method of claim 12, wherein the levels of one or more of the the BBB proteomic module and the collagen proteomic module are measured from the cerebrospinal fluid of the subject.

14. The method of claim 11, wherein the glycolysis proteomic module comprises measurements of one or more protein markers selected from the group consisting of: MAPT, NRGN, GAP43, PKM, GOT1, ALDOA, MDH1, UBB, CALM1, BASP1, HSPA8, SOD1, DDAH1, PITHD1, LDHB, NPEPPS, PEBP1, DYNLL2, RAD23B, CPLX2, GDA, STMN1, GSS, PARK7, GMFB, SYN1, HPRT1, DTD1, SYT1, UBE2V1, AP2B1, SKP1, PKIA, PPP5C, TXN, NSF, SOD2, TALDO1, DLD, SMOC1, TMSB4X, PDXP, NUTF2, ALDOC, DDB1, CEND1, TOLLIP, SMOC2, PRKAR1A, CRYM, NIF3L1, GABARAPL2, PFN2, GGCT, CAMK2A, TPI1, GOT2, NAXE, PREP, NUDC, RTN1, DNM1, CYCS, SPON1, HSPE1, SPP1, ENO2, HDHD2, LAMP5, DLST, DNAJB2, SCN2B, RIDA, PSAT1, EPB41L1, BLMH, RPH3A, MARCKS, CYR61, HK1, CAMK2D, ACYP2, VSNL1, CKMT1A, TXNDC17, MDH2, S100A1, STX1B, CAMK2B, ATP6AP2, LHPP, STX8, EIF4B, TPD52, SNAP91, VAPA, PRKAR1B, TNFRSF12A, SERPINE2, SELENOW, VASN, MT2A, AP1B1, DBI, HSPA2, C1orf232, NAV1, SLC1A2, QDPR, B2M, PGM1 and CAMK2G.

15. The method of claim 11, wherein the postsynaptic proteomic module comprises measurements of one or more protein markers selected from the group consisting of: C1QL3, C4orf48, DPP10, ENDOD1, and SUSD5.

16. The method of claim 11, wherein the axonogenesis proteomic module comprises measurements of one or more protein markers selected from the group consisting of: GALNT18, PVR, SEMA4D, CNTNAP2, and RTN4RL2.

17. The method of claim 12, wherein the BBB proteomic module comprises measurements of one or more protein markers selected from the group consisting of: OGN, SFRP4, PDGFRL, METRNL, and MYOC.

18. The method of claim 12, wherein the collagen proteomic module comprises measurements of one or more protein markers selected from the group consisting of: MATN2, PTK7, FMOD, SEMA3D, and B3GNT9.

19. The method of claim 10, wherein the delaying progression comprises one or more of ameliorating or delaying symptoms of Alzheimer's Disease compared to one or more of a subject not receiving the compound represented by Formula (Ia) or a pharmaceutically acceptable salt thereof.

20-21. (canceled)

22. The method of claim 1, wherein the subject exhibits one or both of improved cognition or improved function compared to a subject not receiving the compound represented by Formula (Ia) or a pharmaceutically acceptable salt thereof.

23. The method of claim 22, wherein improved cognition comprises improved visuospatial ability.

24-25. (canceled)

26. The method of claim 1, wherein the subject does not have amyloid pathology.

27. (canceled)

28. The method of claim 1, wherein the level of p-tau217 is measured relative to total tau protein or as an absolute value.

29. The method of claim 1, wherein the p-tau217 is measured by a method comprising Lumipulse, Elecsys, Simoa, ELISA, immunoblot, immunoprecipitation, electrochemiluminescence magnetic pull-down, or liquid chromatography-mass spectrometry.

30. The method of claim 1, wherein the subject is identified to have a measurement of at least 4.2% p-tau217 relative to total tau as measured by mass spectrometry.

31. The method of claim 1, wherein the subject is identified to have a measurement of at least 0.180 pg/mL as measured by Lumipulse.

32. The method of claim 1, wherein the subject is identified to have a measurement of at least 0.27 pg/mL as measured by an electrochemiluminescence immunoassay.

33. The method of claim 1, wherein the subject is identified to have a measurement of at least 0.273 pg/mL as measured by an electrochemiluminescence immunoassay.

34. The method of claim 1, wherein the subject is identified to have a measurement of at least 0.399 pg/mL as measured by an electrochemiluminescence immunoassay.

35. The method of claim 1, wherein the subject is identified to have a measurement of at least 7.3 pg/mL as measured by an electrochemiluminescence immunoassay.

36. (canceled)

37. The method of claim 1 wherein the compound represented by Formula (Ia) or a pharmaceutically acceptable salt thereof is administered in an amount of about 400 mg.

38-54. (canceled)

55. The method of claim 1, wherein a pharmaceutically acceptable salt of the compound represented by Formula (Ia) is administered to the subject.

56. The method of claim 55, wherein the pharmaceutically acceptable salt is an adipic salt.

57. The method of claim 55, wherein the pharmaceutically acceptable salt is a gentisic salt.
